(19) United States
(12) Patent Application Publication
(10) Pub. No.: US 2025/0099061 A1
(43) Pub. Date: Mar. 27, 2025

(54) VISUALIZATIONS FOR DENTAL DIAGNOSTICS

(71) Applicant: Align Technology, Inc., San Jose, CA (US)

(72) Inventors: Sreelakshmi Kolli, Freemont, CA (US); Ema Patki, Los Altos, CA (US); Christopher E. Cramer, Durham, NC (US); Joachim Krois, Berlin (DE); Martin Dreher, Berlin (DE); Falk Schwendicke, Berlin (DE)

(21) Appl. No.: 18/895,013

(22) Filed: Sep. 24, 2024

Related U.S. Application Data
(60) Provisional application No. 63/540,363, filed on Sep. 25, 2023.
(60) Provisional application No. 63/575,549, filed on Apr. 5, 2024.

(51) Int. Cl. A61B 6/51; A61B 6/00; A61B 6/46; G06T 7/00; G06T 7/11; G16H 15/00

(52) U.S. Cl. CPC A61B 6/51; A61B 6/463; A61B 6/5217; A61B 6/5229; G06T 7/0012; G06T 7/11; G16H 15/00; G06T 2207/10116; G06T 2207/20081; G06T 2207/20092; G06T 2207/20224; G06T 2207/30036

(57) ABSTRACT

A method includes receiving image data of a current state of a dental site of a patient and processing the image data using a segmentation pipeline to generate an output comprising segmentation information for one or more teeth in the image data and at least one of identifications or locations of one or more oral conditions observed in the image data, wherein each of the one or more oral conditions is associated with a tooth of the one or more teeth. The method includes generating a visual overlay comprising visualizations for each of the one or more oral conditions, outputting the image data to a display, and outputting the visual overlay to the display over the image data.

### RELATED APPLICATIONS

This patent application claims the benefit under 35 U.S.C. § 119 (e) of U.S. Provisional Application No. 63/540,363, filed Sep. 25, 2023, and further claims the benefit under 35 U.S.C. § 119 (e) of U.S. Provisional Application No. 63/575,549, filed Apr. 5, 2024, both of which are incorporated by reference herein.

### TECHNICAL FIELD

Embodiments of the present disclosure relate to the field of dental diagnostics and, in particular, to a system and method for improving the process of identifying and/or diagnosing oral conditions and/or oral health problems.

### BACKGROUND

For a typical dental practice, a patient visits the dentist twice a year for a cleaning and an examination. A dental office may or may not generate a set of x-ray images of the patient's teeth during the patient visit. The dental hygienist additionally cleans the patient's teeth and notes any possible problem areas, which they convey to the dentist. The dentist then reviews the patient history, reviews the new x-rays (if any such x-rays were generated), and spends a few minutes examining the patient's teeth in a patient examination process. During the patient examination process, the dentist may follow a checklist of different areas to review. The examination can start with examining the patient's teeth for cavities, then reviewing existing restorations, then checking the patient's gums, then checking the patients' head, neck and mouth for pathologies or tumors, then checking the jaw joint, then checking the bite relationship and/or other orthodontic problems, and then checking any x-rays of the patient. Based on this review, the dentist makes a determination as to whether there are any oral conditions that need to be dealt with immediately and whether there are any other oral conditions that are not urgent but that should be dealt with eventually and/or that should be monitored. The dentist then needs to explain the identified oral conditions to the patient, talk to the patient about potential treatments, and convince the patient to make a decision on treatment for the patient's health. It can be challenging for the dentist to identify all problem oral conditions, dental health problems, etc., and to convey the information about the oral conditions, dental health problems, and their treatments to the patient in the short amount of time that the dentist has allotted for that patient. The challenge is exacerbated by the fact that information about the patient's teeth can be fragmented and siloed, requiring the dentist to open and review multiple different applications and data sources to gain a full understanding of the patient's dental health and gum health. This can lead to misdiagnosis of oral conditions and also increase the amount of time that the dentist must spend with each patient.

### SUMMARY

A few example implementations of the present disclosure are described.

In a 1^{st} implementation, a method comprises: receiving data of a current state of a dental site of a patient, the data comprising a plurality of data items generated from a plurality of oral state capture modalities; processing the data using a plurality of models (e.g., trained machine learning models), wherein each model of the plurality of models is configured to process one or more data items generated from one or more oral state capture modalities of the plurality of oral state capture modalities, wherein the plurality of models output estimations of one or more oral conditions; processing at least one of the data or the estimations of the one or more oral conditions to generate at least one of a) one or more actionable symptom recommendations for one or more oral health problems associated with the one or more oral conditions or b) one or more diagnoses of the one or more oral health problems; and generating one or more treatment recommendations for treatment of at least one oral health problem of the one or more oral health problems based on at least one of the one or more actionable symptom recommendations or the one or more diagnoses.

A 2^{nd} implementation may extend the 1^{st} implementation. In the 2^{nd} implementation, the plurality of oral state capture modalities comprise a plurality of image modalities.

A 3^{rd} implementation may extend the 2^{nd} implementation. In the 3^{rd} implementation, the data generated using the plurality of image modalities comprises at least one of a cone beam computed tomography (CBCT) scan, a radiograph, a computed tomography (CT) scan, an optical intraoral scan, a three-dimensional (3D) model, a color image, a near-infrared (NIR) image, or an image generated using fluorescence imaging.

A 4^{th} implementation may extend the 2^{nd} or 3^{rd} implementation. In the 4^{th} implementation, the plurality of oral state capture modalities further comprises at least one of data from an electronic compliance indicator, patient input data, data from a consumer health monitoring tool, or data from a sensor of a dental appliance worn by the patient; and the data comprises at least one of blood pressure, body temperature, heart rate, saliva pH, saliva bacterial data, or patient pain data.

A 5^{th} implementation may extend the 4^{th} implementation. In the 5^{th} implementation, the dental appliance comprises a palatal expander, an orthodontic aligner, a retainer, or a sleep apnea device.

A 6^{th} implementation may extend any of the 1^{st} through 5^{th} implementations. In the 6^{th} implementation, the method further comprises: predicting a future state of the dental site based on processing at least one of the data, the estimations of the one or more oral conditions, the one or more actionable symptom recommendations, or the one or more diagnoses of the one or more oral health problems, wherein the predicted future state of the dental site comprises a future state of at least one of the one or more oral conditions or the one or more oral health problems.

A 7^{th} implementation may extend the 6^{th} implementation. In the 7^{th} implementation, the method further comprises: generating a first simulation of at least one of an image of the predicted future state of the dental site, a 3D model of the predicted future state of the dental site, or a video showing a progression over time to the predicted future state of the dental site.

An 8^{th} implementation may extend the 7^{th} implementation. In the 8^{th} implementation, the method further comprises: estimating a second future state of the dental site expected to occur after a treatment of at least one of the one or more oral conditions or the one or more oral health problems; generating a second simulation of at least one of an image of the second future state of the dental site or a 3D model of the second future state of the dental site; and generating a presentation showing the first simulation and the second simulation.

A 9^{th} implementation may extend any of the 6^{th} through 8^{th} implementations. In the 9^{th} implementation, the method further comprises receiving additional data of the patient, the additional data comprising at least one of patient age or underlying patient health conditions; wherein the additional data of the patient is used in predicting the future state of the dental site.

A 10^{th} implementation may extend any of the 6^{th} through 9^{th} implementations. In the 10^{th} implementation, the method further comprises predicting one or more ancillary current or future health conditions of the patient.

An 11^{th} implementation may extend any of the 1^{st} through 10^{th} implementations. In the 11^{th} implementation, the method further comprises: receiving additional data of one or more prior states of the dental site of the patient; processing the additional data using the plurality of trained machine learning models, wherein the plurality of trained machine learning models output additional estimations of prior states of the one or more oral conditions; processing at least one of the additional data or the additional estimations of the prior states of the one or more oral conditions to generate at least one of a) one or more prior actionable symptom recommendations or b) one or more prior state diagnoses of the one or more oral health problems; and determining a change in the one or more oral health problems over a time period based on a comparison of the one or more oral conditions to the prior states of the one or more oral conditions.

A 12^{th} implementation may extend the 11^{th} implementation. In the 12^{th} implementation, the method further comprises generating at least one of an image, a 3D model, or a video showing the change in the one or more oral health problems over the time period.

A 13^{th} implementation may extend the 12^{th} implementation. In the 13^{th} implementation, the at least one of the image, the 3D model or the video is generated by processing at least one of the data, the estimations of the one or more oral conditions, the one or more actionable symptom recommendations, or the one or more diagnoses of the one or more oral health problems, and further based on processing at least one of the additional data, the additional estimations of the prior states of the one or more oral conditions, the one or more prior actionable symptom recommendations, the one or more prior state diagnoses of the one or more oral health problems, or the change in the one or more oral health problems over the time period, using a generative model.

A 14^{th} implementation may extend any of the 11^{th} through 13^{th} implementations. In the 14^{th} implementation, the method further comprises: predicting a future state of the dental site based on processing at least one of the data, the estimations of the one or more oral conditions, the one or more actionable symptom recommendations, or the one or more diagnoses of the one or more oral health problems, and further based on processing at least one of the additional data, the additional estimations of the prior states of the one or more oral conditions, the one or more prior actionable symptom recommendations, or the one or more prior state diagnoses of the one or more oral health problems, or the change in the one or more oral health problems over the time period, wherein the predicted future state of the dental site comprises a future state of at least one of the one or more oral conditions or the one or more oral health problems.

A 15^{th} implementation may extend the 14^{th} implementation. In the 15^{th} implementation, the method further comprises: generating at least one of an image of the predicted future state of the dental site, a 3D model of the predicted future state of the dental site, or a video showing a progression from the one or more prior states of the dental site of the patient to the predicted future state of the dental site.

A 16^{th} implementation may extend any of the 11^{th} through 15^{th} implementations. In the 16^{th} implementation, the method further comprises: performing a trend analysis on at least one of the one or more oral conditions or the one or more oral health problems.

A 17^{th} implementation may extend any of the 1^{st} through 16^{th} implementations. In the 17^{th} implementation, the method further comprises: receiving initial image data of the current state of the dental site; processing the initial image data using a first trained machine learning model, wherein the first trained machine learning model outputs an initial estimation of the one or more oral conditions that are insufficient to diagnose the one or more oral health problems; and determining, based on the initial estimation, to at least one of perform additional analysis of the initial image data or recommend generation of the data of a current state of a dental site.

An 18^{th} implementation may extend the 17^{th} implementation. In the 18^{th} implementation, processing of the initial image data is performed on a patient device, and wherein processing the data using the plurality of trained machine learning models is performed on a server device.

A 19^{th} implementation may extend the 18^{th} implementation. In the 19^{th} implementation, the initial image data is generated by the patient device, wherein the patient device comprises a mobile computing device of the patient.

A 20^{th} implementation may extend any of the 1^{st} through 19^{th} implementations. In the 20^{th} implementation, the data comprises first data generated using a first oral state capture modality and second data generated using a second oral state capture modality, the method further comprising: processing the first data using one or more of the plurality of trained machine learning models, wherein the one or more of the plurality of trained machine learning models output first estimations of the one or more oral conditions; and processing at least one of the first data or the first estimations of the one or more oral conditions to output at least one of a) one or more initial actionable symptom recommendations or b) one or more initial diagnoses of the one or more oral health problems, and further to output a recommendation to generate the second data.

A 21^{st} implementation may extend the 20^{th} implementation. In the 21^{st} implementation, the method further comprises: receiving the second data responsive to outputting the recommendation to generate the second data; and replacing at least one of a) the first estimations with the estimations, b) the one or more initial actionable symptom recommendations with the one or more actionable symptom recommendations, or c) the one or more initial diagnoses with the one or more diagnoses based on additional processing of the second data.

A 22^{nd} implementation may extend any of the 20^{th} through 21^{st} implementations. In the 22^{nd} implementation, the method further comprises: performing at least one of a) confirming the one or more initial diagnoses based on processing of the second data, b) determining a severity of the one or more oral health problems based on processing of the second data, or c) generating the one or more treatment recommendations based on processing of the second data.

A 23^{rd} implementation may extend any of the 1^{st} through 22^{nd} implementations. In the 23^{rd} implementation, the one or more oral conditions and the one or more oral health problems comprise a caries, wherein the data comprises a) at least an occlusal portion of a three-dimensional (3D) surface of the dental site or a color image of the dental site and b) an x-ray image of the dental site, the method further comprising: determining a depth of the caries based on analysis of the x-ray image; determining a surface area of the caries based on analysis of the occlusal portion of the 3D surface or the color image; and estimating a volume of the caries based on the depth of the caries and the surface area of the caries.

A 24^{th} implementation may extend the 23^{rd} implementation. In the 24^{th} implementation, the method further comprises: determining whether to treat the caries with a crown or a filling based on the estimated volume of the caries.

A 25^{th} implementation may extend any of the 1^{st} through 24^{th} implementations. In the 25^{th} implementation, the method further comprises: determining at least one of a doctor or a group practice treating the patient; and determining one or more treatment preferences associated with at least one of the doctor or the group practice, wherein the one or more treatment recommendations are generated in view of the one or more treatment preferences.

A 26^{th} implementation may extend any of the 1^{st} through 25^{th} implementations. In the 26^{th} implementation, the one or more oral conditions and the one or more oral health problems comprise one or more caries, and wherein the data comprises at least two of an intraoral scan of the dental site, a near infrared (NIR) image of the dental site, or an x-ray image of the dental site.

A 27^{th} implementation may extend any of the 1^{st} through 26^{th} implementations. In the 27^{th} implementation, the one or more oral health problems comprise at least one of caries, periodontal disease, a tooth root issue, a cracked tooth, a broken tooth, oral cancer, a cause of bad breath, or a cause of a malocclusion.

A 28^{th} implementation may extend any of the 1^{st} through 27^{th} implementations. In the 28^{th} implementation, the method further comprises: determining a severity of each of the one or more oral conditions and/or oral health problems; and ranking the one or more oral conditions and/or oral health problems based at least in part on the severity.

A 29^{th} implementation may extend any of the 1^{st} through 28^{th} implementations. In the 29^{th} implementation, each trained machine learning model of the plurality of trained machine learning model is trained at least one of a) to process data from a particular oral state capture modality or b) to output estimations of a particular oral condition.

A 30^{th} implementation may extend any of the 1^{st} through 29^{th} implementations. In the 30^{th} implementation, generating the one or more diagnoses of the one or more oral health problems comprises performing a differential diagnosis of the one or more oral health problems.

A 31^{st} implementation may extend any of the 1^{st} through 30^{th} implementations. In the 31^{st} implementation, the method further comprises: determining one or more questions for a doctor to ask the patient based on processing the at least one of the data or the estimations of the one or more oral conditions; outputting the one or more questions; receiving answers to the one or more questions; and updating at least one of a) the one or more actionable symptom recommendations, b) the one or more diagnoses of the one or more oral health problems or c) the one or more treatment recommendations based on the received answers.

A 32^{nd} implementation may extend any of the 1^{st} through 31^{st} implementations. In the 32^{nd} implementation, the method further comprises: generating a post treatment simulation of the dental site; and outputting the post treatment simulation of the dental site.

A 33^{rd} implementation may extend the 32^{nd} implementation. In the 33^{rd} implementation, the post treatment simulation comprises at least one of a simulated two-dimensional (2D) color image, a simulated three-dimensional (3D) model, a simulated x-ray image, or a simulated CBCT scan.

A 34^{th} implementation may extend any of the 1^{st} through 33^{rd} implementations. In the 34^{th} implementation, at least one of the plurality of trained machine learning models comprises a trained machine learning model that has been trained to receive one or more dental x-rays as an input and to generate an output indicating at least one of probabilities that the patient has at least one of the one or more oral conditions or locations in the dental site at which the at least one oral condition was detected.

A 35^{th} implementation may extend any of the 1^{st} through 34^{th} implementations. In the 35^{th} implementation, the one or more oral conditions are selected from a group consisting of caries, gum recession, gingival swelling, tooth wear, bleeding, malocclusion, tooth crowding, tooth spacing, plaque, tooth stains, and tooth cracks.

A 36^{th} implementation may extend any of the 1^{st} through 35^{th} implementations. In the 36^{th} implementation, the method further comprises: receiving a selection of one or more recommended treatments; and generating a presentation comprising the one or more selected treatments and associated prognoses of the one or more selected treatments, the presentation comprising talking points for a doctor.

A 37^{th} implementation may extend the 36^{th} implementation. In the 37^{th} implementation, the method further comprises: predicting a first future state of the dental site associated with a first treatment of the one or more recommended treatments; predicting a second future state of the dental site associated with a second treatment of the one or more recommended treatments; generating a first simulation of at least one of an image or a 3D model of the first future state of the dental site; generating a second simulation of at least one of an image or a 3D model of the second future state of the dental site; and presenting the first simulation and the second simulation.

A 38^{th} implementation may extend the 37^{th} implementation. In the 38^{th} implementation, the first simulation and the second simulation are generated using one or more generative models.

A 39^{th} implementation may extend any of the 1^{st} through 38^{th} implementations. In the 39^{th} implementation, processing at least one of the data or the estimations of the one or more oral conditions to generate at least one of a) the one or more actionable symptom recommendations or b) the one or more diagnoses of one or more oral health problems associated with the one or more oral conditions comprises: processing a plurality of the estimations of the one or more oral conditions using a first trained machine learning model that outputs a first actionable symptom recommendation or a first diagnosis of a first dental health condition; and processing the plurality of the estimations of the one or more oral conditions using a second trained machine learning model that outputs a second actionable symptom recommendation or a second diagnosis of a second dental health condition.

A 40^{th} implementation may extend the 39^{th} implementation. In the 40^{th} implementation, the first trained machine learning model and the second trained machine learning model share one or more model layers.

A 41^{st} implementation may extend any of the 1^{st} through 40^{th} implementations. In the 41^{st} implementation, processing at least one of the data or the estimations of the one or more oral conditions to generate at least one of a) actionable symptom recommendation or b) the one or more diagnoses of the one or more oral health problems is performed using one or more additional trained machine learning models.

A 42^{nd} implementation may extend any of the 1^{st} through 41^{st} implementations. In the 42^{nd} implementation, at least one of a) the actionable symptom recommendation, b) the one or more diagnoses of the one or more oral health problems or c) the one or more treatment recommendations are generated using a decision tree or a random forest model.

A 43^{rd} implementation may extend any of the 1^{st} through 42^{nd} implementations. In the 43^{rd} implementation, the method further comprises: determining that a treatment of the one or more treatment recommendations was performed on the patient; automatically generating an insurance claim for the treatment; and submitting the insurance claim to an insurance carrier.

A 44^{th} implementation may extend the 43^{rd} implementation. In the 44^{th} implementation, automatically generating the insurance claim comprises: selecting or generating an image of the dental site of the patient; and annotating the image based on at least one of the estimations of the one or more oral conditions, the one or more actionable symptom recommendation, the one or more diagnoses of the one or more oral health problems, or the treatment performed on the patient.

A 45^{th} implementation may extend the 44^{th} implementation. In the 45^{th} implementation, the method further comprises: determining a cost breakdown for the treatment, the cost breakdown comprising a total cost, an insurance carrier portion of the total cost and a patient portion of the total cost; and adding data from the cost breakdown to the insurance claim.

A 46^{th} implementation may extend any of the 44^{th} through 45^{th} implementations. In the 46^{th} implementation, the image is an x-ray image.

A 47^{th} implementation may extend any of the 44^{th} through 46^{th} implementations. In the 47^{th} implementation, automatically generating the insurance claim comprises: processing at least one of the data, the estimations of the one or more oral conditions, the treatment, or an insurance carrier identification using an additional trained machine learning model, wherein the additional trained machine learning model outputs the insurance claim.

A 48^{th} implementation may extend any of the 1^{st} through 47^{th} implementations. In the 48^{th} implementation, the plurality of trained machine learning models output locations of areas of interest associated with the one or more oral conditions, the method further comprising: displaying the areas of interest on at least one of an image or a 3D model of a dental arch of the patient, wherein the areas of interest are displayed using a visualization that is coded based on classes of the one or more oral conditions that the areas of interest are associated with.

A 49^{th} implementation may extend any of the 1^{st} through 48^{th} implementations. In the 49^{th} implementation, the method further comprises: receiving a selection of a treatment recommendation of the one or more treatment recommendations; and performing automated treatment planning based at least in part on the selected treatment recommendation.

A 50^{th} implementation may extend any of the 1^{st} through 49^{th} implementations. In the 50^{th} implementation, the one or more treatment recommendations comprise at least one of one or more restorative treatment recommendations or one or more orthodontic treatment recommendations, the method further comprising: receiving a selection of at least one of a restorative treatment recommendation of the one or more restorative treatment recommendations or an orthodontic treatment recommendation of the one or more orthodontic treatment recommendations; and generating a treatment plan that is one of a restorative treatment plan, an orthodontic treatment plan, or an ortho-restorative treatment plan based on the selection, the generating comprising: determining staging for the treatment plan; receiving modifications to one or more stages of the treatment plan; and outputting an updated treatment plan.

In a 51^{st} implementation, a method comprises: determining a dental practice; analyzing patient case details for a plurality of patients of the dental practice, the patient case details each comprising data of a pre-treatment state of a dental site of a patient of the plurality of patients and data of a treatment performed on the patient; determining statistics about the patient case details; and generating one or more recommendations for changes to treatments performed on patients for the dental practice based on the statistics.

A 52^{nd} implementation may extend the 51^{st} implementation. In the 52^{nd} implementation, analyzing patient case details for a patient comprises: processing the data of the pre-treatment state of the dental site using one or more trained machine learning models, wherein each trained machine learning model of the one or more trained machine learning models is trained to process one or more data items generated from one or more oral state capture modalities, wherein the one or more trained machine learning models output estimations of one or more oral conditions; processing at least one of the data or the estimations of the one or more oral conditions to generate one or more diagnoses of one or more oral health problems associated with the one or more oral conditions; generating one or more treatment recommendations for treatment of at least one oral health problem of the one or more oral health problems; and comparing the one or more treatment recommendations to the treatment performed on the patient.

A 53^{rd} implementation may extend the 52^{nd} implementation. In the 53^{rd} implementation, the method further comprises: determining, for a plurality of patients of the dental practice, a delta between the one or more treatment recommendations and the treatments performed on the patients, wherein the one or more recommendations are based at least in part on the delta.

A 54^{th} implementation may extend any of the 52^{nd} through 53^{rd} implementations. In the 54^{th} implementation, the method further comprises: determining a subset of the plurality of patients for which a treatment recommendation for a particular treatment was generated but for which the particular treatment was not performed, wherein the one or more recommendations comprise a recommendation to perform the particular treatment.

A 55^{th} implementation may extend any of the 52^{nd} through 54^{th} implementations. In the 55^{th} implementation, the method further comprises: determining a subset of the plurality of patients for which a treatment recommendation for a particular treatment was not generated but for which the particular treatment was performed, wherein the one or more recommendations comprise a recommendation not to perform the particular treatment.

A 56^{th} implementation may extend any of the 51^{st} through 55^{th} implementations. In the 56^{th} implementation, the dental practice comprises a group practice, the method further comprising: determining, for each patient of the plurality of patients, a doctor of the group practice who treated the patient; determining doctor specific statistics about the patient case details of patients treated by a particular doctor of the group practice; comparing the doctor specific statistics with the statistics of the dental practice; determining a delta between the doctor specific statistics and the statistics of the dental practice; and generating one or more recommendations for changes to treatments performed on patients for the doctor based on the delta.

A 57^{th} implementation may extend the 56^{th} implementation. In the 57^{th} implementation, determining the delta comprises at least one of the following: determining that the particular doctor applies a different standard for when to drill a caries than an average of the group practice; determining that the particular doctor applies a different standard for when to perform a particular treatment than the average of the group practice; or determining that the particular doctor applies a different standard for when to generate data of patients using a particular oral state capture modality than the average of the group practice.

A 58^{th} implementation may extend any of the 56^{th} through 57^{th} implementations. In the 58^{th} implementation, the method further comprises: generating a report showing the delta and the one or more recommendations for the changes to the treatments performed on patients for the doctor.

A 59^{th} implementation may extend any of the 56^{th} through 58^{th} implementations. In the 59^{th} implementation, the doctor specific statistics comprise statistics on treatment results for treatments performed by the particular doctor, and wherein the statistics of the dental practice comprise statistics on treatment results for treatments performed by the dental practice, the method further comprising: determining a treatment result standard based on the statistics on treatment results for treatments performed by the dental practice; and determining that the particular doctor has failed to meet the treatment result standard based on a determination that the treatment results for treatments performed by the particular doctor fall below the treatment result standard.

A 60^{th} implementation may extend any of the 51^{st} through 59^{th} implementations. In the 60^{th} implementation, the method further comprises generating a report showing the one or more recommendations.

In a 61^{st} implementation, a method comprises: receiving first data of a current state of a dental site of a patient, the first data generated from a first oral state capture modality; processing the first data using one or more trained machine learning models, wherein the one or more trained machine learning models output estimations of one or more oral conditions; and processing at least one of the first data or the estimations of the one or more oral conditions to generate an additional output comprising one or more diagnoses of one or more oral health problems associated with the one or more oral conditions, wherein the additional output further comprises a recommendation to use second data from a second oral state capture modality to verify the one or more diagnoses of one or more oral health problems associated with the one or more oral conditions.

A 62^{nd} implementation may extend the 61^{st} implementation. In the 62^{nd} implementation, the method further comprises: receiving the second data generated from the second oral state capture modality; processing the second data, using at least one of a) the one or more trained machine learning models or b) one or more additional trained machine learning models, to output additional estimations of the one or more oral conditions; and processing at least one of the second data or the additional estimations of the one or more oral conditions to verify the one or more diagnoses of one or more oral health problems associated with the one or more oral conditions.

A 63^{rd} implementation may extend the 62^{nd} implementation. In the 63^{rd} implementation, the method further comprises: updating the one or more diagnoses of one or more oral health problems associated with the one or more oral conditions based at least in part on the additional estimations of the one or more oral conditions.

A 64^{th} implementation may extend any of the 61^{st} through 63^{rd} implementations. In the 64^{th} implementation, the first oral state capture modality and the second oral state capture modality are each selected from the group consisting of: a periapical x-ray, a bite wing x-ray, a panoramic x-ray, a near infrared (NIR) image, a CBCT scan, data from an electronic compliance indicator, or a color image.

A 65^{th} implementation may extend any of the 61^{st} through 64^{th} implementations. In the 65^{th} implementation, the one or more oral health problems comprise at least one of caries, periodontal disease, a tooth root issue, a cracked tooth, a broken tooth, oral cancer, a cause of bad breath, or a cause of a malocclusion.

In a 66^{th} implementation, an method comprises: receiving a radiograph of a dental site; processing the radiograph using a segmentation pipeline to segment the radiograph into a plurality of constituent dental objects, wherein processing the radiograph using the segmentation pipeline comprises: processing the radiograph using one or more first models that generate one or more first outputs comprising one or more regions of interest associated with the plurality of constituent dental objects, the one or more first models comprising one or more first trained machine learning models; and processing the one or more regions of interest of the radiograph using a first plurality of additional models to generate a first plurality of additional outputs each comprising at least one of first identifications or first locations of at least a first subset of the plurality of constituent dental objects, the first plurality of additional models comprising a first plurality of additional trained machine learning models; and generating a dental chart comprising the plurality of constituent dental objects.

A 67^{th} implementation may extend the 66^{th} implementation. In the 67^{th} implementation, the one or more regions of interest comprises a region of a jaw, and wherein the first subset of the plurality of constituent dental objects comprises a plurality of teeth in the jaw.

A 68^{th} implementation may extend any of the 66^{th} through 67^{th} implementations. In the 68^{th} implementation, the one or more regions of interest comprises regions of one or more teeth, and wherein the first subset of the plurality of constituent dental objects comprises at least one of caries, a periapical radiolucency, a restoration, or a periodontal bone loss location associated with the one or more teeth.

A 69^{th} implementation may extend any of the 66^{th} through 68^{th} implementations. In the 69^{th} implementation, the one or more regions of interest comprise at least one of: a region of interest for a lower jaw; a region of interest for a convex hull around the lower jaw; a region of interest for one or more tooth segments; a region of interest for a periodontal bone line; or a region of interest for jaws and teeth.

A 70^{th} implementation may extend any of the 66^{th} through 69^{th} implementations. In the 70^{th} implementation, the method further comprises: determining a radiograph type of the radiograph from a plurality of radiograph types; and selecting the segmentation pipeline from a plurality of distinct segmentation pipelines based on the radiograph type, wherein each of the plurality of distinct segmentation pipelines comprises a different combination of trained machine learning models.

A 71^{st} implementation may extend the 70^{th} implementation. In the 71^{st} implementation, the plurality of radiograph types comprise a bite-wing x-ray, a panoramic x-ray and a periapical x-ray.

A 72^{nd} implementation may extend any of the 66^{th} through 71^{st} implementations. In the 72^{nd} implementation, the method further comprises: processing the radiograph using one or more second models that generate one or more second outputs comprising identifications and locations of a plurality of teeth of the plurality of constituent dental objects, the one or more second models comprising one or more second trained machine learning models.

A 73^{rd} implementation may extend the 72^{nd} implementation. In the 73^{rd} implementation, the one or more second trained machine learning models comprise a first segmentation model that performs semantic segmentation of the plurality of teeth in the radiograph and a second segmentation model that performs instance segmentation of the plurality of teeth in the radiograph.

A 74^{th} implementation may extend the 73^{rd} implementation. In the 74^{th} implementation, the first segmentation model outputs a mask indicating pixels classified as the plurality of teeth, and wherein the second segmentation model outputs, for each tooth of the plurality of teeth, a bounding box for the tooth and a tooth number of the tooth.

A 75^{th} implementation may extend any of the 73^{rd} through 74^{th} implementations. In the 75^{th} implementation, the method further comprises: determining, based on an output of at least one of the first segmentation model or the second segmentation model, tooth numbering of the plurality of teeth in the radiograph; determining whether the tooth numbering satisfies one or more constraints; and updating the tooth numbering using a statistical model responsive to determining that the tooth numbering fails to satisfy the one or more constraints.

A 76^{th} implementation may extend any of the 72^{nd} through 75^{th} implementations. In the 76^{th} implementation, the one or more second trained machine learning models comprises an ensemble model comprising a plurality of machine learning models trained to perform segmentation and classification of teeth in parallel.

A 77^{th} implementation may extend the 76^{th} implementation. In the 77^{th} implementation, the ensemble model comprises a first model that generates a pixel-level mask for all teeth and a second model that identifies individual teeth and generates separate bounding boxes for each identified tooth.

A 78^{th} implementation may extend any of the 77^{th} implementation. In the 78^{th} implementation, each bounding box is assigned a tooth number.

A 79^{th} implementation may extend any of the 72^{nd} through 78^{th} implementations. In the 79^{th} implementation, the method further comprises: for each output of the first plurality of additional outputs, performing postprocessing of the output based on data from the one or more second outputs to at least one of augment, verify or correct at least one of the first identifications or the first locations of at least the first subset of the plurality of constituent dental objects.

An 80^{th} implementation may extend the 79^{th} implementation. In the 80^{th} implementation, the first plurality of additional trained machine learning models comprise a machine learning model trained to detect periapical radiolucency, and wherein performing the postprocessing comprises assigning periapical radiolucency information to each of the plurality of teeth.

An 81^{st} implementation may extend the 80^{th} implementation. In the 81^{st} implementation, performing the postprocessing further comprises: determining inflammation at apexes of a plurality of neighboring teeth based on the periapical radiolucency at the plurality of neighboring teeth; and assigning a lesion to the plurality of neighboring teeth based on the determined inflammation and segmentation information of the plurality of teeth output by the one or more second models.

An 82^{nd} implementation may extend any of the 79^{th} through 81^{st} implementations. In the 82^{nd} implementation, the first plurality of additional trained machine learning models comprise a machine learning model trained to detect caries, and wherein performing the postprocessing comprises assigning detected caries to one or more of the plurality of teeth.

An 83^{rd} implementation may extend the 82^{nd} implementation. In the 83^{rd} implementation, performing the postprocessing further comprises determining a severity for each of the detected caries.

An 84^{th} implementation may extend the 83^{rd} implementation. In the 84^{th} implementation, the severity of a detected caries is computed by: determining at least one of a size or a depth of the caries, wherein the severity is based on at least one of the size or the depth.

An 85^{th} implementation may extend any of the 79^{th} through 84^{th} implementations. In the 85^{th} implementation, the first plurality of additional trained machine learning models comprise a machine learning model trained to detect a periodontal bone loss, and wherein performing the postprocessing comprises determining a severity of the periodontal bone loss.

An 86^{th} implementation may extend the 85^{th} implementation. In the 86^{th} implementation, the severity of the periodontal bone loss is determined for a tooth by: determining an enamel line at which enamel of the tooth ends; determining a root bottom of the tooth; determining a first distance between the root bottom and the enamel line for the tooth; determining a second distance between the enamel line and a periodontal bone line of the one or more regions of interest; and determining a ratio between the first distance and the second distance.

An 87^{th} implementation may extend any of the 85^{th} through 86^{th} implementations. In the 87^{th} implementation, the severity of the periodontal bone loss is determined for a tooth by: determining an enamel line at which enamel of the tooth ends; and determining a distance between the enamel line and a periodontal bone line of the one or more regions of interest.

An 88^{th} implementation may extend any of the 85^{th} through 87^{th} implementations. In the 88^{th} implementation, the method further comprises: color coding at least a portion of the tooth in the dental chart based on the severity of the periodontal bond loss.

An 89^{th} implementation may extend any of the 85^{th} through 88^{th} implementations. In the 89^{th} implementation, the method further comprises: receiving a selection of thresholds for values associated with one or more severity levels of the periodontal bone loss, wherein the determined severity of the periodontal bone loss is determined based on comparing determined values for one or more teeth to selected thresholds.

A 90^{th} implementation may extend any of the 79^{th} through 89^{th} implementations. In the 90^{th} implementation, the first plurality of additional trained machine learning models comprise a machine learning model trained to detect restorations, and wherein performing the postprocessing comprises assigning detected restorations to one or more of the plurality of teeth.

A 91^{st} implementation may extend the 90^{th} implementation. In the 91^{st} implementation, performing the postprocessing further comprises determining a restoration type for one or more of the detected restorations.

A 92^{nd} implementation may extend the 91^{st} implementation. In the 92^{nd} implementation, the restoration type is one of a filling, a crown, a root canal filing, an implant, or a bridge.

A 93^{rd} implementation may extend any of the 91^{st} through 92^{nd} implementations. In the 93^{rd} implementation, determining the restoration type comprises: determining, based on tooth segmentation information output by the one or more second models, a supporting tooth of the restoration; determining that a size of the supporting tooth is greater than a size of the restoration; and determining that the restoration is a crown.

A 94^{th} implementation may extend any of the 91^{st} through 93^{rd} implementations. In the 94^{th} implementation, determining the restoration type comprises: determining, based on tooth segmentation information output by the one or more second models, that the restoration is associated with one or more teeth; determining that a size of the restoration is approximately equal to a size of the one or more teeth; and determining that the restoration is a bridge.

A 95^{th} implementation may extend any of the 79^{th} through 94^{th} implementations. In the 95^{th} implementation, the first plurality of additional models comprise a model trained to identify impacted teeth, and wherein performing the postprocessing comprises performing the following for each tooth identified by the model as an impacted tooth: comparing a location of the tooth to a location of a determined periodontal bone line output by the one or more first models; and confirming that the tooth is an impacted tooth responsive to determining that a crown of the tooth is at or below the periodontal bone line.

A 96^{th} implementation may extend any of the 79^{th} through 95^{th} implementations. In the 96^{th} implementation, the first plurality of additional models comprise a model trained to identify partially erupted teeth, wherein performing the postprocessing comprises performing the following for each tooth identified by the model as a partially erupted tooth: comparing a location of the tooth to a location of a determined periodontal bone line output by the one or more first models; and confirming that the tooth is a partially erupted tooth responsive to determining that enamel of the tooth interests the periodontal bone line.

A 97^{th} implementation may extend any of the 79^{th} through 96^{th} implementations. In the 97^{th} implementation, the method further comprises: combining postprocessed outputs of two or more of the first plurality of additional outputs; and performing additional postprocessing on the combined postprocessed outputs to resolve any discrepancies therebetween.

A 98^{th} implementation may extend the 97^{th} implementation. In the 98^{th} implementation, the additional postprocessing is performed using a rules-based engine, and wherein performing the additional postprocessing comprises: identifying one or more teeth that were classified both as having caries and as restorations; and removing caries classifications for the one or more teeth.

A 99^{th} implementation may extend any of the 72^{nd} through 98^{th} implementations. In the 99^{th} implementation, the one or more second outputs comprise an assignment of tooth numbers to a plurality of teeth in the radiograph.

A 100^{th} implementation may extend the 99^{th} implementation. In the 100^{th} implementation, the method further comprises: processing the one or more second outputs using at least one of a statistical model or one or more rules to at least one of verify or correct the assignment of the tooth numbers to the plurality of teeth of the radiograph.

A 101^{st} implementation may extend the 100^{th} implementation. In the 101^{st} implementation, processing the one or more second outputs comprises at least one of: identifying and removing any duplicate tooth numbers; removing one or more tooth identifications responsive to determining that more than 32 teeth were identified; or updating tooth numbering assigned to one or more teeth responsive to determining that assigned tooth numbers are not left to right sorted and ordered.

A 102^{nd} implementation may extend any of the 72^{nd} through 101^{st} implementations. In the 102^{nd} implementation, the method further comprises: waiting for a first one of the first plurality of additional outputs to be generated by a first one of the first plurality of additional trained machine learning models before processing an input comprising the radiograph and data from the one or more first outputs using a second one of the first plurality of additional trained machine learning models, wherein the input for the second one of the first plurality of additional trained machine learning models further comprises data output by the first one of the first plurality of additional trained machine learning models.

A 103^{rd} implementation may extend any of the 66^{th} through 102^{nd} implementations. In the 103^{rd} implementation, the method further comprises: receiving an additional data item generated from a second oral state capture modality; processing the additional data item using one or more further trained machine learning models to generate one or more further outputs each comprising at least one of second identifications or second locations of at least a second subset of the plurality of constituent dental objects; and combining the one or more further outputs with the first plurality of additional outputs.

A 104^{th} implementation may extend the 103^{rd} implementation. In the 104^{th} implementation, the radiograph is a first type of radiograph, and wherein the additional data item is a second type of radiograph.

A 105^{th} implementation may extend any of the 103^{rd} through 104^{th} implementations. In the 105^{th} implementation, the second oral state capture modality comprises one of an intraoral scan, a three-dimensional model, a color image, a near infrared image, a CT scan, or a CBCT scan.

A 106^{th} implementation may extend any of the 103^{rd} through 105^{th} implementations. In the 106^{th} implementation, the method further comprises: registering the additional data item and the radiograph based on shared features of the dental site.

A 107^{th} implementation may extend any of the 66^{th} through 106^{th} implementations. In the 107^{th} implementation, the method further comprises: processing the radiograph using a second trained machine learning model that generates a second output comprising at least one of an identification or a location of a mandibular nerve canal.

A 108^{th} implementation may extend the 107^{th} implementation. In the 108^{th} implementation, the method further comprises: determining locations of roots of one or more teeth; determining a distance between the mandibular nerve canal and the roots of the one or more teeth; and responsive to determining that the distance is below a distance threshold for a tooth of the one or more teeth, generating a notice that the root of the tooth is near the mandibular nerve canal.

A 109^{th} implementation may extend the 108^{th} implementation. In the 197^{th} implementation, the method further comprises: responsive to determining that the distance is below a distance threshold and that surgery is to be performed on the one or more teeth, recommending three-dimensional imaging of the one or more teeth.

A 110^{th} implementation may extend any of the 66^{th} through 109^{th} implementations. In the 110^{th} implementation, the method further comprises: determining a plurality of teeth, wherein the plurality of constituent dental objects comprise the plurality of teeth; determining, for each dental object in the first subset of the plurality of constituent dental objects, a tooth associated with the dental object; determining respective values for one or more dental objects of the first subset of the plurality of constituent dental objects; providing visualizations of the plurality of teeth on the dental chart; and providing, for one or more teeth of the plurality of teeth, one or more additional visualizations of the one or more dental objects of the first subset of the plurality of constituent dental objects over the one or more teeth on the dental chart based on the respective values.

A 111^{th} implementation may extend the 110^{th} implementation. In the 111^{th} implementation, the one or more dental objects of the first subset of the plurality of constituent dental objects comprise one or more regions comprising one or more oral conditions, and wherein the respective values comprise respective severity levels of the one or more oral conditions.

In a 112^{th} implementation, a method comprises: receiving a radiograph of a dental site; processing the radiograph using a segmentation pipeline to identify one or more oral conditions for the dental site, wherein processing the radiograph using the segmentation pipeline comprises: processing the radiograph using one or more first models that perform tooth segmentation, wherein the one or more first models generate a first output of tooth segmentation information comprising identifications and locations of a plurality of teeth in the radiograph; processing the radiograph using one or more second models that generate a second output comprising at least one of identifications or locations of the one or more oral conditions; and performing postprocessing to combine the first output and the second output, wherein as a result of the postprocessing each of the one or more oral conditions is assigned to one or more teeth of the plurality of teeth in the radiograph.

A 113^{th} implementation may extend the 112^{th} implementation. In the 113^{th} implementation, the method further comprises: generating a dental chart comprising the plurality of teeth and the one or more oral conditions.

A 114^{th} implementation may extend any of the 112^{th} through 113^{th} implementations. In the 114^{th} implementation, processing the radiograph using the one or more first models comprises: processing the radiograph using a first trained machine learning model that generates a first preliminary output comprising tooth numbers for the plurality of teeth according to physiological heuristics; processing the radiograph using a second trained machine learning model that generates a second preliminary output comprising a jaw side associated with the radiograph; and processing an input comprising the radiograph, the first preliminary output and the second preliminary output using a third trained machine learning model to generate the first output.

A 115^{th} implementation may extend the 114^{th} implementation. In the 115^{th} implementation, the first trained machine learning model comprises a random forest model, wherein the second trained machine learning model comprises a neural network, and wherein the third trained machine learning model comprises an instance segmentation model.

A 116^{th} implementation may extend any of the 112^{th} through 115^{th} implementations. In the 116^{th} implementation, the method further comprises: determining a radiograph type of the radiograph from a plurality of radiograph types; and selecting the segmentation pipeline from a plurality of distinct segmentation pipelines based on the radiograph type, wherein each of the plurality of distinct segmentation pipelines comprises a different combination of trained machine learning models.

A 117^{th} implementation may extend the 116^{th} implementation. In the 117^{th} implementation, the plurality of radiograph types comprise a bite-wing x-ray, a panoramic x-ray and a periapical x-ray.

A 118^{th} implementation may extend any of the 112^{th} through 117^{th} implementations. In the 118^{th} implementation, the one or more oral conditions comprise caries and the one or more second models comprise a machine learning model trained to detect caries, wherein the machine learning model outputs segmentation information of one or more caries.

A 119^{th} implementation may extend the 118^{th} implementation. In the 119^{th} implementation, the one or more oral conditions further comprise dentin and the one or more second models further comprise an additional machine learning model trained to detect dentin, wherein the additional machine learning model outputs segmentation information of the dentin.

A 120^{th} implementation may extend the 119^{th} implementation. In the 120^{th} implementation, performing the postprocessing further comprises: determining, for a tooth of the plurality of teeth, a distance between a caries on the tooth and the dentin of the tooth based on a comparison of the caries segmentation information and the dentin segmentation information; and determining a severity of the caries for the tooth at least in part based on the distance.

A 121^{st} implementation may extend the 120^{th} implementation. In the 121^{st} implementation, performing the postprocessing further comprises: determining whether the caries penetrates the dentin for the tooth; responsive to determining that the caries penetrates the dentin, classifying the caries for the tooth as a dentin caries; and responsive to determining that the caries does not penetrate the dentin, classifying the caries as an enamel caries.

A 122^{nd} implementation may extend any of the 112^{th} through 121^{st} implementations. In the 122^{nd} implementation, the one or more second models further comprise an additional machine learning model trained to assign localization to caries, wherein the additional machine learning model is to receive as an input the caries segmentation information and to provide as an output one or more localization classes for one or more caries.

A 123^{rd} implementation may extend the 122^{nd} implementation. In the 123^{rd} implementation, the one or more localization classes comprises at least one of tooth left surface, tooth right surface, tooth top surface, tooth mesial surface, tooth distal surface, tooth lingual surface, or tooth buccal surface.

A 124^{th} implementation may extend any of the 112^{th} through 123^{rd} implementations. In the 124^{th} implementation, the one or more oral conditions comprise calculus and the one or more second models comprise a machine learning model trained to detect calculus, wherein the machine learning model outputs at least one of identifications or locations of calculus.

A 125^{th} implementation may extend any of the 112^{th} through 124^{th} implementations. In the 125^{th} implementation, the one or more oral conditions comprise one or more restorations and the one or more second models comprise a machine learning model trained to detect restorations, wherein the machine learning model outputs segmentation information of one or more restorations.

A 126^{th} implementation may extend the 125^{th} implementation. In the 126^{th} implementation, performing the postprocessing further comprises determining a restoration type for one or more of the detected restorations.

A 127^{th} implementation may extend the 126^{th} implementation. In the 127^{th} implementation, the restoration type is one of a filling, a crown, a root canal filing, an implant, or a bridge.

A 128^{th} implementation may extend any of the 126^{th} through 127^{th} implementations. In the 128^{th} implementation, determining the restoration type comprises: determining, based on the tooth segmentation information, a supporting tooth of the restoration; determining that a size of the supporting tooth is greater than a size of the restoration; and determining that the restoration is a crown.

A 129^{th} implementation may extend any of the 126^{th} through 128^{th} implementations. In the 129^{th} implementation, determining the restoration type comprises: determining, based on the tooth segmentation information, that the restoration is associated with one or more teeth; determining that a size of the restoration is approximately equal to a size of the one or more teeth; and determining that the restoration is a bridge.

A 130^{th} implementation may extend any of the 112^{th} through 129^{th} implementations. In the 130^{th} implementation, the one or more second models comprise at least two of a first trained machine learning model trained to detect caries, a second trained machine learning model trained to detect calculus, or a third trained machine learning model trained to detect restorations, the method further comprising: combining postprocessed outputs of the one or more second models; and performing additional postprocessing on the combined postprocessed outputs to resolve any discrepancies therebetween.

A 131^{st} implementation may extend the 130^{th} implementation. In the 131^{st} implementation, the one or more second models comprise the first trained machine learning model trained to detect caries and the third trained machine learning model trained to detect restorations, and wherein performing the additional postprocessing comprises: identifying one or more teeth that were classified both as having caries and as restorations; and removing caries classifications for the one or more teeth.

A 132^{nd} implementation may extend any of the 112^{th} through 131^{st} implementations. In the 132^{nd} implementation, the one or more oral conditions comprise periodontal bone loss and the one or more second models comprise a machine learning model trained to detect the periodontal bone loss, wherein the machine learning model outputs periodontal bone loss information.

A 133^{rd} implementation may extend the 132^{nd} implementation. In the 133^{rd} implementation, performing the postprocessing further comprises determining a severity of the periodontal bone loss.

A 134^{th} implementation may extend the 133^{rd} implementation. In the 134^{th} implementation, the severity of the periodontal bone loss is determined for a tooth by: determining a centoenamel junction (CEJ) for the tooth; determining a periodontal bone line (PBL) for the tooth; determining a root apex of the tooth; determining a first distance between the CEJ and the PBL; determining a second distance between the CEJ and the root apex; and determining a ratio between the first distance and the second distance.

A 135^{th} implementation may extend any of the 133^{rd} through 134^{th} implementations. In the 135^{th} implementation, the method further comprises: receiving an age of a patient, wherein the severity of the periodontal bone loss is determined based at least in part on the age of the patient.

A 136^{th} implementation may extend any of the 133^{rd} through 135^{th} implementations. In the 136^{th} implementation, the severity of the periodontal bone loss is determined for a tooth by: determining an enamel line at which enamel of the tooth ends; determining a periodontal bone line for the tooth; and determining a distance between the enamel line and the periodontal bone line.

A 137^{th} implementation may extend any of the 133^{rd} through 136^{th} implementations. In the 137^{th} implementation, the method further comprises: determining at least one of a) whether a patient has periodontitis or b) a stage of the periodontitis based at least in part on the bone loss value.

A 138^{th} implementation may extend the 137^{th} implementation. In the 138^{th} implementation, the method further comprises: receiving additional patient information comprising at least one of pocket depth information for one or more teeth, bleeding information for the one or more teeth, plaque information for the one or more teeth, infection information for the one or more teeth, smoking status for the patient, or medical history for the patient, wherein the additional patient information is used in determining at least one of a) whether the patient has periodontitis or b) a stage of the periodontitis.

A 139^{th} implementation may extend the 138^{th} implementation. In the 139^{th} implementation, at least a portion of the additional patient information is from intraoral scanning of the dental site using an intraoral scanner.

A 140^{th} implementation may extend any of the 132^{nd} through 139^{th} implementations. In the 140^{th} implementation, performing the postprocessing further comprises: determining bone loss values for each of the one or more teeth; and determining, based on the bone loss values, whether a patient has at least one of horizontal bone loss, vertical bone loss, generalized bone loss, or localized bone loss.

A 141^{st} implementation may extend the 140^{th} implementations. In the 142^{st} implementation, the method further comprises: determining an angle of a periodontal bone line for the patient at the one or more teeth, wherein the angle of the periodontal bone line is used to identify at least one of horizontal bone loss or vertical bone loss.

A 142^{nd} implementation may extend any of the 132^{nd} through 141^{st} implementations. In the 142^{nd} implementation, the radiograph comprises a bite-wing x-ray that fails to show a root of a tooth of the one or more teeth, the method further comprising: determining a tooth number of the tooth in the bite-wing x-ray; determining a tooth length from a previous periapical x-ray, a previous CBCT or a previous panoramic x-ray comprising a representation of the tooth; determining a centoenamel junction (CEJ) for the tooth from the bite-wing x-ray; determining a periodontal bone line (PBL) for the tooth from the bite-wing x-ray; determining a bone loss length between the CEJ and the PBL from the bite-wing x-ray; and determining a ratio between the bone loss length and the tooth length.

A 143^{rd} implementation may extend any of the 132^{nd} through 142^{nd} implementations. In the 143^{rd} implementation, the radiograph comprises a bite-wing x-ray that fails to show a root of a tooth of the one or more teeth, the method further comprising: determining a tooth number of the tooth in the bite-wing x-ray; determining a tooth size from a three-dimensional model of the dental site generated from intraoral scanning of the dental site; registering the bite-wing x-ray to the three-dimensional model for the tooth; determining a conversion between pixels of the bite-wing x-ray and physical units of measurement based on the registration; determining a centoenamel junction (CEJ) for the tooth from the bite-wing x-ray; determining a periodontal bone line (PBL) for the tooth from the bite-wing x-ray; determining a distance between the CEJ and the PBL in pixels from the bite-wing x-ray; and converting the distance in pixels into a distance in the physical units of measurement based on the conversion.

A 144^{th} implementation may extend any of the 112^{th} through 143^{rd} implementations. In the 144^{th} implementation, the one or more oral conditions comprise periapical radiolucency and the one or more second models comprise a machine learning model trained to detect periapical radiolucency, wherein the machine learning model outputs periapical radiolucency information.

A 145^{th} implementation may extend the 144^{th} implementation. In the 145^{th} implementation, performing the postprocessing further comprises: determining inflammation at apexes of a plurality of neighboring teeth based on the periapical radiolucency at the one or more teeth; and assigning a lesion to the plurality of teeth based on the determined inflammation and segmentation information of the plurality of teeth output by the one or more second models.

A 146^{th} implementation may extend any of the 112^{th} through 145^{th} implementations. In the 146^{th} implementation, the method further comprises: receiving a three-dimensional (3D) model of the dental site generated from a plurality of intraoral scans of the dental site; performing tooth segmentation of the 3D model of the dental site using one or more trained machine learning models; and merging information of the one or more oral conditions with the 3D model.

A 147^{th} implementation may extend any of the 112^{th} through 146^{th} implementations. In the 147^{th} implementation, the method further comprises: determining at least one of an orthodontic treatment or a restorative treatment to be performed on the dental site; and generating a treatment plan comprising at least one of the orthodontic treatment or the restorative treatment.

A 148^{th} implementation may extend the 147^{th} implementation. In the 148^{th} implementation, determining at least one of the orthodontic treatment or the restorative treatment comprises determining staging for at least one of the orthodontic treatment or the restorative treatment.

A 149^{th} implementation may extend the 148^{th} implementation. In the 149^{th} implementation, the method further comprises: receiving one or more modifications to at least one of the orthodontic treatment or the restorative treatment; and updating the treatment plan.

A 150^{th} implementation may extend any of the 112^{th} through 149^{th} implementations. In the 150^{th} implementation, the one or more second models comprise a machine learning model trained to detect a mandibular nerve canal, wherein the machine learning model outputs segmentation information of the mandibular nerve canal, and wherein performing the postprocessing comprises: determining locations of roots of the one or more teeth; determining a distance between the mandibular nerve canal and the roots of the one or more teeth; and responsive to determining that the distance is below a distance threshold for a tooth of the one or more teeth, generating a notice that the root of the tooth is near the mandibular nerve canal.

A 151^{st} implementation may extend the 150^{th} implementation. In the 151^{st} implementation, the method further comprises: responsive to determining that the distance is below a distance threshold and that surgery is to be performed on the one or more teeth, recommending three-dimensional imaging of the one or more teeth.

In a 152^{nd} implementation, a method of training one or more machine learning models comprises: receiving a plurality of images of dental sites; for one or more images of the plurality of images, performing the following, comprising: providing the image to a plurality of experts, wherein the image is presented to each of the plurality of experts via an image annotation user interface, and wherein each expert of the plurality of experts independently labels one or more dental conditions on the image via the image annotation user interface; receiving a plurality of annotated versions of the image; and determining a combined annotated version of the image based at least in part on the plurality of annotated versions of the image; and training the one or more machine learning models using the combined annotated version of each image of the plurality of images.

A 153^{rd} implementation may extend the 152^{nd} implementation. In the 153^{rd} implementation, the plurality of images comprise a plurality of radiographs.

A 154^{th} implementation may extend the 153^{rd} implementation. In the 154^{th} implementation, the method further comprises: determining that there is disagreement in the one or more dental conditions between the plurality of annotated versions of the image; providing the plurality of annotated versions of the image to one or more additional experts, wherein the plurality of annotated versions of the image are presented to each of the one or more additional experts via the image annotation user interface, and wherein each expert of the one or more additional experts labels the one or more dental conditions on the image to generate a new annotated version of the image; and receiving the new annotated version of the image, wherein the combined annotated version of the image corresponds to the new annotated version of the image.

A 155^{th} implementation may extend any of the 153^{rd} through 154^{th} implementations. In the 155^{th} implementation, the method further comprises: presenting the image in the image annotation user interface; receiving user input that generates the labels of the one or more dental conditions on the image; and saving an annotated version of the image that comprises the labels.

A 156^{th} implementation may extend the 155^{th} implementation. In the 156^{th} implementation, the method further comprises: presenting options for changing at least one of a brightness, a contrast, a marker opacity or a box opacity in the image annotation user interface; receiving selection of one of the options; and changing at least one of the brightness, the contrast, the marker opacity or the box opacity based on the selection.

A 157^{th} implementation may extend any of the 153^{rd} through 156^{th} implementations. In the 157^{th} implementation, the method further comprises: determining patient case details for the plurality of images; determining that training data items already included in a training dataset for training the machine learning model lack a sufficient number of images having the patient case details of the one or more images; and selecting the one or more images for annotation.

A 158^{th} implementation may extend any of the 153^{rd} through 157^{th} implementations. In the 158^{th} implementation, the method further comprises: automatically annotating one or more of the plurality of images; providing the automatically annotated images to one or more experts; and receiving updates to annotations for at least one of the one or more automatically annotated images.

A 159^{th} implementation may extend any of the 153^{rd} through 158^{th} implementations. In the 159^{th} implementation, determining the combined annotated version of the image based at least in part on the plurality of annotated versions of the image comprises determining a union of labels of the plurality of annotated versions of the image.

A 160^{th} implementation may extend any of the 153^{rd} through 159^{th} implementations. In the 160^{th} implementation, determining the combined annotated version of the image based at least in part on the plurality of annotated versions of the image comprises determining an intersection of labels of the plurality of annotated versions of the image.

In a 161^{st} implementation, a method comprises: receiving image data of a current state of a dental site of a patient; processing the image data using a segmentation pipeline to generate an output comprising segmentation information for one or more teeth in the image data and at least one of identifications or locations of one or more oral conditions observed in the image data, wherein each of the one or more oral conditions is associated with a tooth of the one or more teeth; generating a visual overlay comprising visualizations for each of the one or more oral conditions; outputting the image data to a display; and outputting the visual overlay to the display over the image data.

A 162^{nd} implementation may extend the 161^{st} implementation. In the 162^{nd} implementation, the image data comprises a radiograph.

A 163^{rd} implementation may extend the 162^{nd} implementation. In the 163^{rd} implementation, processing the image data using the segmentation pipeline comprises: processing the image data using one or more first trained machine learning models to generate a first output comprising the segmentation information for the one or more teeth in the image data; and processing the image data using one or more additional trained machine learning models to generate a second output comprising at least one of the identifications or the locations of the one or more oral conditions.

A 164^{th} implementation may extend the 163^{rd} implementation. In the 164^{th} implementation, for an oral condition of the one or more oral conditions an additional trained machine learning model of the one or more additional trained machine learning models outputs one or more bounding boxes for an instance of the oral condition, the method further comprising: determining a tooth associated with the bounding box; determining an intersection of data from the bounding box and a segmentation mask for the tooth from the segmentation information; and determining a pixel-level mask for the instance of the oral condition based at least in part on the intersection of the data from the bounding box and the segmentation mask.

A 165^{th} implementation may extend the 164^{th} implementation. In the 165^{th} implementation, the oral condition comprises a caries or a restoration, the method further comprising: subtracting the data from the bounding box that does not intersect with the segmentation mask; wherein the pixel-level mask is provided as a layer of the visual overlay representing the oral condition within the tooth.

A 166^{th} implementation may extend any of the 164^{th} through 165^{th} implementations. In the 166^{th} implementation, the oral condition comprises calculus, the method further comprising: drawing an ellipse within the bounding box, wherein the intersection of the data from the bounding box and the segmentation mask comprises an intersection of the ellipse and the segmentation mask; and subtracting the data from the bounding box that intersects with the segmentation mask; wherein the pixel-level mask is provided as a layer of the visual overlay representing the calculus around the tooth.

A 167^{th} implementation may extend any of the 163^{rd} through 166^{th} implementations. In the 167^{th} implementation, for an oral condition of the one or more oral conditions an additional trained machine learning model of the one or more additional trained machine learning models outputs a plurality of bounding boxes for an instance of the oral condition, the method further comprising: determining that a first bounding box of the plurality of bounding boxes encapsulates one or more additional bounding boxes of the plurality of bounding boxes; and removing the one or more additional bounding boxes.

A 168^{th} implementation may extend any of the 163^{rd} through 167^{th} implementations. In the 168^{th} implementation, for an oral condition of the one or more oral conditions an additional trained machine learning model of the one or more additional trained machine learning models outputs a bounding box for an instance of the oral condition, the method further comprising: determining a tooth associated with the bounding box; determining an overlap between the bounding box and a segmentation mask for the tooth from the segmentation information; and determining a location of the oral condition on the tooth based at least in part on the overlap.

A 169^{th} implementation may extend the 168^{th} implementation. In the 169^{th} implementation, the method further comprises: marking the location of the oral condition on the tooth on at least one of the image data or a dental chart.

A 170^{th} implementation may extend any of the 168^{th} through 169^{th} implementations. In the 170^{th} implementation, the location comprises at least one of a mesial location, a distal location, or an occlusal location.

A 171^{st} implementation may extend any of the 168^{th} through 170^{th} implementations. In the 171^{st} implementation, determining the location comprises: performing principal component analysis of the segmentation mask or a bounding box for the tooth from the segmentation information to determine at least a first principal component; determining a first line between a tooth occlusal surface and a tooth root apex based on the first principal component; determining a first portion of the bounding box that is on a mesial side of the first line and a second portion of the bounding box that is on a distal side of the first line; and determining whether the oral condition is on the mesial side of the tooth or the distal side of the tooth based on the first portion and the second portion.

A 172^{nd} implementation may extend the 171^{st} implementation. In the 172^{nd} implementation, a second principal component is further determined from the principal component analysis, the method further comprising: determining a second line that extends in the mesial to distal direction based on the second principal component.

A 173^{rd} implementation may extend any of the 162^{nd} through 172^{nd} implementations. In the 173^{rd} implementation, the method further comprises: generating a dental chart for the patient; populating the dental chart based on data for the one or more oral conditions; and outputting the dental chart to the display.

A 174^{th} implementation may extend the 173^{rd} implementation. In the 174^{th} implementation, each instance of one or more oral conditions is provided as a distinct layer of the visual overlay, the method further comprising: receiving a selection of a tooth based on user interaction with at least one of the tooth in the dental chart or the tooth in the image data; outputting detailed information for instances of each of the one or more oral conditions identified for the selected tooth; receiving an instruction to remove an instance of an oral condition of the tooth; and marking the tooth as not having the instance of the oral condition.

A 175^{th} implementation may extend the 174^{th} implementation. In the 175^{th} implementation, the method further comprises: receiving input to add a new instance of the oral condition to the tooth, the input comprising an indication of pixels of the image data comprising the oral condition; and marking the tooth as having the new instance of the oral condition.

A 176^{th} implementation may extend any of the 173^{rd} through 175^{th} implementations. In the 176^{th} implementation, each instance of one or more oral conditions is provided as a distinct layer of the visual overlay, the method further comprising: receiving a selection of a tooth based on user interaction with at least one of the tooth in the dental chart or the tooth in the image data; outputting detailed information for instances of each of the one or more oral conditions identified for the selected tooth; receiving an instruction to add an instance of an oral condition to the tooth, the instruction comprising an indication of pixels of the image data comprising the oral condition; and marking the tooth as having the instance of the oral condition.

A 177^{th} implementation may extend the 176^{th} implementation. In the 177^{th} implementation, the method further comprises: determining a location on the tooth for the instance of the oral condition; and indicating the location on the tooth for the instance of the oral condition in the dental chart.

A 178^{th} implementation may extend any of the 173^{rd} through 177^{th} implementations. In the 178^{th} implementation, the one or more oral conditions comprise one or more instances of caries, the method further comprising: for each instance of caries, determining whether the instance of the caries is an enamel caries or a dentin caries; marking instances of caries identified as enamel caries using a first visualization; and marking instances of caries identified as dentin caries using a second visualization.

A 179^{th} implementation may extend any of the 162^{nd} through 178^{th} implementations. In the 179^{th} implementation, the method further comprises: determining at least one of diagnoses or treatment options for the one or more oral conditions; and showing at least one of the diagnoses or the treatment options in a dropdown menu.

A 180^{th} implementation may extend any of the 162^{nd} through 179^{th} implementations. In the 180^{th} implementation, the method further comprises determining dental codes associated with the one or more oral conditions; and assigning the dental codes to the one or more oral conditions.

A 181^{st} implementation may extend the 180^{th} implementation. In the 181^{st} implementation, the method further comprises: determining a treatment that was performed on the patient; and automatically generating an insurance claim for the treatment, the insurance claim comprising the image data, at least a portion of the visual overlay comprising the one or more oral conditions that were treated, and the dental codes associated with the one or more oral conditions.

A 182^{nd} implementation may extend any of the 162^{nd} through 181^{st} implementations. In the 182^{nd} implementation, the at least one of the identifications or the locations of the one or more oral conditions comprises a probability map indicating, for each pixel of the image data, a probability of the pixel corresponding to at least one oral condition of the one or more oral conditions, the method further comprising: determining, for the at least one oral condition and for a first tooth, a pixel-level mask indicating pixels having a probability that exceeds a first threshold, wherein the visual overlay comprises the pixel-level mask for the first tooth.

A 183^{rd} implementation may extend the 182^{nd} implementation. In the 183^{rd} implementation, the method further comprises: receiving an instruction to activate a high sensitivity mode for oral condition detection; activating the high sensitivity mode, wherein activating the high sensitivity mode comprises replacing the first threshold with a second threshold that is lower than the first threshold; and determining, for the at least one oral condition and for the first tooth, a new pixel-level mask indicating pixels having a probability that exceeds the second threshold, wherein the visual overlay comprises the new pixel-level mask in the high sensitivity mode.

A 184^{th} implementation may extend the 183^{rd} implementation. In the 184^{th} implementation, prior to activating the high sensitivity mode the at least one oral condition was not identified for a second tooth, the method further comprising: determining, for the at least one oral condition and for the second tooth, a second new pixel-level mask indicating additional pixels having the probability that exceeds the second threshold, wherein the visual overlay comprises the second new pixel-level mask for the second tooth in the high sensitivity mode.

A 185^{th} implementation may extend the 184^{th} implementation. In the 185^{th} implementation, the at least one oral condition for the second tooth is identified as a potential instance of the at least one oral condition in view of the at least one oral condition being identified only in the high sensitivity mode.

A 186^{th} implementation may extend the 185^{th} implementation. In the 186^{th} implementation, the method further comprises: presenting one or more tools for reclassifying the at least one oral condition for the second tooth; receiving an instruction to reclassify the at least one oral condition for the second tooth based on user interaction with the one or more tools; and reclassifying the at least one oral condition for the second tooth as a verified instance of the at least one oral condition.

A 187^{th} implementation may extend any of the 183^{rd} through 186^{th} implementations. In the 187^{th} implementation, the method further comprises: outputting a visual indication that provides notification that the high sensitivity mode has been activated.

A 188^{th} implementation may extend any of the 162^{nd} through 187^{th} implementations. In the 188^{th} implementation, the method further comprises: receiving a command to generate a report; and generating the report comprising the image data, the visual overlay, and a dental chart showing, for each tooth of the patient, any oral conditions identified for that tooth.

A 189^{th} implementation may extend the 188^{th} implementation. In the 189^{th} implementation, the method further comprises: receiving additional data for the patient from a dental practice management system, the additional data including at least one of pocket depths, patient age, or patient underlying health conditions, wherein the additional data is incorporated into the report.

A 190^{th} implementation may extend any of the 188^{th} through 189^{th} implementations. In the 190^{th} implementation, the method further comprises: formatting the report in a structured data format ingestible by a dental practice management system; and adding the report to the dental practice management system.

A 191^{st} implementation may extend any of the 188^{th} through 190^{th} implementations. In the 191^{st} implementation, the method further comprises:
receiving at least one of annotations, the image data, the visual overlay or the dental chart by a model trained to format reports tailored for at least one of a doctor or a dental practice, wherein the model outputs the report.

A 192^{nd} implementation may extend the 191^{st} implementation. In the 192^{nd} implementation, the method further comprises: training the model to generate reports formatted for at least one of a doctor or a dental practice based on a training data set comprising past reports generated by at least one of the doctor or the dental practice.

A 193^{rd} implementation may extend any of the 188^{th} through the 192^{nd} implementations. In the 193^{rd} implementation, the method further comprises: inputting the report into a treatment planning system; and developing a treatment plan for treating the one or more oral conditions of the patient using the treatment planning system based at least in part on the report.

A 194^{th} implementation may extend any of the 188^{th} through the 193^{rd} implementations. In the 194^{th} implementation, the method further comprises: comparing the report to an earlier report for the patient generated at an earlier time; determining an oral condition of the one or more oral conditions that was also included in the earlier report and was not treated; and generating a notice of the untreated oral condition.

A 195^{th} implementation may extend any of the 188^{th} through the 194^{th} implementations. In the 195^{th} implementation, the method further comprises: comparing the report to an earlier report for the patient generated at an earlier time; determining a difference between a current severity of an oral condition of the one or more oral conditions from the report and a past severity of the oral condition from the earlier report; and generating a notice comprising the difference.

A 196^{th} implementation may extend any of the 188^{th} through the 195^{th} implementations. In the 196^{th} implementation, the method further comprises: comparing the report to reports of the one or more oral conditions for a plurality of additional patients; determining comparative severity levels of the one or more oral conditions between the patient and the plurality of additional patients based on the comparing; and prioritizing treatment of the patient based on the comparative severity levels.

A 197^{th} implementation may extend any of the 162^{nd} through the 196^{th} implementations. In the 197^{th} implementation, the one or more oral conditions comprise a bone loss value.

A 198^{th} implementation may extend the 197^{th} implementation. In the 198^{th} implementation, determining the bone loss value for a tooth comprises: determining a centoenamel junction (CEJ) for the tooth; determining a periodontal bone line (PBL) for the tooth; determining a root apex of the tooth; determining a first distance between the CEJ and the PBL; determining a second distance between the CEJ and the root apex; and determining a ratio between the first distance and the second distance.

A 199^{th} implementation may extend the 198^{th} implementation. In the 199^{th} implementation, the visual overlay comprises a pixel-level overlay over an area of the tooth between the CEJ and the PBL, wherein the pixel-level overlay graphically shows an amount of bone loss for the tooth.

A 200^{th} implementation may extend any of the 198^{th} through the 199^{th} implementations. In the 200^{th} implementation, the method further comprises: determining a severity for bone loss of the tooth based on the bone loss value and an age of the patient.

A 201^{st} implementation may extend any of the 197^{th} through the 200^{th} implementations. In the 201^{st} implementation, the method further comprises: determining at least one of a) whether the patient has periodontitis or b) a stage of the periodontitis based at least in part on the bone loss value.

A 202^{nd} implementation may extend the 201^{st} implementation. In the 202^{nd} implementation, the method further comprises: receiving additional patient information comprising at least one of pocket depth information for one or more teeth, bleeding information for the one or more teeth, plaque information for the one or more teeth, infection information for the one or more teeth, smoking status for the patient, or medical history for the patient, wherein the additional patient information is used in determining at least one of a) whether the patient has periodontitis or b) a stage of the periodontitis.

A 203^{rd} implementation may extend the 202^{nd} implementation. In the 203^{rd} implementation, at least a portion of the additional patient information is from intraoral scanning of the dental site using an intraoral scanner.

A 204^{th} implementation may extend any of the 197^{th} through the 203^{rd} implementations. In the 204^{th} implementation, the method further comprises: determining bone loss values for each of the one or more teeth of the patient; determining, based on the bone loss values, whether the patient has at least one of horizontal bone loss, vertical bone loss, generalized bone loss, or localized bone loss.

A 205^{th} implementation may extend the 204^{th} implementation. In the 205^{th} implementation, the method further comprises: determining an angle of a periodontal bone line for the patient at the one or more teeth, wherein the angle of the periodontal bone line is used to identify at least one of horizontal bone loss or vertical bone loss.

A 206^{th} implementation may extend any of the 197^{th} through the 205^{th} implementations. In the 206^{th} implementation, the method further comprises: generating an insurance report for at least one of periodontal scaling or root planing based at least in part on the bone loss value and an amount of calculus detected on the one or more teeth.

A 207^{th} implementation may extend any of the 162^{nd} through the 206^{th} implementations. In the 207^{th} implementation, the image data comprises a bite-wing x-ray that fails to show a root of a tooth of the one or more teeth, the method further comprising: determining a tooth number of the tooth in the bite-wing x-ray; determining a tooth length from a previous periapical x-ray, a previous CBCT or a previous panoramic x-ray comprising a representation of the tooth; determining a centoenamel junction (CEJ) for the tooth from the bite-wing x-ray; determining a periodontal bone line (PBL) for the tooth from the bite-wing x-ray; determining a bone loss length between the CEJ and the PBL from the bite-wing x-ray; and determining a ratio between the bone loss length and the tooth length.

A 208^{th} implementation may extend any of the 162^{nd} through the 207^{th} implementations. In the 208^{th} implementation, the image data comprises a bite-wing x-ray that fails to show a root of a tooth of the one or more teeth, the method further comprising: determining a tooth number of the tooth in the bite-wing x-ray; determining a tooth size from a three-dimensional model of the dental site generated from intraoral scanning of the dental site; registering the bite-wing x-ray to the three-dimensional model for the tooth; determining a conversion between pixels of the bite-wing x-ray and physical units of measurement based on the registration; determining a centoenamel junction (CEJ) for the tooth from the bite-wing x-ray; determining a periodontal bone line (PBL) for the tooth from the bite-wing x-ray; determining a distance between the CEJ and the PBL in pixels from the bite-wing x-ray; and converting the distance in pixels into a distance in the physical units of measurement based on the conversion.

A 209^{th} implementation may extend any of the 1^{st} through 208^{th} implementations. In the 209^{th} implementation, a non-transitory computer readable medium comprises instructions that, when executed by one or more processing devices, cause the one or more processing devices to perform the method of any of the 1^{st} through 208^{th} implementations.

A 210^{th} implementation may extend any of the 1^{st} through 208^{th} implementations. In the 210^{th} implementation, a system comprises a computing device comprising a memory and one or more processing devices, wherein the computing device is configured to perform the method of any of the 1^{st} through 208^{th} implementations.

A 211^{th} implementation may extend any of the 1^{st} through 52^{nd} implementations. In the 211^{th} implementation, a system comprises: a computing device comprising a memory and one or more processing devices, wherein the computing device is configured to perform the method of any of the 1^{st} through 52^{nd} implementations; and an additional computing device configured to send the data to the computing device.

A 212^{th} implementation may extend the 211^{th} implementation. In the 212^{th} implementation, the system further comprises a storage device configured to store at least one of the estimations of the one or more oral conditions, the one or more actionable symptom recommendations for the one or more oral health problems, the one or more diagnoses of the one or more oral health problems, or the one or more treatment recommendations.

A 213^{th} implementation may extend the 211^{th} or 212^{th} implementations. In the 213^{th} implementation, the system further comprises one or more radiography machines configured to generate at least a subset of the data.

A 214^{th} implementation may extend any of the 51^{st} through 60^{th} implementations. In the 214^{th} implementation, a system comprises: a computing device comprising a memory and one or more processing devices, wherein the computing device is configured to perform the method of any of the 51^{st} through 60^{th} implementations; and a storage device configured to store the one or more recommendations.

A 215^{th} implementation may extend any of the 61^{st} through 65^{th} implementations. In the 215^{th} implementation, a system comprises: a computing device comprising a memory and one or more processing devices, wherein the computing device is configured to perform the method of any of the 61^{st} through 65^{th} implementations; and an additional computing device configured to send at least one of the first data or the second data to the computing device.

A 216^{th} implementation may extend any of the 66^{th} through 111^{th} implementations. In the 216^{th} implementation, a system comprises: a computing device comprising a memory and one or more processing devices, wherein the computing device is configured to perform the method of any of the 66^{th} through 111^{th} implementations; and an additional computing device configured to send the radiograph to the computing device.

A 217^{th} implementation may extend the 216^{th} implementation. In the 217^{th} implementation, the system further comprises a storage device configured to store at least one of the first plurality of additional outputs or the dental chart.

A 218^{th} implementation may extend the 216^{th} or 217^{th} implementations. In the 218^{th} implementation, the system further comprises one or more radiography machines configured to generate the radiograph.

A 219^{th} implementation may extend any of the 112^{th} through 151^{st} implementations. In the 219^{th} implementation, a system comprises: a computing device comprising a memory and one or more processing devices, wherein the computing device is configured to perform the method of any of the 66^{th} through 111^{th} implementations; and an additional computing device configured to send the radiograph to the computing device.

A 220^{th} implementation may extend the 219^{th} implementation. In the 220^{th} implementation, the system further comprises a storage device configured to store information on the plurality of oral conditions identified for the dental site.

A 221^{st} implementation may extend the 216^{th} or 220^{th} implementations. In the 221^{st} implementation, the system further comprises one or more radiography machines configured to generate the radiograph.

A 222^{nd} implementation may extend any of the 161^{st} through 208^{th} implementations. In the 222^{nd} implementation, a system comprises: a computing device comprising a memory and one or more processing devices, wherein the computing device is configured to perform the method of any of the 161^{st} through 208^{th} implementations; and an additional computing device configured to send the image data to the computing device.

A 223^{rd} implementation may extend the 222^{nd} implementation. In the 223^{rd} implementation, the system further comprises a storage device configured to store at least one of the output or the visual overlay.

A 221^{st} implementation may extend the 216^{th} or 220^{th} implementations. In the 221^{st} implementation, the system further comprises one or more radiography machines configured to generate the image data.

### BRIEF DESCRIPTION OF THE DRAWINGS

The present disclosure is illustrated by way of example, and not by way of limitation, in the figures of the accompanying drawings.

FIG. 1 illustrates a workflow for detecting, predicting, diagnosing and reporting on oral conditions and/or oral health problems, in accordance with embodiments of the present disclosure.

FIG. 2 illustrates an architecture comprising a set of systems for detecting, predicting, diagnosing, reporting on and treating oral conditions and/or oral health problems, in accordance with embodiments of the present disclosure.

FIG. 3 illustrates a dental recommendation and diagnosis system, in accordance with embodiments of the present disclosure.

FIG. 4A illustrates a flow diagram for a method of assessing a patient's oral health using data from multiple oral state capture modalities, in accordance with embodiments of the present disclosure.

FIG. 4B illustrates a flow diagram for a method of predicting a future oral health of a patient using data from one or more oral state capture modalities, in accordance with embodiments of the present disclosure.

FIG. 4C illustrates a flow diagram for a method of tracking a patient's oral health over time based on data from one or more oral state capture modalities, in accordance with embodiments of the present disclosure.

FIG. 5A illustrates a flow diagram for a method of verifying and/or updating an estimate of a patient's oral health, as determined from a first oral state capture modality, using data from a second oral state capture modality, in accordance with embodiments of the present disclosure.

FIG. 5B illustrates a flow diagram for a method of using data from a radiograph and data from an image or 3D model to assess a caries severity, in accordance with embodiments of the present disclosure.

FIG. 5C illustrates a flow diagram for a method of updating an estimated oral health of a patient based on patient responses to prompted questions, in accordance with embodiments of the present disclosure.

FIGS. 6A-B illustrate a flow diagram for a method of analyzing a patient's teeth and gums, in accordance with embodiments of the present disclosure.

FIG. 7 illustrates a flow diagram for a method of automatically generating an insurance claim for an oral health treatment, in accordance with embodiments of the present disclosure.

FIGS. 8A-B illustrate a flow diagram for a method of generating a report for a dental practice, in accordance with embodiments of the present disclosure.

FIG. 9 illustrates a segmentation engine of an oral health diagnostics system, in accordance with embodiments of the present disclosure.

FIG. 10 illustrates an example segmentation pipeline for performing segmentation of a first type of dental radiographs, in accordance with embodiments of the present disclosure.

FIG. 11 illustrates an example segmentation pipeline for performing segmentation of a second type of dental radiographs, in accordance with embodiments of the present disclosure.

FIG. 12 illustrates an example segmentation pipeline for performing segmentation of a third type of dental radiographs, in accordance with embodiments of the present disclosure.

FIG. 13 illustrates a flow diagram for a method of processing a radiograph to identify oral conditions of a patient using a segmentation pipeline, in accordance with embodiments of the present disclosure.

FIG. 14A illustrates a flow diagram for a method of processing a radiograph to identify oral conditions of a patient using a segmentation pipeline, in accordance with embodiments of the present disclosure.

FIG. 14B illustrates a flow diagram for a method of processing a radiograph to identify oral conditions of a patient using a segmentation pipeline, in accordance with embodiments of the present disclosure.

FIG. 15A illustrates a flow diagram for a method of processing a radiograph to identify lesions around teeth of a patient using a segmentation pipeline, in accordance with embodiments of the present disclosure.

FIG. 15B illustrates a flow diagram for a method of processing a radiograph to identify periodontal bone loss of a patient using a segmentation pipeline, in accordance with embodiments of the present disclosure.

FIG. 15C illustrates a flow diagram for a method of processing a radiograph to identify restorations of a patient using a segmentation pipeline, in accordance with embodiments of the present disclosure.

FIG. 15D illustrates a flow diagram for a method of processing a radiograph to identify impacted and/or partially erupted teeth of a patient using a segmentation pipeline, in accordance with embodiments of the present disclosure.

FIG. 15E illustrates a flow diagram for a method of processing a radiograph to correct false positives with respect to identified caries for a patient using a segmentation pipeline, in accordance with embodiments of the present disclosure.

FIG. 16A illustrates a flow diagram for a method of identifying tooth roots near a mandibular nerve canal of a patient using a segmentation pipeline, in accordance with embodiments of the present disclosure.

FIG. 16B illustrates a flow diagram for a method of processing a radiograph to identify, and determine severity of, caries on teeth of a patient using a segmentation pipeline, in accordance with embodiments of the present disclosure.

FIG. 16C illustrates a flow diagram for a method of processing a radiograph to identify, and determine severity of, calculus on teeth of a patient using a segmentation pipeline, in accordance with embodiments of the present disclosure.

FIG. 16D illustrates a flow diagram for a method of processing a radiograph to identify lesions around teeth of a patient using a segmentation pipeline, in accordance with embodiments of the present disclosure.

FIG. 17A illustrates a flow diagram for a method of determining periodontal bone loss for a tooth of a patient from a bite-wing x-ray, in accordance with embodiments of the present disclosure.

FIG. 17B illustrates a flow diagram for a method of determining periodontal bone loss for a tooth of a patient from a bite-wing x-ray, in accordance with embodiments of the present disclosure.

FIG. 18 illustrates a model training workflow and a model application workflow for training and executing one or more models of a segmentation pipeline for an oral health diagnostics system, in accordance with an embodiment of the present disclosure.

FIG. 19A illustrates a flow diagram for a method of generating a training dataset for training one or more machine learning models of a segmentation pipeline, in accordance with embodiments of the present disclosure.

FIG. 19B illustrates a flow diagram for a method of generating a training dataset for training one or more machine learning models of a segmentation pipeline, in accordance with embodiments of the present disclosure.

FIG. 20 illustrates a flow diagram for a method of altering images and/or radiographs to be included in a training dataset for training one or more machine learning models of a segmentation pipeline, in accordance with embodiments of the present disclosure.

FIG. 21 illustrates a flow diagram for a method of selecting images and/or radiographs to be labeled and included in a training dataset for training one or more machine learning models of a segmentation pipeline, in accordance with embodiments of the present disclosure.

FIG. 22 illustrates a visualization engine of an oral health diagnostics system, in accordance with embodiments of the present disclosure.

FIG. 23A illustrates a flow diagram for a method of providing visualizations of oral conditions of a patient, in accordance with embodiments of the present disclosure.

FIG. 23B illustrates a flow diagram for a method of providing visualizations of oral conditions of a patient and of generating a report for the patient, in accordance with embodiments of the present disclosure.

FIG. 23C illustrates a flow diagram for a method of updating identified oral conditions of a patient based on doctor interaction with a user interface of an oral health diagnostics system, in accordance with embodiments of the present disclosure.

FIG. 23D illustrates a flow diagram for a method of determining visualizations for one or more oral conditions to be presented in a user interface of an oral health diagnostics system, in accordance with embodiments of the present disclosure.

FIG. 23E illustrates a flow diagram for a method of determining a region of a tooth that an oral condition is associated with, in accordance with embodiments of the present disclosure.

FIG. 23F illustrates a flow diagram for a method of a high sensitivity mode for detection of oral conditions, in accordance with embodiments of the present disclosure.

FIG. 24A illustrates a flow diagram for a method of comparing reports for a patient generated by an oral health diagnostics system, in accordance with embodiments of the present disclosure.

FIG. 24B illustrates a flow diagram for a method of prioritizing patient treatments based on generated reports of different patients of a dental practice, in accordance with embodiments of the present disclosure.

FIG. 24C illustrates a flow diagram for a method of identifying periodontitis, in accordance with embodiments of the present disclosure.

FIG. 24D illustrates a flow diagram for a method of generating a report of oral conditions of a patient, in accordance with embodiments of the present disclosure.

FIG. 25A illustrates a user interface of an oral health diagnostics system showing a panoramic x-ray, in accordance with embodiments of the present disclosure.

FIG. 25B illustrates a user interface of an oral health diagnostics system showing a bitewing dental x-ray, in accordance with embodiments of the present disclosure.

FIG. 25C illustrates a user interface of an oral health diagnostics system showing a periapical dental x-ray, in accordance with embodiments of the present disclosure.

FIG. 26A illustrates a tooth chart of a set of teeth without oral health condition detections, in accordance with embodiments of the present disclosure.

FIG. 26B illustrates a tooth chart of a set of teeth with oral condition detections, in accordance with embodiments of the present disclosure.

FIG. 27A illustrates a detailed view of oral conditions for a selected tooth, in accordance with embodiments of the present disclosure.

FIG. 27B illustrates a user interface for reassigning oral conditions to neighboring teeth, in accordance with embodiments of the present disclosure.

FIG. 28A illustrates a tool bar for an oral health diagnostics system showing a plurality of virtual buttons, in accordance with embodiments of the present disclosure.

FIG. 28B illustrates different rotation options for an x-ray, in accordance with embodiments of the present disclosure.

FIG. 28C shows a legend of more detailed caries information that is shown on a tooth chart when a caries pro mode is active, in accordance with embodiments of the present disclosure.

FIG. 28D shows a portion of a detailed view for a tooth while a caries pro mode is active, in accordance with embodiments of the present disclosure.

FIG. 28E illustrates a status bar for a patient record of an oral health diagnostics system, in accordance with embodiments of the present disclosure.

FIG. 28F illustrates a user interface for moving a detected tooth, in accordance with embodiments of the present disclosure.

FIG. 28G illustrates a user interface for moving a set of detected teeth, in accordance with embodiments of the present disclosure.

FIG. 29 illustrates a legend for an oral health diagnostics system, in accordance with embodiments of the present disclosure.

FIG. 30 illustrates a list of oral condition detections for an oral health diagnostics system, in accordance with embodiments of the present disclosure.

FIG. 31A illustrates a user interface for generating an oral conditions report for a patient, in accordance with embodiments of the present disclosure.

FIGS. 31B-C illustrate an oral conditions report for a patient, in accordance with embodiments of the present disclosure.

FIGS. 32A-B illustrate a legend of different types of oral condition overlays usable by a oral health diagnostics system, in accordance with embodiments of the present disclosure.

FIG. 33A illustrates a bitewing radiograph with overlays of detected oral conditions and an associated tooth chart in a standard sensitivity mode, in accordance with embodiments of the present disclosure.

FIG. 33B illustrates the bitewing radiograph with overlays of detected oral conditions and an associated tooth chart of FIG. 33A, in a high sensitivity mode, in accordance with embodiments of the present disclosure.

FIG. 33C illustrates the bitewing radiograph with overlays of detected oral conditions and an associated tooth chart of FIG. 33A, in a standard sensitivity mode after a caries detected in the high sensitivity mode has been verified, in accordance with embodiments of the present disclosure.

FIG. 34 shows the same radiograph and tooth of FIG. 25A, but with different overlays on the radiograph and on the tooth chart after a periodontal bone loss mode has been activated, in accordance with embodiments of the present disclosure.

FIGS. 35A-C illustrate interactive elements of a periodontal mode loss mode of an oral health diagnostics system, in accordance with embodiments of the present disclosure.

FIG. 36 illustrates a flow diagram for a method of analyzing a patient's dentition, in accordance with embodiments of the present disclosure.

FIG. 37 illustrates a flow diagram for a method of generating a report of identified dental and/or gum conditions of a patient, in accordance with embodiments of the present disclosure.

FIG. 38 illustrates a block diagram of an example computing device comprising instructions for an oral health diagnostics system, in accordance with embodiments of the present disclosure.

### DETAILED DESCRIPTION

Described herein are embodiments of an oral health diagnostics system. In embodiments, a dentist or doctor (terms used interchangeably herein) and/or their technicians may gather various information about a patient. Such information may include intraoral 3D scans of the patient's dental arches, x-rays (also referred to herein as radiographs) of the patient's teeth (e.g., optionally including bitewing x-rays of the patient's teeth, panoramic x-rays of the patient's teeth, periapical x-rays of the patient's teeth, etc.), cone-beam computed tomography (CBCT) scans of the patient's jaw, infrared images of the patient's teeth, color 2D images of the patient's teeth and/or gums, biopsy information, malocclusion information, observation notes about the patient's teeth and/or gums, information from sensors of worn devices, patient input about their oral health and/or general health, and so on. The intraoral scans may be generated by an intraoral scanner, the x-rays may be generated by x-ray machines, and so on. Additionally, different data may be gathered at different times.

Each of the different data points may be useful for determining whether the patient has one or more types of oral conditions and/or oral health problems. The oral health diagnostics system provided in embodiments analyzes one or more of the types of data that is available for a given patient, and uses that data to assess numerous different types of oral conditions and/or oral health problems for the patient and identify locations of oral conditions and/or oral health problems. The oral health diagnostics system may further classify and/or rank identified oral conditions and/or oral health problems based on severity in embodiments. In embodiments, the oral health diagnostics system includes a pre-established list of clinical areas for review and systematically processes the scan data and/or other data to populate the list with actual patient data and/or analysis of the actual patient data.

Received data may be processed to systematically detect, localize and classify dental abnormalities and other findings. The oral health diagnostics system further provides a user interface that presents a unified view of each of the types of analyzed oral conditions and/or oral health problems, showing which of the types of oral conditions and/or oral health problems might be of concern and which of the types of oral conditions and/or oral health problems might not be of concern for the patient. Once analysis is complete, results may be displayed visually in the form of a graphical overlay on image data (e.g., a radiograph) and may be summarized in a tooth chart and/or list of detections. A user may be provided with functions such as adding, deleting, correcting and modifying detections. In addition, the user can interact with the user interface to move a tooth or row of teeth, view or change the location and depth of caries (e.g., on bitewing and periapical radiographs) and/or other oral conditions, and perform other actions. Once the user has completed and saved the evaluation, a report can be generated.

In embodiments, overall system design may follow a client-server architecture, in which a frontend component that interacts with the oral health diagnostics system may be or include a web browser, dental software such as a dental practice management system (DPMS) or image management software, which may be external to the oral health diagnostics system. A server may contain a backend component, which may manage communication between remaining components of an interface engine.

In embodiments, the oral health diagnostics system brings together all of the disparate types of information associated with a patient's dental health. The oral health diagnostics system further performs automated analysis for each of the different types of oral conditions and/or oral health problems. For example, the oral health diagnostics system may analyze bitewing radiographs, periapical radiographs, panoramic radiographs, intraoral scans, CBCT scans, and so on using a set of artificial intelligence-based algorithms to assist in the detection of suspected abnormalities (e.g., caries, periapical radiolucencies, periodontal bone loss and dental calculus), and other findings (e.g., dental fillings, dental crowns, dental implants, dental bridges and root-canal fillings, proximity of lower molars to mandibular canal, and impacted teeth). The localization and classification of teeth may be used for tooth charting and reporting. A result of the various automated analyses may then be shown together in a graphical user interface (GUI). The oral health diagnostics system may display an x-ray (e.g., a panoramic x-ray, a bitewing x-ray, a periapical x-ray, etc.) with one or more overlays showing the size, type and location of one or more oral conditions over the x-ray. The oral health diagnostics system may further display a tooth chart with information showing each tooth number and the oral condition or conditions identified for each tooth in the tooth chart.

In embodiments, the oral health diagnostics system greatly increases the speed, accuracy and efficiency of diagnosing oral conditions and/or oral health problems of patients. The oral health diagnostics system enables a dentist to determine, at a single glance of the GUI for the oral health diagnostics system, all of the oral conditions and/or oral health problems that might be of concern for a patient. It enables the dentist to easily and quickly prioritize oral conditions and/or oral health problems to be addressed. Additionally, the oral health diagnostics system may compare different identified oral conditions to determine any correlations between different identified oral conditions. As a result, the oral health diagnostics system may identify some oral conditions as symptoms of other underlying root cause oral conditions, and may identify oral health problems tied to the multiple oral conditions. For example, the oral health diagnostics system may identify tooth crowding and caries formation that results from the tooth crowding.

Additionally, the oral health diagnostics system in embodiments creates presentations and/or reports of oral conditions. Generated presentations may include predictive analysis that shows what will happen if the oral conditions and/or oral health problems are untreated. Generated reports and/or presentations may additionally or alternatively include information on root causes of the patient's oral conditions, treatment plan options, and/or simulations of treatment results. Such presentations and/or reports may be shown to the patient to educate the patient about the condition of their dentition and their options for treating the problems and/or leaving the problems untreated.

A dental practitioner may generate one or more types of relevant dental health information using one or more oral state capture modalities. Examples of oral state capture modalities include x-rays of the patient's teeth (e.g., optionally including bitewing x-rays of the patient's teeth, panoramic x-rays of the patient's teeth, periapical x-rays of the patient's teeth, etc.), intraoral scans of a patient's upper and/or lower dental arches generated by an intraoral scanner (e.g., such as the iTero scanner or Lumina scanner manufactured by Align Technologies®), 2D images of the upper and/or lower dental arch generated by an intraoral scanner (e.g., color 2D images, infrared (IR) 2D images, 2D images generated under specific lighting conditions, etc.), cone-beam computed tomography (CBCT) scans of the patient's jaw, color 2D images of the patient's teeth and/or gums not generated by an intraoral scanner (e.g., from photos taken by a camera), biopsy information, malocclusion information, 3D models of the patent's dental arches generated from intraoral scans, observation notes about the patient's teeth and/or gums, and so on. For example, a dental practitioner may perform an intraoral scan of the oral cavity during an annual or semi-annual dentist appointment to generate a 3D model of the patient's dental arches. In another example, the dental practitioner may additionally or alternatively generate one or more x-rays of the patient's oral cavity (e.g., bite-wing x-rays and/or periapical x-rays) during a dentist appointment. Additional types of dental information may also be gathered when the dentist deems it appropriate to generate such additional information. For example, the dentist may take biopsy samples and send them to a lab for testing and/or may generate a panoramic x-ray and/or a CBCT scan of the patient's oral cavity.

The oral health diagnostics system may register data from multiple different imaging modalities together in embodiments. For example, processing logic may register x-ray images, CBCT scan data, ultrasound images, panoramic x-ray images, 2D color images, NIRI images, and so on to each other and/or to one or more 3D models of the patient's upper and/or lower dental arches. Each of the different imaging modalities may contribute different information about the patient's dentition. For example, NIRI images and x-ray images may identify caries and color images may be used to determine accurate color data, which is usable to determine tooth staining. Additionally, dental x-rays may indicate bone density, bone loss, caries, fillings, bridges, crowns, implants, calculus, and so on. The registered intraoral data from the multiple imaging modalities may be presented together and/or side-by-side. The data from different imaging modalities may be provided as different layers, where each layer may be for a particular imaging modality. This may enable a doctor to turn on or off specific layers to visualize the dental arch with or without information from those particular imaging modalities.

Embodiments provide a general digital workflow covering use of radiographs and/or other oral state capture modalities within a digital platform of integrated products/services to identify oral conditions, and to provide actionable symptom recommendations and/or diagnoses of oral conditions and/or oral health problems. Embodiments further provide software for segmenting image data (e.g., 2D images, radiographs, 3D models, etc.) of a patient's oral cavity (e.g., of dental sites within an oral cavity), identifying oral conditions, and generating actionable symptom recommendations and/or diagnoses of oral health problems. Embodiments further provide software for validation of identified oral conditions, diagnoses of oral health problems, actionable symptom recommendations, and so on using data from multiple different oral state capture modalities. Embodiments further use pattern recognition software (e.g., machine learning/artificial intelligence) to segment teeth of a patient, identify dental conditions associated with specific teeth, generate actionable symptom recommendations and/or diagnoses of oral problems, and so on using data from radiographs, and optionally one or more additional oral state capture modalities.

A few example working definitions are provided below. As used herein, the term “diagnosis” may mean an identification of a nature of an oral health problem by examination of symptoms and/or oral conditions. In some implementations, diagnosis may have the same as the medical term of diagnosis. However, it should be understood that for various contexts the term diagnosis should be afforded its plain and ordinary meaning and not be limited to the medical definition of diagnosis. As used here, the term “actionable symptom recommendations” refers to provided tools for treatment professionals or others (e.g., laypeople, any user of software, etc.) to associate symptoms and/or dental conditions with the nature of oral health problems. Actionable symptom recommendations can be recommendations that do not amount to a full diagnosis of oral health problems of a patient, and may instead provide information usable by a doctor to ultimately make a diagnosis. As used herein, the term “oral state capture modality” may refer to any way to capture the state of a person's oral cavity. Examples of oral state capture modalities include radiographs (e.g., periodontal radiographs, panoramic radiographs, bite-wing radiographs, etc.), intraoral scans, images (e.g., taken by an intraoral scanner, a mobile device of a patient, a camera, etc.), 3D models, data captured by sensors (e.g., an electronic compliance indicator of a dental appliance), patient input, doctor observations, data from health accessories (e.g., from a fitness monitor), etc. As used herein, the term “oral condition” refers to any condition related to the oral cavity. Examples of oral conditions include caries (also referred to as tooth decay and/or cavities), gum inflammation, gum recession, lesions around a tooth (e.g., around a tooth root), cracked or broken teeth, tooth wear, restorations, and so on. Oral conditions may be usable to determine oral health problems. As used herein, the term “oral health problem” may refer to an oral condition that itself constitutes an oral health problem (e.g., caries), or an oral health problem that is associated with an oral condition. For example, gum disease or gingivitis may be associated with inflammation of gums. Periodontal disease may be associated with gum recession and/or other gum issues. Lesions around teeth may be indicative of tooth root issues such as an infection. Other types of oral health problems include oral cancer, causes of bad breath, causes of malocclusion, and so on.

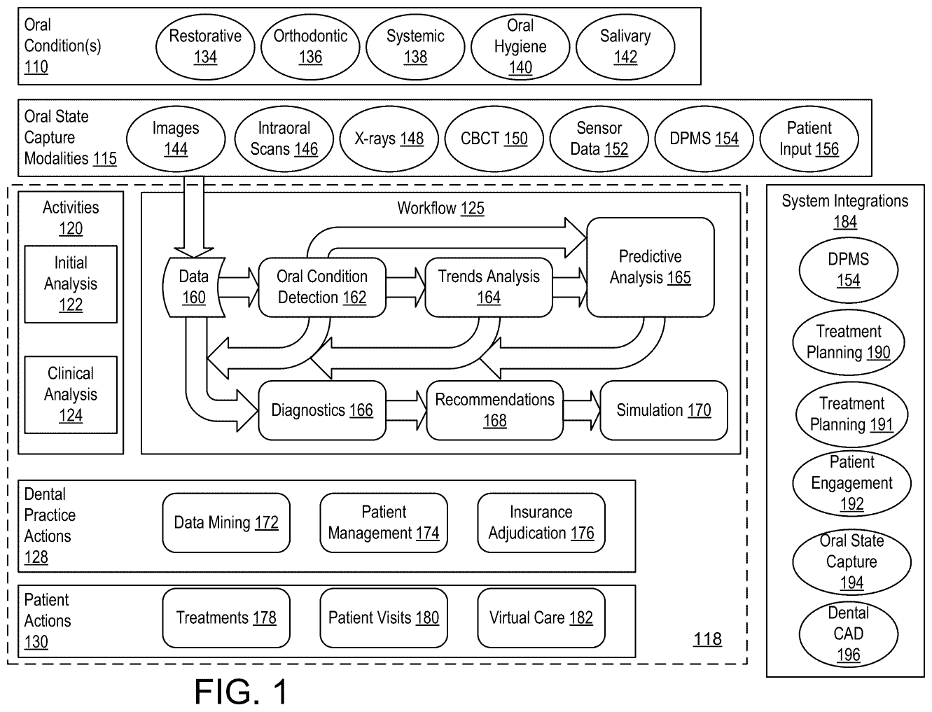

FIG. 1 illustrates a workflow 125 for detecting, predicting, diagnosing and reporting on oral conditions (e.g., oral health conditions) by an oral health diagnostics system 118, in accordance with embodiments of the present disclosure. The workflow 125 may be a general digital workflow covering use of radiographs and/or other oral state capture modalities within a digital platform of integrated products/services to provide identifications of oral conditions and/or actionable symptom recommendations and/or diagnoses of oral health problems associated with such oral conditions. The workflow 125 may be used to assist doctors and/or users of an oral health diagnostics system 118 to assess a patient's oral health, identify oral conditions, diagnose dental health problems, provide actionable symptom recommendations, provide treatment recommendations, and so on. The workflow 125 may be executed by a digital platform of integrated products that provide dental condition identifications, actionable symptom recommendations and/or diagnoses of oral health problems using analysis of data from one or more oral state capture modalities, including radiographs (e.g., generated by radiography machines).

A patient may have one or more oral conditions 110. Oral conditions 110 may include or be related to caries, gum recession, gingival swelling, tooth wear, bleeding, malocclusion, tooth crowding, tooth spacing, plaque, tooth stains, and/or tooth cracks, for example. In some embodiments, the oral conditions 110 may include restorative conditions 134, orthodontic conditions 136, systematic conditions 138, oral hygiene conditions 140, salivary conditions 142, and so on. Restorative conditions 134 may include conditions such as caries that are addressable by performing restorative dental treatment. Such restorative dental treatment may include drilling and filling caries, performing root canals, forming preparations of teeth and applying caps or crowns to the preparations, pulling teeth, adding bridges to teeth, and so on. Restorative conditions may also include results of past restorative treatments of the patient's oral cavity. Examples of past restorations include fillings, caps, crowns, bridges, and so on. Orthodontic conditions may include conditions treatable via orthodontic treatment. Such orthodontic conditions may include a malocclusion (e.g., tooth crowding, overbite, underbite, posterior crossbite, posterior open bite, tooth gaps, etc.). Orthodontic conditions may be associated with restorative conditions in some instances. For example, tooth crowding may cause caries, which results in restorative treatment. Systematic conditions 138 may include conditions such as periodontitis, periodontal bone loss, gum recession, tooth wear, and so on. Systematic conditions 138 may be associated with restorative conditions 134 and/or orthodontic conditions 136. Oral hygiene conditions 140 may include brushing and flossing related conditions, such as development of calculus on teeth, caries, and so on. Oral hygiene conditions 140 may be related to restorative conditions 134, orthodontic conditions 136 and/or systematic conditions 138 in embodiments. Salivary conditions 142 may include a pH level of a patient's mouth that is outside of normal, a low level of saliva, and so on. Salivary conditions 142 may be related to restorative conditions 134, orthodontic conditions 136, systematic conditions 138 and/or oral hygiene conditions 140 in embodiments. For example, the detection and identification of salivary conditions may be used as an input to an ML model that can use such information to assess periodontal disease, acid reflux, vomiting, poor diet, oral cancer, and/or oropharyngeal cancer. For example, biomarkers of saliva may be used to assist in the assessment and/or management of periodontal disease. Tooth erosion, caries and/or saliva biomarkers may be used to identify acid reflux, vomiting and/or poor diet. In some instances, an oral condition of a patient may include a cross-classification. Such oral conditions may belong to multiple different categories of oral conditions 110. For example, caries may be a restorative condition 134, an orthodontic condition 136 and an oral hygiene condition 140.

A patient may have one or more oral health problems that may be root problems for the oral conditions and/or that may be caused by the oral conditions. In some embodiments, an oral condition also constitutes an oral health problem. Examples of oral health problems include caries, periodontal disease, a tooth root issue, a cracked tooth, a broken tooth, oral cancer, a cause of bad breath, and/or a cause of a malocclusion.

A dental practice (e.g., a group practice or solo practice) may capture data about a patient's oral state using one or more oral state capture modalities 115. A common oral state capture modality used by dental practices are radiographs (i.e., x-rays) 148 generated by radiography machines. There are multiple different types of x-rays that a genal practice may capture of a patient's oral cavity, including bite-wing x-rays, panoramic x-rays and periapical x-rays.

A bite-wing x-ray is a type of dental radiograph used to detect dental caries (cavities) and monitor the health of teeth and supporting bone. During a bite-wing x-ray, the patient bites down on a small tab or wing-shaped device attached to the x-ray film or sensor. This helps keep the film or sensor in place while the x-ray is taken. An x-ray machine (also referred to as a radiography machine) is positioned outside the mouth to capture images of the upper and lower teeth on one side of the mouth at a time. Accordingly, a bite-wing x-ray includes upper and lower teeth of one side of a patient's mouth. In embodiments, bite-wing x-rays are useful for detecting cavities between teeth and for assessing the fit of dental fillings and crowns. Bite-wing x-rays may also be used to help in diagnosing gum disease and/or to monitor bone levels around the teeth in embodiments.

A periapical x-ray, also known as a periapical radiograph, is a type of dental x-ray that focuses on specific areas of the mouth, particularly individual teeth and the surrounding bone. During a periapical x-ray, the dentist or dental radiographer positions an x-ray machine so that it captures detailed images of one or more teeth from crown to root, as well as the surrounding bone structure and supporting tissues. Periapical x-rays may provide a comprehensive view of the entire tooth, including the root tip (apex) and the bone around the tooth's root. In embodiments, periapical x-rays may be used to help diagnose oral health problems such as tooth decay (caries), infections or abscesses at the root of a tooth, bone loss around a tooth due to periodontal (gum) disease, abnormalities in the root structure or surrounding bone, evaluation of dental trauma or injuries, and so on. Periapical x-rays may also be used to assist in assessment of the status of teeth prior to dental procedures such as root canal treatment or extraction.

A panoramic x-ray, also known as a panoramic radiograph or orthopantomogram (OPG), is a type of dental radiograph that provides a comprehensive view of the entire mouth, including the teeth, jaws, temporomandibular joints (TMJ), and surrounding structures in a single image. During a panoramic x-ray, the patient stands or sits in an upright position while an x-ray machine rotates around their head in a semi-circle. The x-ray machine captures a continuous image as it moves, creating a detailed panoramic view of the entire oral and maxillofacial region. In embodiments, a panoramic x-ray may be used to assist in evaluation of the development and position of teeth, including impacted teeth, assessing the health of the jawbone and surrounding structures, detecting cysts, tumors, or other abnormalities in the jaw or adjacent tissues, planning orthodontic treatment by assessing tooth alignment and development, evaluating the placement and condition of dental implants, and/or diagnosing temporomandibular joint (TMJ) disorders or other jaw-related issues.

Another oral state capture modality that is increasingly common in dental practices are intraoral scans 146, and three-dimensional (3D) models of dental arches (or portions thereof) based on such intraoral scans. Intraoral scans are produced by an intraoral scanning system that generally includes an intraoral scanner and a computing device connected to the intraoral scanner by a wired or wireless connection. The intraoral scanner is a handheld device equipped with one or more small cameras and/or optical sensors. The dentist or dental professional moves the intraoral scanner around the patient's mouth, capturing multiple 3D images or scans of the teeth and surrounding structures from various angles. As the intraoral scanner captures the images or scans, they may be processed and displayed on a computer screen in real-time or near real-time. The collected images or scans are stitched together to create a complete 3D digital model of the patient's teeth and oral cavity. This digital impression can be manipulated, analyzed, and shared electronically with dental laboratories or specialists as needed.

An intraoral scan application executing on the computing device of an intraoral scanning system may generate a 3D model (e.g., a virtual 3D model) of the upper and/or lower dental arches of the patient from received intraoral scan data (e.g., images/scans). To generate the 3D model(s) of the dental arches, the intraoral scan application may register and stitch together the intraoral scans generated from an intraoral scan session. In one embodiment, performing image registration includes capturing 3D data of various points of a surface in multiple intraoral scans, and registering the intraoral scans by computing transformations between the intraoral scans. The intraoral scans may then be integrated into a common reference frame by applying appropriate transformations to points of each registered intraoral scan.

In one embodiment, registration is performed for each pair of adjacent or overlapping intraoral scans. Registration algorithms may be carried out to register two adjacent intraoral scans for example, which essentially involves determination of the transformations which align one intraoral scan with the other. Registration may involve identifying multiple points in each intraoral scan (e.g., point clouds) of a pair of intraoral scans, surface fitting to the points of each intraoral scans, and using local searches around points to match points of the two adjacent intraoral scans. For example, the intraoral scan application may match points, edges, curvature features, spin-point features, etc. of one intraoral scan with the closest points, edges, curvature features, spin-point features, etc. interpolated on the surface of the other intraoral scan, and iteratively minimize the distance between matched points. Registration may be repeated for each adjacent and/or overlapping scans to obtain transformations (e.g., rotations around one to three axes and translations within one to three planes) to a common reference frame. Using the determined transformations, the intraoral scan application may integrate the multiple intraoral scans into a first 3D model of the lower dental arch and a second 3D model of the upper dental arch.

The intraoral scan data may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Oral state capture modalities 115 may additionally or alternatively include one or more types of images 144 (e.g., 2D and/or 3D images) of a patient's oral cavity. In addition to generating intraoral scans, intraoral scanning systems may additionally be used to generate color 2D images of a patient's oral cavity. These color 2D images may be registered to the intraoral scans generated by the intraoral scanning system, and may be used to add color information to 3D models of a patient's dental arches. Intraoral scanning systems may additionally or alternatively generate 2D near infrared (NIR) images, images generated using fluorescent imaging, images generated under particular wavelengths of light, and so on. Such image generation may be interleaved with 3D image or intraoral scan generation by an intraoral scanner.

Dental practices may additionally include cameras for generating 3D images of a patient's oral cavity and/or cameras for generating 2D images of a patient's oral cavity. Additionally, a patient may generate images of their own oral cavity using personal cameras, mobile devices (e.g., tablet computers or mobile phones), and so on. In some instances, patients may generate images of their oral cavity based on the instruction of an application or service such as a virtual dental care application or service. In some cases, images of a patient's oral cavity (e.g., those taken by a dental practitioner or by a patient themselves) may be taken while the patient wears a cheek retractor to retract the lips and cheeks of the patient and provide better access for dental imaging (i.e., for intraoral photography).

Some dental practices also use cone beam computed tomography (CBCT) 150 as an oral state capture modality 115. CBCT is a medical imaging technique that uses a cone-shaped X-ray beam to create detailed 3D images of the dental and maxillofacial structures. CBCT scanners may be specifically designed for imaging the head and neck region, including the teeth, jawbones, facial bones, and surrounding tissues. A CBCT machine emits a cone-shaped X-ray beam that rotates around the patient's head. A detector on the opposite side of the machine captures a sequence of X-ray images from different angles. The x-ray images are processed to reconstruct them into a detailed 3D volumetric dataset. This dataset provides a comprehensive view of the patient's oral anatomy in three dimensions. CBCT scans may facilitate accurate diagnosis of various dental and maxillofacial conditions, including impacted teeth, dental infections, bone abnormalities, and temporomandibular joint disorders. In embodiments, CBCT imaging may be used for various dental and maxillofacial applications, including implant planning, orthodontic treatment planning, endodontic evaluations, oral surgery, and periodontal assessments.

For image-based oral state capture modalities, multiple depictions and views of the oral cavity and internal structures can be captured (e.g., in radiographs, intraoral scans, etc.). Examples of views include occlusal views, buccal views, lingual views, proximal-distal views, panoramic views, periapical views, bitewings views, and so on.

Oral state capture modalities 115 may additionally or alternatively include sensor data 152 from one or more worn sensors. In some instances, a patient may be prescribed a compliance device (e.g., an electronic compliance indicator), an orthodontic aligner, a palatal expander, a sleep apnea device, a night guard, a retainer, or other dental appliance to be worn by the patient. Any such dental appliance may include one or more integrated sensors, which may include force sensors, pressure sensors, pH sensors, sensors for measuring saliva bacterial content, temperature sensors, contact sensors, bio sensors, and so on. Sensor data from the sensor(s) of a dental appliance worn by a patient may be reported to oral health diagnostics system 118 in embodiments. Additionally, or alternatively, a patient may wear one or more consumer health monitoring tools or fitness tracking devices, such as a watch, ring, etc. that includes sensors for tracking patient activity, heartbeat, blood pressure, electrical heart activity (e.g., generates an electrocardiogram), breathing, sleep patterns, body temperature, and so on. Data collected by such fitness tracking devices may also be reported to the oral health diagnostics system 118 in embodiments.

Oral state capture modalities 115 may additionally or alternatively include patient input 156. Patient input may include patient complaints of pain, numbness, bleeding, swelling, clicking, etc. in one more regions of their mouth. Patient input may further include input on overall health, such as information on underlying health conditions (e.g., diabetes, high blood pressure, etc.), on patient age, and so on. Such patient input may be captured and input into an oral health diagnostics system 118 in embodiments. For example, a doctor or patient may type up notes or annotations indicating the patient input, which may be ingested by the oral health diagnostics system 118 with other oral state capture modalities 115.

In some embodiments, an oral health diagnostics system 118 may include one or more system integrations 184 with external systems, which may or may not be dental related. Such system integrations 184 may be for data to be provided to the oral health diagnostics system 118 and/or for the oral health diagnostics system 118 to provide data to the other system(s).

Dental practices generally use a dental practice management system (DPMS) 154 for managing the dental practices. A DPMS 154 is a software solution designed to streamline and automate various administrative and clinical tasks within a dental practice. DPMS 154 are tailored for the needs of dental offices and help dentists and their staff manage patient information, appointments, billing, and other aspects of dental practice management efficiently. A DPMS 154 allows a dental practice to maintain comprehensive patient records, including demographic information, medical history, treatment plans, and clinical notes. The DPMS 154 provides a centralized database that enables dental staff to access patient information quickly and efficiently. DPMS 154 generally includes features for scheduling patient appointments, managing appointment calendars, and sending appointment reminders to patients. DPMS 154 provides tools for creating and managing treatment plans for patients, including digital charting of dental procedures, diagnoses, and treatment progress. This helps dentists and hygienists track patient care effectively and ensure continuity of treatment. DPMS 154 may help to automate billing processes, including generating invoices, processing payments, and managing insurance claims. It can also verify patient insurance coverage, estimate treatment costs, and submit claims electronically to insurance providers for faster reimbursement. DPMS 154 may generate financial reports and analytics to help dental practices track revenue, expenses, and profitability.

In embodiments, data from a DPMS 154 is used as one type of oral state capture modality 115. Oral health diagnostics system 118 may interface with a DPMS 154 to retrieve patient records for a patient, including past oral conditions of the patient, doctor notes, patient information (e.g., name, gender, age, address, etc.), and so on.

In addition to an ability to ingest data from a DPMS 154, oral health diagnostics system 118 in embodiments may be able to generate reports and/or other outputs that can be ingested by the DPMS 154. Accordingly, once the oral health diagnostics system 118 performs an assessment of a patient's oral conditions, oral health problems, treatment recommendations, etc., the oral health diagnostics system 118 may format such data into a format that can be understood by the DPMS 154. The oral health diagnostics system may then automatically add new data entries to the DPMS 154 for a patient based on an analysis of patient data from one or more oral state capture modalities 115.

The oral health diagnostics system 118 may have a system integration with one or more oral state capture systems (e.g., such as an intraoral scanner or intraoral scanning system) 194, from which intraoral scans 146, images 144, 3D models, and/or data from other oral state capture modalities may be received. Examples of oral state capture systems include an intraoral scanning system, a radiograph system or machine, a CBCT machine, and so on.

In embodiments, an output of oral health diagnostics system 118 may be provided to a dental computer aided drafting (CAD) system 196, such as Exocad® by Align Technology. The dental CAD system 196 may be used for designing dental restorations such as crowns, bridges, inlays, onlays, veneers, and dental implant restorations. The dental CAD system 196 may provide a comprehensive suite of tools and features that enable dental professionals to create precise and customized dental restorations digitally. The dental CAD system 196 may import digital impressions (e.g., 3D digital models of a patient's dental arches) captured using intraoral scanners, and may further import data on a patient's oral health from oral health diagnostics system 118. For example, the oral health diagnostics system 118 may export a report on a patient's oral health to the dental CAD system 196, which may be used together with a digital impression of the patient's dental arches to develop an appropriate restoration for the patient, for implant planning, for planning of surgery for implant placement, and so on.

In embodiments, oral health diagnostics system 118 may have a system integration 184 with a patient engagement system (e.g., which may include a patient portal and/or patient application) 192. The patient portal may be a portal to an online patient-oriented service. Similarly, the patient application may be an application (e.g., on a patient's mobile device, tablet computer, laptop computer, desktop computer, etc.) that interfaces with a patient-oriented service.

In an example, oral health diagnostics system 118 may integrate with a virtual care system. The virtual care system may provide a suite of digital tools and services designed to enhance patient care and communication between orthodontists/dentists and their patients. The virtual care system may leverage technology to facilitate remote monitoring, consultation, and treatment planning, allowing patients to receive dental care more conveniently and effectively.

In one embodiment, the patient engagement system 192 is or includes a virtual care system that may provide remote monitoring, teleconsultation, treatment planning, patient education and engagement, data management, and data analytics. With respect to remote monitoring, the virtual care system enables orthodontists and dentists to remotely monitor their patients' treatment progress (e.g., for orthodontic treatment) using advanced digital tools. This may include the use of smartphone apps, patient portals, or other software platforms that allow patients to capture and upload photos or videos of their teeth and orthodontic appliances. Such patient uploaded data may be provided to oral health diagnostics system 118 for automated assessment in embodiments. With regards to patient education and engagement, the virtual care system may provide reports, presentations, etc. generated by oral health diagnostics system 118 to patients (e.g., via a patient portal and/or application). For example, the dental health diagnostics system 118 may automatically generate informational videos, treatment progress trackers, compliance reminders, reports, presentations, and so on that are tailored to a patient's oral health, which may be provided to the patient via the patient portal and/or application.

In embodiments, oral health diagnostics system 118 may have a system integration 184 with one or more treatment planning system 190 and/or treatment management system 191 such as ClinCheck® provided by Align Technology®. For example, oral health diagnostics system 118 may have a system integration with an orthodontic treatment planning system and/or with a restorative dental treatment planning system. A treatment planning system 190 may use digital impressions and/or a report output by oral health diagnostics system 118 to plan an orthodontic treatment and/or a restorative treatment (e.g., to plan an ortho-restorative treatment). The treatment planning system 190 may plan and simulate orthodontic and/or restorative treatments. Treatment management system 191 may then receive data during treatment and determine updates to the treatment based on the treatment plan and the updated data.

In an example, an orthodontic treatment planning system may use advanced 3D imaging technology to create virtual models of patients' teeth and jaws based on digital impressions or intraoral scans. These digital models may be used to plan and simulate the entire course of orthodontic treatment, including the movement of individual teeth and the progression of treatment over time. Orthodontists can specify the desired tooth movements, treatment duration, and other parameters, taking into account a report provided by oral health diagnostics system 118, to create personalized treatment plans tailored to each patient's unique anatomy, oral health, and preferences. The orthodontic treatment planning system enables orthodontists to simulate the step-by-step progression of orthodontic treatment virtually, showing patients how their teeth will gradually move and align over the course of treatment. Orthodontists can visualize the planned tooth movements in 3D and make adjustments as needed to optimize treatment outcomes. The orthodontic treatment planning system may provide orthodontists and patients with visualizations of the predicted treatment outcomes, including before-and-after simulations that demonstrate the expected changes in tooth position and alignment, and how those changes might affect the patient's overall oral health as optionally predicted by the oral health diagnostics system 118. These visualizations help patients understand the proposed treatment plan and make informed decisions about their orthodontic care.

During treatment, updated data may be gathered about a patient's dentition, and such data (e.g., in the form of one or more oral state capture modalities 115) may be processed by the oral health diagnostics system 118, optionally in view of an already generated orthodontic treatment plan, to generate an updated report of the patient's overall oral health. The updated report may be provided by the oral health diagnostics system 118 to the orthodontic treatment planning system and/or orthodontic treatment management system to enable the orthodontic treatment planning/management system to perform informed modifications to the treatment plan. Thus, integration of the oral health diagnostics system with the orthodontic treatment planning system and/or treatment management system supports an iterative design process, allowing orthodontists to review and refine treatment plans based on patient feedback, clinical considerations, treatment progress, and automated reports output by oral health diagnostics system 118. This enables orthodontists to make adjustments to the treatment plan within the orthodontic treatment planning system and/or treatment management system and generate updated simulations to assess the impact of these changes on the final treatment outcome.

Accordingly, oral health diagnostics system 118 may perform treatment planning and/or management on its own and/or based on integration with one or more treatment planning systems for planning and/or managing orthodontic treatment, restorative treatment, and/or ortho-restorative treatment. An output of such planning may be an orthodontic treatment plan, a restorative treatment plan, and/or an ortho-restorative treatment plan. A doctor may provide one or more modifications to the generated treatment plan, and the treatment plan may be updated based on the doctor modifications.

In addition to those systems mentioned herein that oral health diagnostics system 118 may integrate with, oral health diagnostics system 118 may integrate with any system, application, etc. related to dentistry and/or orthodontics.

Oral health diagnostics system 118 may execute a workflow 125 that includes processing and analysis of data 160 from one or more oral state capture modalities 115. The workflow 125 may be roughly divided into activities 120 associated with an initial analysis 122 of a patient's oral health and operations associated with a clinical analysis 124 of the patient's oral health in some embodiments. One of more of the operations of the workflow may be performed by and/or assisted by application of artificial intelligence and/or machine learning models in embodiments. Multiple embodiments are discussed with reference to machine learning models herein. It should be understood that such embodiments may also implement other artificial intelligence systems or models, such as large language models in addition to or instead of traditional machine learning models such as artificial neural networks.

The workflow may include performing oral condition detection at block 162. To perform oral condition detection, a segmentation pipeline may process the data 160 to segment the data into one or more teeth and into one or more oral conditions that may be associated with the one or more of the teeth. The segmentation pipeline may include multiple different trained machine learning models and additional logic that operate on the data and/or on outputs of other trained machine learning models and/or logic to identify specific teeth and apply tooth numbering to the teeth, identify oral conditions, associate the oral conditions to specific teeth, determine locations on the teeth at which the oral conditions are identified, and so on. The output of block 162 may include masks indicating pixels of input image data (e.g., radiographs, 3D models, 2D images, etc.) associated with particular dental conditions, indications of which teeth have detected oral conditions, masks indicating, for each tooth in the input data, which pixels represent that tooth, and so on. The output of block 162 may be input into one or more of block 164, block 165 and/or block 166 in embodiments.

At block 164, trends analysis may be performed based on the output of block 162 and on prior oral conditions of the patient detected at one or more previous times. Trends analysis may include comparing oral conditions at one or more previous times to current oral conditions of the patient. Based on the comparison, an amount of change of one or more of the oral conditions may be determined, a rate of change of the one or more oral conditions may be determined, and so on. Trends analysis may be performed using traditional image processing and image comparison. Additionally, or alternatively, trends analysis may be performed by inputting current and past oral conditions and/or data from one or more oral state capture modalities into one or more trained machine learning models. An output of block 164 may be provided to block 165 and/or block 166 in embodiments.

At block 165, predictive analysis may be performed on the output of block 162, on the output of block 164 and/or on prior oral conditions of the patient detected at one or more previous times. Predictive analysis may include predicting future oral conditions of the patient based on input data. Predictive analysis may be performed with or without an input of prior oral conditions. If prior oral conditions are used in addition to current oral conditions to predict future conditions, then the accuracy of the prediction may be increased in embodiments. In some embodiments, predictive analysis is performed by projecting identified trends determined from the trends analysis into the future. In some embodiments, predictive analysis is performed by inputting the current and/or past oral conditions into one or more trained machine learning models that output predictions of future dental conditions. Predictive analysis may be performed using traditional image processing and image comparison. Additionally, or alternatively, predictive analysis may be performed by inputting current and/or past oral conditions, trends and/or data from one or more oral state capture modalities into one or more trained machine learning models. In embodiments, the predictive analysis generates synthetic image data, which may include panoramic views, periapical views, bitewing views, buccal views, lingual views, occlusal views, and so on of the predicted future oral conditions. Generated synthetic image data may be in the form of synthetic radiographs, synthetic color images, synthetic 3D models, and so on. An output of block 165 may be provided to block 166 in embodiments.

At block 166, automated diagnostics of a patient's oral health may be performed based on data 160 and/or based on outputs of block 162, block 164 and/or block 165 in embodiments. In embodiments, one or more trained machine learning (ML) models and/or artificial intelligence (AI) models may process input data to perform the diagnostics. An output of the ML models and/or AI models may include actionable symptom recommendations usable to diagnose oral health problems and/or actual diagnoses of oral health problems associate with the detected oral conditions.

At block 168, based on the data 160, oral conditions identified at block 162, output of trends analysis performed at block 164, output of predictive analysis performed at block 165 and/or output of diagnostics performed at block 166, processing logic may generate one or more treatment recommendations for a patient. The treatment recommendations may include multiple different treatment options, with different probabilities of success associated with the different treatment options.

At block 170, processing logic may generate one or more treatment simulations based on one or more of the treatment recommendations. The treatment simulations may include an alternative predictive analysis that shows predicted states of oral conditions and/or oral health problems of the patient after treatment is performed, or after one or more stages of treatment are performed. Treatment simulations may include generated synthetic image data, which may be in the form of synthetic radiographs, synthetic color images, synthetic 3D models, and so on. The synthetic image data may show what a patient's oral cavity would look like after treatment and/or after one or more intermediate and/or final stages of a multi-stage treatment (e.g., such as orthodontic treatment or ortho-restorative treatment).

Post treatment simulations may be compared to predicted simulations of the predicted states of the oral conditions absent treatment (e.g., as determined at block 165) in embodiments.

In embodiments, a report may be generated including the data 160 and/or outputs of one or more of blocks 162, 164, 165, 166, 168 and/or 170. The report may include labeled images, a dental chart, notes, annotations, and/or other information. The report may include a dynamic presentation (e.g., a video) that shows progression of dental conditions over time in some embodiments. The report may be stored in a data store and/or exported to one or more other systems (e.g., DPMS 154, treatment planning system 190, patient engagement system 192, dental CAD system 196).

The oral health diagnostics system 128 may perform multiple dental practice actions 128 and/or patient actions 130 in addition to, or instead of, storing a generated report and/or exporting the report to other systems. Examples of dental practice actions 128 that may be performed include data mining 172, patient management 174 and/or insurance adjudication 176. Examples of patient actions 128 that may be performed include treatments 178, patient visits 180 and/or virtual care 182. One or more of the actions may be performed based on leveraging external systems in embodiments. For example, virtual care 182 may be performed based on leveraging a patient portal and/or application of a virtual dental care system. Patient visits 180 may be performed based on leveraging a DPMS 154. Treatments 178 may be performed based on leveraging a treatment planning system 190 for planning, tracking and/or management of a treatment. Patient management 174 and/or insurance adjudication 176 may be performed based on leverage of a DPMS 154.

Data mining 172 may include analysis of patient data of a dental practice in embodiments. Data mining may be performed for a single dental practice or for multiple different dental practices. Data mining may be performed to determine strengths and weaknesses of a dental practice relative to other dental practices and/or to determine strengths and weaknesses of individual doctors relative to other doctors within a dental practice and/or outside of a dental practice (e.g., in a geographic region). As a result of data mining 172, a report may be generated indicating things for a doctor to focus on, types of procedures that a doctor should perform more, oral state capture modalities that a doctor should use more frequently, and so on.

Patient management 174 for a dental practice may include a range of tasks and processes aimed at providing quality care and ensuring positive experiences for patients throughout their interactions with the dental practice. Patient management may include appointment scheduling, patient registration and check-in, medical and dental history and records management (e.g., including information about past treatments, allergies, medications, and relevant medical conditions for each patient), treatment planning and coordination, financial management and billing (e.g., including collecting payments, processing insurance claims, providing cost estimates, and discussing payment options or financing arrangements with patients), patient communication and education (e.g., providing information about treatments, procedures, and oral hygiene instructions, as well as addressing patient concerns, answering questions, and maintaining open lines of communication throughout the treatment process), follow-up and recall, and patient satisfaction and feedback management.

Insurance adjudication 176 for a dental practice refers to the process of evaluating and determining the coverage and reimbursement for dental services provided to patients by their dental insurance carriers. Insurance adjudication 176 involves submitting claims to insurance companies, reviewing the claims for accuracy and completeness, and processing them according to the terms of the patient's insurance policy. After providing dental services (e.g., treatment) to a patient, the dental practice submits a claim to the patient's insurance company electronically or via paper. The claim includes information such as the patient's demographic details, treatment provided, diagnosis codes, procedure codes (CPT or ADA codes), and any other relevant documentation. In embodiments, such documentation is automatically prepared by oral health diagnostics system 118. Upon receiving an insurance claim, the insurance company reviews the claim to determine coverage eligibility and benefits according to the terms of the patient's insurance policy. The insurance company evaluates the claim and calculates the amount of coverage and reimbursement based on the patient's benefits plan, contractual agreements with the dental office, and applicable fee schedules. The adjudication process may involve verifying the accuracy of the submitted information, applying deductibles, copayments, and coinsurance, and determining the allowed amount for each covered service. In embodiments, oral health diagnostics system 118 may automatically generate responses to inquiries from insurance companies about already submitted claims. After adjudicating a claim, the insurance company sends an Explanation of Benefits (EOB) to the dental office and the patient. The EOB outlines the details of the claim, including the services rendered, the amount covered by insurance, any patient responsibility (such as copayments or deductibles), and the reason for any denials or adjustments. If the claim is approved, the insurance company issues payment to the dental office for the covered services. The dental office then reconciles the payment received with the treatment provided and updates the patient's financial records accordingly. If there are any discrepancies or denials, the dental office may need to follow up with the insurance company to resolve issues or appeal denied claims. In embodiments, oral health diagnostics system 118 automatically handles such follow-ups. After insurance adjudication, the dental office bills the patient for any remaining balance or patient responsibility not covered by insurance, such as deductibles, copayments, or non-covered services. The patient is responsible for paying these amounts according to the terms of their insurance policy and the dental office's financial policies.

In some embodiments, the workflow 125 can be implemented with just a few clicks of a web portal or dental practice application to enable doctors to purchase and activate one or more oral health diagnostics services. When patient records (e.g., data from one or more oral state capture modalities 115, such as intraoral scans 146, virtual care images 144, digital x-rays 148, etc.) are collected as a routine part of a dental appointment, these records may be uploaded to a digital platform of the oral health diagnostics system 118. The oral health diagnostics system 118 may start an analysis for the different oral (e.g., clinical) conditions that have been activated for the patient by the doctor, and may generate a report on the different identified oral conditions. In seconds the doctor may receive a report that has visual indications with colored clues of assessments for a number of possible dental conditions, dental health problems, and so on. As an example, the oral health diagnostics system 118 can send this data to the treatment planning system 190 or treatment management system 191 to process.

In some embodiments, the treatment planning system 190 can integrate this information with an orthodontic treatment plan. The doctor can share the analysis visually chairside with the patient and provide treatment recommendations based on the diagnosis. This can occur on the treatment planning and/or management system 190, 191 or on an application on an intraoral scanning system or x-ray system, for example. The doctor can also share the analysis with the patient and send visual assessments via patient engagement system 192. Integrated education modules may provide interactive context sensitive education tools designed to help the doctor diagnose and help convert the patient to the treatment in embodiments.

Some of the analyses that are performed to assess the patient's dental health are oral health condition progression analyses that compare oral conditions of the patient at multiple different points in time. For example, one carries assessment analysis may include comparing caries at a first point in time and a second point in time to determine a change in severity of the caries between the two points in time, if any. Other time-based comparative analyses that may be performed include a time-based comparison of gum recession, a time-based comparison of tooth wear, a time-based comparison of tooth movement, a time-based comparison of tooth staining, and so on. In some embodiments, processing logic automatically selects data collected at different points in time to perform such time-based analyses. Alternatively, a user may manually select data from one or more points in time to use for performing such time-based analyses.

In one embodiment, the different types of oral conditions for which analyses are performed and that are included in the detected oral conditions include tooth cracks, gum recession, tooth wear, occlusal contacts, crowding and/or spacing of teeth and/or other malocclusions, plaque, tooth stains, calculus, bone loss, bridges, fillings, implants, crowns, impacted teeth, root-canal fillings, and caries. Additional, fewer and/or alternative oral conditions may also be analyzed and reported. In embodiments, multiple different types of analyses are performed to determine presence, location and/or severity of one or more of the oral conditions. One type of analysis that may be performed is a point-in-time analysis that identifies the presence and/or severity levels of one or more oral conditions at a particular point-in-time based on data generated at that point-in-time (e.g., at block 162). For example, a single x-ray image of a dental arch may be analyzed to determine whether, at a particular point-in-time, a patient's dental arch included any caries, gum recession, tooth wear, problem occlusion contacts, crowding, spacing or tooth gaps, plaque, tooth stains, and/or tooth cracks. Another type of analysis that may be performed is a time-based analysis that compares oral conditions at two or more points in time to determine changes in the oral conditions, progression of the oral conditions and/or rates of change of the oral conditions (e.g., at block 164). For example, in embodiments a comparative analysis is performed to determine differences between x-rays taken at different points in time. The differences may be measured to determine an amount of change, and the amount of change together with the times at which the intraoral scans that were used to generate the x-rays were taken may be used to determine a rate of change. This technique may be used, for example, to identify an amount of change and/or a rate of change for tooth wear, staining, plaque, crowding, spacing, gum recession, caries development, tooth cracks, and so on.

In embodiments, one or more trained models are used to perform at least some of the one or more oral condition analyses. The trained models may include physics models and/or machine learning models, for example. In one embodiment, a single model may be used to perform multiple different analyses (e.g., to identify any combination of tooth cracks, gum recession, tooth wear, occlusal contacts, crowding and/or spacing of teeth and/or other malocclusions, plaque, tooth stains, and/or caries). Additionally, or alternatively, different models may be used to identify different oral conditions. For example, a first model may be used to identify tooth cracks, a second model may be used to identify tooth wear, a third model may be used to identify gum recession, a fourth model may be used to identify problem occlusal contacts, a fifth model may be used to identify crowding and/or spacing of teeth and/or other malocclusions, a sixth model may be used to identify plaque, a sixth model may be used to identify tooth stains, and/or a seventh model may be used to identify caries.

In one embodiment, at block 162 intraoral data from one or more points in time are input into one or more trained machine learning models that have been trained to receive the intraoral data as an input and to output classifications of one or more types of oral conditions. In one embodiment, the trained machine learning model(s) is trained to identify areas of interest (AOIs) from the input intraoral data and to classify the AOIs based on oral conditions. The AOIs may be or include regions associated with particular oral conditions. The regions may include nearby or adjacent pixels or points that satisfy some criteria, for example. The intraoral data that is input into the one or more trained machine learning model may include three-dimensional (3D) data and/or two-dimensional (2D) data. The intraoral data may include, for example, one or more 3D models of a dental arch, one or more projections of one or more 3D models of a dental arch onto one or more planes (optionally comprising height maps), one or more x-rays of teeth, one or more CBCT scans, a panoramic x-ray, near-infrared and/or infrared imaging data, color image(s), ultraviolet imaging data, intraoral scans, one or more bitewing x-rays, one or more periapical x-rays, and so on. If data from multiple imaging modalities are used (e.g., panoramic x-rays, bitewing x-rays, periapical x-rays, CBCT scans, 3D scan data, color images, and NIRI imaging data), then the data may be registered and/or stitched together so that the data is in a common reference frame and objects in the data are correctly positioned and oriented relative to objects in other data. One or more feature vectors may be input into the trained model, where the feature vectors include multiple channels of information for each point or pixel of an image. The multiple channels of information may include color channel information from a color image, depth channel information from intraoral scan data, a 3D model or a projected 3D model, intensity channel information from an x-ray image, and so on.

The trained machine learning model(s) may output a probability map, where each point in the probability map corresponds to a point in the intraoral data (e.g., a pixel in an intraoral image or point on a 3D surface) and indicates probabilities that the point represents one or more dental classes. In one embodiment, a single model outputs probabilities associated with multiple different types of dental classes, which includes one or more oral health condition classes. In an example, a trained machine learning model may output a probability map with probability values for a teeth dental class and a gums dental class. The probability map may further include probability values for tooth cracks, gum recession, tooth wear, occlusal contacts, crowding and/or spacing of teeth and/or other malocclusions, plaque, tooth stains, healthy area (e.g., healthy tooth and/or healthy gum) and/or caries. In the case of a single machine learning model that can identify each of tooth cracks, gum recession, tooth wear, occlusal contacts, crowding and/or spacing of teeth and/or other malocclusions, plaque, tooth stains, and caries, eleven valued labels may be generated for each pixel, one for each of teeth, gums, healthy area, tooth cracks, gum recession, tooth wear, occlusal contacts, crowding and/or spacing of teeth and/or other malocclusions, plaque, tooth stains, and caries. The corresponding predictions have a probability nature: for each pixel there are multiple numbers that may sum up to 1.0 and can be interpreted as probabilities of the pixel to correspond to these classes. In one embodiment, the first two values for teeth and gums sum up to 1.0 and the remaining values for healthy area, tooth cracks, gum recession, tooth wear, occlusal contacts, crowding and/or spacing of teeth and/or other malocclusions, plaque, tooth stains, and/or caries sum up to 1.0.

In some instances, multiple machine learning models are used, where each machine learning model identifies a subset of the possible oral conditions. For example, a first trained machine learning model may be trained to output a probability map with three values, one each for healthy teeth, gums, and caries. Alternatively, the first trained machine learning model may be trained to output a probability map with two values, one each for healthy teeth and caries. A second trained machine learning model may be trained to output a probability map with three values (one each for healthy teeth, gums and tooth cracks) or two values (one each for healthy teeth and tooth cracks). One or more additional trained machine learning models may each be trained to output probability maps associated with identifying specific types of oral conditions.

In embodiments, image processing and/or 3D data processing may be performed on radiographs and/or other dental data. Such image processing and/or 3D data processing may be performed using one or more algorithms, which may be generic to multiple types of oral conditions or may be specific to particular oral conditions. For example, a trained model may identify regions on a dental radiograph that include caries, and image processing may be performed to assess the size and/or severity of the identified caries. The image processing may include performing automated measurements such as size measurements, distance measurements, amount of change measurements, rate of change measurements, ratios, percentages, and so on. Accordingly, the image processing and/or 3D data processing may be performed to determine severity levels of oral conditions identified by the trained model(s). Alternatively, the trained models may be trained both to classify regions as caries and to identify a severity and/or size of the caries.

The one or more trained machine learning models that are used to identify, classify and/or determine a severity level for oral conditions may be neural networks such as deep neural networks or convolutional neural networks. Such machine learning models may be trained using supervised training in embodiments.

A dentist, after a quick glance at the dental diagnostics summary, may determine that a patient has carries, clinically significant tooth wear, and crowding/spacing and/or other malocclusions and/or oral conditions.

In embodiments, the oral health diagnostics system, and in particular the dental diagnostics summary, helps a doctor to quickly detect oral conditions (e.g., oral health conditions) and/or oral health problems and their respective severity levels, helps the doctor to make better judgments about treatment of oral conditions and/or oral health problems, and further helps the doctor in communicating with a patient that patient's oral conditions and/or oral health problems and possible treatments. This makes the process of identifying, diagnosing, and treating oral conditions and/or oral health problems easier and more efficient. The doctor may select any of the oral conditions and/or oral health problems to determine prognosis of that condition as it exists in the present and how it will likely progress into the future. Additionally, the oral health diagnostics system may provide treatment simulations of how the oral conditions and/or oral health problems will be affected or eliminated by one or more treatments.

In embodiments, a doctor may customize the oral conditions, oral health problems and/or areas of interest by adding emphasis or notes to specific oral conditions, oral health problems and/or areas of interest. For example, a patient may complain of a particular tooth aching. The doctor may highlight that particular tooth on the radiograph. Oral conditions that are found that are associated with the particular highlighted or selected tooth may then be shown in the dental diagnostics summary. In a further example, a doctor may select a particular tooth (e.g., lower left molar), and the dental diagnostics summary may be updated by modifying the severity results to be specific for that selected tooth. For example, if for the selected tooth an issue was found for caries and a possible issue was found for tooth stains, then the dental diagnostics summary would be updated to show no issues found for tooth wear, occlusion, crowding/spacing, plaque, tooth cracks, and gum recession, to show a potential issue found for tooth stains and to show an issue found for caries. This may help a doctor to quickly identify possible root causes for the pain that the patient complained of for the specific tooth that was selected. The doctor may then select a different tooth to get a summary of dental issues for that other tooth.

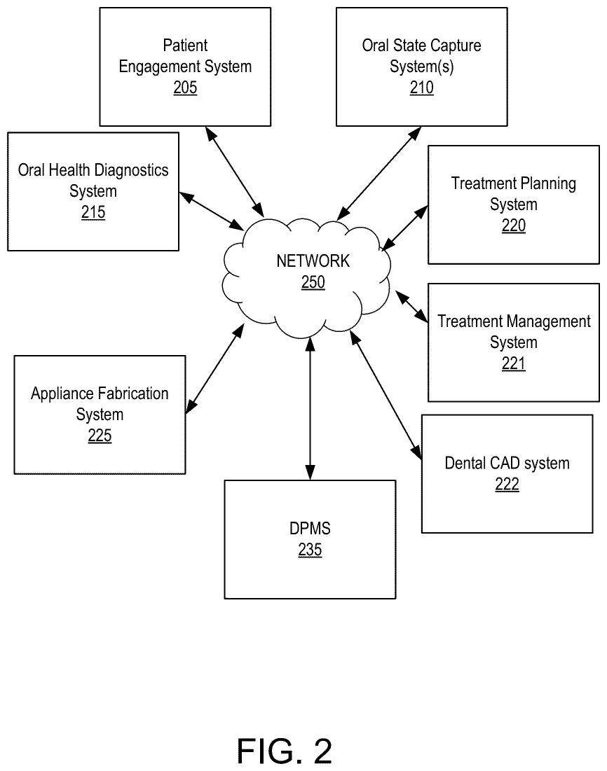

FIG. 2 illustrates an architecture comprising a set of systems for detecting, predicting, diagnosing, reporting on and treating oral conditions and/or oral health problems, in accordance with embodiments of the present disclosure. The systems in one embodiment include a patient engagement system 205, one or more oral state capture systems 210, a treatment planning and/or management system 220, a DPMS 235, an appliance fabrication system 225 and/or an oral health diagnostics system 215.

In embodiments, oral health diagnostics system 215 corresponds to oral health diagnostics system 118 of FIG. 1.

Furthermore, in embodiments patient engagement system 205 corresponds to corresponds to patient engagement system 192.

In further embodiments, treatment planning system 220 may correspond to treatment planning system 190 and/or treatment management system 221 may correspond to treatment management system 191 of FIG. 1. The treatment planning and/or management systems 220, 221 may provide treatment plans, treatment recommendations, orthodontic/restorative integration capabilities, and/or other capabilities. These systems may, in some implementations, take in representations of dentition, identify (through human activities and/or automation) orthodontic/restorative treatments to dentition, provide staging/intermediate positioning/final positioning capabilities of orthodontic/restorative treatments, receive and/or process modifications to the treatment plan, provide updated treatments, support appliance design, etc. In some implementations, the treatment planning systems implement an end-to-end digital treatment planning workflow.

Treatment planning system 220 may provide controls for modifying and/or moving oral structures, teeth, etc. In some embodiments, treatment planning system 220 Includes hard limits on some movements, oral structure positions, etc., and may determine when and/or where certain types of interactions are permitted. This may include comparison of an instructed movement/position of one or more oral structures against entries in hard limit databases that store information about movements that are and/or not feasible.

The treatment management system 221 and/or oral health diagnostics system 215 may provide interactive tools to allow users to plan/manage treatments, examine the state of a person's dentition, evaluate data from x-rays and various oral state capture modalities, and so on. The treatment management system 221 and/or oral health diagnostics system 215 can provide users with an immersive experience where they can evaluate and/or annotate a person's dentition, plan/implement possible treatments for the person's dentition, review/approve/implement actions/recommendations, etc. In some implementations, the treatment management system 221 and/or oral health diagnostics system 215 implements one or more standalone tools related to interaction with a segmented radiographic representation of the oral cavity. The oral health diagnostics system 215 may communicate to the treatment planning system 220 and/or treatment management system 221 for ortho-restorative capabilities through APIs or other architecture.

In some implementations, the oral health diagnostics system 215 is combined with ortho-restorative capabilities (e.g., e.g., treatment planning and/or treatment management functionalities). In such implementations, a user might be presented with ortho-restorative capabilities on one or more 3D models of a dental arch (e.g., generated from an intraoral scan scan) of the oral cavity. The 3D model(s) may show teeth represented from an intraoral scan, and may show teeth and other oral structures and/or conditions as determined from a segmented radiographic representation of the oral cavity. In these examples, aspects of the segmented radiographic representation of the oral cavity and the depiction of the oral cavity from the intraoral scan may be overlaid, blended, reconciled, etc. so that a single model would be shown. Staging capabilities for different stages of a treatment plan may be shown. Various annotation tools may be be available to interact with the depiction of the oral cavity.

In further embodiments, oral state capture system(s) 210 may correspond to one or more oral state capture systems 194 of FIG. 1.

In further embodiments, dental CAD system 222 may correspond to dental CAD system 154 of FIG. 1.

In further embodiments, DPMS 235 may correspond to DPMS 154 of FIG. 1.

The appliance fabrication system 225 may include one or more systems that allow appliance design and/or fabrication in embodiments. Appliance fabrication system 225 may include systems for manufacturing dental appliances and/or orthodontic appliances for patients, for example. Such dental and/or orthodontic appliances may include orthodontic aligners (e.g., clear polymeric aligners), palatal expanders, sleep apnea devices, retainers, mouth guards, night guards, and so on. In some embodiments, treatment planning and/or management system 220 generates digital models for one or more molds and/or dental appliances. The digital models for dental appliances may be used to directly print dental appliances using additive manufacturing such as 3D printings. Digital models for molds may be used to print molds associated with dental appliances. Polymeric sheets may then be thermoformed over the printed molds and trimmed to form the dental appliances in embodiments. Appliance fabrication system 225 may include 3D printers, thermoforming machines, automation machines for part movement and handling, quality control stations, and so on.

Some or all of the indicated systems may be connected via a network 250, which may include one or more public networks (e.g., the Internet) and/or private networks (e.g., intranets). Systems may exchange information such are reports, 3D model files, standard tessellation language interface (STI) files for use in 3D printing, and so on.

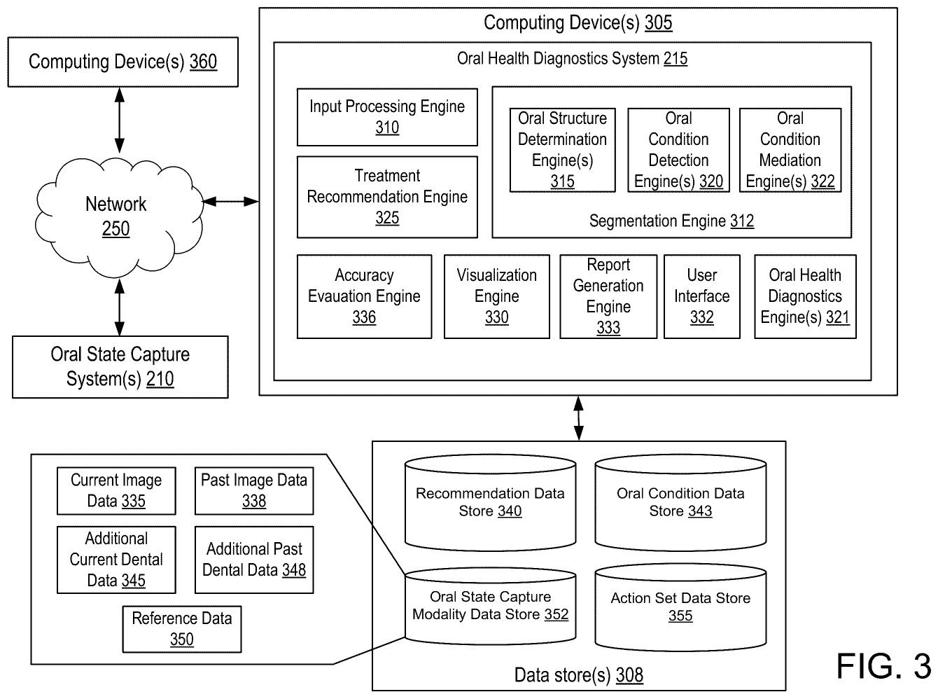

FIG. 3 illustrates an oral health diagnostics system 215, in accordance with embodiments of the present disclosure. Oral health diagnostics system 215 may execute on one or more computing devices 305 that may be coupled to one or more additional computing devices 360, one or more oral state capture systems 210 and/or a data store 308 directly and/or via network 250. The network 250 may be a local area network (LAN), a public wide area network (WAN) (e.g., the Internet), a private WAN (e.g., an intranet), or a combination thereof.

Computing device(s) 305 may include one or more processing devices, memory, secondary storage, one or more input devices (e.g., such as a keyboard, mouse, tablet, and so on), one or more output devices (e.g., a display, a printer, etc.), and/or other hardware components. Computing device(s) 305 may include one or more physical machines and/or one or more virtual machines hosted by one or more physical machines. The physical machine(s) may be a rackmount server, a desktop computer, or other computing device. In one embodiment, computing device(s) 305 can include a virtual machine managed and provided by a cloud provider system. Each virtual machine offered by a cloud service provider may be hosted on a physical machine configured as part of a cloud. Such physical machines are often located in a data center. The cloud provider system and cloud may be provided as an infrastructure as a service (IaaS) layer. One example of such a cloud is Amazon's® Elastic Compute Cloud (EC2®). In one embodiment, oral health diagnostics system 215 is provided as software as a service (Saas), which dental practices may subscribe to. In one embodiment, oral health diagnostics system 215 is an application that runs on a computing device (e.g., computing device 305) of a dental practice.

Data store 310 may be an internal data store, or an external data store that is connected to computing device 305 directly or via a network. Examples of network data stores include a storage area network (SAN), a network attached storage (NAS), and a storage service provided by a cloud computing service provider. Data store 310 may include a file system, a database, or other data storage arrangement.

In one embodiment, oral health diagnostics system 215 includes an input processing engine 310, a segmentation engine 312, an oral health diagnostics engine 321, a treatment recommendation engine 325, an accuracy evaluation engine 336, a visualization engine 330, a report generation engine 333 and/or a user interface 332. Each of the engines may include logic for performing one or more operations or sets of operations associated with different capabilities of the oral health diagnostics system. The division of the oral health diagnostics system 215 into various engines is provided for ease of explanation, and does not necessarily represent an actual arrangement or structure of software. For example, the operations and/or functionality described with reference to particular engines may instead be performed by other engines or modules in embodiments, and the functionality of different engines may be combined and/or divided into still further engines in embodiments. In embodiments, one or more of the engines may function as independent threads, and/or separate processes of one or more engines may function as independent threads.

Input processing module 310 may receive and process data from one or more oral state capture modalities. Processing of such data may include performing pre-processing of such data prior to providing the data to segmentation engine 312. In some embodiments, input processing module 310 processes the input image data to determine whether it satisfies one or more image quality criteria. For example, input processing module 310 may process the image data to determine whether a blurriness of a received image is below a blurriness threshold, whether the image data is of a proper size for processing by the segmentation engine, and so on. If the data is too small, then input processing engine 310 may add dummy pixels to the image to cause it to comply with size criteria. Similarly, if the image is too large, input processing engine may crop the image so that it satisfies size criteria. Input processing engine 310 may additionally perform one or more other alterations to input image data to place the image data into a state for improved processing by segmentation engine 312.

Segmentation engine 312 may include one or more oral structure determination engines 315. One or more of the oral structure determination engines 315 may be configured to process image data of a particular oral state capture modality to segment the image data into teeth and/or gingiva, and to label each tooth with a tooth number (e.g., according to a standard tooth numbering system such as the universal numbering system for teeth, international (FDI) tooth numbering system, the Zsigmondy-Palmer system for tooth numbering, etc.). Oral structure determination engines 315 may deconstruct input image data into constituent parts of oral anatomy and/or other structures in the oral cavity in embodiments, such as jaws, teeth, crowns, roots, gums, sinuses, soft tissues, and so on.

For example, a first oral structure determination engine 315 may be configured for processing 3D models of dental arches, a second oral structure determination engine 315 may be configured for processing bite-wing radiographs, a third oral structure determination engine 315 may be configured for processing periapical radiographs, a fourth oral structure determination engine 315 may be configured for processing panoramic radiographs, a fifth oral structure determination engine 315 may be configured for processing 2D images of oral cavities, and so on. In general, the oral structure determination engines 315 each include one or multiple trained machine learning models trained to perform one or more segmentation tasks, such as for tooth segmentation. In some embodiments, one or more oral structure determination engines 315 include an ensemble of machine learning models and one or more post-processing modules for reconciling disagreement between the multiple machine learning models of the ensemble.

Segmentation engine 312 may further include one or a collection of different oral condition detection engines 320. Each of the oral condition detection engines 320 may include one or more trained machine learning models trained to perform segmentation and/or object detection with respect to one or more types of oral conditions and based on data from a particular oral state capture modality. For example, segmentation engine 312 may include separate oral condition detection engines for performing pixel-level detection of one or more of calculus, caries, inflammation around tooth apexes, restorations, periodontal bone lines, a mandibular nerve, a cento-enamel junction, a cracked tooth, gingival swelling, an impacted tooth, a partially erupted tooth, and/or other oral conditions. Alternatively, one or more oral condition detection engines may include a trained machine learning model capable of segmenting and/or identifying multiple different oral conditions.

Segmentation engine 312 may further include one or more oral condition mediation engines 332. Oral condition mediation engines 322 may be configured to receive oral condition information from multiple different oral condition detection engines 320, and to generate updated and/or improved oral condition detections based on the combined outputs of the different oral condition detection engines 320. Different oral condition mediation engines 322 may be configured to receive different sets of inputs. For example, a first oral condition mediation engine 322 may be configured to receive first oral condition information about caries generated by a first oral condition detection engine from first data of a first oral state capture modality and second oral condition information about caries generated by a second oral condition engine from second data of a second oral state capture modality. In another example, a second oral condition mediation engine 322 may be configured to combine first oral condition information about a first type of oral condition and second oral condition information about a second type of oral condition, and may update the first oral condition information based on the second oral condition information and/or vice versa. For example, if first oral condition information indicates that a tooth is a restoration and second oral condition information indicates that the tooth has a caries, then the oral condition mediation engine may update the caries information to remove the indication of the caries for the tooth since a restoration cannot have a caries. Many other decisions may be made about updating oral condition information based on other oral condition information by one or more oral condition mediation engines 322 in embodiments. Ultimately, the oral condition mediation engine(s) 322 may determine discrepancies and/or different results from the different oral condition information, and resolve the discrepancies and/or combine the different oral condition information to result in superior results.

In embodiments, each of the oral condition detection engines 320 may be responsible for performing an analysis associated with a different type of oral condition. For example, oral health condition engines 320 may separately analyze for tooth cracks, gum recession, tooth wear, occlusal contacts, crowding of teeth and/or other malocclusions, plaque, tooth stains, calculus, caries, and so on. In embodiments, current image data 335 (e.g., current radiographs, color images, 3D models, etc.), past image data 338, additional current dental data 345 (e.g., patient input, doctor input, data from a DPMS, data from sensors, etc.), additional past dental data 348 and/or reference data 350 may be used to perform one or more dental analysis. Such data may be stored in an oral state capture modality data store 352 in embodiments. In an example, the data regarding an at-hand patient may include X-rays, 2D intraoral images, 3D intraoral images, 2D models, CBCT scans and/or virtual 3D models corresponding to the patient visit during which the scanning occurs. The data regarding the at-hand patient may additionally include past X-rays, 2D intraoral images, 3D intraoral images, 2D models, CBCT scans, and/or virtual 3D models of the patient (e.g., corresponding to past visits of the patient and/or to dental records of the patient). All such data may be stored in an oral condition data store 352 in embodiments.

One or more oral condition detection engines 320 may perform one or more types of oral condition analyses using intraoral data (e.g., current image data 335, past image data 338, additional current dental data 345, additional past dental data 348 and/or reference data 350), as discussed herein. As a result, segmentation engine 312 may determine multiple different oral conditions and severity levels of each of those types of identified health conditions. In embodiments, oral conditions may be ranked based on severity.

In some embodiments, different ML models (e.g., of different oral condition detection engines 320) are used for detection of different types of oral conditions. In some embodiments, one or more ML models (e.g., of different oral condition detection engines 320) share one or more model layers. For example, ML models trained to process a same type of image data may share one or more lower layers, but may have different upper layers. This may optimize performance of the ML models while reducing an amount of memory and/or processing power used by the ML models.

Oral health diagnostics engines 321 may process outputs of the segmentation engine 312 and/or data from one or more oral state capture modalities to generate actionable symptom recommendations and/or diagnoses of oral health problems associated with identified oral conditions. In some embodiments, oral health diagnostics engines 321 include one or more trained machine learning models trained to receive inputs of identified oral conditions and/or data from one or more oral state capture modalities, and to output actionable symptom recommendations and/or diagnoses of oral health problems. In some embodiments, oral health diagnostics engines 321 include one or more decision trees and/or random forest models. Oral health diagnostics engines 312 may or may not include AI models and/or ML models. Oral health diagnostics engines 321 may receive oral condition information for multiple oral conditions, and based on the combined oral condition information may determine underlying oral health problems that may be root causes of the oral conditions or otherwise associated with the oral conditions. For example, oral health diagnostics engine(s) 321 may identify oral cancer, periodontitis, gum disease, caries, an infection around a root canal of a tooth, and/or other oral health problems.

Different oral conditions may correlate with and/or cause different oral health problems. Accordingly, information on the types of oral conditions, severity of oral conditions, etc. may be used determine actionable symptom recommendations and/or to diagnose oral health problems.

Additionally, different oral conditions and/or oral health problems may correlate with different overall health problems. Accordingly, if certain oral conditions and/or oral health problems are detected, then this may be evidence that a patient might be suffering from one or more general health conditions. For example, periodontitis has been shown to correlate with diabetes, cardiovascular disease, dementia, psoriasis, lung cancer and chronic obstructive pulmonary disease (COPD). In another example, tooth loss has been found to correlate with cardiovascular disease, COPD, and dementia. Additionally, caries has been found to correlate with diabetes and cardiovascular disease. Accordingly, detections of periodontitis, tooth loss and caries may be indicative of diabetes and/or cardiovascular disease. Similarly, detections of periodontitis and tooth loss may be indicative of COPD and dementia. If periodontitis, tooth loss and/or caries of a threshold level of severity are determined for a patient, then processing logic may output a recommendation that the patient be referred for testing to assess whether the patient has one or more of diabetes, cardiovascular disease, COPD, dementia, psoriasis and/or lung cancer in embodiments.

In an example, intraoral scans of a patient's oral cavity may be received from an intraoral scanner and one or more radiographs of the patient's oral cavity may be received from an x-ray system. One or more oral structure determination engines 315, oral condition detection engines 320 and/or oral condition mediation engines 322 configured to operate on intraoral scan data and/or 3D models generated from intraoral scan data may process the intraoral scans and/or a 3D model generated from the intraoral scans to identify gum recession, gum swelling, bleeding, caries, and/or plaque (also known as calculus) on the patient's teeth. Additionally, one or more oral structure determination engines 315, oral condition detection engines 320 and/or oral condition mediation engines 322 configured to operate on radiographs may process the radiograph(s) to identify information related to periodontal bone loss, caries, and/or calculus with respect to one or more of the patient's teeth. Oral condition detection engine 320 and/or oral condition mediation engine 322 may, for example, combine tooth segmentation information and periodontal bone loss segmentation information from analysis of a radiograph to determine the level of a periodontal bone line at one or more teeth. Oral condition detection engine 320 and/or oral condition mediation engine 322 may determine bone loss values for each tooth and/or region. Based on these bone loss values, oral condition detection engine 320 and/or oral condition mediation engine 322 may determine whether the patient has horizontal bone loss and/or vertical bone loss at one or more teeth or areas. Additionally, or alternatively, oral condition detection engine 320 and/or oral condition mediation engine 322 may determine whether the patient has generalized bone loss (e.g., that applies to all teeth in the upper and/or lower jaw) and/or whether the patient has localized bone loss (e.g., at one or more specific teeth, a particular area of the patient's jaw, etc.). Oral condition detection engine 320 and/or oral condition mediation engine 322 may additionally or alternatively determine an angle of a periodontal bone line for the patient at the one or more teeth, wherein the angle of the periodontal bone line may be used to identify at least one of horizontal bone loss or vertical bone loss.

Based on the determined periodontal bone loss information (e.g., severity of periodontal bone loss for one or more teeth, information on general vs. localized bone loss, angle of bone line, horizontal vs. vertical bone loss, etc.) from analysis of the radiograph and gum recession, gum swelling and/or plaque information from the analysis of the intraoral scan data, and optionally further based on other information such as patient age, patient habits, patient health conditions, etc., oral health diagnostics engine 321 may determine whether a patient has periodontitis and/or a stage of the periodontitis. Other received information used to assess periodontitis may include, for example, pocket depth information received from a DPMS for one or more teeth, smoking status for the patient, and/or medical history for the patient.

In embodiments, oral health diagnostics engines 321 may use information of multiple different types of identified oral conditions and/or associated severity levels to determine correlations and/or cause and effect relationships between two or more of the identified oral conditions. Multiple oral conditions may be caused by the same underlying root cause. Additionally, some oral conditions may serve as an underlying root cause for other oral conditions. Treatment of the underlying root cause oral conditions may mitigate or halt further development of other oral conditions. For example, malocclusion (e.g., tooth crowding and/or tooth spacing or gaps), tooth wear and caries may all be identified for the same tooth or set of teeth. Oral health diagnostics engine(s) 321 may analyze these identified oral conditions that have a common, overlapping or adjacent area of interest, and determine a correlation or causal link between one or more of the oral conditions. In example, oral health diagnostics engine(s) 321 may determine that the caries and tooth wear for a particular group of teeth is caused by tooth crowding for that group of teeth. By performing orthodontic treatment for that group of teeth, the malocclusion may be corrected, which may prevent or reduce further caries progression and/or tooth wear for that group of teeth. In another example, plaque, tooth staining, and gum recession may be identified for a region of a dental arch. The tooth staining and gum recession may be symptoms of excessive plaque. The oral health diagnostics engine(s) 321 may determine that the plaque is an underlying cause for the tooth staining and/or gum recession.

In embodiments, currently identified oral conditions and/or oral health problems may be used by the oral health diagnostics engine(s) 321 to predict future oral conditions that are not presently indicated. For example, a heavy occlusal contact may be assessed to predict tooth wear and/or a tooth crack in an area associated with the heavy occlusal contact. Such analysis may be performed by inputting intraoral data (e.g., current intraoral data and/or past intraoral data) and/or the oral conditions identified from the intraoral data into a trained machine learning model that has been trained to predict future oral conditions based on current oral conditions and/or current dentition (e.g., current 3D surfaces of dental arches). The machine learning model may be any of the types of machine learning models discussed elsewhere herein. The machine learning model may output a probability map indicating predicted locations of oral conditions and/or types of oral conditions. Alternatively, the machine learning model may output a prediction of one or more future oral conditions without identifying where those oral conditions are predicted to be located.

Oral health condition assessment tools may enable doctors to view and perform assessments of various types of oral conditions. Each type of oral condition may be associated with its own unique oral condition assessment tool or set of oral condition assessment tools in embodiments.

Accuracy evaluation engine 336 may determine an accuracy of one or more identified oral conditions, actionable symptom recommendations, diagnoses of oral health problems, and so on in embodiments. Alternatively, or additionally, each of the outputs of oral structure determination engines 315, oral condition detection engines 320, oral condition mediation engines 322, and/or oral health diagnostics engines 321 may include probability and/or confidence information indicating a confidence that detected oral conditions, oral health problems, etc. are correct. Confidence information may be generated for segmented objects and/or for individual pixels in embodiments.

In some embodiments, accuracy evaluation engine 336 applies one or more confidence thresholds to detections (e.g., outputs of oral condition detection engines 320). Based on the thresholds, accuracy evaluation engine 336 may determine whether objects amount to detections of oral condition classifications, and/or determine sizes of oral condition classifications in embodiments. Confidence thresholds may be automatically or manually adjusted to change the sensitivity of the system to detecting one or more types of oral conditions. In one embodiment, the oral health diagnostics system 215 may be toggled between a standard sensitivity mode and a high sensitivity mode. One or more confidence thresholds may be reduced for the high sensitivity mode as compared to the standard sensitivity mode, increasing instances of detected oral conditions and/or sizes of detected oral conditions.

In some embodiments, accuracy evaluation engine 336 may flag one or more detected oral conditions and/or diagnoses of oral health problems as suspected oral conditions and/or suspected diagnoses. Suspected oral conditions and/or diagnoses may correspond to edge cases that may be actual oral conditions/diagnoses, or may be false positives. In some embodiments, multiple different suspected diagnoses or oral condition options for a same oral condition/oral health problem may be presented, and a user may select from the multiple options. Suspected diagnoses/oral condition options may be presented as bounding boxes in embodiments. In some embodiments, suspected oral conditions/diagnoses may be presented to a user for acceptance or denial. In some embodiments, a bounding box around suspected oral conditions are transformed into a segmentation mask for the oral condition.

Treatment recommendation engine 325 may provide treatment recommendations based on detected oral conditions and/or based on determined oral health problems and/or actionable symptom recommendations. Treatment recommendation engine 325 may or may not include one or more trained machine learning models. In one embodiment, treatment recommendation engine 325 includes a decision tree that receives an input of oral condition information, diagnosed oral health problems and/or actionable symptom recommendations, and that outputs one or more treatment recommendations. Treatment recommendations may be based on combinations of different oral conditions and/or oral health problems, severity of oral conditions and/or oral health problems, patient age, patient health, and/or other parameters in embodiments.

Visualization engine 330 may output received image data of one or more oral state capture modalities to a display of a user interface 322. Visualization engine 330 may additionally generate overlays associated with each detected instance of an oral condition and output the overlays over the image data in the display. In embodiments, each instance of an oral condition may be associated with a separate layer of the overlay, and may be turned on or off individually. Visualization engine 330 may additionally output a view of a dental chart populated with oral condition information as generated by segmentation engine 312. In embodiments, visualizations may include overlays having shapes of areas of interest for identified oral conditions. These areas of interest may be displayed using a visualization that is coded based on classes of the one or more oral conditions that the areas of interest are associated with.

In some embodiments, visualization engine 330 generates bone measure visualizations, which may show an amount of periodontal bone loss of a patient. Oral condition detection engines 320, oral condition mediation engines 322 and/or oral health diagnostics engines 321 may operate on data to perform bone measure analysis in embodiments. The result of such analysis may then be shown by the visualization engine 330. Such bone measure visualizations may show how a patients' teeth retain or lose bone density over time using analysis of data from radiographs and/or other oral state capture modalities. In embodiments, bone loss may be measured and shown on panoramic x-rays or periapical x-rays, and/or from current bite-wing x-rays combined with data from older panoramic or periapical x-rays or older intraoral scans.

The user interface 332 may be a graphical user interface and may include icons, buttons, graphics, menus, windows and so on for controlling and navigating the oral health diagnostics system 215. A user may interact with user interface 332 to select individual teeth, to modify instances of oral conditions (e.g., by redrawing their shape), to remove instances of oral conditions, to add instances of oral conditions, to turn on and off overlay layers, and so on. The user interface 332 may include tools that a doctor or other user can use to model, annotate, and/or otherwise interact with various oral structures and/or oral conditions that are imaged through various oral structure capture modalities, including radiographs.

User interface 332 may provide visualizations generated by visualization engine 330 about oral structures (e.g., teeth), or oral conditions, oral health problems, and so on. The visualizations may be associated with tools for manipulating oral structures and/or oral conditions, for selecting oral structures, oral conditions, actionable symptom recommendations, diagnoses, and so on. Via the user interface 332, doctors may provide input about oral structures, oral conditions, actionable recommendations, diagnoses, and so on.

User interface 332 enables users to interact with various forms of data that capture the state of a patient's dentition. User interface 332 may additionally enable users to plan treatments for a patient's dentition with various oral state capture modalities, including x-rays. User interface 332 may provide multiple visualization tools that a doctor or other user can use to model, annotate, and/or otherwise interact with various oral structures that are imaged through various oral structure capture modalities, including radiographs. User interface 332 may also provide treatment planning tools for planning of patient treatments. For example, user interface 332 may receive a selection of a treatment recommendation, and oral health diagnostics system 215 may initiate and/or perform automated treatment planning based on the selected treatment recommendation (e.g., optionally including interfacing with a treatment planning system).

In an example, one or more treatment recommendations comprise at least one of one or more restorative treatment recommendations or one or more orthodontic treatment recommendations. Oral health diagnostics system 215 may receive a selection of at least one of a restorative treatment recommendation of the one or more restorative treatment recommendations or an orthodontic treatment recommendation of the one or more orthodontic treatment recommendations based on user interaction with user interface 332. Oral health diagnostics system 215 may then generate a treatment plan that is one of a restorative treatment plan, an orthodontic treatment plan, or an ortho-restorative treatment plan based on the selection. Generating the treatment plan may include determining staging for the treatment plan, optionally receiving modifications to one or more stages of the treatment plan, and outputting an updated treatment plan in an example.

In some embodiments, user interface 332 provides one or more interactive elements to facilitate interaction with a segmented radiographic representation of the oral cavity. User interface 332 may receive one or more interactions with the segmented radiographic representation through the one or more interactive elements, and oral health diagnostics system 215 may take one or more actions, implement one or more recommendations, take one or more treatment steps, etc. based on the one or more interactions.

Via the user interface 332, a user may additionally cause a report to be generated, cause data to be exported to one or more other systems, cause data to be stored, toggle between a standard sensitivity mode and a high sensitivity mode, and so on. The user interface 332 can provide a platform for a doctor or other user to model, annotate, and/or interact with various oral structures depicted through processing of the oral state capture modalities.

User interface 332 may provide multiple different types of interactions that users can have with the depictions of oral structures and oral conditions identified in image data. Such interactions may include rotations, movements of jaws, zooming in, zooming out, panning in one or more directions, and so on. In some embodiments, user interface 332 provides staging information, treatment planning information (e.g., for ortho-restorative treatment) and/or controls for treatment planning and/or treatment management based on integration with a treatment planning system and/or treatment management system. User interface 332 may output photo-realistic depictions of treatment and/or staging based on data generated by visualization engine 330 in embodiments.

In some embodiments, user interface 332 provides information about oral conditions and/or oral structures (e.g., teeth) responsive to a user causing a pointer to hover over the oral conditions and/or oral structures in a presented image. Controls for modifying oral conditions, sizing oral conditions, etc. may additionally or alternatively be presented responsive to a user hovering a pointer over an oral condition or tooth and/or responsive to a user selecting (e.g., via clicking, double clicking, etc.) an oral condition or tooth. User interface 332 may provide controls for sharing a state of a treatment (e.g., with a patient, another doctor, etc.). User interface 332 may provide controls for making changes or modifications to treatments. In an example, user interface 332 may provide controls for moving attachments, teeth, etc. In embodiments, user interface 332 may provide controls for designing restorations or other dental appliances. In embodiments, user interface 332 may provide controls for sending fabrication instructions to fabricate dental appliances, restorations, orthodontic aligners, palatal expanders, wires and brackets, and so on.

In some embodiments, user interface 332 presents one or more radiographs of a patient's oral cavity, and provides controls for annotating the radiograph(s). In some embodiments, user interface 332 outputs one or more visual overlays over a radiograph or other image data based on visualizations generated by visualization engine 330. The visual overlays may convey information about oral conditions, severity of oral conditions, locations of oral conditions, types of oral conditions, and so on. The visual overlays may additionally be interactive, and may be manipulated in embodiments. In some embodiments, one or more visual overlays include controls for modifying represented oral conditions. In one embodiment, a 3D model of a patient's upper and/or lower patient's dental arches are output to a display by visualization engine 330, and user interface 332 may provide controls for interacting with and/or changing a view of the 3D model(s). As the 3D model(s) are rotated, for example, visualization engine 330 may scroll a panoramic x-ray, or update a highlighted or emphasized region of the panoramic x-ray currently shown in a view of the 3D model(s) to show those teeth that are aligned with current view of the 3D model(s). In some embodiments, visualization engine 330 may determine a bite-wing and/or periapical x-ray that most closely aligns with a current view of the 3D model(s), and may display the determined x-ray(s) in the user interface 332. In some embodiments, multiple views of the patient's dentition are shown together (e.g., in different regions of a display).

Report generation engine 333 may generate reports for patients based on the outputs of segmentation engine 312, oral health diagnostics engine 321 and/or treatment recommendation engine 325. In embodiments, reports can be generated upon a click of a button or other graphical user interface (GUI) element (e.g., via a selection from an element of a menu), an entry from a command line, etc. Once a report generation request is received, report preference information (patient information, doctor preference information, etc.) may be gathered from various databases that store that information. Such preference information may include doctor preferences and/or dental practice preferences. Preference information may include treatment preferences, preferred treatment modalities, preferred views (e.g., panoramic, bitewing, periapical, occlusal, buccal, lingual, etc.), preferred imaging modalities (e.g., radiograph, CBCT, color image, etc.), preferred arrangements of data, and so on. Preference information can be learned based on prior reports generated by and/or for doctors and/or dental practices in embodiments. Doctors and/or dental practices may additionally input their preferences. In some embodiments, requests to generate reports may include one or more parameters for report generation. Such report preference information and/or parameters may be used to configure the report in some embodiments. A request to run a report may trigger one or more report generation processes. Such processes may be independent processes and/or may be dependent on processes of other engines (e.g., visualization engine 330, segmentation engine 312, treatment recommendation engine 325, oral health diagnostics engine 321, and so on).

In some embodiments, report generation engine 333 stores report templates in a data store, and may use such report templates to generate and/or manage reports. In some implementations, report templates include one or more sets of report fields that are to be populated, e.g., at runtime, to generate a report.

A report generation request may, but need not, include report parameters. An example of report parameters that may be specified include limits to the numbers and/or types of oral conditions, actionable recommendations, diagnoses, and/or treatment recommendations in a report. Another example of report parameters that may be specified include limits to report length, format parameters (e.g., colors, fonts, locations of various elements, etc.), locations and/or attributes of interactive elements, security and/or encryption parameters (e.g., anything related to access rights to a report and/or ability to share a report), etc.

Generated reports may include image data from one or more image modalities that were evaluated to generate the outputs. The report may include the image data along with an overlay of one or more identified oral conditions over the image data. Additionally, the report may include a dental chart with oral conditions indicated for teeth having those oral conditions. The report may additionally include a list of oral conditions (e.g., in text form). The reports may be diagnostic data reports that summarize oral conditions detected from relevant oral state capture modalities, actionable symptom recommendations, and/or diagnoses of oral health problems associated with the oral conditions. The report may include treatment recommendations, which may include one more actions to take to effectuate treatment of the oral conditions and/or oral health problems. In embodiments, reports can be generated and/or prioritized based on various factors, such as doctor preferences, issue importance, patient historical factors, etc.

Report generation engine 333 can operate to prioritize diagnostic report elements based on attributes of radiographs and/or other oral state capture modalities, user preferences, relevance of patient information, and/or other information. The report generation engine 333 can use trained models (e.g., ML models) to automatically generate reports in some embodiments. The trained models may be trained for a specific doctor and/or dental practice, and may generate reports in a format preferred by the doctor and/or dental practice. Accordingly, the report generation engine 333 can present issues, actionable recommendations, diagnoses, actions to effectuate treatment, proposed treatments, etc. in a manner preferred by the doctor and/or dental practice.

Generated reports can be presented as a document (e.g., a Word document or PDF document), as a webpage, as a page of an application, and/or in another format. Reports may or may not be interactive. Interactive reports may include interactive elements that a user may interact with to modify the report, provide additional information about aspects of the report, and so on. Reports may be formatted according to preferences of a doctor, dental practice, insurance company, and so on. Information related to report preferences may help to prioritize the data, organize the data, and/or present the data in a diagnostic data report.

The report generation engine 325 may use attributes of detected issues (e.g., oral conditions, actionable symptom recommendations, diagnoses of oral health problems, etc.) and/or report preference information to prioritize identified issues. This may involve AI/ML-based understanding of the types of issues that are likely to be relevant to users and/or specific oral conditions, actionable recommendations, diagnoses, actions to effectuate treatment, and so on. In some implementations, the report generation engine 333 uses one or more AI and/or ML models to intelligently prioritize and display oral conditions, actionable recommendations, diagnoses, and/or treatment recommendations.

The report generation engine 333 can operate to render reports in an interactive manner. Rendering techniques can involve interactive displays where users interact with reports. Alternatively, reports may be static diagnostic reports with text, images/views, treatment options presented to a user. The user interface 332 can operate to facilitate user interactions with diagnostic reports in embodiments.

Once a report is generated, in some embodiments, the report generation engine 333 may receive and/or process user interactions with the generated report based on a user interaction with user interface 332, and provide these user interactions to other systems to update issues related to oral conditions, actionable recommendations, diagnoses, actions to effectuate treatment, etc. In some embodiments, other engines and/or integrated systems may use the reports and/or interactions with them for treatment planning, etc.

One or more data stores 308 may store input data (e.g., data captured using one or more oral state capture modalities) and output data (e.g., reports, determined oral state conditions, diagnoses of oral health problems, actionable symptom recommendations, treatment recommendations, and so on). In some embodiments, treatment recommendations are stored in a recommendation data store 340, actionable symptom recommendations are stored in an action set data store 355 and/or determined oral conditions are stored in oral condition data store 343. Alternatively, the varies types of detections, analysis results, etc. may be stored together in a single data store.

Oral state capture modality data store 352 may store captured data from one or more oral state capture modalities, as indicated above. Reference data 350 in the oral state capture modality data store 352 may include pooled patient data, which may include X-rays, 2D intraoral images, 3D intraoral images, 2D models, and/or virtual 3D models regarding a multitude of patients. Such a multitude of patients may or may not include the at-hand patient. The pooled patient data may be anonymized and/or employed in compliance with regional medical record privacy regulations (e.g., the Health Insurance Portability and Accountability Act (HIPAA). The pooled patient data may include data corresponding to scanning of the sort discussed herein and/or other data. Reference data may additionally or alternatively include pedagogical patient data, which may include X-rays, 2D intraoral images, 3D intraoral images, 2D models, virtual 3D models, and/or medical illustrations (e.g., medical illustration drawings and/or other images) employed in educational contexts.

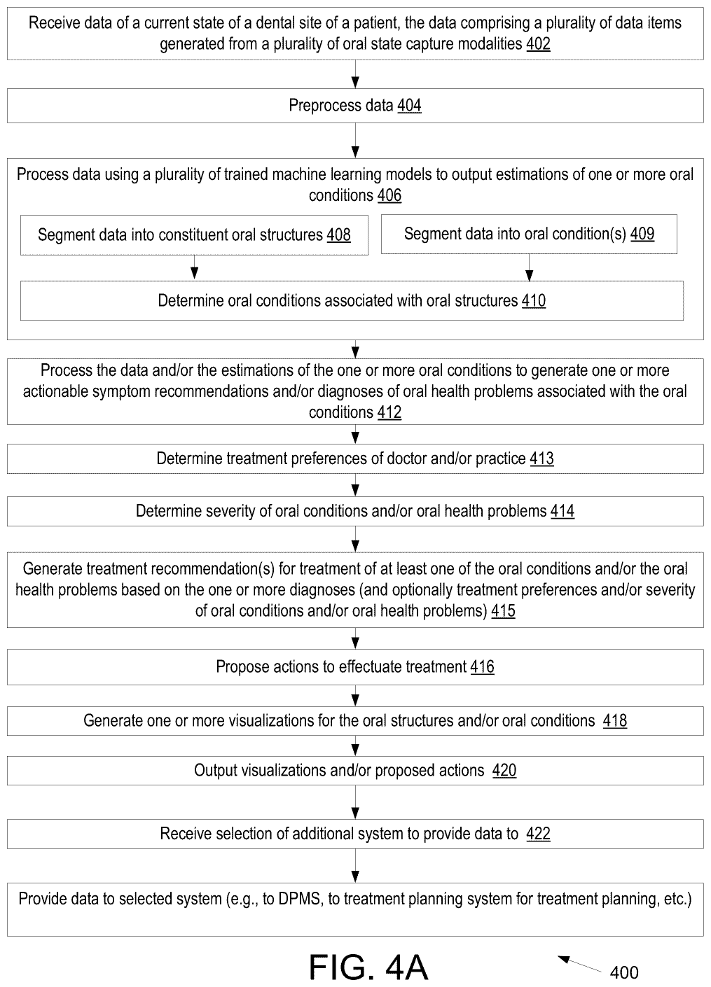

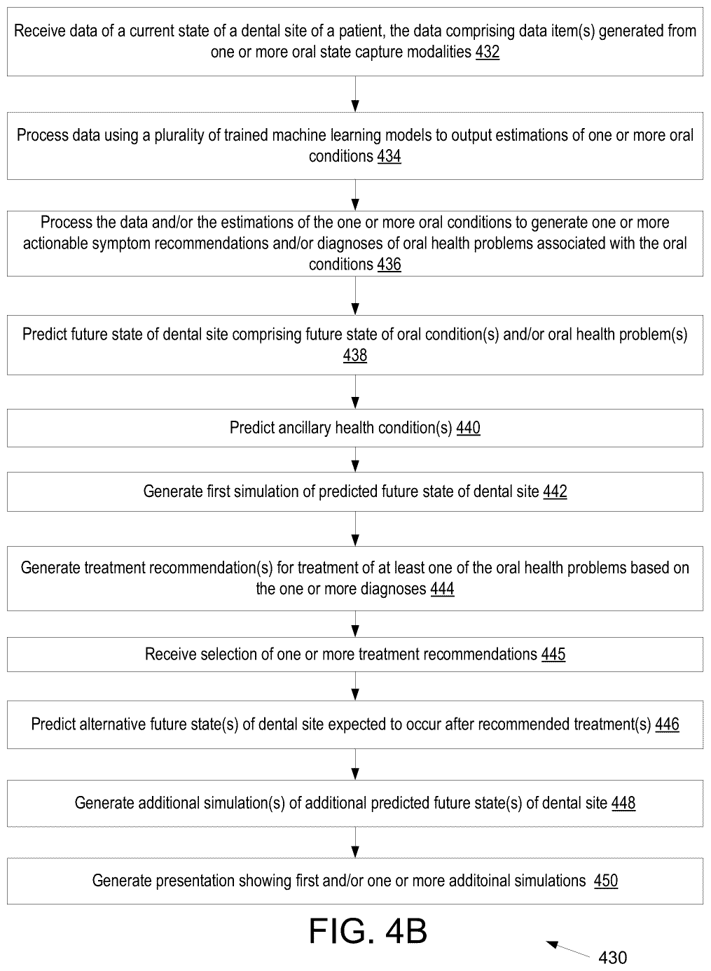

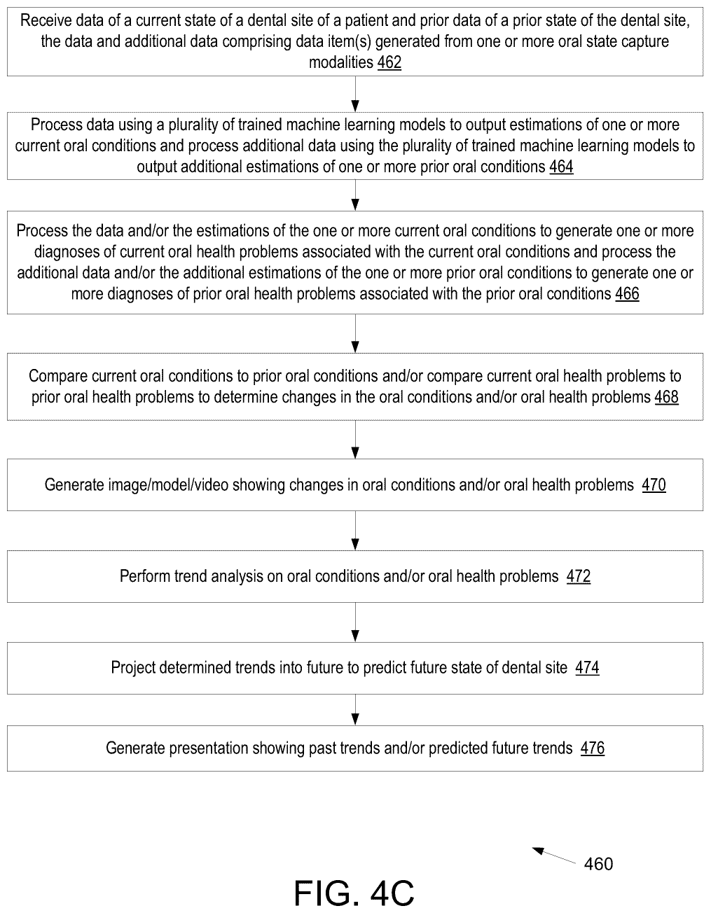

FIGS. 4A-8B illustrate flow diagrams of methods performed by an oral health diagnostics system, in accordance with embodiments of the present disclosure. These methods may be performed by processing logic that comprises hardware (e.g., circuitry, dedicated logic, programmable logic, microcode, etc.), software (such as instructions run on a processing device), or a combination thereof. In one embodiment, processing logic corresponds to computing device 305 of FIG. 3 (e.g., to a computing device 305 executing an oral health diagnostics system 215).

FIG. 4A illustrates a flow diagram for a method 400 of assessing a patient's oral health using data from multiple oral state capture modalities, in accordance with embodiments of the present disclosure. At block 402 of method 400, processing logic receives data of a current state of a dental site of a patient, the data comprising a plurality of data items generated from a plurality of oral state capture modalities. For example, the data may include a first type of radiograph (e.g., a bite-wing radiograph) and a second type of radiograph (e.g., a periapical radiograph or a panoramic radiograph). In another example, the data may include a radiograph and one or more intraoral scans or 3D models of an upper and/or lower dental arch generated from intraoral scans. In other examples, the data may include one or more radiographs and non-image data, such as sensor data from a fitness device and/or a dental appliance, patient input, doctor input, and so on. Many other combinations of data from different oral state capture modalities may also be used. Some oral state capture modalities may be better for estimating some types of oral conditions and/or oral health problems, and other oral state capture modalities may be better for estimating other types of oral conditions and/or oral health problems. Additionally, an accuracy of identifications of oral conditions and/or oral health problems may be improved by combining data from multiple different oral state capture modalities. Accordingly, by combining analysis of data from multiple oral state capture modalities, the number and/or types of oral conditions and/or oral health problems that can be identified is increased and the accuracy of identifications of such oral conditions and/or oral health problems may be increased. Moreover, the effectiveness of recommended treatments may also be improved by using data from multiple oral state capture modalities.

At block 404, processing logic preprocesses the received data. The preprocessing operations that are performed may depend on the oral state capture modalities of the received data. For example, image processing may be performed on received images (e.g., color images, radiographs, etc.), while natural language processing may be performed on received patient input and/or doctor input received in the form of text and/or audio. Examples of image processing that may be performed include resizing of images (e.g., resizing images to a standard size to ensure uniformity across a dataset and to fit a machine learning model's input size requirements), normalization (e.g., scaling pixel values to a common scale (e.g., [0, 1] or [−1, 1]) to make computations easier and faster to process), mean subtraction, standardization, contrast adjustment (e.g., adjusting the contrast of images to make features more distinguishable, such as via techniques like histogram equalization or adaptive histogram equalization), noise reduction (e.g., removing noise from images using techniques like Gaussian blur, median filtering, or denoising autoencoders), color space conversion (e.g., converting images from RGB to grayscale or HSV), edge detection (e.g., enhancing edges in images using techniques like the Sobel operator or Canny edge detector), and so on.

In some embodiments, preprocessing images includes assessing input images to determine whether they are suitable for analysis. This may include determining whether input image data satisfies one or more quality criteria. Quality criteria may include a sharpness criterion, a blurriness criterion, a resolution criterion, an oral structure criterion, and so on. For example, processing logic may measure a blurriness or sharpness of an image, and may compare the measured blurriness or sharpness to a threshold. If the image data has a blurriness that is above a blurriness threshold or a sharpness that is below a sharpness threshold, then the image data may be rejected from further processing. In another example, processing logic may process image data to ensure that the image data does not show improperly cut-off oral structures. For example, if a bite-wing radiograph was poorly positioned and fails to adequately show upper teeth or lower teeth, then the radiograph may be rejected from further processing.

Once the data is preprocessed, the data may then be processed using a plurality of trained machine learning models. Each of the machine learning models may be trained to process data from a particular oral state capture modality in embodiments. For example, a first ML model may be trained to process bite-wing radiographs, a second ML model may be trained to process periapical radiographs, a third ML model may be trained to process panoramic radiographs, a fourth ML model may be trained to process color 2D images (e.g., a particular type of color 2D images, such as occlusal 2D images, front-vie 2D images, side-view 2D images, etc.), a fifth ML model may be trained to process NIR images, a sixth ML model may be trained to process images generated using fluorescence imaging, a seventh ML model may be trained to process 3D models of dental arches, an eighth ML model may be trained to process CBCT scans, and so on. In some embodiments, one or more machine learning models may be trained to receive data from particular combinations of oral state capture modalities. For example, an ML model may be trained to receive a combination of a color 2D image of a dental site and a radiograph of the dental site. In some embodiments, different ML models are trained to perform object detection and/or segmentation of different types of oral structures and/or oral conditions. For example, one or more first ML models may be trained to perform tooth segmentation and/or identify tooth numbers of segmented teeth, one or more second ML models may be trained to perform jaw detection and/or segmentation, one or more third ML models may be trained to perform detection and/or segmentation of caries, one or more fourth ML models may be trained to perform detection and/or segmentation of dentin, one or more fifth ML models may be trained to detect periapical radiolucency, one or more sixth ML models may be trained to perform detection and/or segmentation of a convex hull around a lower jaw, one or more seventh ML models may be trained to identify a periapical bone line, one or more eighth ML models may be trained to perform detection and/or segmentation of lesions around tooth roots, one or more ninth ML models may be trained to identify a CEJ line, one or more tenth ML models may be trained to perform detection and/or segmentation of restorations, one or more eleventh ML models may be trained to perform detection and/or segmentation of impacted teeth, one or more twelfth ML models may be trained to perform detection and/or segmentation of unerupted teeth and/or partially erupted teeth, and so on. Other ML models may additionally or alternatively be trained to identify cracked teeth, tooth erosion, gum erosion, tooth stains, tooth crowding, various classes of malocclusion, and so on. Each of the ML models may be trained to receive one or more types of image data (e.g., radiographs, color images, NIR images, etc.) and to generate an output indicating at least one of probabilities that the patient has at least one or more oral conditions or locations in a dental site at which the one or more oral conditions are detected.

In embodiments, some trained ML models are trained to receive and operate on outputs of other ML models in addition to, or instead of, image data. For example, first ML models may process image data to identify regions of interest (e.g., which may be associated with a lower jaw and/or upper jaw, teeth, etc.), and second ML models may process cropped image data that has been cropped to only include one or more identified regions of interest.

In some embodiments, at block 408 input image data is processed using one or more first ML models that segment the image data into constituent oral structures, such as individual teeth, gingiva, and so on. At block 409, one or more second ML models may segment the image data into oral conditions (e.g., such as caries, restorations, lesions around roots, etc.). At block 410, processing logic may then combine the outputs of blocks 408 and 409 to associate detected oral conditions to identified teeth. Moreover, for an oral condition that is associated with a tooth, processing logic may determine a semantic understanding of a location on the tooth that the oral condition has been detected, such as a right of tooth, left of tooth, top of tooth, at tooth root, in enamel of tooth, in dentin of tooth, at a mesial surface of a tooth, at a distal surface of a tooth, at an occlusal surface of a tooth, and so on.

At block 412, processing logic may further process the data and/or the estimations of oral condition(s) to output diagnoses of one or more oral health problems and/or one or more actionable symptom recommendations associated with one or more of the determined oral conditions. Examples of diagnoses include diagnoses of root caries, enamel caries, gingivitis, periodontitis, oral cancer, xerostomia (dry mouth), candidiasis (oral thrush), bruxism, malocclusion, dental trauma, dental abscess, enamel erosion, impacted wisdom teeth, oral ulcers, hypodontia, mucositis, dental fluorosis, amelogenesis imperfecta, and so on.

In some embodiments, processing logic performs a differential diagnosis of one or more oral health problems of the patient. In medicine, a “differential diagnosis” refers to a systematic method used by healthcare providers to identify and evaluate possible medical conditions or diseases that may explain a patient's symptoms or clinical findings. It involves considering a wide range of potential diagnoses and then narrowing down the list based on various factors such as the patient's medical history, presenting symptoms, physical examination findings, diagnostic test results, and risk factors.

The process of differential diagnosis typically begins with gathering patient information (e.g., as performed at block 402). Processing logic may collect comprehensive information about the patient's medical history, including past illnesses, medications, surgeries, allergies, and family history of diseases. Processing logic may also obtain details about current symptoms, their onset, duration, severity, and any associated factors. The data may further include doctor input based on a physical examination of the patient performed by the doctor. The examination may be performed to assess the patient's vital signs, general appearance, and specific signs or abnormalities related to the presenting symptoms.

Based on the operations of blocks 406 and/or 412, processing logic may generate a list of possible diagnoses. Based on the patient's history, symptoms, physical examination findings, and identified oral conditions, processing logic may create a list of potential diagnoses of oral health problems (e.g., root causes) that could explain the presenting symptoms. This list may include common and uncommon oral health problems or conditions that need to be considered during the diagnostic process. Processing logic may prioritize and narrow down the differential diagnosis based on factors such as the likelihood of each condition, the severity of the symptoms, the urgency of treatment, and the availability of diagnostic tests. In embodiments, AI and/or ML models are trained based on clinical reasoning, medical knowledge, and experience of many doctors to systematically rule out less likely diagnoses and focus on those that are more probable.

In some embodiments, processing logic may recommend the ordering of one or more diagnostic tests to further refine the differential diagnosis and confirm or rule out specific conditions. These may include blood tests, imaging studies (such as X-rays, CT scans, MRI), electrocardiogra biopsy, microbiological cultures, or other specialized tests depending on the suspected diagnoses. Based on the newly received data, operations of blocks 402, 404, 406 and/or 412 may be repeated. As new information becomes available from diagnostic tests or as the patient's condition evolves over time, processing logic may continuously reassess and revise the differential diagnosis. Processing logic may add or remove potential diagnoses from the list based on the evolving clinical picture and diagnostic findings.

Ultimately, processing logic may formulate a final diagnosis. Alternatively, processing logic may output a final set of one or more actionable symptom recommendations.

At block 413, processing logic may determine treatment preferences of a doctor and/or a dental practice (e.g., which may be a solo practice or a group practice). The treatment preferences may include preferences to fill caries that meet certain criteria, preferences to monitor caries that meet certain criteria, preferences to use particular types of filling material (e.g., composite, silver, gold, etc.), preferences to perform root canals under particular conditions, and so on. Treatment preferences of doctors and/or practices may be input by the doctors/practices and/or may be learned based on processing of past oral conditions and treatments performed by the doctors/practices for those oral conditions.

At block 414, processing logic may determine a severity of one or more of the oral conditions, the actionable symptom recommendations, and/or the diagnosed oral health problems. In some embodiments, severity of oral conditions, actionable symptom recommendations, oral health problems, etc. is output by one or more ML models, which may be the ML models that output the indications of the oral conditions, actionable symptom recommendations, and/or diagnoses of oral health problems or by other ML models that may receives as inputs the data from the one or more oral state capture modalities and/or the oral conditions, actionable symptom recommendations and/or diagnoses of oral health problems. Additionally, or alternatively, image data, identified oral conditions, identified oral health problems, etc. may be processed to measure properties such as area, volume, height, length, distance, size, shape and/or other properties associated with the oral conditions and/or oral health problems. Severity of the oral conditions and/or oral health problems may then be determined based on the measured properties. Processing logic may rank oral conditions and/or oral health problems based at least in part on severity in embodiments.

At block 415, processing logic generates one or more treatment recommendations for treatment of at least one of the oral conditions and/or the oral health problems based on the diagnoses of oral health problems, the actionable symptom recommendations and/or the oral conditions. Identified severity of oral conditions and/or oral health problems may be taken into account for treatment recommendations. Additionally, determined doctor and/or practice preferences may be taken into account for treatment recommendations. In some embodiments, treatment recommendations are generated based on a lookup into a database based on identified oral condition(s), oral health problem(s), and so on. In some embodiments, treatment recommendations are generated by inputting the oral condition(s), oral health problem(s), actionable symptom recommendation(s), etc. into a trained ML model, which may output the one or more treatment recommendations. The one or more treatment recommendations may be tailored to the specific diagnosis of one or more oral health problems, which may include medication, surgery, lifestyle modifications, and/or other interventions aimed at managing the underlying condition and improving patient outcomes. Examples of treatment recommendations include drilling and applying a filling, drilling and applying a crown, tooth extraction, root canal surgery, applying veneers, performing orthodontic treatment, performing scaling and root planing, providing antibiotic therapy, performing pocket reduction surgery, performing bone grafting, performing guided tissue regeneration, performing dental crown lengthening, and performing laser therapy, to name a few.

At block 416, processing logic may propose actions to effectuate treatment. The proposed actions may include actions associated with one or more recommended treatments in embodiments. Proposed actions may include a sequence of treatments and/or a sequence of actions associated with a single treatment in embodiments.

At block 418, processing logic generates one or more visualizations for the determined oral structures and/or oral conditions. The visualizations may include overlays or masks that may be shown over received image data. In one embodiment, a separate layer or overlay is generated for each instance of one or more oral conditions. Different visualizations may include different colors, different fill patterns, different amounts of transparency, and so on.

At block 420, processing logic outputs the visualizations to a display. The visualizations may be output as an overlay over image data, such as over a radiograph or over a 3D model of a dental arch. A user may turn on or off visualizations associated with a particular class of oral condition and/or may turn on or off visualizations of specific instances of one or more oral conditions in embodiments. In some embodiments, processing logic additionally outputs one or more proposed actions, diagnoses of oral health problems, actionable symptom recommendations and/or treatment recommendations to the display. A user may interact with the visualizations and/or with any other output data. For example, a user may select a treatment recommendation and/or one or more proposed actions. A user may also interact with a user interface to generate a report comprising all or selected image data, oral segments, oral conditions, oral health problems, and so on.

In some embodiments, at block 422 processing logic receives a selection of an additional system to provide data to. The data may include any of the received data from the one or more oral state capture modalities, oral segment information, oral condition information, oral health problem information, treatment recommendations, selected treatments, and so on. The additional system may be any of the additional systems indicated in FIG. 2, for example, such as a patient engagement system, a treatment planning system, a treatment management system, a dental CAD system, a DPMS, an appliance fabrication system, and so on. At block 424, processing logic may provide the data to the selected system or systems. This may include formatting the data for ingestion by the selected system or systems in embodiments (e.g., organizing the data into a structured data set that may be used to update patient information of a DPMS). In some embodiments, processing logic may be configured to automatically provide data to one or more preselected systems.

FIG. 4B illustrates a flow diagram for a method 430 of predicting a future oral health of a patient oral health using data from one or more oral state capture modalities, in accordance with embodiments of the present disclosure. At block 432 of method 430, processing logic receives data of a current state of a dental site of a patient, the data comprising one or more data items generated from one or more oral state capture modalities. At block 434, processing logic processes the data using a plurality of trained machine learning models to output estimations of one or more oral conditions. At block 436, processing logic processes the data and/or the estimations of one or more oral conditions to generate one or more actionable symptom recommendations and/or diagnoses of oral health problems associated with the one or more oral conditions.

At block 438, processing logic predicts a future state of the dental site comprising a future state of the oral condition(s) and/or oral health problem(s). In some cases, the future state of the dental site is determined by inputting the current data from the one or more oral state capture modalities, the oral condition(s) and/or the oral health problem(s) into a trained machine learning model. The trained machine learning model may be a generative model that generates a prediction of the future state of the dental site, including future states of oral conditions and/or oral health problems. In some embodiments, the generative model outputs a labeled synthetic image (e.g., synthetic radiograph) of the future state of the dental site, in which predicted oral conditions are labeled.

In some embodiments, the predicted future condition of the dental site is determined based on processing of current data of the dental site (e.g., current image data, current identified oral structures, current identified oral conditions, etc.) and one or more sets of past data of the dental site. Past conditions of the dental site may be compared to current conditions of the dental site to determine trends associated with the oral structures and/or oral conditions, for example. For example, processing logic may determine an amount of change in an oral structure and/or oral condition, and may extrapolate that amount of change into the future. This may include determining time stamps of the current data and the past data, determining an amount of change and/or a rate of change of oral structures and/or oral conditions between the current time and the past time, and then projecting the determined amount of change and/or rate of change to one or more future time. In some embodiments, predictions of a future state of a dental site at multiple different future points in time may be determined.

In some embodiments, at block 440 processing logic predicts ancillary health conditions of a patient. Ancillary health conditions may include health conditions for patient anatomy outside of the oral cavity and/or general patient health conditions. For example, there is a correlation between oral health and cardiovascular health, between oral health and diabetes, between oral health and respiratory infections, between oral health and pregnancy complications, between oral health and osteoporosis, between oral health and kidney disease, between gum disease and rheumatoid arthritis, between oral health and eating disorders, and so on. For example, based on a detection of chronic inflammation of the gums of a patient, processing logic may predict cardiovascular conditions such as heart disease, stroke and/or atherosclerosis. Additionally, diabetes and gum disease have a bidirectional relationship. Accordingly, diabetes may increase the risk of gum disease, and gum disease may make it more difficult to control blood sugar levels, leading to complications in diabetes management. In another example, based on a detection of periodontal bone loss, processing logic may predict bone loss elsewhere in the body (e.g., osteoporosis). In some embodiments, processing logic inputs current and/or past oral conditions into a trained machine learning model, which may output predictions of one or more ancillary health conditions.

At block 442, processing logic may generate a simulation of a future state of the dental site. The simulation may include one or more synthetic images showing predicted future states of oral structures and/or oral conditions as determined at block 428 in embodiments. The simulation may be a static simulation (e.g., one or more labeled images or radiographs) and/or a dynamic simulation (e.g., a video showing progression of one or more oral conditions over time).

At block 444, processing logic may generate one or more treatment recommendations for treatment of at least one of the identified oral health problems and/or oral conditions based on the determined diagnoses, determined actionable symptom recommendations and/or determined oral conditions in embodiments. The treatment recommendations may additionally be based at least in part on predicted future conditions of oral conditions and/or oral structures in some embodiments.

At block 445, processing logic receives a selection of one or more treatment recommendations. Processing logic may present tools for selecting treatment recommendations and/or for inputting non-recommended treatments. Based on user interaction with such tools (e.g., buttons, drop down menus, etc.), selection of treatment recommendations may be received.

At block 446, processing logic may predict one or more alternative future states of the dental site expected to occur after performance of the selected treatment(s). In some embodiments, an alternative future state of the dental site may be generated by inputting data for a current state of the dental site and a proposed treatment into a trained machine learning model (e.g., a generative model), which may output an image (e.g., color 2D image or radiograph) of the predicted future state of the dental site after treatment.

At block 448, processing logic may generate a simulation of an alternative future state of the dental site after treatment. The simulation may include one or more synthetic images showing predicted future states of oral structures and/or oral conditions as determined at block 446 in embodiments. The simulation may be a static simulation (e.g., one or more labeled images or radiographs) and/or a dynamic simulation (e.g., a video showing progression of one or more oral conditions over time). In some embodiments, at block 445 multiple different alternative treatments may be selected, at block 446, alternative future states associated with each of the alternative treatments may be generated, and at block 448 different simulations associated with each of the alternative future states may be generated.

At block 450, processing logic may generate a presentation showing the first simulation generated at block 442 and/or the additional simulation(s) generated to block 448.

FIG. 4C illustrates a flow diagram for a method 460 of tracking a patient's oral health over time based on data from one or more oral state capture modalities, in accordance with embodiments of the present disclosure. At block 462 of method 460, processing logic receives data of a current state of a dental site of a patient and prior data of a prior state of the dental site. The data and additional data may comprise data items generated from one or more oral state capture modalities.

At block 464, processing logic may process the received data using a plurality of trained machine learning models to output estimations of one or more current oral conditions, and additionally processes the additional data using the plurality of trained machine learning models to output additional estimations of one or more prior oral conditions.

At block 466, processing logic processes the data and/or the estimations of the one or more current oral conditions to generate one or more diagnoses of current health problems associated with the current oral conditions. Additionally, processing logic processes the additional data and/or the additional estimations of the one or more prior oral conditions to generate one or more diagnoses of prior health problems associated with the prior oral conditions.

At block 468, processing logic compares the current oral conditions to the prior oral conditions and/or compares the current oral health problems to the prior oral health problems to determine changes in the oral conditions and/or the oral health problems.

At block 470, processing logic generates an image, 3D model and/or video showing changes in the oral conditions and/or oral health problems. In the case of a video, multiple frames of the video may be generated, each comprising a predicted intermediate state of the oral conditions and/or oral health problems between the current state and the prior state. The multiple frames may form a video showing a progression from the prior state to the current state of the oral conditions and/or oral health problems.

At block 472, processing logic performs a trend analysis on the oral conditions and/or oral health problems. Performing the trend analysis may include determining changes to oral conditions/health problems over time. Trend analysis may identify a rate of change of oral conditions, an acceleration or deceleration in a rate of change of oral conditions (e.g., if there are multiple sets of prior data, each from a different prior time period), and so on.

At block 474, processing logic may project determined trends into the future to predict a future state of the dental site (e.g., of the oral conditions and/or oral health problems). At block 476, processing logic may then generate a presentation showing past trends for the dental site and/or predicted future trends for the dental site in embodiments.

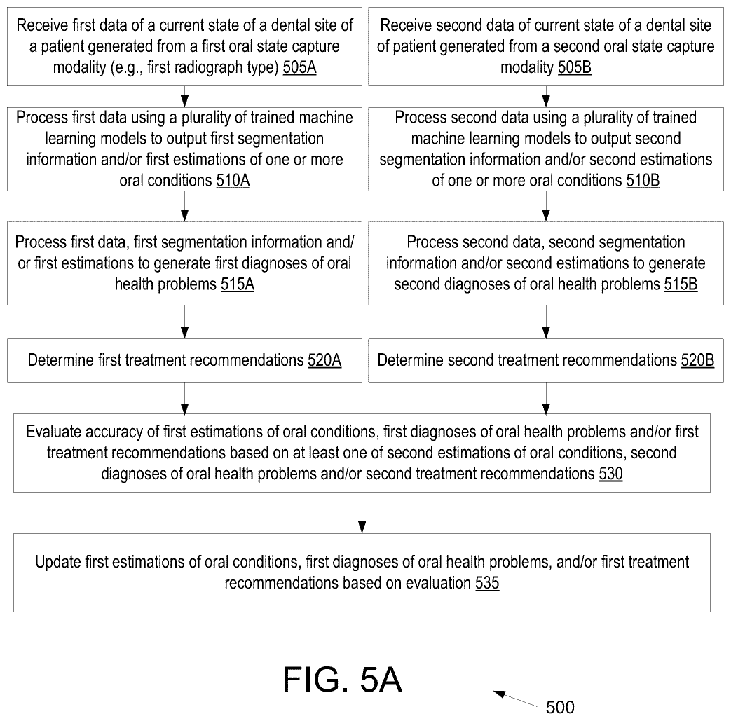

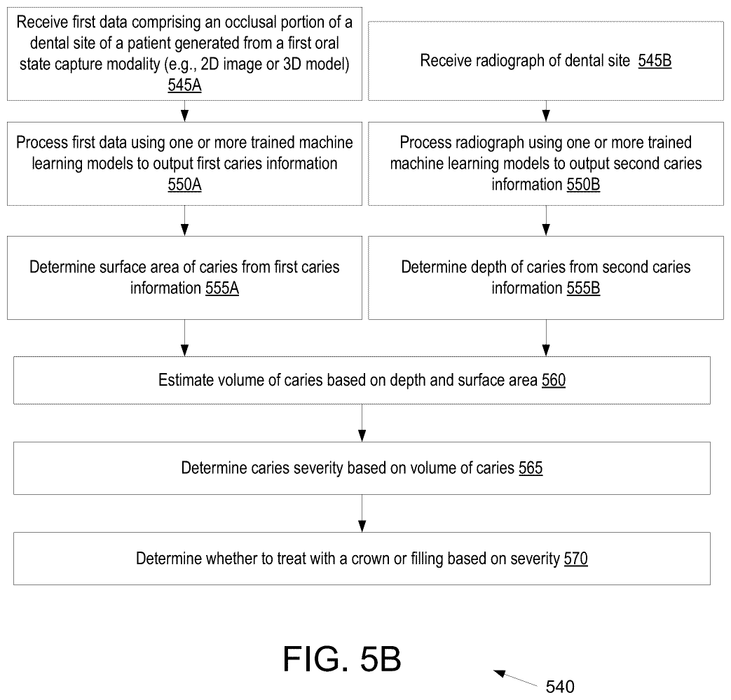

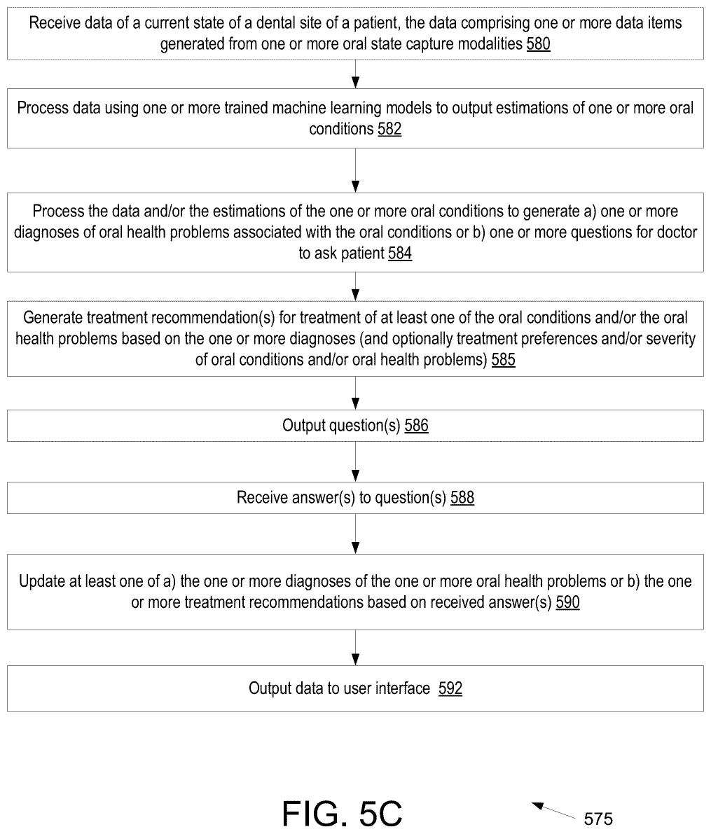

FIG. 5A illustrates a flow diagram for a method 500 of verifying and/or updating an estimate of a patient's oral health, as determined from a first oral state capture modality, using data from a second oral state capture modality, in accordance with embodiments of the present disclosure. In some instances, analysis of data from one or oral state capture modality may be insufficient to accurately diagnose an oral health problem or identify an oral condition. In such instances, processing logic may identify that a confidence of an estimated oral condition and/or oral health problem is low, and may automatically recommend capture of additional data from another oral state capture modality that is typically able to provide information for identifying the oral health problem and/or oral condition in question. Additional data from the recommended oral state capture modality may be gathered and processed to update an estimation of an oral condition and/or oral health problem. In other instances, data from multiple oral state capture modalities may be received and processed in parallel. The outputs generated from processing the data from the multiple oral state capture modalities may be combined to generate more accurate estimates of oral conditions and/or oral health problems.

At block 505A, processing logic receives first data of a current state of a dental site of a patient generated using a first oral state capture modality. At block 510A, processing logic processes the first data using a first plurality of trained machine learning models to output first segmentation information and/or first estimations of one or more oral conditions. At block 515A, processing logic may process the first data, the first segmentation information, and/or the first estimations of oral conditions to generate first diagnoses of oral health problems and/or first actionable symptom recommendations. At block 520A, processing logic may determine first treatment recommendations based on the first data, first segmentation information, first estimates of oral conditions, first actionable symptom recommendations and/or first diagnoses of oral health problems. Based on the output of blocks 510A, 515A and/or 520A, processing logic may determine that estimated oral conditions, actionable symptom recommendations, diagnoses of oral health problems and/or treatment recommendations have a confidence that is lower than a confidence threshold. Based on such a low confidence, processing logic may output a recommendation to gather and process second data from a second oral state capture modality. Alternatively, such second data may be generated without processing logic outputting a recommendation to generate the second data.

At block 505B, processing logic receives second data of the current state of the dental site of the patient generated using a second oral state capture modality (e.g., which may be a recommended oral state capture modality). At block 510B, processing logic processes the second data using a second plurality of trained machine learning models to output second segmentation information and/or second estimations of the one or more oral conditions. At block 515B, processing logic may process the second data, the second segmentation information, and/or the second estimations of oral conditions to generate second diagnoses of oral health problems and/or second actionable symptom recommendations. At block 520B, processing logic may determine second treatment recommendations based on the second data, second segmentation information, second estimates of oral conditions, second actionable symptom recommendations and/or second diagnoses of oral health problems.

At block 530, processing logic may evaluate an accuracy of the first estimations of oral conditions, first diagnoses of oral health problems, first actionable symptom recommendations, and/or first treatment recommendations. The accuracy of the first estimations of oral conditions, first diagnoses of oral health problems, first actionable symptom recommendations, and/or first treatment recommendations may be determined based on confidence values (e.g., as output by ML models) and/or on comparison with of the second estimations of oral conditions, second diagnoses of oral health problems, second actionable symptom recommendations, and/or second treatment recommendations. For example, analysis of the second data may be used to verify the results of the analysis of the first data. The higher the similarity between an estimated oral condition output by block 510A and a same estimated oral condition output by block 510B, the higher the confidence that the estimated oral condition is a true oral condition, for example. In some instances, processing logic may determine to perform additional analysis on the first data and/or may recommend generation of the second data based on the evaluated accuracy of the first estimations of oral conditions, first diagnoses of oral health problems, first actionable symptom recommendations, and/or first treatment recommendations.

In one embodiment, the first data (e.g., one or more 2D color images) is captured by a device of a patient (e.g., a mobile device of the patient), and is initially assessed using software executing on the device of the patient (e.g., a minimal oral health diagnostics system executing on the mobile device of the patient) at blocks 505A, 510A, 515A and/or 520A. The software executing on the device of the patient may have a low processing capacity as compared to a full oral health diagnostics system running on, for example, one or more server machines. After processing the first data, the software executing on the device of the patient may determine that further processing of the data is recommended. Responsive to such a determination, the mobile device of the patient may send the first data to a server system, and a full oral health diagnostics system executing on the server system may repeat the operations of one or more of blocks 505A, 510A, 515A and/or 520A to generate a more accurate assessment of the oral conditions, oral health problems, treatment recommendations, etc. for the patient. In some instances, based on processing the first data generated by the device of the patient and/or by a server system, processing logic may recommend that more accurate data of the same or a different oral state capture modality be captured. Often this results in the patient visiting the doctor, and the doctor using one or more medical-grade tools to capture image data of the patients' oral cavity. For example, radiographs, intraoral scans, CBCT scans, etc. may be performed on the patient at the doctor office to generate second data.

At block 535, processing logic may update the first estimations of oral conditions, first diagnoses of oral health problems, first actionable symptom recommendations and/or first treatment recommendations based on the second estimations of oral conditions, second diagnoses of oral health problems, second actionable symptom recommendations and/or second treatment recommendations. In some embodiments, the first estimations of oral conditions, first diagnoses of oral health problems, first actionable symptom recommendations and/or first treatment recommendations are combined (e.g., averaged) with the second estimations of oral conditions, second diagnoses of oral health problems, second actionable symptom recommendations and/or second treatment recommendations. Since different oral state capture modalities may be better at capturing different oral conditions, a weighted average of oral conditions may be generated in some embodiments, where the weights may be based on how well the first and second oral state capture modalities are at capturing the oral condition in question. Accordingly, for different oral conditions, different weights may be applied to the values associated with the first oral state capture modality and to the second oral state capture modality. In some instances, analysis of the second data confirms the results of analysis of the first data.

In some instances, data from a first oral state capture modality may provide first information about an oral condition, and data from a second oral state capture modality may provide second information about the oral condition. The first information and second information may be complementary, and may inform on different aspects of the oral condition. For example, a radiograph may indicate a depth of a caries, but may not indicate a surface area of the caries. However, a 2D image or 3D model (e.g., generated from intraoral scans of a tooth) may indicate a surface area of the caries but may not indicate a depth of the caries. Such data may be combined in embodiments to determine additional information about an oral condition that may not be determinable from a single oral state capture modality on its own.

FIG. 5B illustrates a flow diagram for a method 540 of using data from a radiograph and data from an image or 3D model to assess a caries severity, in accordance with embodiments of the present disclosure. At block 545A of method 540, processing logic receives first data comprising an occlusal portion of a dental site (e.g., a tooth) of a patient generated from a first oral state capture modality. The first oral state capture modality may be a 3D model of the dental site, a 2D image of the dental site (e.g., a color image, NIR image, etc.), or other oral state capture modality. At block 550A, processing logic processes the first data using one more trained ML models to output first caries information. At block 555A, processing logic determines a surface area of the caries based on the caries information. The caries information may include a pixel-level or point-level mask of the caries indicating each pixel or point of the image or 3D model that is part of a caries instance. The surface area of the caries may be determined by measuring the caries. In some embodiments, the surface area is measured in pixels and is converted to a physical measurement (e.g., mm²).

At block 545B of method 540, processing logic receives a radiograph of the dental site. The radiograph may be, for example, a bite-wing radiograph, a panoramic radiograph, or a periapical radiograph. At block 550B, processing logic processes the radiograph using one more additional trained ML models to output second caries information. At block 555B, processing logic determines a depth of the caries based on the second caries information. The second caries information may include a pixel-level mask of the caries indicating each pixel radiograph that is part of the caries instance. The depth of the caries may be determined by measuring the caries. In some embodiments, the depth is measured in pixels and is converted to a physical measurement (e.g., mm).

At block 560, processing logic estimates a volume of the cares based on the depth and the surface area. For example, processing logic may multiply the depth by the surface area to estimate the volume of the caries. At block 565, processing logic may determine a caries severity based at least in part on the volume or the caries. The caries severity may also be determined based at least in part on a determination of whether the caries is only in a tooth enamel and/or whether the caries has penetrated a dentin of the tooth. Caries identified as enamel caries may be identified as such, for example, in a tooth chart or report of a patient's oral health. Caries identified as dentin caries may be identified as such, for example, in a tooth chart or report of a patient's oral health. Identification of enamel caries vs. dentin caries is described elsewhere herein. At block 570, processing logic may determine whether to recommend treating the caries with a crown or a filling based on the determined severity. For example, if the volume of the caries meets a threshold volume, then a crown may be recommended. However, if the volume of the caries is lower than the threshold volume, then a filling may be recommended. The threshold volume may be based on a size of the tooth in question in some embodiments. For example, the threshold volume may be a percentage of a size of the tooth.

Similar methods may be performed for combining data from other different oral state capture modalities to arrive at a more complete picture of an oral condition of a patient. For example, data used to assess a caries may include at least two of an intraoral scan of a dental site, a near infrared image of the dental site, or an x-ray image of the dental site.

In some instances, processing logic may evaluate oral state capture data to identify one or more oral conditions, generate actionable symptom recommendations, diagnose oral health problems and/or generate treatment recommendations, but may determine that a confidence of the one or more oral conditions, actionable symptom recommendations, oral health problems and/or treatment recommendations is below a confidence threshold. Processing logic may additionally determine additional information that, if the additional information were provided to processing logic, would cause the confidence to exceed the threshold. Additionally, processing logic may generate an output in which two options are equally likely or near to equally likely, but where a selection between the two options may be made if particular additional information were made available. In such instances, processing logic may output a recommendation to gather the additional information. In some instances, the additional information may be patient answers to one or more questions, such as how often do your gums bleed, or do you feel pain in a region of your mouth, or do you have trouble biting. Accordingly, it can be beneficial to automatically ask a patient questions and/or to prompt a doctor to ask the patient such questions based on processing of data from one or more oral state capture modalities.

FIG. 5C illustrates a flow diagram for a method 575 of updating an estimated oral health of a patient based on patient responses to prompted questions, in accordance with embodiments of the present disclosure. At block 580 of method 575, processing logic receives data of a current state of a dental site of a patient, the data comprising one or more data items generated from one or more oral state capture modalities. At block 582, processing logic processes the data using one or more trained machine learning models to output estimations of one or more oral conditions.

At block 582, processing logic may further process the data and/or the estimations of oral condition(s) to output diagnoses of one or more oral health problems, actionable symptom recommendations and/or one or more questions for a doctor to ask a patient (or for processing logic to output to the patient directly).

At block 585, processing logic may generate one or more treatment recommendations for treatment of at least one of the oral conditions and/or the oral health problems based on the diagnoses of oral health problems, the actionable symptom recommendations and/or the oral conditions. Identified severity of oral conditions and/or oral health problems may be taken into account for treatment recommendations. Additionally, determined doctor and/or practice preferences may be taken into account for treatment recommendations.

At block 586, processing logic may output the one or more determined questions. The questions may be output audially via one or more speakers using a text to speech engine. Alternatively, or additionally, the questions may be output as text to a display. The questions may be output to a doctor and/or to a patient. The questions may be questions about gum bleeding, pain or discomfort, trouble chewing or moving a patient's jaw, patient habits (e.g., tooth brushing habits, flossing habits, whether the patient wears a night guards, etc.), and so on.

At block 588, processing logic receives answers to one or more asked questions. At block 590, processing logic may process the answers. Based on the answers, processing logic may update at least one of the one or more oral conditions, one or more diagnoses of oral health problems, one or more actionable symptom recommendations, and/or one or more treatment recommendations. At block 592, processing logic may output data to a user interface. The output data may include the data from the oral state capture modalities, the estimated oral conditions, the actionable symptom recommendations, the diagnoses of oral health problems, and/or the treatment recommendations.

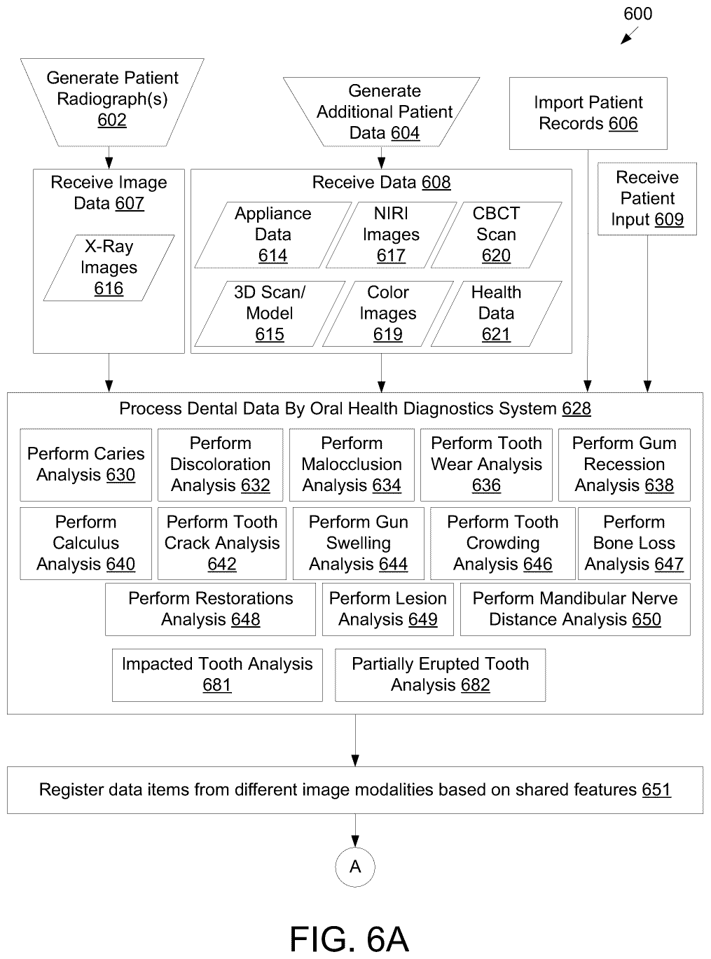

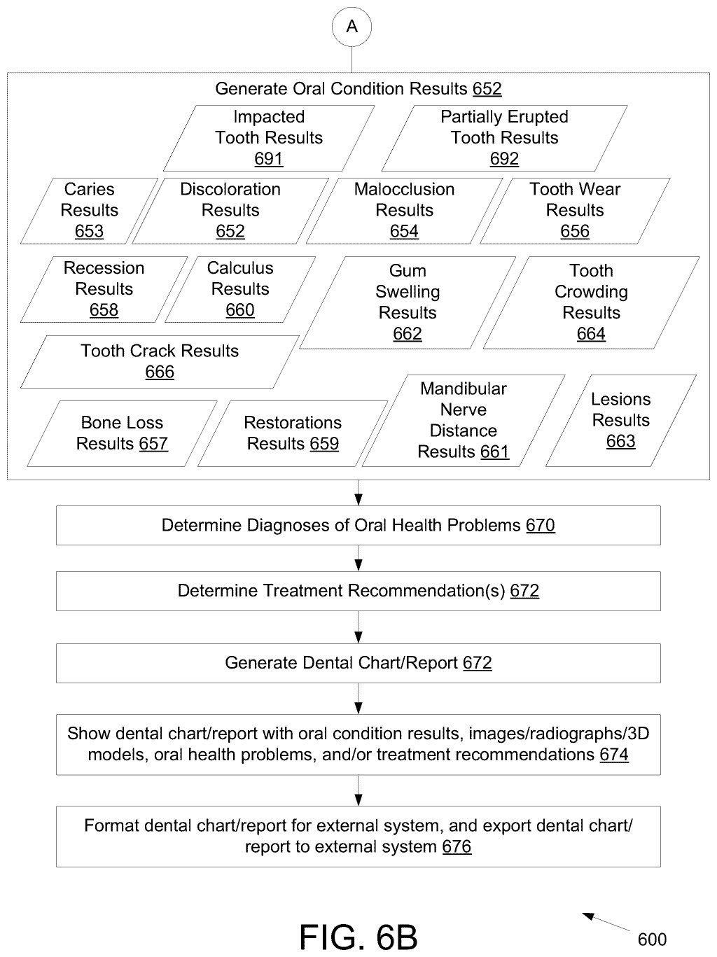

FIGS. 6A-B illustrate a flow diagram for a method 600 of analyzing a patient's teeth and gums, in accordance with embodiments of the present disclosure. At block 602, a doctor or dental practitioner generates one or more patient x-rays (radiographs) of a patient's teeth. At block 607, processing logic receives the x-ray data from the imaging performed at block 602. The x-ray data may include one or more panoramic x-rays, bitewing x-rays and/or periapical x-rays 616.

At block 604, a doctor or dental practitioner may generate additional patient data. At bock 608, processing logic receives the additional patient data. The additional patient data may include 3D scan data (e.g., intraoral scans and/or a 3D model generated from intraoral scans) 615, NIRI images 617, color images 619, CBCT scans 620, appliance data 614 (e.g., sensor data from a dental appliance worn by the patient), and/or health data 614 (e.g., such as observations of the dental practitioner, biopsy results, data from a fitness tracker, etc.). At block 606, processing logic may import patient records for the patient. In some embodiments, the patient records are imported from a DPMS. Patient records may include a patient age, underlying health conditions previously recorded for the patient, prior oral conditions and/or oral health problems of the patient, and so on. At block 606, processing logic may receive the imported patient records. The imported patient records may include historical patient data, for instance. At block 609, processing logic may receive patient input, such as indications of pain, bleeding, concerns, and so on.

At block 628, processing logic processes the received dental data (including the data received at blocks 606, 607, 608, and/or 609) using one or more data analysis engine (e.g., segmentation engine 312, oral health diagnostics engine 321, input processing engine 310, treatment recommendation engine 325, etc. of FIG. 3) to identify the presence of one or more types of oral conditions and/or severity levels for the one or more detected oral conditions. Processing logic may perform a caries analysis 630, a discoloration analysis 632, a malocclusion analysis 634, a tooth wear analysis 636, a gum recession analysis 638, a plaque or calculus analysis 640, a tooth crack analysis 642, a gum swelling analysis 644, a tooth crowding analysis 646 (and/or tooth spacing analysis), a periodontal bone loss analysis 647, a restorations analysis 648, a lesion analysis (e.g., a periodontal radiolucency analysis) 649, a mandibular nerve distance analysis 650, an impacted tooth analysis 681, a partially erupted tooth analysis 682, and/or one or more other types of analyses, as discussed herein above and below. The various analyses may include point-in-time analyses as well as time-dependent analyses that are based on data from multiple different times. For example, older data may be compared to recent data to determine whether oral conditions have improved, stayed the same, worsened, etc. Additionally, trajectories for the various oral conditions at various areas of interest where the oral conditions were identified may be determined and projected into the future and/or past. In some embodiments, the outputs of one or more dental health analyzers are used (alone or together with intraoral data) as inputs to other dental health analyzers. For example, the presence or absence of malocclusion may be used as in input into a dental health analyzer that performs the caries analysis 630 and/or the dental health analyzer that performs plaque analysis 640.

At block 651, processing logic may register data items of different image modalities together based on shared features. Such registration may enable improved correlation and/or comparison of oral conditions detected from different oral state capture modalities.

At block 652, processing logic generates diagnostics results based on an outcome of the oral condition analyses performed at block 628. Oral condition results may be generated based on combining oral condition results from analysis of data from multiple different oral state capture modalities in embodiments. Processing logic may generate caries results 653, discoloration results 652, malocclusion results 654, tooth wear results 656, gum recession results 658, calculus results 660, gum swelling results 662, tooth crowding and/or spacing results 664, tooth crack results 666, periodontal bone loss results 657, restorations results 659, mandibular nerve distance results 651, lesions results 663, impacted tooth results 691, partially erupted tooth results 692 and/or other results in embodiments. The oral conditions results may include detected AOIs associated with each of the types of oral conditions, and severity levels of the oral conditions for the AOIs. The oral condition results may include qualitative measurements, such as size of an AOI, an amount of recession for a gum region, an amount of wear for a tooth region, an amount of change (e.g., for a caries, tooth wear, gum swelling, gum recession, tooth discoloration, etc.), a rate of change (e.g., for a caries, tooth wear, gum swelling, gum recession, tooth discoloration, etc.), and so on. The oral condition results may further include qualitative results, such as indications as to whether an oral condition at an AOI has improved, has stayed the same, or has worsened, indications as to the rapidity with which the oral condition has improved or worsened, an acceleration in the improvement or worsening of the oral condition, and so on. An expected rate of change may have been determined (e.g., automatically or with doctor input), and the measured rate of change for an oral condition at an AOI may be compared to the expected rate of change. Differences between the expected rate of change and the measured rate of change may be recorded and included in the oral condition results. Each of the oral condition results may be automatically assigned a code on dental procedures and nomenclature (CDT) code or other procedural code for health and adjunctive services provided in dentistry. Each of the oral condition results may automatically be assigned an appropriate insurance code and related financial information in embodiments.

At block 670, processing logic may determine diagnoses and/or actionable symptom recommendations of one or more oral health problems based on the determined oral conditions. At block 672, processing logic may determine one or more treatment recommendations based on the oral conditions and/or diagnoses of oral health problems. Each of the types of oral conditions, actionable symptom recommendations and/or oral health problems may be associated with one or more standard treatments that are performed in dentistry and/or orthodontics to treat that type of oral condition and/or oral health problem. Based on the locations of identified AOIs, the oral conditions for the identified AOIs, the number of AOIs having oral conditions, the severity levels of the oral conditions, and/or the identified oral health problems, a treatment plan may be suggested. A doctor may review the treatment plan and/or adjust the treatment plan based on their practice and/or preferences. In some embodiments, the doctor may customize the oral health diagnostics system to give preference to some types of treatment options over other types of treatment options based on the doctor's preferences. Treatments may be determined for each of the identified oral conditions and/or oral health problems that are determined to have clinical significance.

At block 672, processing logic may generate a dental chart, report, insurance claim, and/or other output. In embodiments, images and/or documentation of one or more identified procedures may be exported from image data and/or determined oral conditions and associated with specific treatment codes (e.g., CDT codes) for justification of a procedure performed or to be performed, such as for insurance coverage purposes. In an example, images of gingival inflammation and/or calculus (supragingival or subgingival if detectable) can go with (or be user configured to be attached to) periodontal procedure codes. In another example, images of caries can go with or be attached to restorative codes, images of fractures or abfractions can go with or be attached to occlusion and prosthodontic codes, images of crowding/spacing can go with or be attached to orthodontic codes, and so on. This may satisfy the requirements of some insurance companies, which may require documentation prior to authorizing certain procedures.

At block 674, processing logic presents clinical indications of the oral condition analysis results, actionable symptom recommendations, diagnoses of oral health problems, and/or treatment recommendations in a user interface of an oral health diagnostics system, such as shown in FIG. 3. The clinical indications may additionally be automatically added to a patient chart. For example, a patient chart may automatically be updated to identify each identified oral condition, a tooth and/or gum region affected by the oral condition, a severity level of the oral condition, and/or other information about an AOI at which the oral condition was identified. The doctor may add notes about the oral conditions as well, which may also be added to the patient dental chart.

The information presented in the user interface may include qualitative results and/or quantitative results of the various analyses. All of the results of the analyses may be presented together in a unified view that improves clinical efficiency and provides for improved communication between the doctor and patient about the patient's oral health and how best to treat oral conditions and/or oral health problems. The summary of the oral condition results may include or display specifics on where AOIs associated with particular oral conditions were identified and/or how many such AOIs were identified.

The oral conditions may be ranked based on severity level in embodiments. In embodiments, the oral conditions are grouped into multiple classifications, where one classification may indicate that no issues were found (indicating that there is no need for the doctor to review those oral conditions), one classification may indicate that potential issues were found (indicating that the doctor might want to review those oral conditions, but that such review is not urgent), and/or one classification may indicate that issues were found (indicating that the doctor is recommended to immediately review those oral conditions).

The information presented in the user interface may include information identifying one or more new oral conditions that were detected in the current or most recent patient visit but not in the prior patient visit. The information presented in the user interface may include information identifying one or more preexisting oral conditions that have improved between the prior patient visit and the current or most recent patient visit. The information presented in the user interface may include information identifying one or more preexisting oral conditions that have worsened between the prior patient visit and the current or most recent patient visit. The information presented in the user interface may include information identifying one or more preexisting oral conditions that have not changed between the prior patient visit and the current or most recent patient visit.

At block 676, processing logic may format the dental chart and/or report for ingestion by an external system (e.g., a DPMS, treatment planning system, treatment management system, etc.), and may provide the formatted report, dental chart, etc. to the external system.

In some instances, processing logic may receive a selection of a type of oral condition to review, of a particular tooth or area of interest to review, etc. For example, a doctor may select caries indications to review. Via the user interface, the doctor may review detailed information about the type of oral condition that was selected, the tooth that was selected, etc. Processing logic may receive user input (e.g., from the doctor) regarding a selected oral condition via the user interface. The user input may include a user input defining one or more case specific areas of interest and/or issues (e.g., oral conditions) of interest for follow-up in future visits. In a future patient visit, the dentist may generate new data for the patient, and that new data may be used along with the definition of the AOIs and/or issues of interest when performing future analysis of the patient's dental health. The user input may additionally or alternatively include a user input defining customization for AOIs and/or issues (e.g., oral conditions) of interest for all patients. For example, the doctor may define criteria (e.g., thresholds) for detecting oral conditions and/or for assessing the severity of oral conditions. The doctor may additionally or alternatively override analysis results, such as by manually updating an AOI that was indicated as being an issue for a particular class of oral condition so that it is not labeled as an issue. The oral health diagnostics system may be customized for and/or by a doctor to enable that doctor to develop their own workflows to help walk a patient through their oral health, detected oral conditions, and options for addressing the oral conditions.

In embodiments, processing logic may receive a request for a prognosis simulation. Processing logic may simulate a prognosis of the oral condition with and/or without treatment. The prognosis simulation may be based on the determined AOIs and a selected oral condition. If a treatment was selected and/or suggested, then the suggested and/or selected treatment option may be used in determining the prognosis. In embodiments, a first prognosis without treatment may be generated to show a likely course of the oral condition without treatment and a second prognosis with treatment may be generated to show a likely course of the oral condition with treatment. The generated prognosis (or multiple prognoses) may be output via a user interface. The prognosis or prognoses may be shown to a patient and/or may be sent to the patient for consideration (e.g., a link to the prognosis may be sent to the computing device of the patient).

The prognosis may include automatically generated patient communication data (also referred to as educational information) that facilitates the doctor communicating with the patient about the prognosis and possible treatments. Patient communication data may be generated for each of the types of detected oral conditions and/or oral health problems, and may be presented to the patient together via a unified presentation or separately as discrete presentations for one or more types of oral conditions and/or oral health problems. The patient communication data may include textual and/or graphical information explaining and highlighting the findings and prognoses in an easy way to understand for non-clinicians. The patient communication data may show prognoses of the patient using the patient's own dentition, projected into the future with and/or without treatment. The patient communication data may include data for one or a number of selected AOIs and/or oral conditions, or may include data for each of the AOIs and/or oral conditions or for each of the AOIs and/or oral conditions that exceed a particular severity level threshold or thresholds.

In an example, the patient communication data may include some or all of the oral conditions that were identified that the doctor agreed should be addressed and/or monitored. The patient communication data may further include a comparison to oral conditions and/or AOIs of the patient at prior visits, and indications of how the AOIs and/or oral conditions have changed between visits. The patient communication data may include indications as to whether an AOI and/or oral condition was discussed previously, and a decision that was made about the AOI and/or oral condition. For example, the patent communication data may indicate that the patient already has been informed of a problem and that the doctor and/or patient are keeping an eye on the problem but are not planning on treating the problem at the present time.

Educational information may be presented, which may or may not be tailored based on the patient's dentition (e.g., using 3D models of the patient's dental arches). The educational information may show progression of the patient through different severity levels of an oral condition and/or oral health problem, using that patient's own dentition. In embodiments, the oral condition analysis tools may be used to segment the patient's dental arches into teeth and gingiva, to identify oral conditions in the teeth and in the gingiva, and to predict and provide animations for progression of the various oral conditions for the patient's dental arches. Educational information may also show how the progression of oral conditions may be stopped or reversed with treatment options and/or with changes in patient behavior (e.g., brushing twice daily, flossing, wearing a night guard, etc.). Such educational information may be shown for each of the types of oral conditions discussed herein.

The information about oral conditions and/or AOIs to monitor, and the information about oral conditions and/or AOIs to treat, may be used to generate a customized report for the patient in an automated manner, with little or no doctor input. The patient communication data may further include sequencing information identifying first treatments to be performed and/or oral conditions to be addressed, subsequent treatments to be performed and/or oral conditions to be addressed, and so on. The patient communication data may indicate which treatments are optional and which treatments are necessary for the patient's dental health, and may further indicate urgency associated with each of oral conditions and associated treatments. For example, oral conditions that are emergencies may be identified, those oral conditions that should be addressed in the near future (e.g., next few months) may be identified, and those oral conditions that are not urgent but that should be addressed eventually may be identified. This enables the doctor and patient to prioritize treatment options. The patient communication data may further include information on the percentage of doctors that treat specific oral conditions, the types of treatments for those oral conditions that are performed and the rates at which those treatments are performed, and so on.

In one embodiment, processing logic may receive an indication of one or more AOIs and/or oral conditions to monitor and/or to treat. Processing logic may update a patient record to follow-up regarding the AOIs and/or oral conditions that were identified for monitoring. At a next patient visit, processing logic will flag those AOIs and/or oral conditions that were marked for follow-up.

Processing logic may output visualizations of the indications and/or prognosis for patient review, as discussed above. The presented information may include information regarding insurance information (e.g., whether insurance covers a treatment) and/or cost. For example, the presented information may include a cost breakdown of the costs for each of the treatments to be performed to treat the one or more oral conditions. The patient may accept or decline one or more treatment options. Responsive to acceptance of a treatment option (or multiple treatment options), processing logic may automatically populate insurance paperwork with information about the oral condition(s), oral health problem(s) and/or treatment(s), and may automatically deliver the filled out insurance paperwork to an insurance company (e.g., via a discretionary portfolio management solution (DPMS) system) and/or obtain pre-authorizations from the insurance company (e.g., via a response received from the insurance company) prior to commencement of one or more treatments.

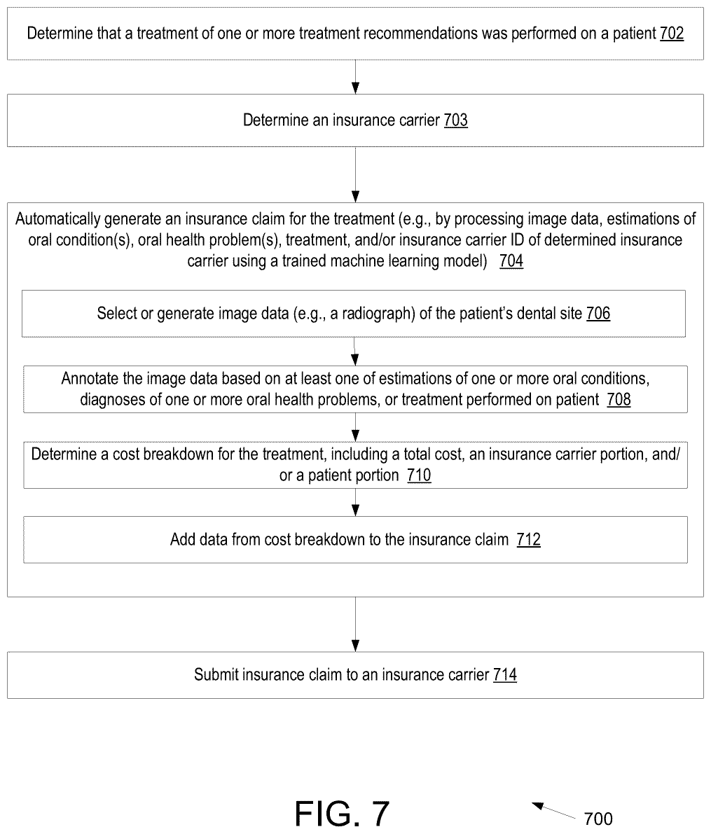

FIG. 7 illustrates a flow diagram for a method 700 of automatically generating an insurance claim for an oral health treatment, in accordance with embodiments of the present disclosure. At block 702 of method 700, processing logic determines that a treatment of one or more treatment recommendations was performed on a patient. The treatment recommendations may have been generated, for example, by an oral health diagnostics engine in accordance with any of methods 400-600. At block 703, processing logic may determine an insurance carrier of a patient on which the treatment was performed. The insurance carrier may be determined, for example, based on an inquiry to a DPMS.

At block 704, processing logic automatically generates an insurance claim for the treatment. The insurance claim may be generated, for example, by processing image data, estimations of oral conditions, diagnoses of oral health problems, data on the performed treatment, and/or an insurance carrier identifier (ID) of the determined insurance carrier. In some embodiments, some or all of this information is input into a trained ML model trained to generate insurance claims. The ML model may generate an insurance claim for the treatment of the oral conditions that is formatted for the indicated insurance carrier in embodiments. The ML model may have been trained based on training data of insurance claims that were granted and insurance claims that were denied so as to generate insurance claims that are more likely to be granted in embodiments.

In one embodiments, at block 706 processing logic selects or generates image data (e.g., a radiograph) of the patient's dental site (e.g., jaw, dental arch, one or more teeth, etc.) that was treated. For example, a received radiograph that shows the dental site may be selected. Alternatively, a generative model may generate a synthetic image representative of the dental site prior to treatment and/or after treatment. At block 708, processing logic may automatically annotate the image data based on at least one of the estimations of one or more oral conditions that were treated, diagnoses of one or more oral health problems, and/or treatment(s) performed on the patient. At block 710, processing logic may determine a cost breakdown for the treatment, optionally including a total cost of the treatment, an insurance carrier portion and/or a patient portion of the cost. At block 712, processing logic may add data from the cost breakdown to the insurance claim.

At block 714, processing logic may submit the insurance claim to the insurance carrier. In embodiments, processing logic accesses information on how and where to electronically send insurance claims, and may use such information to send out the insurance claim.

A dental health diagnostics system may gather information and statistics about many different doctors, dental practices, and so on for one or more geographic regions. Such data may include pre-treatment patient records and post-treatment patient records for many patients. The dental diagnostics system may evaluate such information to make determinations about common practices in dentistry, success rates of various treatments based on differing starting oral conditions, types of oral state capture modalities used to gather information about patients and assess their oral health, and so on. Based on such determinations, the oral health diagnostics system may provide recommendations to individual doctors and/or to dental practices.

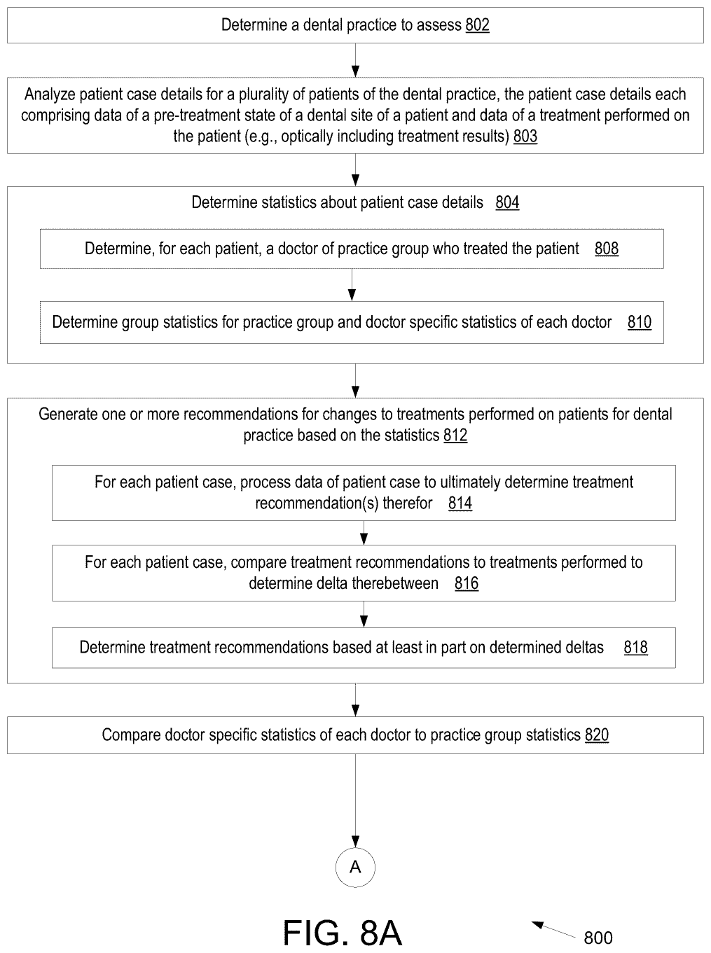

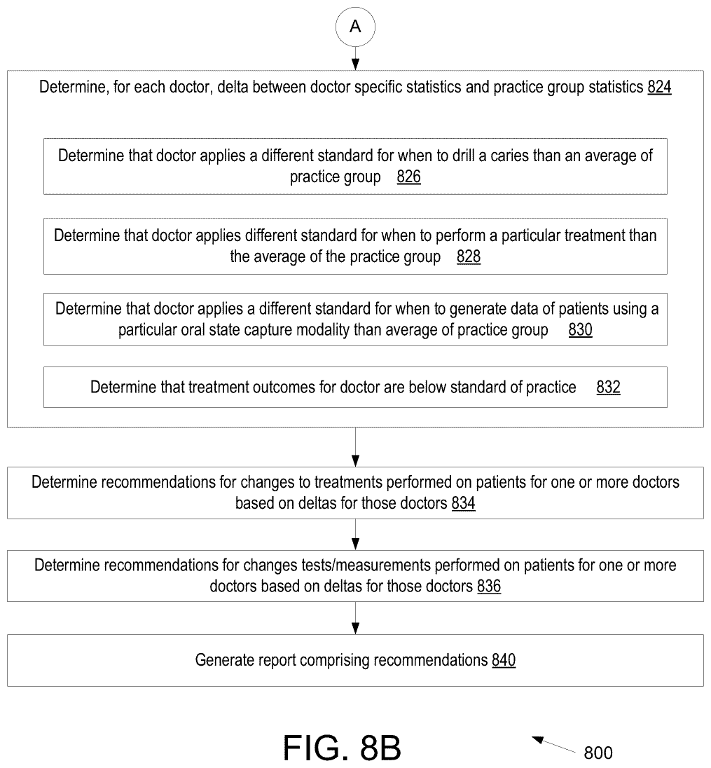

FIGS. 8A-B illustrate a flow diagram for a method 800 of generating a report for a dental practice, in accordance with embodiments of the present disclosure. At block 802 of method 800, processing logic determines a dental practice to assess. The dental practice may be selected from a list of dental practices that use an oral health diagnostics system in accordance with embodiments described herein. In embodiments, the dental practice may be selected by selecting a geographic region, which may cause dental practices of that region to be displayed, and then selecting one or more displayed dental practices. In some embodiments, a dental practice to assess is automatically selected based on a user identifier and/or dental practice identifier associated with a request to assess the dental practice. For example, a dental practice may be limited to assessment of their own practice, and may not have access to information about other practices, or may have anonymized information about other practices.

At block 803, processing logic analyzes patient case details for a plurality of patients of the dental practice. The patient case details may each comprise data of a pre-treatment state of a dental site of a patient and data of a treatment performed on the patient. The patient case details may additionally include data on post-treatment results of the dental site. Other data that may be included in the patient case details include additional information about the patient (e.g., health conditions, patient habits, doctor observations, patient history, etc.), treatment recommendations that were provided by an oral health diagnostics system, information on tests performed on the patient and/or types of oral state capture modalities used to assess an oral health of the patient, and/or other information.

At block 804, processing logic determines statistics about the patient case details for the plurality of patients of the dental practice. Statistics may be generated based on aggregated patient case details of the patients for the dental practice. Statistics may include numbers of patients having particular oral conditions and/or particular diagnosed oral health problems, numbers of various treatments performed to treat each of the oral conditions and/or oral health problems, success rates of performed treatments, and so on. In one embodiment, at block 808 processing logic determines, for each patient, a doctor of the practice group who treated the patient. At block 810, processing logic may determine group statistics for the dental practice group, and may further determine doctor specific statistics for one or more doctor of the practice. In an example, processing logic may determine a subset of the plurality of patients for which a treatment recommendation for a particular treatment was generated but for which the particular treatment was not performed, wherein the one or more recommendations comprise a recommendation to perform the particular treatment. In another example, processing logic may determine a subset of the plurality of patients for which a treatment recommendation for a particular treatment was not generated but for which the particular treatment was performed, wherein the one or more recommendations comprise a recommendation not to perform the particular treatment.

At block 812, processing logic generates one or more recommendations for changes to treatments performed on patients for the dental practice based on the statistics. In one embodiment, at block 814, for each patient case, processing logic may process data of the patient case to ultimately determine treatment recommendations therefor. The patient data may be processed by the oral health diagnostics system of FIG. 3 according to any of methods 400-600, for example. The treatment recommendations may be based on oral conditions identified in patient data and/or based on diagnoses for oral health problems associated with such identified oral conditions, and may be further based on best practices for treating various oral conditions and/or various diagnosed oral health problems. At block 816, for each patient case, processing logic may compare the treatment recommendations to treatments that were actually performed on the patient of the patient case, and may determine a delta between the performed treatments and the recommended treatments. At block 818, processing logic may determine recommendations for changes to treatments performed on patients based on the determined deltas. The proposed changes to treatments may be provided at a dental practice group level (e.g., based on overall statistics for the dental practice group) and/or at an individual doctor level (e.g., based on statistics for an individual doctor and/or based on comparison of statistics for the individual doctor to statistics for the dental practice group).

At block 820, processing logic may compare doctor specific statistics for each doctor to the practice group statistics. At block 824, processing logic may determine, for each doctor of the dental practice group, a delta between doctor specific statistics of the doctor and practice group statistics for the dental practice group.

In one embodiment, at block 826 processing logic determines that a doctor applies a different standard for when to drill a caries than an average of the practice group. For example, the doctor may drill caries having particular properties, whereas a rest of the practice group may choose to monitor a caries having the particular properties. In another example, the doctor may choose to monitor a caries having particular properties, whereas the rest of the practice group may choose to drill caries having the particular properties.

In one embodiment, at block 828 processing logic determines that a doctor applies a different standard for when to perform a particular treatment than an average of the practice group. Examples of dental treatments that processing logic may consider include dental cleaning, applying dental fillings, applying dental crowns, performing root canal therapy, applying dental implants, applying dental bridges, extracting teeth, applying dentures, performing orthodontic treatment, performing gum disease treatment (periodontal therapy), performing tooth whitening, performing dental bonding, applying dental veneers, performing oral surgery, and so on.

In one embodiment, at block 830 processing logic determines that a doctor applies a different standard for when to generate data of patients using a particular oral state capture modality than an average of the dental group practice. For example, a doctor may not perform CBCT scans as frequently as a remainder of the dental practice, or may not perform intraoral scans as frequently as a remainder of the dental practice, or may not generate panoramic dental radiographs as frequently as a remainder of the dental practice, or may perform one or more tests/measurements using an expensive oral capture state modality more frequently than an average of the dental practice group.

In one embodiment, at block 832 processing logic determines that treatment outcomes for a doctor are above or below a standard of the dental practice group. For example, processing logic may determine that a particular doctor's patients needed to come in for follow-up visits to fix prior work more often than a remainder of the dental practice group. In another example, processing logic may determine that crowns, fillings and/or other restorations of a particular doctor have a shorter lifespan than those of other doctors.

At block 834, processing logic may determine recommendations for treatments performed on patients for one or more doctors based on the deltas between statistics for those doctors and group statistics. At block 836, processing logic may determine recommendations for changes in tests/measurements performed on patients for one or more doctors based on deltas between statistics for those doctors and group statistics. For example, processing logic may recommend that a doctor perform intraoral scans, perform CBCT scans, generate panoramic x-rays, etc. in certain use cases for which the doctor generally does not perform intraoral scans, perform CBCT scans, and/or generate panoramic x-rays.

At block 840, processing logic may generate a report comprising recommendations for changes to a dental practice group and/or for individual doctors. The report may additionally or alternatively show, for one or more doctors of a group practice, a delta between the doctor specific statistics for those doctors and the statistics of the dental practice. Based on the report the dental practice group and/or doctor may update their habits, treatments, tests, patient imaging, etc. to improve their practice.

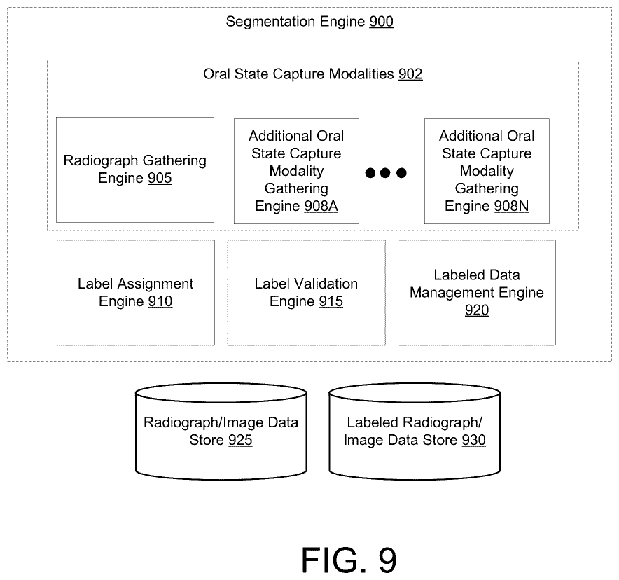

FIG. 9 illustrates a segmentation engine 900 of an oral health diagnostics system, in accordance with embodiments of the present disclosure. Segmentation engine 900 may correspond to segmentation engine 312 of FIG. 3 in an embodiment. In the example segmentation engine 900, a radiograph gathering engine 905 can gather unsegmented or raw radiographs from a relevant source. For instance, unsegmented radiographs can be gathered from a radiograph datastore 925 and/or from a physical x-ray device (e.g. an oral state capture system) coupled to a computing device executing or in communication with the oral health diagnostics system. That is, the radiograph gathering engine 905 can retrieve stored radiographs or can receive radiographs in real-time or near real-time as they are generated by an x-ray machine, for example. In some implementations, the radiograph gathering engine 905 receives radiographs from a networked resource, such as a website, intranet, or shared folder. In practice, the radiograph gathering engine 905 may implement instructions that obtain radiographs by time, date, patient name, and/or another identifier. The radiograph gathering engine 905 can store raw radiographs in the radiograph datastore 925 in embodiments. One or more additional oral state capture modality gathering engines 908A-N can similarly receive data of various other oral state capture modalities. The data can be received from and/or stored in data store 925 and/or another data store. In some embodiments, a shared data store is used for multiple oral state capture modalities. Alternatively, different data stores may be used for different oral state capture modalities. Additional oral state capture modality gathering engines 908A-N may also receive data in real time or near real time as the data is generated by, for example, electronic compliance indicators, intraoral scanners, cameras, mobile phones, CBCT machines, etc.

A label assignment engine 910 can implement processes to identify and/or review features of radiographs and/or data from other image modalities for the presence/absence of oral anatomical structures and/or oral conditions. Label assignment engine 910 may include one or more segmentation pipelines, which may each include one or more logics for performing image processing and/or one or more AI models and/or ML models for processing radiographs/images to perform segmentation, object detection, and so on. The label assignment engine 910 can assign labels to specific pixels in the radiograph/image. The labels can represent characteristics that all pixels with that label would have, for example. In some implementations, the label assignment engine 910 gets features from a feature datastore to evaluate the pixels of radiographs. The label assignment engine 910 may also get labels from a label datastore. A label validation engine 915 may validate labels and perform quality assessment through a variety of techniques. In one example, label validation engine 915 performs comparison of labels from a radiograph/image with data from other images, radiographs and/or other oral state capture modalities.

Once labels are assigned, a labeled data management engine 920 can create, store, and/or share labeled (e.g., segmented) data (e.g., radiographic representations) of an oral cavity. For example, labeled data management engine 920 can store labeled radiographs, labeled images, etc., in a labeled radiograph/image datastore 930, share these with one or more visualization engines (e.g., visualization engine 330 of FIG. 3), share these with modules that use segmented radiographic representations and/or image representations of an oral cavity for various reasons, etc.

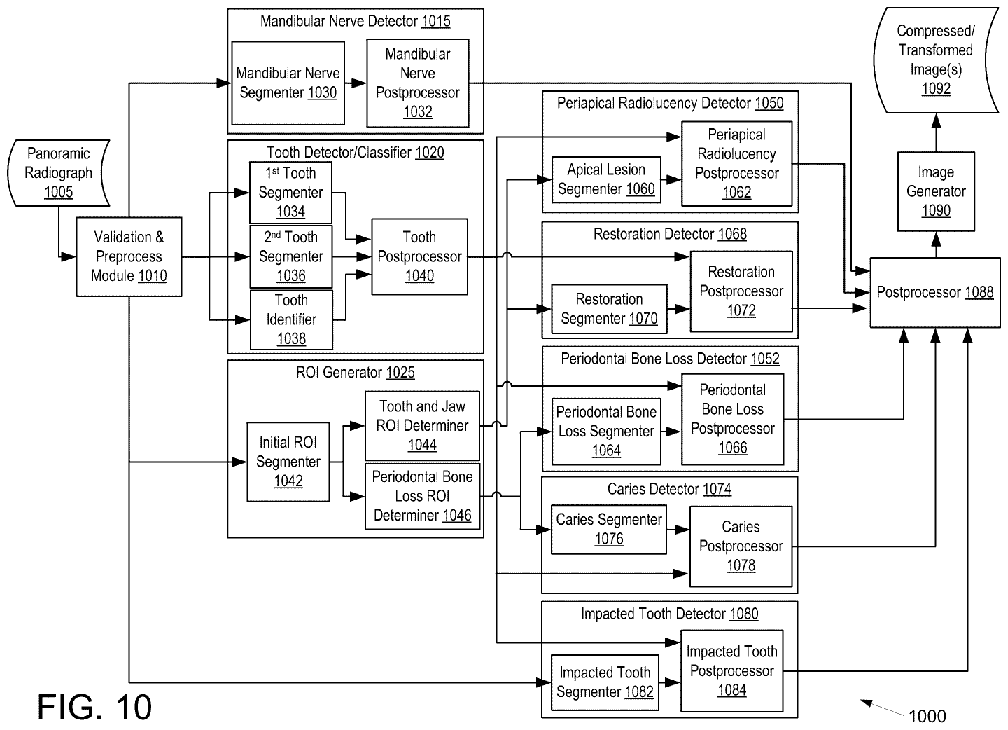

FIG. 10 illustrates an example segmentation pipeline 1000 for performing segmentation of a first type of dental radiographs, in accordance with embodiments of the present disclosure. Segmentation pipeline 1000 may be used for processing panoramic radiographs (e.g., panoramic radiograph 1005) in embodiments. While example segmentation pipeline 1000 is shown for panoramic radiographs, a same or similar segmentation pipeline may also be used for processing other types of radiographs and/or other types of image data (e.g., CBCT scans, 2D images of oral cavities, 3D models of dental arches, and so on).

Segmentation pipeline 1000 may include multiple modules (e.g., mandibular nerve detector 1015, tooth detector/classifier 1020, region of interest (ROI) generator 1025, etc.) that may separately operate on an input radiograph in parallel, and may further include one or more additional modules (e.g., periapical radiolucency detector 1050, restoration detector 1068, periodontal bone loss detector 1052, caries detector 1074, impacted tooth detector 1080, etc.) that operate on a radiograph and/or on an output of one or more of the first modules, optionally in parallel. Segmentation pipeline 1000 further includes one or more additional modules (e.g., postprocessor 1088, image generator 1090, etc.) that may operate on combinations of outputs from other modules to ultimately generate one or more images and/or overlays (e.g., a transformed and/or compressed radiograph or other image 1092 having one or more overlay layers of detected dental conditions, segmented and identified teeth, and so on).

A validation and preprocess module 1010 of segmentation pipeline 1000 may receive a panoramic radiograph 1005. Validation may include processing the radiograph to ensure that it satisfies one or more radiograph criteria, and to ensure that it is suitable for processing by the segmentation pipeline 1000. For example, validation and preprocess module 1010 may measure a blurriness of the radiograph and determine whether the blurriness is below a blurriness threshold. Preprocessing operations performed on the radiograph may include any of those previously discussed, such as sharpening, edge detection, cropping, and so on. Once the radiograph 1005 is validated and preprocessed, the preprocessed radiograph may be provided to mandibular nerve detector 1015, tooth detector/classifier, ROI generator 1025 and/or impacted tooth detector 1080.

Mandibular nerve detector 1015 may include a mandibular nerve segmenter 1030 and a mandibular nerve postprocessor 1032. Mandibular nerve detector 1015 may include one or more trained ML models that process the radiograph 1005 to generate an output comprising at least one of an identification or a location of a mandibular nerve canal. The output may include segmentation information of the mandibular nerve canal. In an embodiment, the output of the mandibular nerve segmenter 1030 includes a mask that indicates, for each pixel of the radiograph 1005, whether the pixel is part of a mandibular nerve canal of a patient. In an embodiment, the output of the mandibular nerve segmenter 1030 includes a boundary (e.g., a bounding shape such as a bounding box) around the mandibular nerve canal of the patient.

Mandibular nerve detector 1015 may additionally include a mandibular nerve postprocessor 1032 that updates an output of the mandibular nerve segmenter 1030 to generate postprocessed mandibular nerve segmentation information. The postprocessed mandibular nerve segmentation information may be provided to a postprocessor 1088. In embodiments, updating the output of the mandibular segmenter 1030 includes combining this information with tooth segmentation and tooth classification results to indicate which of the lower molars are potentially in close proximity to the mandibular nerve canal. This warning is relevant for dentists decisions on certain procedures and may be visualized in the GUI on the image as well as on the tooth chart in embodiments.

Tooth detector/classifier 1020 may include a first tooth segmenter 1034, a second tooth segmenter 1036, a tooth identifier 1038, and/or a tooth postprocessor 1040. First tooth segmenter 1034, second tooth segmenter 1036, and/or tooth identifier 1038 may each include one or more trained ML models that process the radiograph 1005 to generate an output. In some embodiments, first tooth segmenter 1034, second tooth segmenter 1036, and/or tooth identifier 1038 form an ensemble ML model for tooth segmentation. In one embodiment, first tooth segmenter 1034 comprises a first ML model that generates a pixel-level segmentation mask of the patient's teeth. The pixel-level segmentation mask may separately identify each tooth, or may provide a single pixel-level identification of teeth, without separately calling out individual teeth. In one embodiment, second tooth segmenter 1036 also generates a pixel-level segmentation mask of the patient's teeth. However, unlike the first pixel-level segmentation mask the second pixel-level segmentation mask may separately identify each tooth. Accordingly, first tooth segmenter 1034 may perform semantic segmentation of the radiograph 1005, and second tooth segmenter 1036 may perform instance segmentation of the radiograph 1005 in embodiments. In one embodiment, tooth identifier 1038 comprises an ML model trained to identify, and output separate bounding shapes (e.g., bounding boxes) around, each tooth in the radiograph 1005. Tooth identifier 1038 may determine a tooth number for each identified tooth, and may assign the determined tooth number to a generated bounding shape. Tooth numbers may correspond to any tooth numbering scheme, some of which are discussed elsewhere herein.

The outputs of first tooth segmenter 1034 (e.g., first tooth segmentation information), second tooth segmenter 1036 (e.g., second tooth segmentation information) and tooth identifier 1038 (e.g., tooth identification information) may be processed by tooth postprocessor 1040 of tooth detector/classifier 1020. Tooth postprocessor 1040 may compare the first tooth segmentation information, second tooth segmentation information, and tooth identification information. Based on the comparison, tooth postprocessor 1040 may identify and reconcile discrepancies between the first tooth segmentation information, second tooth segmentation information, and/or tooth identification information. Tooth postprocessor 1040 may include one or more modules comprising rules for how to reconcile disagreements between the first tooth segmentation information, second tooth segmentation information, and/or tooth identification information. Tooth postprocessor 1040 may additionally include one or more additional models (e.g., ML models) configured to process panoramic radiograph 1005 to determine additional tooth segmentation and/or tooth identification information to generate additional data points for resolving discrepancies.

Based on the combined outputs of first tooth segmenter 1034, second tooth segmenter 1036 and/or tooth identifier 1038, tooth postprocessor 1032 may generate one or more masks for teeth of the patient. Each of the masks may be based on combined information from the multiple outputs, and may be assigned a tooth number. For example, tooth postprocessor 1040 may determine, based on the output of the first tooth segmenter 1034, second tooth segmenter 1036 and/or tooth identifier 1038, tooth numbering for each identified tooth. Tooth postprocessor 1040 may determine whether the tooth numbering applied to the identified teeth satisfy one or more criteria or constraints. In instances where the tooth numbering satisfies one or more tooth numbering rules or constraints, the tooth numbering may be verified. For instances where the tooth numbering fails to satisfy one or more tooth numbering rules or constraints, the tooth numbering may be corrected. For teeth whose tooth numbering does not satisfy one or more tooth numbering constraints, the tooth numbers assigned to such teeth may be updated to cause the updated tooth numbering to satisfy the constraints. In one embodiment, tooth postprocessor 1040 includes a statistical model that processes the radiograph 1005 and/or the first segmentation information, second segmentation information, tooth identification information, and/or combination thereof, to update the tooth numbering of the identified teeth. In one embodiment, tooth postprocessor 1040 identifies duplicate tooth numbers, and removes duplicates of tooth numbers. In one embodiment, tooth postprocessor 1040 counts a number of unique teeth, and removes one or more tooth identifications responsive to determining that more than 32 teeth were identified (e.g., since the human body includes at most 32 teeth for the vast majority of the population). In one embodiment, tooth postprocessor 1040 determines whether tooth numbers are sorted and ordered (e.g., are left to right sorted and ordered). In instances where one or more tooth numbers are out of order, tooth postprocessor 1040 may update the tooth numbering to cause the tooth numbers to be sorted and ordered (e.g., left to right sorted and ordered).

Tooth postprocessor 1040 may output refined tooth segmentation information that includes instance segmentation and accurate assigned tooth numbering for each tooth in the radiograph 1005. The refined tooth segmentation information may be provided, for example, to periapical radiolucency detector 1050, periodontal bone loss detector 1052, caries detector 1074, and/or impacted tooth detector 1080 for further processing.

ROI generator 1025 may include an initial ROI segmenter 1042, a tooth and jaw line ROI determiner 1044, and/or a periodontal bone loss ROI determiner 1046 in embodiments. The initial ROI segmenter 1042 may include one or more trained ML models that perform segmentation in embodiments. The initial ROI segmenter 1025 may segment the radiograph 1005 into one or more regions of interest. The regions of interest may be overlapping and/or non-overlapping. For example, some regions of interest may be within and/or overlapping with other regions of interest. Examples of regions of interest that may be identified include ROIs for individual teeth (e.g. individual teeth segments), an ROI of a lower jaw, an ROI of a convex hull around the lower jaw, an ROI of a periodontal bone line, an ROI of jaws and teeth, and so on. Initial ROI segmenter 1042 may include one or more trained ML models trained to perform segmentation of the radiograph 1005 to segment the radiograph 1005 into a lower jaw, a convex hull around the lower jaw, one or more binary tooth segments, and/or periodontal bone level information, for example.

Tooth and jaw ROI determiner 1044 may process one or more outputs of initial ROI segmenter 1042 to determine ROIs of one or more teeth and/or of the patient's jaw. These ROIs may be used as inputs into further detectors (e.g., periapical radiolucency detector 1050, restoration detector 1070, etc.). In one embodiment, tooth an jaw ROI determiner generates one or more clipped versions of radiograph 1005 based on generated ROI segmentation information. For example, tooth and jaw ROI determiner may generate a clipped version of the radiograph 1005 in which areas outside of the jaw are removed, may generate a clipped version of the radiograph 1005 in which areas outside of the jaw plus a convex hull around the jaw are removed, may generate clipped versions of the radiograph each including information for an individual tooth, and so on. Periodontal bone loss ROI determiner 1046 may process one or more outputs of initial ROI segmenter 1042 to determine an ROI in the radiograph that includes information from the radiograph useful for determining periodontal bone loss. The periodontal bone loss ROI may be a region that includes a periodontal bone line, a cemento-enamel junction (CEJ), and/or one or more other areas useful for determining bone loss (e.g., such as tooth roots, tooth root apexes, etc.). The CEJ may be a region (e.g., a line) at which the enamel of a tooth ends, and may also be referred to as an enamel line. For example, the periodontal bone loss ROI may include an area of the radiograph between the crowns and root apexes of the teeth in the upper jaw and/or lower jaw of the patient. Accordingly, the periodontal bone loss ROI may be a region within which the periodontal bone line and/or CEJ can be found, and may not include regions that cannot reasonably contain the periodontal bone line or CEJ. In embodiments, the periodontal bone loss ROI is output by the initial ROI segmenter 1042. The periodontal bone loss ROI information may be provided to one or more additional detectors, such as periodontal bone loss detector 1052 and/or caries detector 1074.

Periapical radiolucency is the radiographic sign of inflammatory bone lesions around the apex of the tooth. Periapical radiolucency detector 1050 may receive tooth and jaw ROI information from ROI generator 1025, and may process areas of the radiograph 1005 indicated in the tooth and jaw ROI information. Areas of the radiograph outside of the teeth and jaw may not experience an apical lesion. Accordingly, by limiting the information that is considered by apical lesion segmenter 1060 to only information of the teeth and/or jaw the accuracy of the segmentation performed by the apical lesion segmenter 1060 may be improved.

Apical lesion segmenter 1060 may include one or more trained ML models that have been trained to segment a radiograph (or an ROI of a radiograph) into one or more apical lesions. The trained MLs may perform instance segmentation or semantic segmentation in some embodiments. In some embodiments, the trained MLs perform apical lesion detection, and identify bounding boxes around individual instances of apical lesions. Apical lesion segmenter 1060 outputs segmentation information identifying apical lesions within the teeth and/or jaw ROI(s). Areas exhibiting periapical radiolucency may correspond to areas of apical lesions. Accordingly, apical lesions may be detected based on detection of radiolucency by the apical lesion segmenter 1060 at or around one or more teeth in embodiments. Apical lesions may be detected for regions of the jaw and/or for individual teeth in embodiments. In one embodiment, a jaw ROI of the radiograph 1005 is processed by apical lesion segementer 1060. Additionally, or alternatively, each individual tooth ROI of the radiograph may be processed by apical lesion segmenter 1060 to determine whether an apical lesion is detected for an associated tooth.

Periapical radiolucency postprocessor 1062 may receive apical lesion segmentation information from apical lesion segmenter 1060. Additionally, periapical radiolucency postprocessor 1062 may receive tooth segmentation information from tooth detector/classifier 1020. Periapical radiolucency postprocessor 1062 may combine the tooth segmentation information and the apical lesion segmentation information to determine which tooth or teeth detected apical lesions are associated with. For each identified apical lesion, the apical lesion may be assigned to a single tooth or to multiple teeth based on the determined apical lesion segmentation information (e.g., which may be inflammation segmentation information) and tooth segmentation information of the plurality of teeth. If a single apical lesion covers multiple tooth roots, then the apical lesion may be assigned to multiple teeth. If a single apical lesion covers only a single tooth root, then the apical lesion may be assigned to just one tooth. Periapical radiolucency detector 1050 may output information (e.g., one or more masks, one or more bounding shapes/boxes, etc.) for each detected apical lesion, including one or more teeth associated with each apical lesion.

In dentistry, restorations are artificial dental prosthetics used to repair or replace damaged or missing teeth, restore the function of teeth, and/or improve the appearance of teeth. Restorations may include direct restorations (fabricated inside of the mouth) and indirect restorations (fabricated outside of the mouth and then installed in the mouth). Examples of direct restorations include dental fillings and dental bonding. Examples of indirect restorations include dental crowns, dental bridges, inlays and onlays, dental veneers, dental implants, and implant-supported dentures (e.g., all-on-four dentures). Dental fillings may be made of various materials, including amalgam (silver), composite resin, glass ionomer, or gold, and may be fillings in a tooth crown and/or fillings that extend to a tooth root. Dental bonding may include a composite resin material applied to a tooth surface to repair chipped, cracked, or discolored teeth, to close gaps between teeth, to reshape teeth, and so on. Dental crowns are custom-made prosthetic restorations that cover the entire visible surface of a damaged or weakened tooth above the gumline. Crowns restore the tooth's strength, shape, and function and can be made from materials such as porcelain, metal (gold or silver alloy), porcelain-fused-to-metal (PFM), or zirconia. Dental are fixed prosthetic devices used to replace one or more missing teeth by bridging the gap between adjacent teeth. Bridges consist of artificial teeth (pontics) held in place by crowns or metal framework anchored to the neighboring teeth. Inlays and onlays are indirect restorations used to repair larger cavities or tooth damage that cannot be adequately restored with traditional fillings but does not require a full dental crown. Inlays fit inside the cusp tips of the tooth, while onlays extend over one or more cusps. Dental veneers are thin, custom-made shells of porcelain or composite resin bonded to the front surface of teeth to improve their appearance by masking imperfections such as stains, chips, or misalignment. Dental implants are surgical placement of artificial tooth roots (implants) into the jawbone to support replacement teeth (crowns, bridges, or dentures). Implant-supported dentures are dentures that are anchored to dental implants placed in the jawbone, providing increased stability and retention compared to traditional removable dentures. Other types of restorations include partial dentures and full dentures.

Restoration detector 1068 is a module of segmentation pipeline 1000 capable of identifying and distinguishing between the many different types of restorations that are possible. Restoration detector 1068 may include a restoration segmenter 1070 and a restoration postprocessor 1072 in embodiments. Restoration detector 1068 may receive tooth and jaw ROI information from ROI generator 1025, and may process areas of the radiograph 1005 indicated in the tooth and jaw ROI information using restoration segmenter 1070. Areas of the radiograph outside of the teeth and jaw may not include restorations. Accordingly, by limiting the information that is considered by restoration segmenter 1070 to only information of the teeth and/or jaw the accuracy of the segmentation performed by the restoration segmenter 1070 may be improved.

Restoration segmenter 1070 may include one or more trained ML models that have been trained to segment a radiograph (or an ROI of a radiograph) into one or more restorations. The trained ML(s) may perform instance segmentation or semantic segmentation in some embodiments. In some embodiments, the trained ML(s) perform restoration detection, and identify bounding boxes around individual instances of restorations. Restoration segmenter 1070 outputs segmentation information identifying restorations within the teeth and/or jaw ROI(s). Restorations may have a different appearance in radiographs due to differences in material and/or density of restorations as compared to natural teeth. Accordingly, restorations may be detected based on differences in intensity by the restoration segmenter 1070 at or around one or more teeth in embodiments. Restorations may be detected for regions of the jaw and/or for individual teeth in embodiments. In one embodiment, a jaw ROI of the radiograph 1005 is processed by restoration segementer 1070. Additionally, or alternatively, each individual tooth ROI of the radiograph may be processed by restoration segmenter 1070 to determine whether a restoration is detected for an associated tooth.

Restoration postprocessor 1072 may receive restoration segmentation information from restoration segmenter 1070. Additionally, restoration postprocessor 1072 may receive tooth segmentation information from tooth detector/classifier 1020. Restoration postprocessor 1072 may combine the tooth segmentation information and the restoration segmentation information to determine which tooth or teeth detected restorations are associated with. For each identified restoration, the restoration may be assigned to a single tooth or to multiple teeth based on the determined restoration segmentation information and tooth segmentation information of the plurality of teeth. If a single restoration covers multiple teeth, then the restoration may be assigned to multiple teeth. If a single restoration covers only a single tooth root, then the restoration may be assigned to just one tooth.

In some instances restoration segmenter 1070 differentiates between different types of restorations, and outputs segmentation information indicating restoration types for each detected restoration. Additionally, or alternatively, restoration postprocessor 1072 may determine restoration types based on postprocessing of combined restoration segmentation information and tooth segmentation information. Examples of restoration types that may be detected include fillings, crowns, root canal fillings, implants, bridges, inlays, onlays, dental veneers, dentures, and so on.

In one embodiment, to determine a restoration type restoration postprocessor 1072 first determines, based on the tooth segmentation information and restoration segmentation information, a supporting tooth of the restoration. Restoration postprocessor 1072 may then determine a size of the supporting tooth and a size of the restoration. Restoration postprocessor 1072 may compare the size of the supporting tooth to the size of the restoration. Based on the relative sizes of the restoration and the supporting tooth, a restoration type may be determined for the restoration. For example, if a size of the supporting tooth is greater than a size of the restoration, then the restoration may be identified as a crown. If the size of the supporting tooth is substantially greater than the size of the restoration, the restoration may be identified as a filling. If the size of the restoration is determined to be approximately equal to, or greater than, a size of an individual tooth, then the restoration may be identified as a bridge.

Restoration detector 1068 may output information (e.g., one or more masks, one or more bounding shapes/boxes, etc.) for each detected restoration, including one or more teeth associated with each restoration and a restoration type of each restoration.

Periodontal bone loss is associated with periodontitis, and one or both may be detected in embodiments. In order to properly treat periodontitis, it is important to first establish a proper diagnostics. To diagnose periodontitis, multiple aspects may be taken into account, such as a patient's overall health and personal habits (e.g., whether the patient is a smoker, whether the patient has diabetes, information from radiographs, and so on). Radiographs can provide important information for diagnosis of periodontitis, including information on periodontal bone loss. For example, based on analysis of radiographs, periodontal bone loss detector 1052 may assess how much bone is still left that a patient's teeth can sit in. A progression of periodontal bone loss over multiple time periods may be assessed and projected into the future to determine a rate at which a patient's gums and/or bone mass in the jaw are retreating. Based on such information, processing logic may predict a point at which a patient's teeth will begin to fall out.

Periodontal bone loss detector 1052 is a module of segmentation pipeline 1000 capable of identifying and characterizing periodontal bone loss. Periodontal bone loss detector 1052 may include a periodontal bone loss segmenter 1064 and a periodontal bone loss postprocessor 1068 in embodiments. Periodontal bone loss segmenter 1064 may receive periodontal bone loss ROI information from ROI generator 1025, and may process areas of the radiograph 1005 indicated in the periodontal bone loss ROI information using periodontal bone loss segmenter 1064. Areas of the radiograph outside of the periodontal bone loss ROI may not be relevant to periodontal bone loss detection. Accordingly, by limiting the information that is considered by periodontal bone loss segmenter 1064 to only information of the teeth and/or jaw that might possibly include information relevant to periodontal bone loss (e.g., such as the periodontal bone line, tooth roots, tooth root apexes, and/or CEJ line), the accuracy of the segmentation performed by the periodontal bone loss segmenter 1064 may be improved.

Periodontal bone loss segmenter 1064 may include one or more trained ML models that have been trained to segment a radiograph (or an ROI of a radiograph) into one or more of a PBL, CEJ, tooth roots, tooth root apexes, etc. The trained MLs may perform instance segmentation or semantic segmentation in some embodiments. In some embodiments, the trained MLs perform detection of one or more dental classes, such as of a PBL, a CEJ, a tooth root, etc., and identify bounding boxes around individual instances of such dental classes. Periodontal bone loss segmenter 1064 may output segmentation information identifying a periodontal bone line, a CEJ line, tooth roots, and/or root apexes for one or more teeth within the periodontal bone loss ROI. Bone density above the periodontal bone line (PBL) may be lower than bone density below the PBL. Accordingly, the periodontal bone line may be detected based on differences in intensity associated with differences in bone density by the periodontal bone loss segmenter 1064 at or around one or more teeth in embodiments. Similarly, density of enamel may be higher than density of the cementum of a tooth. Accordingly, the CEJ line may be detected based on differences in intensity associated with differences in enamel density and cementum density by the periodontal bone loss segmenter 1064 at or around one or more teeth in embodiments.

Periodontal bone loss postprocessor 1066 may receive periodontal bone loss segmentation information (e.g., including for the PBL, CEJ, roots, root apexes, etc.) from periodontal bone loss segmenter 1064. Additionally, periodontal bone loss postprocessor 1066 may receive tooth segmentation information from tooth detector/classifier 1020. Based on the periodontal bone loss segmentation information and/or tooth segmentation information, periodontal bone loss postprocessor 1066 may determine a severity of the periodontal bone loss for a patient as a whole and/or for one or more teeth and/or regions of a patient's jaw.

In one embodiment, periodontal bone loss postprocessor 1066 identifies a CEJ line, a PBL, and/or tooth roots of one or more teeth based on the periodontal bone loss segmentation information and/or tooth segmentation information. Periodontal bone loss postprocessor 1066 may also identify root apexes (e.g., bottoms) of one or more tooth roots from the received bone loss segmentation information and/or tooth segmentation information (e.g., using image processing techniques). Periodontal bone loss postprocessor 1066 may determine a first distance between the root apex and the CEJ for one or more teeth and/or one or more regions of interest (e.g., which may span a single tooth or multiple teeth). Periodontal bone loss postprocessor 1066 may additionally determine a second distance between the CEJ and the PBL for the one or more teeth and/or one or more regions of interest. Periodontal bone loss postprocessor 1066 may determine a ratio between the first distance and the second distance, where the ratio may represent or correlate with the severity of the periodontal bone loss. Such values may be separately determined for different regions of a tooth in embodiments. For example, such values may be separately determined for a mesial side and for a distal side of each tooth. Additionally, or alternatively, periodontal bone loss postprocessor 1066 may determine a distance between the CEJ and the PBL, and may determine a severity of periodontal bone loss based on the distance between the CEJ and the PBL, where the distance may represent or correlate with the severity of the periodontal bone loss.

Severity of periodontal bone loss may be determined for individual teeth, for regions of individual teeth, for regions of the patient's jaw, for the patient as a whole, and so on. In embodiments, severity of periodontal bone loss is determined based on comparison of the computed distance and/or ratio to one or more thresholds. For example, the more bone level a patient loses, the larger the distance between the CEJ and the PBL, and the less strongly the teeth are connected to the patient's jaw. Accordingly, the larger the distance between the CEJ and the PBL, the greater the severity of the periodontal bone loss. Different distances between the CEJ and the PBL may be associated with different periodontal bone loss severity levels, where greater distances are associated with greater severity levels. Additionally, the ratio of a first distance between the PBL and the root apex and a second distance between the CEJ and the root apex may be used to determine severity of periodontal bone loss. If the ratio of the first distance to the second distance is 1:1 (e.g., 100% or simply 100), this indicates that the PBL is essentially at the root apex and that the patient has no bone left for the tooth to be seated in. If the ratio of the first distance to the second distance is about 0:1 (e.g., 0% or simply 0), this indicates that the PBL is essentially at the CEJ and that there is no bone loss. In some embodiments, the ratio of the first distance to the second distance is a value between zero and 100, where 100 would mean the tooth is not in the bone anymore, and a value of zero would mean the bone line is exactly at the cementoenamel junction.

In some embodiments, periodontal bone loss detector 1052 may receive or include one or more thresholds for values (e.g., distances, ratios, etc.) associated with one or more severity levels of periodontal bone loss. People naturally experience some level of periodontal bone loss over their lifetimes. Accordingly, the same amount of periodontal bone loss may have a different severity depending on the age of the patient for which the periodontal bone loss is detected. For example, a certain amount of periodontal bone loss in an octogenarian may be considered healthy, but the same amount of periodontal bone loss detected in a child may be of concern. Accordingly, in embodiments thresholds for associating levels of periodontal bone loss to severity ratings may be variable. In some embodiments, a doctor selects one or more thresholds to use for assessing severity of periodontal bone loss. In some embodiments, a patient age is provided to periodontal bone loss detector 1052, and thresholds to use for assessing severity of periodontal bone loss are set based on the patient age.

Based on measurements from periodontal bone loss detector 1052 over a time period, processing logic may determine a velocity of a change in periodontal bone loss over the time period. Processing logic may, comprehensively for each tooth surface, for every tooth, determine a trajectory and/or velocity of change for periodontal bone loss. This enables patients to be grouped into different risk profiles that may be used to assess treatments to be performed on the patients, doctor visitation schedules, and so on.

Periodontal bone loss postprocessor 1066 may combine the tooth segmentation information and the periodontal bone loss segmentation information to determine the level of the periodontal bone line at one or more teeth. For example, different portions of the identified periodontal bone line may be associated with different teeth. Based on the determined periodontal bone loss segmentation information and/or tooth segmentation information, periodontal bone loss postprocessor 1066 may determine bone loss values for each tooth and/or region. Based on these bone loss values, periodontal bone loss postprocessor may determine whether the patient has horizontal bone loss and/or vertical bone loss at one or more teeth or areas. Additionally, or alternatively, periodontal bone loss postprocessor 1066 may determine whether the patient has generalized bone loss (e.g., that applies to all teeth in the upper and/or lower jaw) and/or whether the patient has localized bone loss (e.g., at one or more specific teeth, a particular area of the patient's jaw, etc.). Periodontal bone loss postprocessor 1066 may additionally or alternatively determine an angle of a periodontal bone line for the patient at the one or more teeth, wherein the angle of the periodontal bone line may be used to identify at least one of horizontal bone loss or vertical bone loss.

Based on the determined periodontal bone loss information (e.g., severity of periodontal bone loss for one or more teeth, information on general vs. localized bone loss, angle of bone line, horizontal vs. vertical bone loss, etc.) and/or other information such as patient age, patient habits, patient health conditions, etc., processing logic may determine whether a patient has periodontitis and/or a stage of the periodontitis. Other received information used to assess periodontitis may include, for example, pocket depth information for one or more teeth, bleeding information for the one or more teeth, plaque information for the one or more teeth, infection information for the one or more teeth, smoking status for the patient, and/or medical history for the patient. In embodiments, some of the additional information may be from intraoral scanning of the patient's oral cavity and/or from a DPMS.

Caries detector 1074 is a module of segmentation pipeline 1000 capable of identifying and distinguishing between the different types of caries that are possible. Caries detector 1074 may include a caries segmenter 1076 and a caries postprocessor 1078 in embodiments. Caries detector 1074 may receive tooth and jaw ROI information from ROI generator 1025, and may process areas of the radiograph 1005 indicated in the tooth and jaw ROI information using caries segmenter 1070. Alternatively, or additionally, caries detector 1074 may receive periodontal bone loss ROI information from ROI generator 1025, and may process areas of the radiograph 1005 indicated in the periodontal bone loss ROI information using caries segmenter 1070. Areas of the radiograph outside of the teeth and jaw (or outside the periodontal bone loss ROI) may not include caries. Accordingly, by limiting the information that is considered by caries segmenter 1076 to only information of the teeth the accuracy of the segmentation performed by the caries segmenter 1070 may be improved.

Caries segmenter 1076 may include one or more trained ML models that have been trained to segment a radiograph (or an ROI of a radiograph) into one or more caries. The trained ML model(s) may perform instance segmentation or semantic segmentation in some embodiments. In some embodiments, the trained ML model(s) perform caries detection, and identify bounding boxes around individual instances of caries. Caries segmenter 1076 outputs segmentation information identifying caries within the teeth and/or jaw ROI(s) and/or within the periodontal bone loss ROI. Caries may have a different appearance in radiographs due to differences in material and/or density of caries as compared to teeth. Accordingly, caries may be detected based on differences in intensity by the caries segmenter 1076.

Caries postprocessor 1078 may receive caries segmentation information from caries segmenter 1076. Additionally, caries postprocessor 1078 may receive tooth segmentation information from tooth detector/classifier 1020. Caries postprocessor 1078 may combine the tooth segmentation information and the caries segmentation information to determine which tooth each detected caries is associated with. For each identified caries, the caries may be assigned to a single tooth based on the determined caries segmentation information and tooth segmentation information of the plurality of teeth.

In embodiments, caries postprocessor 1078 may determine a severity of one or more caries. In some embodiments, caries severity is determined at least in part on a size of the caries. The larger the size of the caries, the more severe the caries is likely to be. In some embodiments, caries severity is determined at least in part on a depth of the caries. The deeper a caries is, the more likely it is to penetrate a tooth's dentin, and the more severe the caries is likely to be. In embodiments, a size and/or depth of a caries may be determined based on generating one or more measurements of the caries. The measurements may include a depth measurement between a deepest part of the caries and a tooth crown surface. The measurements may additionally or alternatively include an area measurement to indicate a size of the caries. The depth and/or area measurements may be used to determine the caries severity based on comparison to one or more thresholds. Each threshold may be associated with a different severity level. Caries postprocessor may determine a highest threshold that the measurements of a caries meets or exceeds, determine a severity level associated with that highest threshold, and assign a severity associated with that threshold to the caries in question.

In some embodiments, caries segmenter 1076 outputs dentin segmentation information in addition to caries segmentation information. Additionally, or alternatively, tooth postprocessor 1040 may output dentin segmentation information based on segmentation performed by first tooth segmenter 1034 and/or second tooth segmenter 1036 in some embodiments. When dentin segmentation information is available, caries postprocessor 1078 may compare caries segmentation information to dentin segmentation information to determine whether a caries has penetrated a tooth's dentin. If a tooth's dentin has been penetrated, then the caries severity may be increased, and the caries may be labeled as a dentin caries. If the caries is only in the enamel of a tooth, then the caries severity may be lower and the caries may be labeled as an enamel caries. Caries postprocessor 1078 may determine a distance between a caries on a tooth and the dentin of the tooth based on a comparison of the caries segmentation information and the dentin segmentation information. Caries postprocessor 1078 may then determine a severity of the caries for the tooth at least in part based on the distance in some embodiments. The lower the distance, the more severe the caries may be.

In some embodiments, caries postprocessor 1078 determines localization of one or more caries. The localization may include information on a position of a caries on a tooth, such as a left of tooth, a right of tooth, a mesial surface of the tooth, a lingual surface of the tooth, an interproximal surface of the tooth, an occlusal surface of a tooth, and so on. In some embodiments, caries segmenter 1076 includes one or more trained machine learning models that assign localization to caries. Alternatively, caries detector 1074 may include a separate ML model for caries localization that receives as an input the caries segmentation information and provides as an output one or more localization classes for one or more caries. Examples of localization classes comprises at least one of tooth left surface, tooth right surface, tooth top surface, tooth mesial surface, tooth distal surface, tooth lingual surface, or tooth buccal surface.

Impacted tooth detector 1080 is a module of segmentation pipeline 1000 capable of detecting impacted teeth. Impacted tooth detector 1080 may include an impacted tooth segmenter 1082 and an impacted tooth postprocessor 1084 in embodiments. Impacted tooth detector 1080 may receive periodontal bone loss ROI information from ROI generator 1025, and may process areas of the radiograph 1005 indicated in the periodontal bone loss ROI information using caries segmenter 1070. Alternatively, impacted tooth segmenter 1082 may receive radiograph 1005 and process the full radiograph 1005.

Impacted tooth segmenter 1082 may include one or more trained ML models that have been trained to segment a radiograph (or an ROI of a radiograph) into one or more impacted teeth. The trained MLs may perform instance segmentation or semantic segmentation in some embodiments. In some embodiments, the trained MLs perform impacted tooth detection, and identify bounding boxes around individual instances of impacted teeth. Impacted tooth segmenter 1082 outputs segmentation information identifying impacted teeth.

Impacted tooth postprocessor 1084 may receive impacted tooth segmentation information from impacted tooth segmenter 1082. Additionally, impacted tooth postprocessor 1084 may receive tooth segmentation information from tooth detector/classifier 1020. Impacted tooth postprocessor 1084 may combine the tooth segmentation information and the impacted tooth segmentation information to determine which tooth number each detected impacted tooth is associated with. For each identified impacted tooth, the impacted tooth may be assigned to a single tooth number.

In some embodiments, impacted tooth postprocessor 1084 additionally receives periodontal bone loss segmentation information (e.g., such as for a periodontal bone line) from periodontal bone loss segmenter 1064. Impacted tooth postprocessor 1084 may compare a location of a tooth (e.g., from the impacted tooth segmentation information and/or tooth segmentation information) to a location of a determined periodontal bone line (e.g., from periodontal bone loss segmentation information). If the crown of the tooth is determined to be at or below the periodontal bone line, then the tooth may be identified or confirmed as an impacted tooth.

In some embodiments, impacted tooth segmenter 1082 additionally or alternatively outputs partially erupted tooth segmentation information. In some embodiments, impacted tooth segmentation information and partially erupted tooth segmentation information is combined. Partially erupted teeth may be identified in a similar manner to impacted teeth in embodiments. In some embodiments, impacted tooth postprocessor 1084 identifies partially erupted teeth in addition to, or instead of, impacted teeth. For example, impacted tooth postprocessor 1084 may compare a location of a tooth to a location of a determined periodontal bone line. Impacted tooth postprocessor 1084 may identify or confirm the tooth as a partially erupted tooth responsive to determining that enamel of the tooth intersects the periodontal bone line.

In embodiments, outputs of mandibular nerve detector 1015, periapical radiolucency detector 1050, restoration detector 1068, periodontal bone loss detector 1052, caries detector 1074 and/or impacted tooth detector 1080 are input into postprocessor 1088. Postprocessor 1088 may combine mandibular nerve segmentation information, tooth segmentation information, apical lesion segmentation information, restoration segmentation information, periodontal bone loss segmentation information, caries segmentation information and/or impacted tooth segmentation information to make further determinations about one or more oral conditions of the patient's oral cavity.

In one embodiment, postprocessor 1088 combines mandibular nerve segmentation information, tooth segmentation information and/or apical legion segmentation information (e.g., an output of mandibular nerve detector 1015 and periapical radiolucency detector 1050) to determine how close an apical lesion and/or a tooth root is to a patient's mandibular nerve canal. This determination may be made on a tooth-by-tooth basis in embodiments. For any tooth that is close to the mandibular nerve canal, performing surgery on a root of that tooth may be risky due to its closeness to the mandibular nerve canal. For example, during surgery a doctor may accidentally cause a dental tool to come into contact with the mandibular nerve canal. Accordingly, doctors should proceed cautiously when working on tooth roots that are near the mandibular nerve canal. In an embodiment, postprocessor 1088 determines locations of roots of one or more teeth, and for each tooth determines a smallest distance between the tooth root and the mandibular nerve canal. The determined distances may be compared to one or more distance threshold, and teeth having a distance to the mandibular nerve canal that is less than a distance threshold may be flagged. A notice may be output indicating that the root of a tooth is near the mandibular nerve canal. In some instances, responsive to determining that the distance for a tooth is below a distance threshold and that an apical lesion is existent for such a tooth (e.g., an apical lesion that requires surgery), postprocessor 1088 (or a module of oral health diagnostics system 215) may output a recommendation to perform three-dimensional imaging of the tooth. The three dimensional imaging may be, for example, a CBCT scan. Based on the three-dimensional imaging, a distance between the tooth root and the mandibular nerve canal may be more accurately determined than can be done in two dimensions from a radiograph.

Postprocessor 1088 may combine the outputs of multiple detectors (e.g., of multiple trained machine learning models of the detectors), and may perform postprocessing to resolve any discrepancies between the outputs of the multiple detectors. For example, discrepancies with respect to calculus, caries, restorations, periodontal bone loss, impacted teeth, apical lesions, and so on may be determined and resolved.

In one embodiment, postprocessor 1088 removes one or more instances of first identified oral conditions based on one or more instances of second identified oral conditions that conflict with the one or more instances of the first oral conditions. For example, restoration detector 108 may identify one or more teeth as restorations, and caries detector 1074 may identify those same one or more teeth as having caries. A restoration cannot have a caries. Accordingly, for teeth that are identified both as restorations and as having caries, the instances of caries for those teeth may be removed.

Many other postprocessing operations may be performed based on combinations of data from the different detectors. Once postprocessing is complete, postprocessor 1088 may provide an output to image generator 1090. Image generator 1090 may then generate one or more images based on each of the detected oral conditions (e.g., based on detected mandibular nerve canal, teeth, apical lesions, caries, restorations, periodontal bone loss, periodontal bone line, CEJ, impacted teeth, and so on). Image generator may generate a new version of the radiograph 1005 that is labeled with the detected oral conditions. In some embodiments, image generator 1090 generates a separate mask or layer for each instance of each oral condition. Viewing of classes of oral conditions may then be turned on or off, viewing of individual instances of one or more oral conditions may be turned on or off, and so on. The masks and/or layers may be images (e.g., such as partially transparent images) that may be part of or separate from radiograph 1005. For example, a separate image may be generated for each instance of each detected oral condition in embodiments. The location and/or shape of a particular oral condition may be determined based on the segmentation information for that oral condition as output by a postprocessor associated with that oral condition. In some embodiments, image generator 1090 compresses the generated image or images for transmission and/or storage. Accordingly, an output of image generator 1090 may be one or more compressed and/or transformed images 1092 containing information for each of the identified oral conditions.

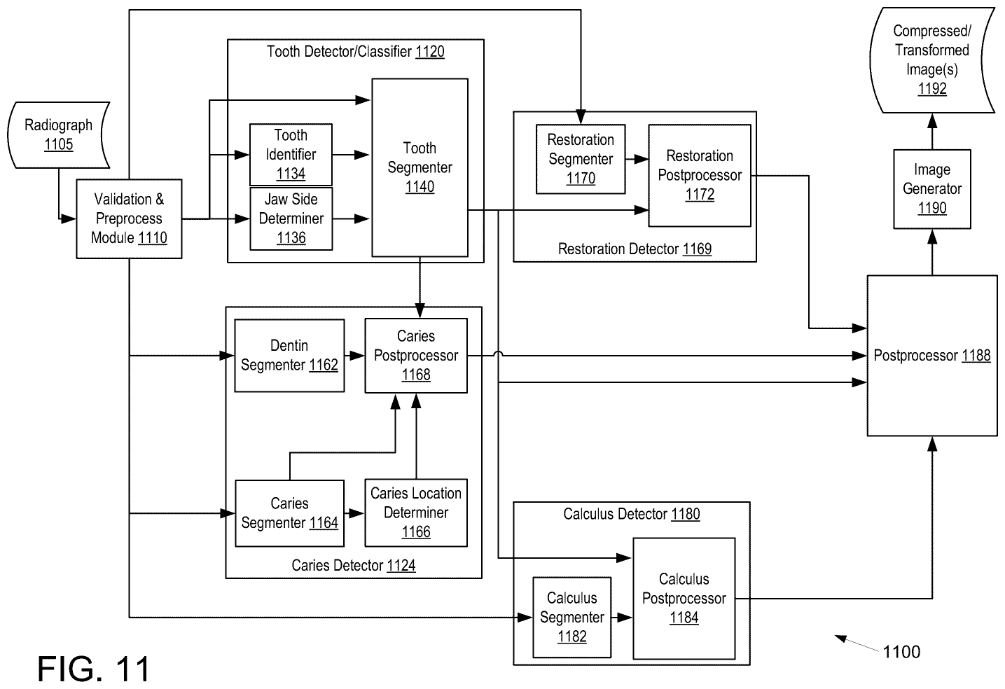

FIG. 11 illustrates an example segmentation pipeline 1100 for performing segmentation of a second type of dental radiographs, in accordance with embodiments of the present disclosure. Segmentation pipeline 1100 may be used for processing bite-wing radiographs (e.g., bite-wing radiograph 1105) in embodiments. While example segmentation pipeline 1100 is shown for bite-wing radiographs, a same or similar segmentation pipeline may also be used for processing other types of radiographs and/or other types of image data (e.g., CBCT scans, 2D images of oral cavities, 3D models of dental arches, and so on).

Segmentation pipeline 1100 may include multiple modules (e.g., tooth detector/classifier 1120, caries detector 1124, etc.) that may separately operate on an input radiograph in parallel, and may further include one or more additional modules (e.g., restoration detector 1169, calculus detector 1180, etc.) that operate on a radiograph and/or on an output of one or more of the first modules, optionally in parallel. Segmentation pipeline 1100 further includes one or more additional modules (e.g., postprocessor 1188, image generator 1190, etc.) that may operate on combinations of outputs from other modules to ultimately generate one or more images (e.g., a transformed and/or compressed radiograph or other image 1192 having one or more overlay layers of detected dental conditions, segmented and identified teeth, and so on).

A validation and preprocess module 1110 of segmentation pipeline 1100 may receive a bite-wing radiograph 1105. Validation may include processing the radiograph to ensure that it satisfies one or more radiograph criteria, and to ensure that it is suitable for processing by the segmentation pipeline 1100. For example, validation and preprocess module 1110 may measure a blurriness of the radiograph and determine whether the blurriness is below a blurriness threshold. Preprocessing operations performed on the radiograph may include any of those previously discussed, such as sharpening, edge detection, cropping, and so on. Once the radiograph 1105 is validated and preprocessed, the preprocessed radiograph may be provided to tooth detector/classifier, caries detector 1124, calculus detector 1180 and/or restoration detector 1169.

Tooth detector/classifier 1120 may include a tooth identifier 1134, a jaw side determiner 1136, and/or a tooth segmenter 1140. Tooth identifier 1134 may include one or more trained machine learning models that generate a first preliminary output comprising tooth numbers of teeth in the radiograph 1105 according to physiological heuristics. In a panoramic radiograph, all of a patient's teeth are visible, making it easier to identify tooth numbers of individual teeth. However, for bite-wing radiographs only a subset of a patient's teeth (e.g., a few teeth of the upper jaw and a few teeth of the lower jaw) are generally visible. This makes it much more challenging to identify which teeth are represented in the bite-wing radiograph. Tooth identifier 1134 may be or include a trained ML model (e.g., a neural network trained as a classifier and/or object detector) trained to determine tooth numbers of teeth based on the shapes of those teeth, the shapes of surrounding teeth, hard and/or soft tissues surrounding the teeth, and/or other visual information. In one embodiment, tooth identifier 1134 includes a random forest classifier.

Jaw side determiner 1136 may include one or more trained machine learning models that generate a second preliminary output comprising an identification of a jaw side represented in the bite-wing radiograph 1105. For example, jaw side determiner 1136 may determine whether radiograph 1105 is of a left jaw side or a right jaw side of the patient. In one embodiment, jaw side determiner 1136 includes a neural network classifier.

Tooth segmenter 1140 may receive as input the radiograph 1105 as well as the output of tooth identifier 1134 (e.g., tooth numbers assigned to each tooth in radiograph 1105) and the output of jaw side determiner 1136 (e.g., a jaw side determination). Tooth segmenter 1140 may include one or more trained ML models that process the input to generate an output. In some embodiments, tooth segmenter 1140 generates a pixel-level segmentation mask of the patient's teeth. The pixel-level segmentation mask may separately identify each tooth, or may provide a single pixel-level identification of teeth, without separately calling out individual teeth. Accordingly, first tooth segmenter 1140 may perform semantic segmentation of the radiograph 1105 or instance segmentation of the radiograph 1105. Based on assistance from the outputs of tooth identifier 1134 and jaw side determiner 1136, tooth segmenter 1140 may determine a pixel-level mask for each tooth, which may be labeled with tooth number. Tooth numbers may correspond to any tooth numbering scheme, some of which are discussed elsewhere herein. Tooth segmenter 1140 may output tooth segmentation information that includes instance segmentation and accurate assigned tooth numbering for each tooth in the radiograph 1105. The tooth segmentation information may be provided, for example, to restoration detector 1169, caries detector 1124, calculus detector 1180, and/or postprocessor 1188 for further processing.

Restoration detector 1169 is a module of segmentation pipeline 1100 capable of identifying and distinguishing between the many different types of restorations that are possible. Restoration detector 1169 may include a restoration segmenter 1170 and a restoration postprocessor 1172 in embodiments. Restoration detector 1169 may receive radiograph 1105, and may process the radiograph 1105 using restoration segmenter 1170. Restoration segmenter 1170 may include one or more trained ML models that have been trained to segment a radiograph (e.g., a bite-wing radiograph) into one or more restorations. The trained MLs may perform instance segmentation or semantic segmentation in some embodiments. In some embodiments, the trained MLs perform restoration detection, and identify bounding boxes around individual instances of restorations. Restoration segmenter 1170 outputs segmentation information identifying restorations within the radiograph 1105.

Restoration postprocessor 1172 may receive restoration segmentation information from restoration segmenter 1170. Additionally, restoration postprocessor 1172 may receive tooth segmentation information from tooth detector/classifier 1120. Restoration postprocessor 1172 may combine the tooth segmentation information and the restoration segmentation information to determine which tooth or teeth detected restorations are associated with. For each identified restoration, the restoration may be assigned to a single tooth or to multiple teeth based on the determined restoration segmentation information and tooth segmentation information of the plurality of teeth. If a single restoration covers multiple teeth, then the restoration may be assigned to multiple teeth. If a single restoration covers only a single tooth root, then the restoration may be assigned to just one tooth.

In some instances restoration segmenter 1170 differentiates between different types of restorations, and outputs segmentation information indicating restoration types for each detected restoration. Additionally, or alternatively, restoration postprocessor 1172 may determine restoration types based on postprocessing of combined restoration segmentation information and tooth segmentation information (e.g., as discussed with reference to restoration postprocessor 1072). Restoration detector 1169 may output information (e.g., one or more masks, one or more bounding shapes/boxes, etc.) for each detected restoration, including one or more teeth associated with each restoration and a restoration type of each restoration.

Caries detector 1124 is a module of segmentation pipeline 1100 capable of identifying and distinguishing between the different types of caries that are possible. Caries detector 1124 may include a caries segmenter 1164, a dentin segmenter 1162, a caries location determiner 1166, and/or a caries postprocessor 1168 in embodiments. Caries detector 1124 may receive an input comprising radiograph 1105 and tooth segmentation information from tooth detector/classifier 1120, and may process the input to determine instances of caries in the radiograph 1105.

Caries segmenter 1164 may include one or more trained ML models that have been trained to segment a radiograph into one or more caries. The trained MLs may perform instance segmentation or semantic segmentation in some embodiments based on processing of radiograph 1105. In some embodiments, the trained MLs perform caries detection, and identify bounding boxes around individual instances of caries. Caries segmenter 1164 outputs segmentation information identifying caries within the radiograph 1105.

Dentin segmenter 1162 may include one or more trained ML models that have been trained to segment a radiograph into tooth areas such as dentin, roots, enamel, and so on. The trained MLs may perform instance segmentation or semantic segmentation in some embodiments based on processing of radiograph 1105. In some embodiments, the trained MLs perform dentin detection, and identify bounding boxes around individual instances of dentin. Dentin segmenter 1162 outputs segmentation information identifying dentin within the radiograph 1105. The output segmentation information from dentin segmenter 1164 may additionally identify other tooth regions, such as enamel and tooth roots. In some embodiments, dentin segmenter 1162 outputs pixel-level masks for the dentin of each tooth in radiograph 1105.

Caries location determiner 1166 may receive caries segmentation information from caries segmenter 1164. Caries location determiner 1166 may include one or more trained ML models (e.g., one or more neural network classifiers) trained to assign localization information to caries identified by caries segmenter 1164. For each instance of an identified caries, caries location determiner 1166 may determine whether the caries is on a left of a tooth, a right of a tooth, a buccal region of a tooth, a lingual region of a tooth, an occlusal region of a tooth, an interproximal region of a tooth, and so on.

Caries postprocessor 1168 may receive caries segmentation information from caries segmenter 1164, dentin segmentation information from dentin segmenter 1162, caries location information from caries location determiner 1166, and/or tooth segmentation information from tooth detector/classifier 1120. Caries postprocessor 1168 may combine the tooth segmentation information, the caries segmentation information, the dentin segmentation information, and/or the caries location information to determine which tooth each detected caries is associated with and to classify and/or assess each caries instance. For each identified caries, the caries may be assigned to a single tooth based on the determined caries segmentation information and tooth segmentation information of the plurality of teeth.

In embodiments, caries postprocessor 1168 may determine a severity of one or more caries. In some embodiments, caries severity is determined at least in part on a size of the caries. The larger the size of the caries, the more severe the caries is likely to be. In some embodiments, caries severity is determined at least in part on a depth of the caries. The deeper a caries is, the more likely it is to penetrate a tooth's dentin, and the more severe the caries is likely to be. In embodiments, a size and/or depth of a caries may be determined based on generating one or more measurements of the caries. The measurements may include a depth measurement between a deepest part of the caries and a tooth crown surface. The measurements may additionally or alternatively include an area measurement to indicate a size of the caries. The depth and/or area measurements may be used to determine the caries severity based on comparison to one or more thresholds. Each threshold may be associated with a different severity level. Caries postprocessor may determine a highest threshold that the measurements of a caries meets or exceeds, determine a severity level associated with that highest threshold, and assign a severity associated with that threshold to the caries in question.

Caries postprocessor 1168 may compare caries segmentation information to dentin segmentation information to determine whether a caries has penetrated a tooth's dentin. If a tooth's dentin has been penetrated, then the caries severity may be increased, and the caries may be labeled as a dentin caries. If the caries is only in the enamel of a tooth, then the caries severity may be lower and the caries may be labeled as an enamel caries. Caries postprocessor 1168 may determine a distance between a caries on a tooth and the dentin of the tooth based on a comparison of the caries segmentation information and the dentin segmentation information. Caries postprocessor 1168 may then determine a severity of the caries for the tooth at least in part based on the distance in some embodiments. The lower the distance, the more severe the caries may be.

In some embodiments, caries postprocessor 1168 assigns localization of one or more caries based on the output of caries location determiner 1166. The localization may include information on a position of a caries on a tooth, such as a left of tooth, a right of tooth, a mesial surface of the tooth, a lingual surface of the tooth, an interproximal surface of the tooth, and so on. Examples of localization classes comprises at least one of tooth left surface, tooth right surface, tooth top surface, tooth mesial surface, tooth distal surface, tooth lingual surface, or tooth buccal surface.

Calculus detector 1180 is a module of segmentation pipeline 1100 capable of identifying calculus in radiographs. Calculus detector 1180 may include a calculus segmenter 1182 and a calculus postprocessor 1184 in embodiments. Calculus detector 1180 may receive radiograph 1105, and may process the radiograph 1105 using calculus segmenter 1182. Calculus segmenter 1182 may include one or more trained ML models that have been trained to segment a radiograph (e.g., a bite-wing radiograph) into one or more calculus instances. The trained MLs may perform instance segmentation or semantic segmentation in some embodiments. In some embodiments, the trained MLs perform calculus detection, and identify bounding boxes around individual instances of calculus. Calculus segmenter 1182 outputs segmentation information identifying calculus instances within the radiograph 1105.

Calculus postprocessor 1184 may receive calculus segmentation information from calculus segmenter 1182. Additionally, Calculus postprocessor 1184 may receive tooth segmentation information from tooth detector/classifier 1120. Calculus postprocessor 1184 may combine the tooth segmentation information and the restoration segmentation information to determine which tooth or teeth detected calculus is associated with. For each identified calculus instance, the calculus may be assigned to a single tooth based on the determined calculus segmentation information and tooth segmentation information of the plurality of teeth. Calculus detector 1180 may output information (e.g., one or more masks, one or more bounding shapes/boxes, etc.) for each detected calculus instance, including a tooth associated with each calculus instance. Accordingly, calculus detector 1180 may output identifications and/or locations of calculus in the radiograph 1105.

Postprocessor 1188 may receive the outputs of restoration detector 1169, tooth detector/classifier 1120, caries detector 1124 and/or calculus detector 1180, and may additionally receive radiograph 1105. Postprocessor 1188 may combine the outputs of the multiple detectors (e.g., of multiple trained machine learning models of the detectors), and may perform postprocessing to resolve any discrepancies between the outputs of the multiple detectors. For example, discrepancies with respect to calculus, caries, restorations, periodontal bone loss, impacted teeth, apical lesions, and so on may be determined and resolved.

In one embodiment, postprocessor 1188 removes one or more instances of first identified oral conditions based on one or more instances of second identified oral conditions that conflict with the one or more instances of the first oral conditions. For example, restoration detector 1169 may identify one or more teeth as restorations, and caries detector 1124 may identify those same one or more teeth as having caries. A restoration cannot have a caries. Accordingly, for teeth that are identified both as restorations and as having caries, the instances of caries for those teeth may be removed.

Many other postprocessing operations may be performed based on combinations of data from the different detectors. Once postprocessing is complete, postprocessor 1188 may provide an output to image generator 1190. Image generator 1190 may then generate one or more images based on each of the detected oral conditions (e.g., based on detected teeth, caries, restorations, calculus, and so on). Image generator 1190 may generate a new version of the radiograph 1105 that is labeled with the detected oral conditions. In some embodiments, image generator 1190 generates a separate mask or layer for each instance of each oral condition. Viewing of classes of oral conditions may then be turned on or off, viewing of individual instances of one or more oral conditions may be turned on or off, and so on. The masks and/or layers may be images (e.g., such as partially transparent images) that may be part of or separate from radiograph 1105. For example, a separate image may be generated for each instance of each detected oral condition in embodiments. The location and/or shape of a particular oral condition may be determined based on the segmentation information for that oral condition as output by a postprocessor associated with that oral condition. In some embodiments, image generator 1190 compresses the generated image or images for transmission and/or storage. Accordingly, an output of image generator 1190 may be one or more compressed and/or transformed images 1192 containing information for each of the identified oral conditions.

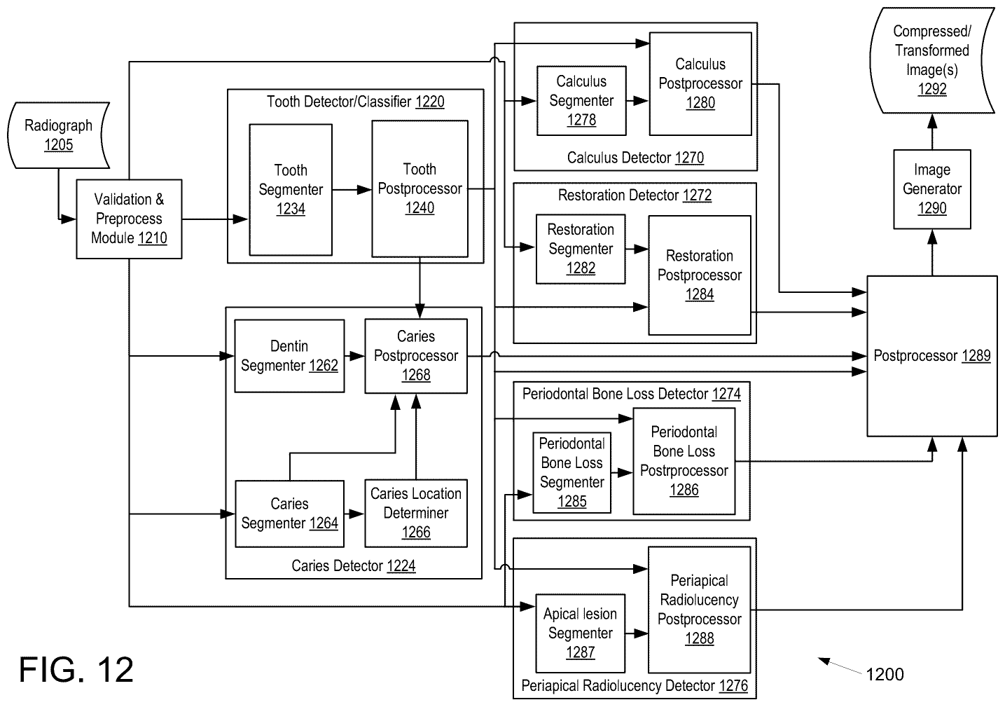

FIG. 12 illustrates an example segmentation pipeline 1200 for performing segmentation of a third type of dental radiographs, in accordance with embodiments of the present disclosure. Segmentation pipeline 1200 may be used for processing periapical radiographs (e.g., periapical radiograph 1205) in embodiments. While example segmentation pipeline 1200 is shown for periapical radiographs, a same or similar segmentation pipeline may also be used for processing other types of radiographs and/or other types of image data (e.g., CBCT scans, 2D images of oral cavities, 3D models of dental arches, and so on).

Segmentation pipeline 1200 may include multiple modules (e.g., tooth detector/classifier 1220, caries detector 1224, etc.) that may separately operate on an input radiograph in parallel, and may further include one or more additional modules (e.g., restoration detector 1269, calculus detector 1280, etc.) that operate on a radiograph and/or on an output of one or more of the first modules, optionally in parallel. Segmentation pipeline 1200 further includes one or more additional modules (e.g., postprocessor 1289, image generator 1290, etc.) that may operate on combinations of outputs from other modules to ultimately generate one or more images (e.g., a transformed and/or compressed radiograph or other image 1292 having one or more overlay layers of detected dental conditions, segmented and identified teeth, and so on).

A validation and preprocess module 1210 of segmentation pipeline 1200 may receive a periapical radiograph 1205. Validation may include processing the radiograph to ensure that it satisfies one or more radiograph criteria, and to ensure that it is suitable for processing by the segmentation pipeline 1200. For example, validation and preprocess module 1210 may measure a blurriness of the radiograph and determine whether the blurriness is below a blurriness threshold. Preprocessing operations performed on the radiograph may include any of those previously discussed, such as sharpening, edge detection, cropping, and so on. Once the radiograph 1205 is validated and preprocessed, the preprocessed radiograph may be provided to tooth detector/classifier 1220, caries detector 1224, calculus detector 1270, restoration detector 1272, periodontal bone loss detector 1274 and/or periapical radiolucency detector 1276.

Tooth detector/classifier 1220 may include a tooth segmenter 1234 and a tooth postprocessor 1240. Tooth segmenter 1234 may receive as input the radiograph 1205. Tooth segmenter 1234 may include one or more trained ML models that process the input to generate an output. In some embodiments, tooth segmenter 1234 generates a pixel-level segmentation mask of the patient's teeth. The pixel-level segmentation mask may separately identify each tooth, or may provide a single pixel-level identification of teeth, without separately calling out individual teeth. Accordingly, tooth segmenter 1234 may perform semantic segmentation of the radiograph 1205 or instance segmentation of the radiograph 1205. Tooth segmenter 1234 may determine a pixel-level mask for each tooth, may determine bounding shapes (e.g., bounding boxes) around each tooth, and/or may determine tooth numbers (e.g., tooth names) for each tooth. Accordingly, tooth segmentation information output by tooth segmenter 1234 may be labeled with tooth numbers. Tooth numbers may correspond to any tooth numbering scheme, some of which are discussed elsewhere herein. Tooth segmenter 1240 may output tooth segmentation information that includes instance segmentation and accurate assigned tooth numbering for each tooth in the radiograph 1205. The tooth segmentation information may be provided, for example, to restoration detector 1272, caries detector 1224, calculus detector 1270, periodontal bone loss detector 1274, periapical radiolucency detector 1276 and/or postprocessor 1289 for further processing.

Restoration detector 1272 is a module of segmentation pipeline 1200 capable of identifying and distinguishing between the many different types of restorations that are possible. Restoration detector 1272 may include a restoration segmenter 1282 and a restoration postprocessor 1284 in embodiments. Restoration detector 1272 may receive radiograph 1205, and may process the radiograph 1205 using restoration segmenter 1282. Restoration segmenter 1282 may include one or more trained ML models that have been trained to segment a radiograph (e.g., a periapical radiograph) into one or more restorations. The trained MLs may perform instance segmentation or semantic segmentation in some embodiments. In some embodiments, the trained MLs perform restoration detection, and identify bounding boxes around individual instances of restorations. Restoration segmenter 1282 outputs segmentation information identifying restorations within the radiograph 1205.

Restoration postprocessor 1284 may receive restoration segmentation information from restoration segmenter 1282. Additionally, restoration postprocessor 1284 may receive tooth segmentation information from tooth detector/classifier 1220. Restoration postprocessor 1284 may combine the tooth segmentation information and the restoration segmentation information to determine which tooth or teeth detected restorations are associated with. For each identified restoration, the restoration may be assigned to a single tooth or to multiple teeth based on the determined restoration segmentation information and tooth segmentation information of the plurality of teeth. If a single restoration covers multiple teeth, then the restoration may be assigned to multiple teeth. If a single restoration covers only a single tooth root, then the restoration may be assigned to just one tooth.

In some instances restoration segmenter 1282 differentiates between different types of restorations, and outputs segmentation information indicating restoration types for each detected restoration. Additionally, or alternatively, restoration postprocessor 1284 may determine restoration types based on postprocessing of combined restoration segmentation information and tooth segmentation information (e.g., as discussed with reference to restoration postprocessor 1072). Restoration detector 1272 may output information (e.g., one or more masks, one or more bounding shapes/boxes, etc.) for each detected restoration, including one or more teeth associated with each restoration and a restoration type of each restoration.

Caries detector 1224 is a module of segmentation pipeline 1200 capable of identifying and distinguishing between the different types of caries that are possible. Caries detector 1224 may include a caries segmenter 1264, a dentin segmenter 1262, a caries location determiner 1266, and/or a caries postprocessor 1268 in embodiments. Caries detector 1224 may receive an input comprising radiograph 1205 and tooth segmentation information from tooth detector/classifier 1220, and may process the input to determine instances of caries in the radiograph 1205.

Caries segmenter 1264 may include one or more trained ML models that have been trained to segment a radiograph into one or more caries. The trained MLs may perform instance segmentation or semantic segmentation in some embodiments based on processing of radiograph 1205. In some embodiments, the trained MLs perform caries detection, and identify bounding boxes around individual instances of caries. Caries segmenter 1264 outputs segmentation information identifying caries within the radiograph 1205.

Dentin segmenter 1262 may include one or more trained ML models that have been trained to segment a radiograph into tooth areas such as dentin, roots, enamel, and so on. The trained MLs may perform instance segmentation or semantic segmentation in some embodiments based on processing of radiograph 1205. In some embodiments, the trained MLs perform dentin detection, and identify bounding boxes around individual instances of dentin. Dentin segmenter 1262 outputs segmentation information identifying dentin within the radiograph 1205. The output segmentation information from dentin segmenter 1264 may additionally identify other tooth regions, such as enamel and tooth roots. In some embodiments, dentin segmenter 1262 outputs pixel-level masks for the dentin of each tooth in radiograph 1205.

Caries location determiner 1266 may receive caries segmentation information from caries segmenter 1264. Caries location determiner 1266 may include one or more trained ML models (e.g., one or more neural network classifiers) trained to assign localization information to caries identified by caries segmenter 1264. For each instance of an identified caries, caries location determiner 1266 may determine whether the caries is on a left of a tooth, a right of a tooth, a buccal region of a tooth, a lingual region of a tooth, an occlusal region of a tooth, an interproximal region of a tooth, and so on.

Caries postprocessor 1268 may receive caries segmentation information from caries segmenter 1264, dentin segmentation information from dentin segmenter 1262, caries location information from caries location determiner 1266, and/or tooth segmentation information from tooth detector/classifier 1220. Caries postprocessor 1268 may combine the tooth segmentation information, the caries segmentation information, the dentin segmentation information, and/or the caries location information to determine which tooth each detected caries is associated with and to classify and/or assess each caries instance. For each identified caries, the caries may be assigned to a single tooth based on the determined caries segmentation information and tooth segmentation information of the plurality of teeth.

In embodiments, caries postprocessor 1268 may determine a severity of one or more caries. In some embodiments, caries severity is determined at least in part on a size of the caries, as described above with reference to caries postprocessor 1168. In some embodiments, caries postprocessor 1268 assigns localization of one or more caries based on the output of caries location determiner 1266. The localization may include information on a position of a caries on a tooth, such as a left of tooth, a right of tooth, a mesial surface of the tooth, a lingual surface of the tooth, an interproximal surface of the tooth, and so on. Examples of localization classes comprises at least one of tooth left surface, tooth right surface, tooth top surface, tooth mesial surface, tooth distal surface, tooth lingual surface, or tooth buccal surface.

Calculus detector 1270 is a module of segmentation pipeline 1200 capable of identifying calculus in radiographs. Calculus detector 1270 may include a calculus segmenter 1278 and a calculus postprocessor 1280 in embodiments. Calculus detector 1270 may receive radiograph 1205, and may process the radiograph 1205 using calculus segmenter 1278. Calculus segmenter 1278 may include one or more trained ML models that have been trained to segment a radiograph (e.g., a periapical radiograph) into one or more calculus instances. The trained MLs may perform instance segmentation or semantic segmentation in some embodiments. In some embodiments, the trained MLs perform calculus detection, and identify bounding boxes around individual instances of calculus. Calculus segmenter 1278 outputs segmentation information identifying calculus instances within the radiograph 1205.

Calculus postprocessor 1280 may receive calculus segmentation information from calculus segmenter 1278. Additionally, Calculus postprocessor 1280 may receive tooth segmentation information from tooth detector/classifier 1220. Calculus postprocessor 1280 may combine the tooth segmentation information and the restoration segmentation information to determine which tooth or teeth detected calculus is associated with. For each identified calculus instance, the calculus may be assigned to a single tooth based on the determined calculus segmentation information and tooth segmentation information of the plurality of teeth. Calculus detector 1270 may output information (e.g., one or more masks, one or more bounding shapes/boxes, etc.) for each detected calculus instance, including a tooth associated with each calculus instance. Accordingly, calculus detector 1270 may output identifications and/or locations of calculus in the radiograph 1205.

Periapical radiolucency detector 1276 may receive radiograph 1205, and may process the radiograph 1205 to identify apical lesions. Periapical radiolucency detector 1276 may include an apical lesion segmenter 1287 and a periapical radiolucency postprocessor 1288. Apical lesion segmenter 1287 may include one or more trained ML models that have been trained to segment a radiograph into one or more apical lesions. The trained MLs may perform instance segmentation or semantic segmentation in some embodiments. In some embodiments, the trained MLs perform apical lesion detection, and identify bounding boxes around individual instances of apical lesions. Apical lesion segmenter 1287 outputs segmentation information identifying apical lesions within the radiograph 1205. Areas exhibiting periapical radiolucency may correspond to areas of apical lesions. Accordingly, apical lesions may be detected based on detection of radiolucency by the apical lesion segmenter 1287 at or around one or more teeth in embodiments.

Periapical radiolucency postprocessor 1288 may receive apical lesion segmentation information from apical lesion segmenter 1287. Additionally, periapical radiolucency postprocessor 1288 may receive tooth segmentation information from tooth detector/classifier 1220. Periapical radiolucency postprocessor 1288 may combine the tooth segmentation information and the apical lesion segmentation information to determine which tooth or teeth detected apical lesions are associated with. For each identified apical lesion, the apical lesion may be assigned to a single tooth or to multiple teeth based on the determined apical lesion segmentation information (e.g., which may be inflammation segmentation information) and tooth segmentation information of the plurality of teeth. If a single apical lesion covers multiple tooth roots, then the apical lesion may be assigned to multiple teeth. If a single apical lesion covers only a single tooth root, then the apical lesion may be assigned to just one tooth. Periapical radiolucency detector 1276 may output information (e.g., one or more masks, one or more bounding shapes/boxes, etc.) for each detected apical lesion, including one or more teeth associated with each apical lesion.

Periodontal bone loss detector 1274 is a module of segmentation pipeline 1200 capable of identifying and characterizing periodontal bone loss. Periodontal bone loss detector 1274 may include a periodontal bone loss segmenter 1285 and a periodontal bone loss postprocessor 1286 in embodiments. Periodontal bone loss segmenter 1285 may include one or more trained ML models that have been trained to segment a radiograph into one or more of a PBL, a CEJ, tooth roots, tooth root apexes, and so on. The trained MLs may perform instance segmentation or semantic segmentation in some embodiments. In some embodiments, the trained MLs perform detection of one or more dental classes, such as of a PBL, a CEJ, a tooth root, etc., and identify bounding boxes around individual instances of such dental classes. Periodontal bone loss segmenter 1285 may output segmentation information identifying a periodontal bone line, a CEJ line, tooth roots, and/or root apexes for one or more teeth.

Periodontal bone loss postprocessor 1286 may receive periodontal bone loss segmentation information (e.g., including for the PBL, CEJ, roots, root apexes, etc.) from periodontal bone loss segmenter 1285. Additionally, periodontal bone loss postprocessor 1286 may receive tooth segmentation information from tooth detector/classifier 1220. Based on the periodontal bone loss segmentation information and/or tooth segmentation information, periodontal bone loss postprocessor 1286 may determine a severity of the periodontal bone loss for a patient as a whole and/or for one or more teeth and/or regions of a patient's jaw. Periodontal bone loss postprocessor 1286 may perform any of the operations for determining a severity of periodontal bone loss described with reference to periodontal bone loss postprocessor 12066 in embodiments. Periodontal bone loss detector 1052 may output periodontal bone loss information.

Postprocessor 1288 may receive the outputs of restoration detector 1272, tooth detector/classifier 1220, caries detector 1224, calculus detector 1270, periodontal bone loss detector 1274, and/or periapical radiolucency detector 1276, and may additionally receive radiograph 1205. Postprocessor 1288 may combine the outputs of the multiple detectors (e.g., of multiple trained machine learning models of the detectors), and may perform postprocessing to resolve any discrepancies between the outputs of the multiple detectors. For example, discrepancies with respect to calculus, caries, restorations, periodontal bone loss, impacted teeth, apical lesions, and so on may be determined and resolved.

In one embodiment, postprocessor 1288 removes one or more instances of first identified oral conditions based on one or more instances of second identified oral conditions that conflict with the one or more instances of the first oral conditions. For example, restoration detector 1272 may identify one or more teeth as restorations, and caries detector 1224 may identify those same one or more teeth as having caries. A restoration cannot have a caries. Accordingly, for teeth that are identified both as restorations and as having caries, the instances of caries for those teeth may be removed.

Many other postprocessing operations may be performed based on combinations of data from the different detectors. Once postprocessing is complete, postprocessor 1288 may provide an output to image generator 1290. Image generator 1290 may then generate one or more images based on each of the detected oral conditions (e.g., based on detected teeth, caries, restorations, calculus, and so on). Image generator 1290 may generate a new version of the radiograph 1205 that is labeled with the detected oral conditions. In some embodiments, image generator 1290 generates a separate mask or layer for each instance of each oral condition. Viewing of classes of oral conditions may then be turned on or off, viewing of individual instances of one or more oral conditions may be turned on or off, and so on. The masks and/or layers may be images (e.g., such as partially transparent images) that may be part of or separate from radiograph 1205. For example, a separate image may be generated for each instance of each detected oral condition in embodiments. The location and/or shape of a particular oral condition may be determined based on the segmentation information for that oral condition as output by a postprocessor associated with that oral condition. In some embodiments, image generator 1290 compresses the generated image or images for transmission and/or storage. Accordingly, an output of image generator 1290 may be one or more compressed and/or transformed images 1292 containing information for each of the identified oral conditions.

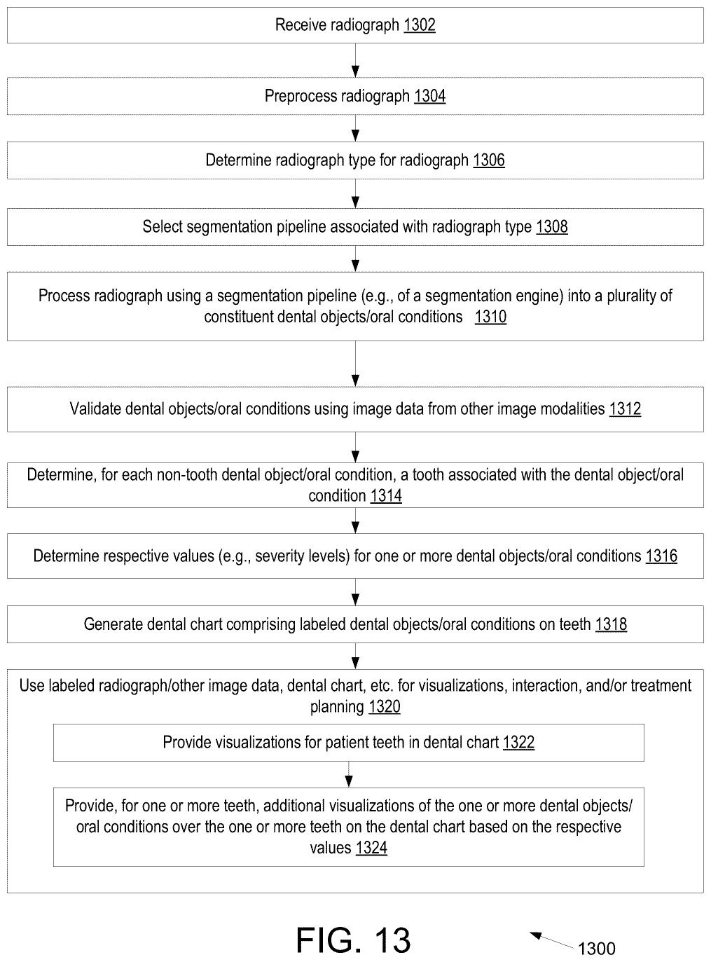

FIGS. 13-17B illustrate flow diagrams of methods performed by an oral health diagnostics system, in accordance with embodiments of the present disclosure. These methods may be performed by processing logic that comprises hardware (e.g., circuitry, dedicated logic, programmable logic, microcode, etc.), software (such as instructions run on a processing device), or a combination thereof. In one embodiment, processing logic corresponds to computing device 305 of FIG. 3 (e.g., to a computing device 305 executing an oral health diagnostics system 215).

FIG. 13 illustrates a flow diagram for a method 1300 of processing a radiograph to identify oral conditions of a patient using a segmentation pipeline, in accordance with embodiments of the present disclosure. Note that while method 1300 is described with reference to radiographs, method 1300 may also be used for other types of image modalities. For example, method 1300 may also be used for different types of 2D images (e.g., color 2D images, NIR 2D images, 2D images generated using fluorescence imaging), 3D images, intraoral scans, CBCT scans, ultrasonic scans, and so on.

At block 1302 of method 1300, processing logic receives a radiograph of at least a portion of a patient's oral cavity. Alternatively, processing logic may receive some other form of image data of at least a portion of the patient's oral cavity. At block 1304, processing logic preprocesses the radiograph (or other image data). Any of the aforementioned preprocessing operations may be performed. At block 1306, processing logic determines a radiograph type for the radiograph. In one embodiment, processing logic determines whether the received radiograph is a bite-wing radiograph, a panoramic radiograph, or a periapical radiograph. In some embodiments, the radiograph is labeled with its radiograph type. In some embodiments, a user indicates a radiograph type. In some embodiments, processing logic processes the radiograph using a trained ML model (e.g., a neural network) that classifies the radiograph type (e.g., classifies the radiograph as a bite-wing radiograph, a panoramic radiograph, or a periapical radiograph). If method 1300 is being used for other image modalities, at block 1306 processing logic may make other determinations about a type of image modality. For example, processing logic may determine whether the input is a color 2D image, a NIR 2D image, a type of radiograph, or other type of image data.

At block 1308, processing logic selects a segmentation pipeline associated with the determined radiograph type. If method 1300 is being used for other image modalities, at block 1308 processing logic may select segmentation pipelines associated with the determined image modality, which may or may not be a radiograph image modality. For example, an oral health diagnostics system may include different segmentation pipelines for different types of 2D images, for CBCT scans, for 3D models of dental arches, and so on.

At block 1310, processing logic processes the radiograph (or other type of image data) using the selected segmentation pipeline. The segmentation pipeline may correspond, for example, to any of segmentation pipelines 1000, 1100, 1200. The segmentation pipeline may include ML models and/or other logic that processes the radiograph (or other image data) to ultimately segment the radiograph (or other image data) into a plurality of constituent dental objects/structures and/or oral conditions. For example, the segmentation pipeline may output identity and location information for a patient's teeth, for restorations on the patient's dental arch, for caries, for regions of periodontal bone loss, for calculus, for a mandibular nerve canal, for apical lesions, for impacted teeth, for partially erupted teeth, and so on.

At block 1312, processing logic may validate the determined dental objects/structures and/or oral conditions using image data from one or more other image modalities. This may include receiving image data from another image modality (e.g., intraoral scan data, a 3D model of a dental arch generated based on intraoral scan data, a 2D color image, a NIR image, a CBCT scan, an ultrasound scan, etc.), and processing the image data using a segmentation pipeline associated with that type of image data. Processing logic may register the image data from the other image modality to the radiograph (or other type of image data) based on shared features between the image data and the radiograph. For example, processing logic may detect features or keypoints in the image data and the radiograph. These features could be corners, edges, blobs, or other distinctive points. Possible techniques for feature detection include Harris corner detection, SIFT (Scale-Invariant Feature Transform), SURF (Speeded-Up Robust Features), or more modern deep learning-based approaches. Once the features are detected, a descriptor may be computed for each feature. Descriptors are representations of the local neighborhood around each feature point, encoding information about the intensity gradients or texture in that region. These descriptors should be distinctive and invariant to changes in rotation, scale, and illumination. The descriptors from the image data and the radiograph may be compared to find matching features. Matching features are pairs of features from the two image modalities that represent the same point or structure in the scene. Various matching techniques can be used, such as nearest neighbor matching, where the closest descriptor in the other image is considered a match. Once matching features are found, a transformation model may be estimated that maps the points from one image to the corresponding points in the other image. Possible transformation models include affine transformations (translation, rotation, scaling, and shearing) or more complex models like projective transformations (homographies). The transformation parameters may be computed using techniques like least squares estimation or RANSAC (Random Sample Consensus) to robustly estimate the transformation even in the presence of outliers. Once the transformation is estimated, the image data from the other image modality may be warped or transformed according to the estimated transformation parameters so that it aligns with the radiograph (or the radiograph may be warped and transformed so that it aligns with the other image data). This involves resampling the intensity values of the pixels in one image onto a new grid determined by the transformation.

Once the radiograph and the other image data are registered, detected oral conditions from the other image data may be mapped onto the radiograph and/or detected oral conditions from the radiograph may be mapped onto the other image data. If the oral conditions from the different image modalities match or approximately match, then the oral conditions of the radiograph may be validated. If there are differences between the oral conditions of the radiograph and the oral conditions of the other image modality, then one or more conflict resolution operations may be performed to resolve the conflicting information. For example, processing logic may average the oral conditions of the radiograph and the other image modality using a weighted or unweighted averaging function. Some image modalities may be better at detecting some types of oral conditions than other image modalities. Accordingly, in some embodiments a weighted average of that type of oral condition may be generated by weighting the image modality that better captures that type of oral condition more heavily than the image modality that is not as accurate in capturing that type of oral condition. In some embodiments, a union of oral conditions is generated and used as final oral conditions. In some embodiments, an intersection of oral conditions is generated and used for final oral conditions.

At block 1314, processing logic determines, for each non-tooth dental object (e.g., for each oral condition), a tooth associated with the oral condition. In some embodiments, associated teeth of oral conditions are included in the segmentation information determined at block 1310. At block 1316, processing logic may determine respective values (e.g., severity levels) for one or more oral conditions. In some embodiments, one or more of the respective values for one or more oral conditions are determined at block 1310. In some embodiments, respective values for one or more oral conditions are determined based on performing postprocessing of the oral conditions. In some embodiments, the postprocessing is based on combining data for one or more oral conditions from the radiograph and additional data about the one or more oral conditions from image data of the another image type (e.g., as determined at block 1312). Severity levels may be based on a type of oral condition. For example, severity level of caries may be base on a size of the caries, a volume of the caries (e.g., based on combining data from the radiograph and an intraoral scan or 2D image of a tooth having the caries), whether the caries penetrates dentin, whether the caries extends into a tooth root, a distance of the caries from the tooth's dentin, a depth of the caries, and so on. A severity of periodontal bone loss may depend on a patient age, a distance between a PBL and a CEJ, a ratio of a distance between the PBL and the CEJ and the distance between the root apex and the CEJ, and/or other information. A severity of calculus may depend on a size and/or amount of calculus. These are just a few examples of types of oral conditions for which a severity may be assessed.

At block 1318, processing logic may generate a dental chart for the patient's teeth. The dental chart may include all or a portion of the patient's teeth. For each tooth, processing logic may add one or more labels to the tooth in the tooth chart based on the detected oral conditions for that tooth, the severity of the detected oral conditions for that tooth, the location(s) of the oral conditions for that tooth, and so on. Processing logic may additionally generate a list of teeth and their associated oral conditions.

At block 1320, processing logic may generate one or more visual overlays for the radiograph based on the detected oral conditions. The radiograph may be displayed along with the visual overlays. The visual overlays may be shown over the radiograph, optionally with some level of transparency so that the radiograph is visible beneath the overlays. Processing logic may use the labeled radiograph (e.g., that has been labeled with the oral conditions) and/or other image data, the dental chart, and/or other received or computed information for visualizations, user interactions and/or treatment planning for treatment of the one or more oral conditions in embodiments. This may include, for example, providing visualizations for the patient teeth and their associated oral conditions in the dental chart at block 1322. This may additionally include providing, for one or more teeth, additional visualizations (e.g., overlays) of the one or more oral conditions over the one or more teeth on the dental chart based on determined values/severity levels/locations, etc. A user may select any instance of an oral condition for a tooth directly from the radiograph and/or from the dental chart. Such selection may cause additional information about the tooth and/or the oral condition to be displayed. In some instances, underlying diagnoses of oral health problems associated with the oral condition and/or proposed treatments for treating the oral condition and/or the underlying oral health problems may be presented. A user may select a treatment option, which may launch an interface for treatment planning and/or launch an external treatment planning system.

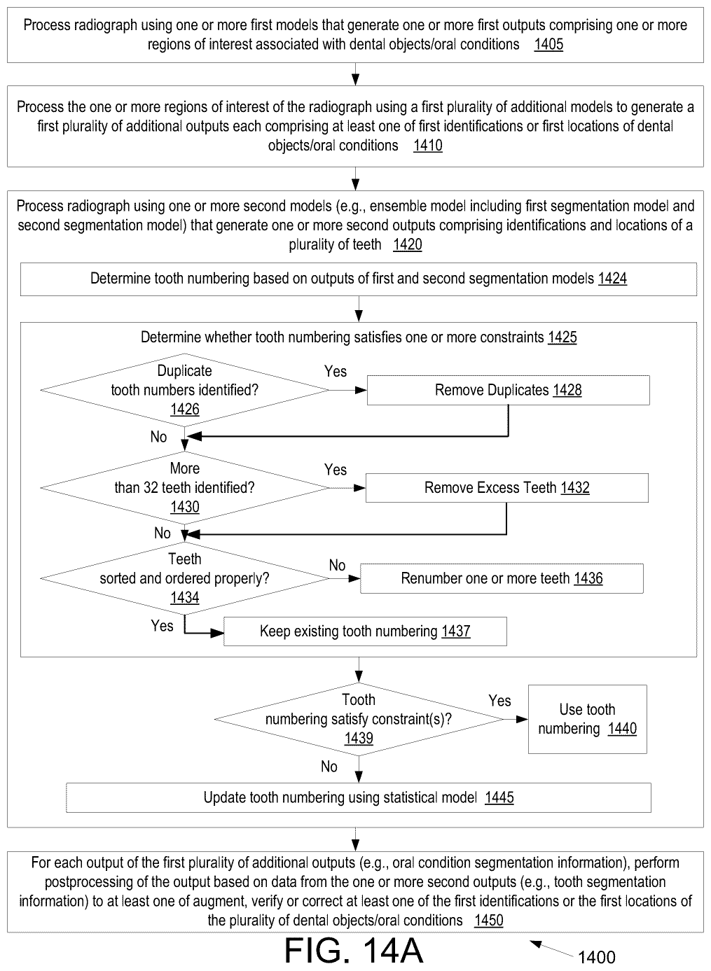

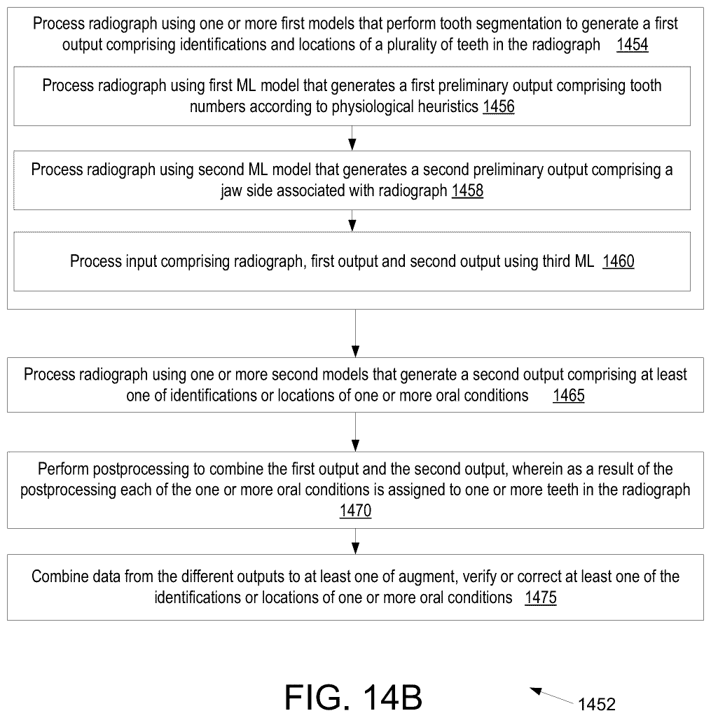

FIG. 14A illustrates a flow diagram for a method 1400 of processing a radiograph to identify oral conditions of a patient using a segmentation pipeline, in accordance with embodiments of the present disclosure. At block 1405 of method 1400, processing logic processes a radiograph using one or more first models (e.g., one or more first trained ML models, one or more physics-based models, one or more rules engines, etc.) that generate one or more first outputs comprising one or more regions of interest (ROIO associated with one or more dental objects and/or oral conditions. For example, the one or more first models may generate an ROI for a lower jaw, for a portion of a lower jaw, for one or more teeth, and so on. The ROI may exclude a portion of the radiograph that is not pertinent to a particular type of oral condition to be assessed. For example, a region of a radiograph that does not depict teeth or jaw may be excluded from a determined ROI. Examples of ROIs that may be determined are those determined by ROI generator 1025 of FIG. 10.

At block 1410, processing logic processes the one or more regions of interest of the radiograph using a first plurality of additional models to generate a first plurality of additional outputs each comprising at least one of first identifications or first locations of dental objects and/or oral conditions. Processing logic may include a plurality of different ML models, each trained to perform object detection, segmentation, classification, etc. for one or more different types of oral conditions and/or oral structures. For example, a first ML model may generate segmentation information for apical lesions, a second ML model may generate segmentation information for restorations, a third ML model may generate segmentation information for periodontal bone loss (e.g., including identifications and/or locations of a PBL, CEJ, roots, etc.), a fourth ML model may generate segmentation information for caries, and so on. In one embodiment, the ROI is a jaw, and the plurality of dental objects comprises a plurality of teeth. In one embodiment, the one or more regions of interest comprise one or more teeth, and the oral conditions comprise at least one of caries, periapical radiolucency, restorations, or periodontal bone loss locations associated with one or more teeth.

At block 1420, processing logic processes the radiograph using one or more second models that generate one or more second outputs comprising identifications and locations of a plurality of teeth. The plurality of teeth and the oral conditions may together constitute detected dental objects). The one or more second models may include one or more second ML models that perform tooth segmentation. In one embodiment, the one or more second models include an ensemble model including at least a first segmentation model that performs instance segmentation and a second segmentation model that performs semantic segmentation. Outputs of the multiple segmentation models may be combined to result in improved tooth segmentation accuracy.

In one embodiment, at block 1424 processing logic determines tooth numbering based on outputs of the first and second segmentation models. Initial tooth numbers may be assigned to each of the detected teeth. However, the initial tooth numbers may not be entirely accurate. Accordingly, one or more additional tooth number verification and/or postprocessing operations may be performed to increase an accuracy of the tooth numbers assigned to detected teeth.

At block 1425, processing logic determines whether the tooth numbering satisfies one or more constraints. The constraints may correspond to those set forth in operations 1426-1437, for example.

In one embodiment, at block 1426 processing logic determines whether duplicate tooth numbers have been identified. A patient may not have two teeth with the same tooth number. Accordingly, if two teeth were assigned the same tooth number, then the method proceeds to block 1428 and a duplicate tooth number is removed. This may include comparing information on the two teeth or objects assigned the same tooth number, determining which of the two teeth/objects is most likely to be the correct tooth having that tooth number, and removing the other tooth number. If no duplicate tooth numbers are identified, the method proceeds to block 1430. Additionally, after removing duplicate tooth numbers, the method proceeds to block 1430.

At block 1430, processing logic determines whether more than 32 teeth have been identified. If more than 32 teeth have been identified, then the method may continue to block 1432, and one or more access teeth may be removed. If 32 or fewer teeth were identified, the method proceeds to block 1434. Additionally, after removing the excess teeth, the method continued to block 1434.

At block 1434, processing logic determines whether the tooth numbers assigned to the teeth are sorted and ordered properly. Multiple tooth numbering schemes may be used, such as the universal tooth numbering (UTN) scheme, the FDI world dental federation notation (ISO 3950), Palmer notation, and so on. In general tooth numbering schemes start with a backmost molar on one dental arch, and count up from 1 for each subsequent tooth. The tooth numbering schemes then continue tooth numbering from a back molar of the opposing dental arch. Accordingly, tooth numbers should be arranged in ascending order. If any tooth number is out of order, then the tooth number is likely incorrect, and tooth numbering may be reassigned to correct the out of sequence tooth numbering. Accordingly, if the teeth are not properly sorted and ordered, the method may continue to block 1436, at which one or more teeth are renumbered. If the teeth are property sorted and ordered, the method may continue to block 1437, and the existing tooth numbering may be maintained. Note that the listed operations for correcting tooth numbering are only examples of rules for updating tooth numbering. Moreover, the order in which these tooth numbering correction operations are performed may be different than what is presented in method 1400.

At block 1439, processing logic may determine whether the determined tooth numbering satisfies one or more constraints, which may be additional constraints than those determined at block 1425. If the tooth numbering satisfies the constraints, the method may continue to block 1440, and the existing tooth numbering may be used. If the tooth numbering fails to satisfy the constraints, then a statistical model may be used to update the tooth numbering at block 1445. In case the contraints of the tooth numbering are not satisfied, processing logic may try to find the most likely arangement of tooth numbers that is realistically possible. This involves a statistical model that can predict for a certain sequence of tooth numbers how likely this sequence is. This statistical model may be used to select the most likely arrangement of tooth numbers that satisfies the logical constraints.

In some embodiments, one or more of the ML or AI models used to process data at any of blocks 1405, 1410 and/or 1420 rely on outputs from other ML or AI models as input. Accordingly, such models that rely on one or more first outputs of one or more other AI or ML models may wait for such first outputs to be generated by the other model(s) before processing an input comprising a radiograph and data from the one or more first outputs.

At block 1450, for each output of the first plurality of additional outputs (e.g., segmentation information of oral conditions) determined at block 1410, processing logic performs postprocessing of the output based on data from the one or more second outputs (e.g., tooth segmentation information). The postprocessing may be performed to combine tooth segmentation information with oral condition segmentation information. In embodiments, the tooth segmentation information may augment, verify and/or correct at least one of the first identifications or the first locations of the plurality of dental objects/oral conditions. For example, processing logic may constrain oral conditions based on tooth segmentation information, may assign oral conditions to specific teeth, and so on.

In some embodiments, multiple layers of postprocessing may be performed. For example, a first layer of postprocessing may be performed on one or more type of oral conditions separately, optionally based on a combination with tooth segmentation information. Then a second layer of postprocessing may be performed that combines information on multiple different oral conditions. The second layer of postprocessing may be performed, for example, on the combined postprocessed outputs from the first layer of postprocessing to resolve any discrepancies.

FIG. 14B illustrates a flow diagram for a method 1452 of processing a radiograph to identify oral conditions of a patient using a segmentation pipeline, in accordance with embodiments of the present disclosure. At block 1454 of method 1452, processing logic processes a radiograph using one or more first models that perform tooth segmentation to generate a first output comprising identifications and locations of a plurality of teeth in the radiograph. In one embodiment, at block 1456 processing logic processes the radiograph using a first ML model that generates a first preliminary output comprising tooth numbers according to physiological heuristics. At block 1458, processing logic processes the radiograph using a second ML model that generates a second preliminary output comprising a jaw side associated with the radiograph. At block 1460, processing logic processes an input comprising the radiograph, the first output and the second output using a third ML model. The third ML model may then output the tooth segmentation information identifying labels (e.g., tooth numbers), shapes, and locations of each tooth. For example, the third ML model may output a separate pixel-level mask for each identified tooth, where each pixel in a mask indicates whether or not that pixel is part of a particular tooth.

At block 1465, processing logic processes the radiograph using one or more second models (e.g., one or more second ML models) that generate a second output comprising at least one of identifications or locations of one or more oral conditions. In embodiments, the radiograph is processed by multiple different ML models, each of which may be trained to perform segmentation and/or object detection for a different subset of oral conditions. For example, a first ML model may be trained to perform segmentation of caries, a second ML models may be trained to perform segmentation of restorations, and so on. Each of the second models may output one or more pixel-level masks associated with one or more types of oral conditions.

At block 1470, processing logic performs postprocessing to combine the first output (e.g., tooth segmentation information) and the one or more second outputs (e.g., oral condition segmentation information). A result of the postprocessing may be an assignment of each of the identified oral conditions to one or more identified teeth in the radiograph.

At block 1475, processing logic may compare and combine data from different outputs (e.g., combine information on identified restorations and information on identified caries). Based on the comparison, processing logic may augment, verify and/or correct the identifications and/or locations of one or more detected oral conditions. For example, if a tooth is detected as a restoration, and a caries is also detected for that same tooth, then the caries information may be corrected by removing the indication of caries for that tooth since a restoration cannot have a caries.

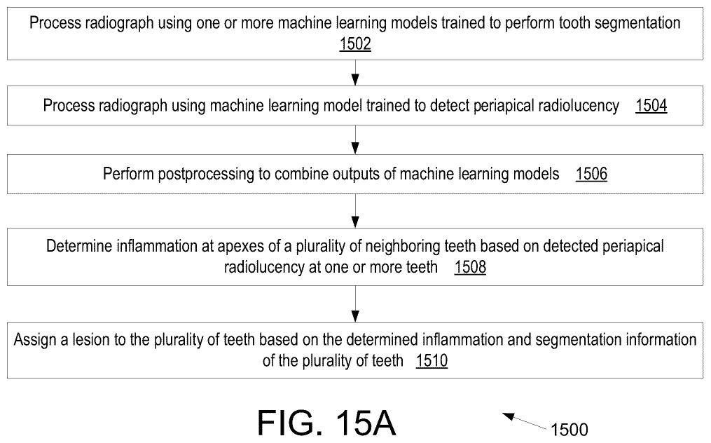

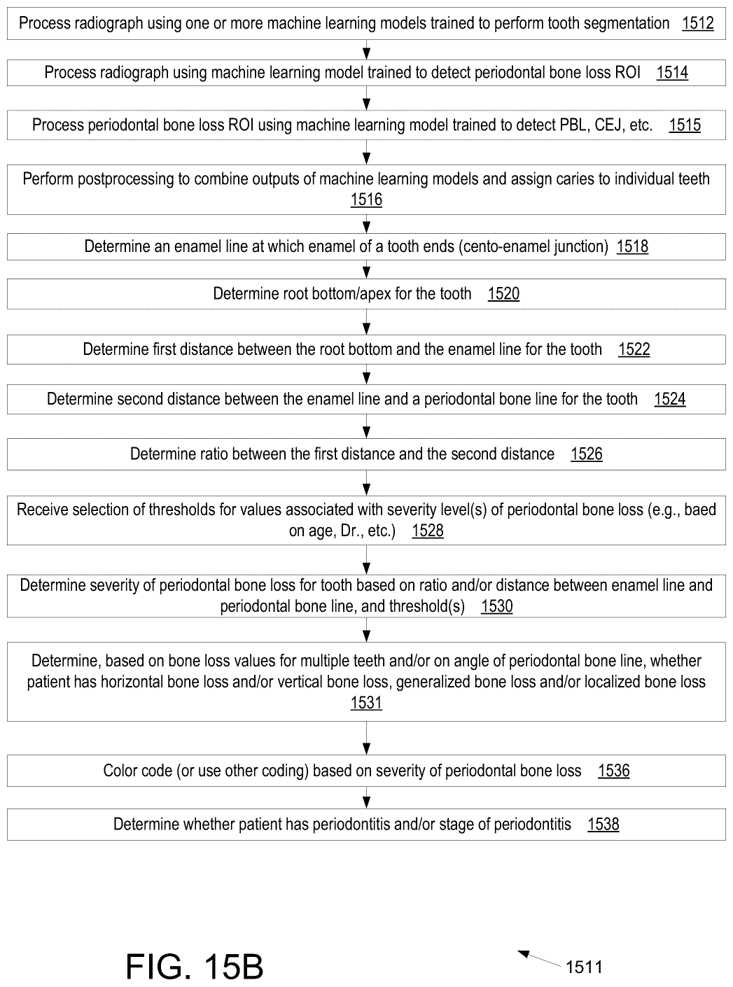

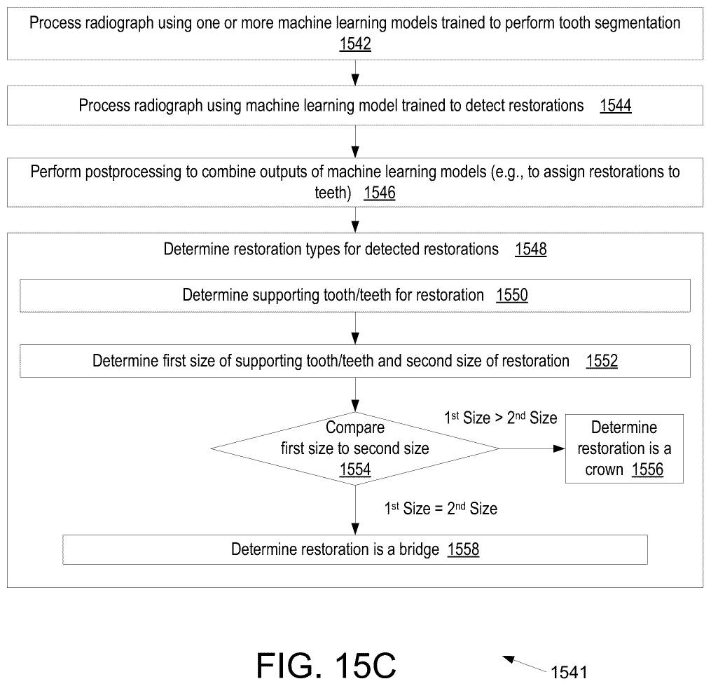

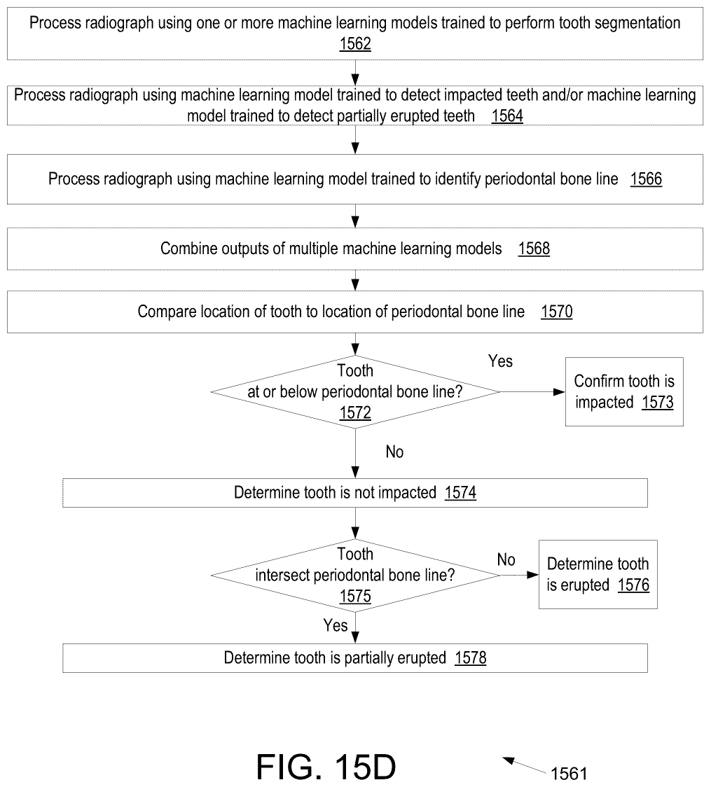

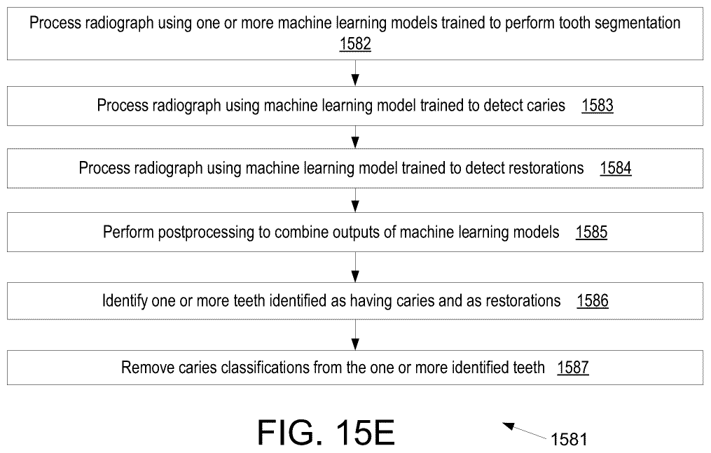

FIG. 15A illustrates a flow diagram for a method 1500 of processing a radiograph to identify lesions around teeth of a patient using a segmentation pipeline, in accordance with embodiments of the present disclosure. At block 1502 of method 1500, processing logic processes a radiograph using one or more trained machine learning models trained to perform tooth segmentation. The ML model(s) may output tooth segmentation information identifying, for each tooth in the radiograph, a tooth number and a pixel-level mask identifying the pixels of the radiograph for that tooth. At block 1504, processing logic processes the radiograph using a machine learning model trained to detect periapical radiolucency. The ML model may output periapical lesion segmentation information identifying each instance of a periapical lesion. The periapical lesion segmentation information may include semantic segmentation information (e.g., a single mask indicating which pixels in the radiograph correspond to periapical lesions) or instance segmentation information (e.g., a separate mask for each detected instance of a periapical lesion).

At block 1506, processing logic may combine the outputs of the ML models. This may include combining the tooth segmentation information with the periapical lesion segmentation information. Based on the combination of the tooth segmentation information and the periapical lesion segmentation information, periapical lesions may be assigned to one or more teeth. For example, the tooth segmentation information may be superimposed with the periapical lesion segmentation information. Periapical lesions that surround and/or are proximate to a tooth may be assigned to that tooth. Periapical lesions may be localized to a single tooth or may be spread across multiple teeth. Accordingly, at block 1508 processing logic may determine inflammation at apexes of a plurality of neighboring teeth based on detected periapical radiolucency (e.g., a periapical lesion) at one or more teeth, and may assign periapical lesions to those teeth that are proximate to the periapical lesions. One or more criteria may be used to determine whether a periapical lesion is associated with a tooth, such as proximity, whether the periapical lesion surrounds the root of the tooth, and so on. For example, a distance between a periapical lesion and a tooth may be determined and compared to a distance threshold. If the distance is less than the distance threshold, then the periapical lesion may be determined to be associated with that tooth. The same periapical lesion may be associated with multiple teeth in embodiments. In one embodiment, at block 1510 processing logic assigns a periapical lesion to a plurality of teeth based on the determined inflammation and segmentation information of the plurality of teeth. For example, if the periapical lesion is within a threshold distance of multiple teeth, then it may be associated with each of those teeth.

FIG. 15B illustrates a flow diagram for a method 1511 of processing a radiograph to identify periodontal bone loss of a patient using a segmentation pipeline, in accordance with embodiments of the present disclosure. At block 1512 of method 1511, processing logic processes a radiograph using one or more ML models trained to perform tooth segmentation. The one or more ML models may output tooth segmentation information. At block 1514, processing logic processes the radiograph using an ML model trained to detect a periodontal bone loss region of interest. At block 1515, processing logic processes the periodontal bone loss ROI using a trained machine learning model trained to generate periodontal bone loss segmentation information. The periodontal bone loss segmentation information may include an identification and location of a periodontal bone line, an identification and location of a cementoenamel junction, optionally identifications and locations of tooth roots, and so on. At block 1518, processing logic may determine the CEJ based on the periodontal bone loss segmentation information. At block 1520, processing logic determines a root apex for one or more teeth based on the tooth segmentation information and/or on the periodontal bone loss segmentation information.

At block 1522, processing logic determines a first distance between the root bottom and the enamel line (CEJ) of a tooth. This may be separately performed for each tooth. At block 1524, processing logic determines a second distance between the enamel line (CEJ) and the periodontal bone line for the tooth. This may separately be performed for each tooth. At block 1526, processing logic may determine a ratio between the first distance and the second distance. This ratio may be determined for each tooth, and may be representative of an amount of periodontal bone loss has occurred at or around the tooth.

At block 1528, processing logic may receive a selection of thresholds to use for values associated with severity levels of periodontal bone loss. An amount of periodontal bone loss may have different importance based on one or more underlying factors, such as patient age. As patients age, periodontal bone loss naturally occurs. Accordingly, what periodontal bone loss values constitute minor periodontal bone loss, moderate periodontal bone loss and severe periodontal bone loss may differ for different patients. To account for this, different periodontal bone loss thresholds may be selected for a patient. In one example, periodontal bone loss thresholds are selected based on patient age. For example, processing logic may receive a patient's age, and may automatically select a periodontal bone loss threshold associated with minor bone loss, a threshold associated with moderate bone loss, and a threshold associated with severe bone loss based on the patient age. Additionally, or alternatively, different doctors may have different views on what constitutes different severity levels of periodontal bone loss, and thresholds may be selected by the doctor or based on an identification of a doctor treating a patient.

At block 1530, processing logic may determine a severity of periodontal bone loss for one or more teeth based on comparison of the ratio of the first distance between the PBL and the CEJ and the second distance between the root apex and the CEJ to one or more ratio thresholds. Additionally, or alternatively, the first distance between the PCL and the CEJ may be compared to one or more distance thresholds to determine severity of the periodontal bone loss.

In one embodiment, at block 1531 processing logic may determine an angle of the periodontal bone line. The angle may be determined relative to the x-axis of a radiograph, relative to a line through the CEJ of multiple teeth, and/or relative to some other line. In one embodiment, processing logic determines, based on bone loss values for multiple teeth and/or the angle of the periodontal bone line, whether the patient has horizontal bone loss (e.g., where the bone loss is affecting most or all teeth approximately equally) and/or vertical bone loss (e.g., where bone loss is affecting one or more teeth or regions more than other teeth or regions). Processing logic may, for example, determine whether a patient suffers from generalized periodontal bone loss and/or localized periodontal bone loss (in addition to or instead of generalized periodontal bone loss).

At block 1536, processing logic may generate visualizations of periodontal bone loss. In one embodiment, an area between the CEJ and PBL for each of the teeth in the radiograph is determined. One or more visual overlay for this area may be generated. In one embodiment, multiple areas between CEJ and PBL for one or more teeth are determined. Areas may be determined based on neighboring teeth that share a similar amount of periodontal bone loss. For example, a first area may include three teeth that have a first amount of periodontal bone loss, and a second area may include four teeth that have a second amount of periodontal bone loss. The determined areas may be color coded based on the amount and/or severity of periodontal bone loss for the areas in embodiments. The visual overlays may be output over the radiograph to show the amount of periodontal bone loss that affects one or more of the patient's teeth in the radiograph.

In some embodiments, processing logic performs further processing based on the amounts and/or severities of the determined periodontal bone loss for one or more teeth to determine whether a patient has periodontitis. This may include using supplemental information such as patient age, patient health, past periodontal bone loss values, etc. to diagnose periodontitis and/or a stage of periodontitis.

FIG. 15C illustrates a flow diagram for a method 1541 of processing a radiograph to identify restorations of a patient using a segmentation pipeline, in accordance with embodiments of the present disclosure. At block 1542 of method 1541, processing logic processes a radiograph using one or more ML models trained to perform tooth segmentation, and outputs tooth segmentation information. At block 1544, processing logic processes the radiograph using an ML model trained to detect restorations. The ML model may output segmentation information on restorations. At block 1546, processing logic performs postprocessing to combine outputs of the ML models (e.g., the tooth segmentation information and restoration segmentation information). This may include assigning each instance of a restoration to one or more teeth.

At block 1548, processing logic determines restoration types for each of the detected restorations. Processing logic may apply one or more rules to determine whether a restoration is a filling, a root filling, a bridge, a crown, an implant, a veneer, a cap, and so on.

In one embodiment, at block 1550 processing logic identifies one or more supporting teeth for a determined restoration. The supporting tooth may be the tooth assigned to the restoration based on an overlap of the restoration from the restoration segmentation information and the tooth from the tooth segmentation information. At block 1552, processing logic determines a size of the supporting tooth and a size of the restoration. Sizes may be measured based on measuring one or more dimensions of a mask associated with the restoration and one or more dimensions of a mask associated with the tooth, for example. At block 1554, processing logic compares the first size of the supporting tooth to the second size of the restoration. If the first size is greater than the second size, then the restoration may be determined to be a crown at block 1556. If the first size is substantially greater than the second size (e.g., 50-99% greater), then the restoration may be identified as a filling in one embodiment. If the size of the restoration is about equal to a size of the supporting tooth, then the restoration may be identified as a bridge at block 1558. Other heuristics may also be used to identify different restoration types.

FIG. 15D illustrates a flow diagram for a method 1561 of processing a radiograph to identify impacted and/or partially erupted teeth of a patient using a segmentation pipeline, in accordance with embodiments of the present disclosure. At block 1562 of method 1561, processing logic may process a radiograph using one or more ML models trained to perform tooth segmentation, and output tooth segmentation information. At block 1564, processing logic processes the radiograph using an ML model trained to detect impacted teeth and/or partially erupted teeth. The ML model may output segmentation information on suspected impacted teeth and/or partially erupted teeth. At block 1566, processing logic may process the radiograph using an ML model trained to identify a periodontal bone line.

At block 1568, processing logic performs postprocessing to combine outputs of the ML models (e.g., the tooth segmentation information, impacted/partially erupted tooth segmentation information and/or periodontal bone line information). This may include assigning each instance of an impacted or partially erupted tooth to one or more teeth identified in the tooth segmentation information.

At block 1570, processing logic compares a location of a tooth identified as a suspected partially erupted or suspected impacted tooth to the periodontal bone line. At block 1572, processing logic determines whether the tooth (e.g., the crown of the tooth) is at or below the periodontal bone line. If so, then the method proceeds to block 1573 and the tooth is confirmed as an impacted tooth. Otherwise, the method continues to block 1574 and the tooth is identified as not being an impacted tooth.

At block 1575, processing logic may determine whether the tooth was identified as not an impacted tooth but might intersect the periodontal bone line. Such teeth may partially lie above the periodontal bone line, and a crown of such a tooth may extend partially above a patient's gingiva. However, some portion of the crown of a partially erupted tooth may intersect with the periodontal bone line. If a crown of a tooth (e.g., a center point of a crown of the tooth) intersects with or is within a threshold distance from the periodontal bone line, the method may continue to block 1578 and the tooth may be identified as a partially erupted. Otherwise, the method may continue to block 1576 and the tooth may be identified as fully erupted.

FIG. 15E illustrates a flow diagram for a method 1581 of processing a radiograph to correct false positives with respect to identified caries for a patient using a segmentation pipeline, in accordance with embodiments of the present disclosure. At block 1582 of method 1581, processing logic may process a radiograph using one or more ML models trained to perform tooth segmentation, and output tooth segmentation information. At block 1583, processing logic processes the radiograph using an ML model trained to detect caries and outputs caries segmentation information. At block 1585, processing logic processes the radiograph using an ML model trained to detect restorations, and outputs restorations segmentation information.

At block 1585, processing logic performs postprocessing to combine the outputs of the multiple ML models (e.g., to combine the tooth segmentation information, the caries segmentation information, and the restoration segmentation information). At block 1586, processing logic identifies one or more teeth that were identified both as having teeth and as restorations. At block 1587, processing logic removes the caries classifications from the one or more identified teeth that were also identified as restorations since restorations cannot have caries.

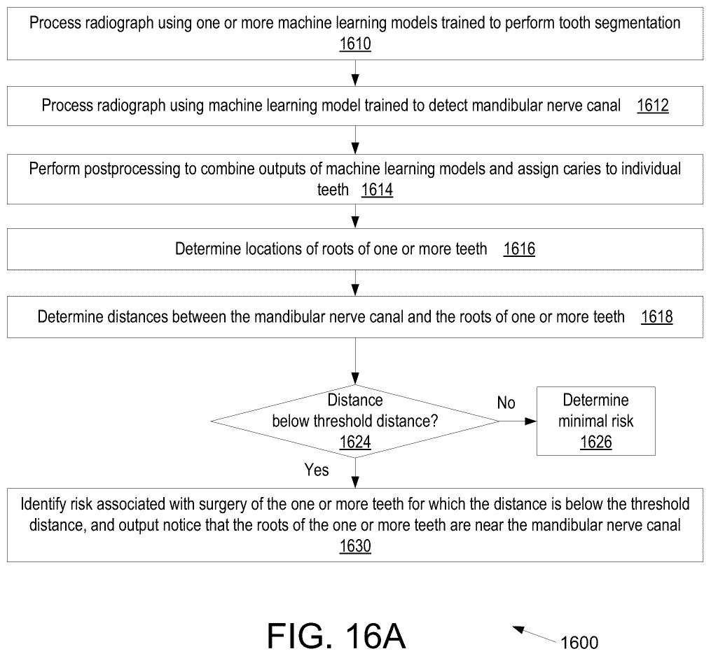

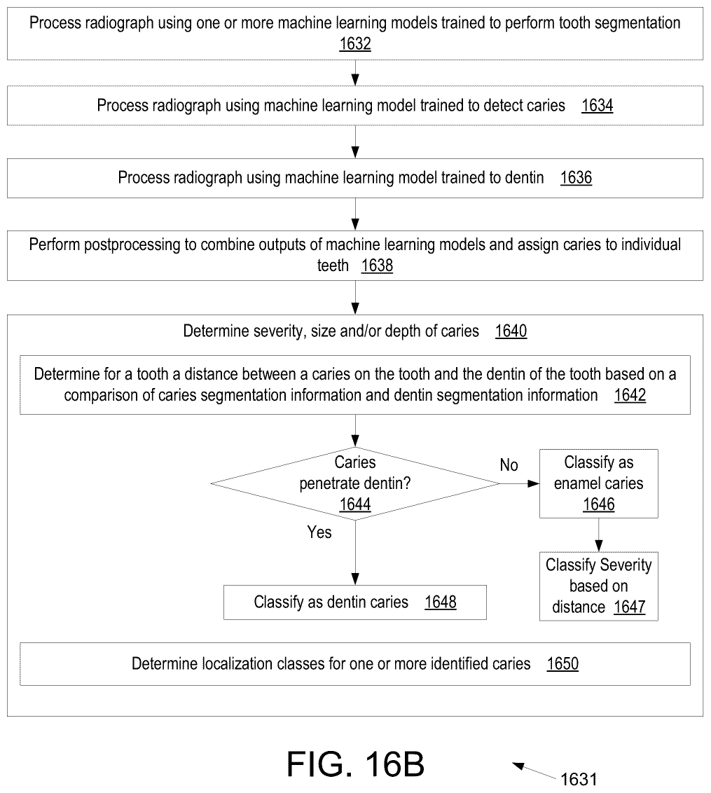

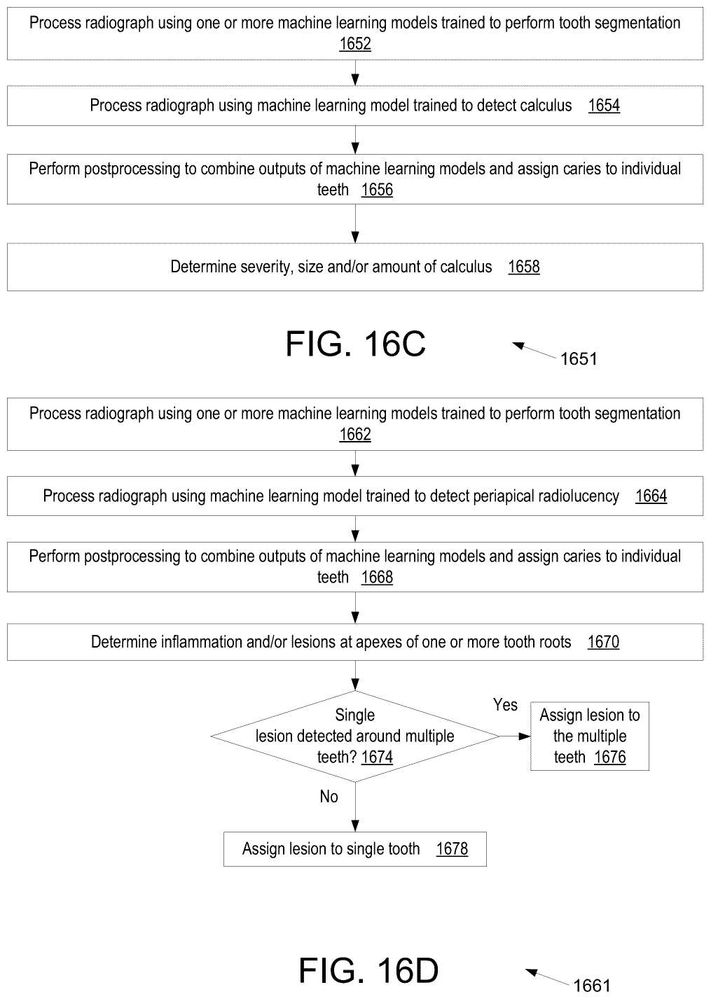

FIG. 16A illustrates a flow diagram for a method 1600 of identifying tooth roots near a mandibular nerve canal of a patient using a segmentation pipeline, in accordance with embodiments of the present disclosure. At block 1610 of method 1600, processing logic may process a radiograph using one or more ML models trained to perform tooth segmentation, and output tooth segmentation information. At block 1612, processing logic processes the radiograph using an ML model trained to detect a mandibular nerve canal. The ML model may output segmentation information on the mandibular nerve canal.

At block 1614, processing logic performs postprocessing to combine outputs of the ML models (e.g., the tooth segmentation information and mandibular nerve canal segmentation information). This may include superimposing the tooth segmentation information and the mandibular nerve segmentation information into a single image, for example.

At block 1616, processing logic determines locations of roots of one or more teeth in the combined segmentation information. At block 1618, processing logic measures distances between the mandibular nerve and the roots of one or more teeth. At block 1624, processing logic determines whether, for any of the teeth, the determined distance between the tooth's root and the mandibular nerve canal is below a distance threshold. If the distance is above the distance threshold for a tooth, then the tooth is determined to be at minimal risk at block 1626. If the distance is at or less than the threshold for a tooth, then at block 1630 processing logic identifies the tooth as close to the mandibular nerve canal. A tooth whose root is close to the mandibular nerve canal may be at increased risk of complications of surgery is performed on that tooth. Accordingly, for any such teeth that are close to the mandibular nerve canal, processing logic may output a notice that the roots of the one or more teeth are near the mandibular nerve canal. If a tooth that is close to the mandibular nerve canal is likely to receive surgery (e.g., if the tooth is associated with an apical lesion that requires surgery to treat), then processing logic may output a recommendation to perform 3D imaging to better determine the separation of the tooth root from the mandibular nerve canal.

FIG. 16B illustrates a flow diagram for a method 1631 of processing a radiograph to identify and determine severity of caries on teeth of a patient using a segmentation pipeline, in accordance with embodiments of the present disclosure. At block 1632 of method 1631, processing logic may process a radiograph using one or more ML models trained to perform tooth segmentation, and output tooth segmentation information. At block 1634, processing logic processes the radiograph using an ML model trained to detect caries. The ML model may output caries segmentation information. At block 1636, processing logic processes the radiograph using an ML model trained to detect dentin. The ML model may output dentin segmentation information.

At block 1638, processing logic performs postprocessing to combine outputs of the ML models (e.g., the tooth segmentation information, caries segmentation information and dentin segmentation information). This may include superimposing the tooth segmentation information, the caries segmentation information, and the dentin segmentation information, for example.

At block 1640, processing logic determines a severity, size and/or depth of the caries based on the combined tooth segmentation information, caries segmentation information, and/or dentin segmentation information. Caries severity may be based on size of caries, depth of caries, distance of the caries from dentin, whether the caries penetrates dentin, and so on. One or more measurements may be performed to measure caries depth, caries size, caries area, distance from caries to dentin, and so on. This information may be used to assign a severity level and/or classification to a caries.

In one embodiment, at block 1642, processing logic determines, for a tooth having a caries, a distance between the caries on the tooth and the dentin of the tooth. At block 1644, processing logic determines whether the caries penetrates the dentin (e.g., distance is zero). If the caries penetrates the dentin, then the caries is classified as a dentin caries at block 1648. A dentin caries may be a high severity caries. If the caries does not penetrate the dentin, then the method may proceed from block 1644 to block 1646. At block 1646, processing logic may classify the caries as an enamel caries. At block 1647, processing logic may then classify a severity of the caries based on the distance. If the distance is less than a threshold distance, then the caries may be classified as medium to high severity caries. If the distance is greater than the threshold distance, then the caries may be classified as a low severity caries.

In one embodiment, at block 1650 processing logic determines one or more localization classes for one or more identified caries. Localization classes may be left of tooth, right of tooth, at occlusal surface, at interproximal surface, a lingual surface, at buccal surface, and so on. In one embodiment, the radiograph is processed using a trained ML model trained to determine localization classes of the caries. Alternatively, localization classes may be determined based on performing one or more measurements of the caries mask for the caries from the caries segmentation information projected onto the tooth mask for the tooth from the tooth segmentation information. For example, processing logic may measure a distance from the caries to the occlusal surface, a distance from the caries to a left of the tooth, a distance from the caries to a right of the tooth, etc. The measured distances may then be used to assign localization information to the caries. For example, if the caries distance to the left of the tooth is less than the caries distance to the right of the tooth, then the caries may be identified as being at the left of the tooth.

FIG. 16C illustrates a flow diagram for a method 1651 of processing a radiograph to identify and determine severity of calculus on teeth of a patient using a segmentation pipeline, in accordance with embodiments of the present disclosure. At block 1652 of method 1651, processing logic may process a radiograph using one or more ML models trained to perform tooth segmentation, and output tooth segmentation information. At block 1654, processing logic processes the radiograph using an ML model trained to detect calculus. The ML model may output calculus segmentation information.

At block 1656, processing logic performs postprocessing to combine outputs of the ML models (e.g., the tooth segmentation information and calculus segmentation information). This may include superimposing the tooth segmentation information and calculus segmentation information, for example.

At block 1658, processing logic determines a severity, size and/or amount of the calculus based on the combined tooth segmentation information and calculus segmentation information. One or more measurements may be performed to measure calculus, calculus area, and/or surface area of tooth affected by calculus. This information may be used to assign a severity level and/or classification to an instance of calculus.

FIG. 16D illustrates a flow diagram for a method 1661 of processing a radiograph to identify lesions around teeth of a patient using a segmentation pipeline, in accordance with embodiments of the present disclosure. At block 1662 of method 1661, processing logic may process a radiograph using one or more ML models trained to perform tooth segmentation, and output tooth segmentation information. At block 1664, processing logic processes the radiograph using an ML model trained to detect periapical radiolucency, and thus periapical lesions. The ML model may output periapical lesion segmentation information.

At block 1668, processing logic performs postprocessing to combine outputs of the ML models (e.g., the tooth segmentation information and periapical lesion segmentation information). This may include superimposing the tooth segmentation information and periapical lesion segmentation information, for example.

At block 1670, processing logic determines inflammation and/or periapical lesions at the apexes of one or more tooth roots from the combined segmentation information. Processing logic may additionally assess a severity, size and/or area of periapical lesions based on the combined tooth segmentation information and/or periapical lesion segmentation information. One or more measurements may be performed to measure one or more dimensions of periapical lesions. This information may be used to assign a severity level and/or classification to a periapical lesion.

At block 1674, processing logic determines whether a periapical lesion is detected around the roots of multiple teeth. If so, then the method continues to block 1676 and the periapical lesion is assigned to multiple teeth. Otherwise, the method continues to block 1678 and the periapical lesion is assigned to a single tooth.

Generally it may not be possible to measure periodontal bone loss from a bite-wing radiograph. However, bite-wing radiographs are one of the most common types of radiographs that are generated during patient visits to a dentist. For example, bite-wing x-rays may be generated once a year, twice a year, or at some other frequency of patient visits. Accordingly, it would be useful to be able to measure periodontal bone loss from bite-wing x-rays.

One reason that bite-wing x-rays are generally not usable to measure periodontal bone loss is because the bite-wing x-rays do not indicate a size of a tooth and do not generally show apexes of tooth roots. In some embodiments, information from a previously generated 3D model of a dental arch of a patient and/or from a previously generated periapical x-ray or panoramic dental x-ray is used together with a current bite-wing x-ray to determine an amount of periodontal bone loss of a patient.

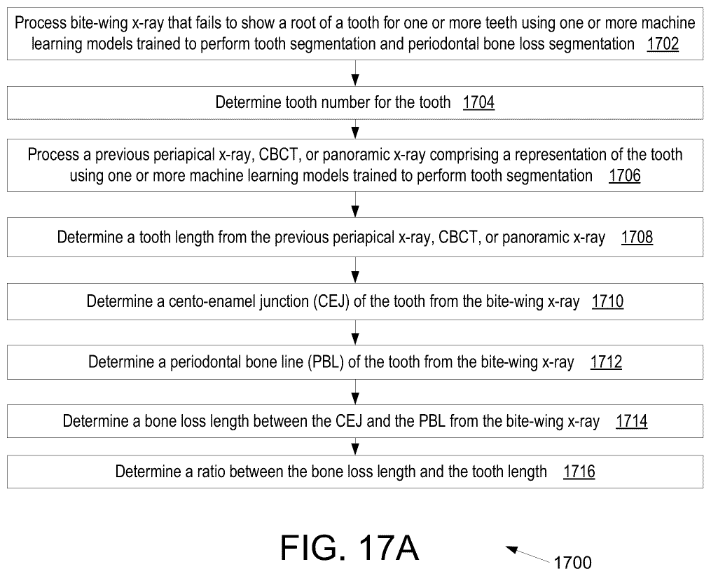

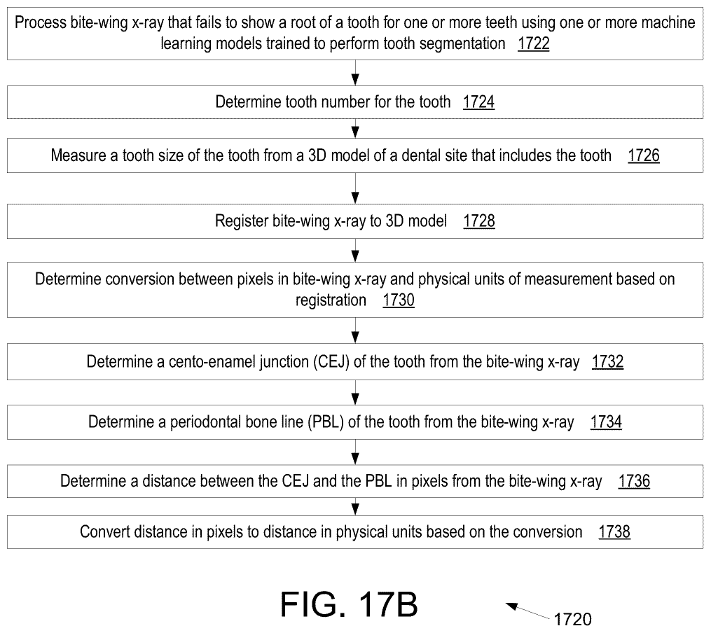

FIG. 17A illustrates a flow diagram for a method 1700 of determining periodontal bone loss for a tooth of a patient from a bite-wing x-ray and a prior periapical x-ray, CBCT or panoramic x-ray, in accordance with embodiments of the present disclosure. At block 1702 of method 1700, processing logic processes a bite-wing x-ray that fails to show a root of a tooth for one or more teeth using one or more trained ML models trained to perform tooth segmentation and/or one or more trained ML models trained to perform periodontal bone loss segmentation (e.g., to identify a periodontal bone line and/or CEJ). At block 1704, processing logic may determine a tooth number for the tooth based on the tooth segmentation information.

At block 1706, processing logic may process a previously generated periapical x-ray, CBCT, or panoramic x-ray for the patient using one or more third ML models trained to perform tooth segmentation and/or one or more fourth ML models trained to perform periodontal bone loss segmentation. The previously generated periapical x-ray, CBCT or panoramic x-ray comprises a representation of the tooth, including the tooth root apex. A size of a tooth (e.g., distance from CEJ to tooth root apex) may not change over time, or may change slowly (e.g., much more slowly than periodontal bone loss may progress). The previously generated periapical x-ray, CBCT or panoramic x-ray may not represent a current state of the patient's teeth, but may still accurately show a distance between the CEJ and the root apex for one or more teeth. Meanwhile, the current bite-wing x-ray may show the CEJ and the periodontal bone line, and may be used to measure a current distance between these two lines, but may not be usable to measure a distance from the CEJ to the root apex since the root apex is not visible in the bite-wing x-ray.

At block 1708, processing logic may determine a tooth length from the previous periapical x-ray, CBCT scan or panoramic x-ray. The determined tooth length may be a distance between the CEJ of the tooth and the root apex of the tooth as measured from the periapical x-ray, CBCT or panoramic x-ray.

At block 1710, processing logic may determine a CEJ of the tooth from the bite-wing x-ray (e.g., from the periodontal bone loss segmentation information). At block 1712, processing logic may determine a periodontal bone line of the tooth from the bite-wing x-ray (e.g., from the periodontal bone loss segmentation information). At block 1714, processing logic determines a bone loss length from the bite-wing x-ray, where the bone loss length is the distance between the CEJ and the periodontal bone line. At block 1716, processing logic may then determine a ratio between the bone loss length (as determined from the bite-wing x-ray) and the tooth length (as determined from the periapical x-ray, CBCT or panoramic x-ray). The ratio between the bone loss length and the tooth length may be compared to one or more ratio thresholds to determine a severity of the periodontal bone loss for the tooth.

FIG. 17B illustrates a flow diagram for a method 1720 of determining periodontal bone loss for a tooth of a patient from a bite-wing x-ray and a prior 3D model of a dental site (e.g., of a dental arch and/or tooth as generated from intraoral scanning of the dental arch), in accordance with embodiments of the present disclosure. At block 1722 of method 1720, processing logic processes a bite-wing x-ray that fails to show a root of a tooth for one or more teeth using one or more trained ML models trained to perform tooth segmentation and/or one or more trained ML models trained to perform periodontal bone loss segmentation (e.g., to identify a periodontal bone line and/or CEJ). At block 1724, processing logic may determine a tooth number for the tooth based on the tooth segmentation information.

At block 1726, processing logic may process a previously generated 3D model of the patient's dental arch. The previously generated 3D model of the dental arch comprises a true-scale representation of the portion of the tooth not covered by gingiva. A size of a tooth (e.g., a size of the tooth crown) may not change over time, or may change slowly (e.g., much more slowly than periodontal bone loss may progress). The previously generated 3D model of the patient's dental arch may not represent a current state of the patient's teeth, but may still accurately represent the size of the tooth. Meanwhile, the current bite-wing x-ray may show the CEJ and the periodontal bone line, and may be used to measure a current distance between these two lines, but may not be usable to determine a physical measurement or scale to assign to the current distance (which may be measured in pixels). Accordingly, processing logic may process the previously generated 3D model of the patient's dental arch using one or more ML or AI models trained to perform tooth segmentation of 3D models of dental arches. The one or more ML or AI models may output segmentation information identifying each tooth in the dental arch by tooth number. In one embodiment, 2D projections of the 3D model(s) of the patient's dental arch are generated, and the 2D projections are processed the ML or AI model(s) to perform tooth segmentation in the 2D projections.

Once the 3D model(s) and radiograph have been registered to one another, information of one or more oral conditions from the 3D model(s) may be merged with information of the one or more oral conditions from the radiograph.

At block 1728, processing logic may register the bite-wing x-ray to the 3D model of the dental arch using one or more registration techniques as previously discussed herein. The registration may include adjusting a scale of the tooth in the bite-wing x-ray to match a scale of the tooth in the 3D model. The registration may be assisted based on the tooth number labels of the 3D model and of the radiograph. For example, registration may be performed by registering one or more teeth having particular tooth numbers from the 3D model to the teeth having the same tooth numbers from the radiograph. Based on the registration, at block 1730 processing logic may determine a conversion between pixels in the bite-wing x-ray and physical units of measurement for length or distance (e.g., mm).

At block 1732, processing logic may determine a CEJ of the tooth from the bite-wing x-ray (e.g., from the periodontal bone loss segmentation information). At block 1734, processing logic may determine a periodontal bone line of the tooth from the bite-wing x-ray (e.g., from the periodontal bone loss segmentation information). At block 1736, processing logic determines a bone loss length from the bite-wing x-ray, where the bone loss length is the distance between the CEJ and the periodontal bone line. At block 1738, processing logic may then convert the bone loss length from pixels to physical units of measurement based on the conversion parameters determined at block 1730. The bone loss length in physical units of measurement may then represent an amount of current periodontal bone loss associated with the one or more teeth in the bite-wing x-ray. The bone loss length may be compared to one or more bone loss length thresholds to determine a severity of the periodontal bone loss for the one or more teeth.

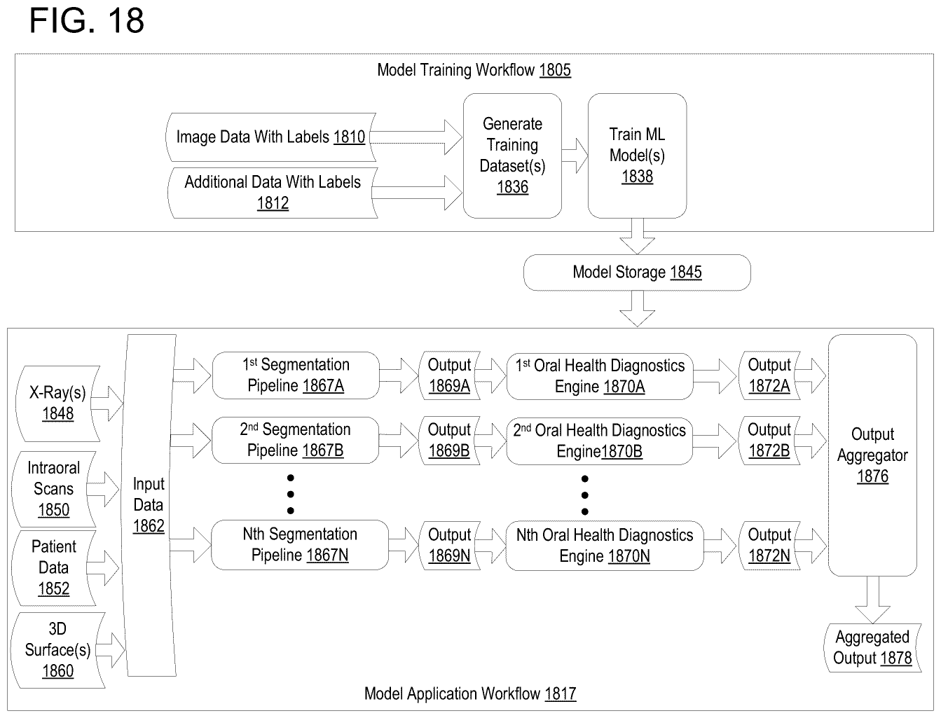

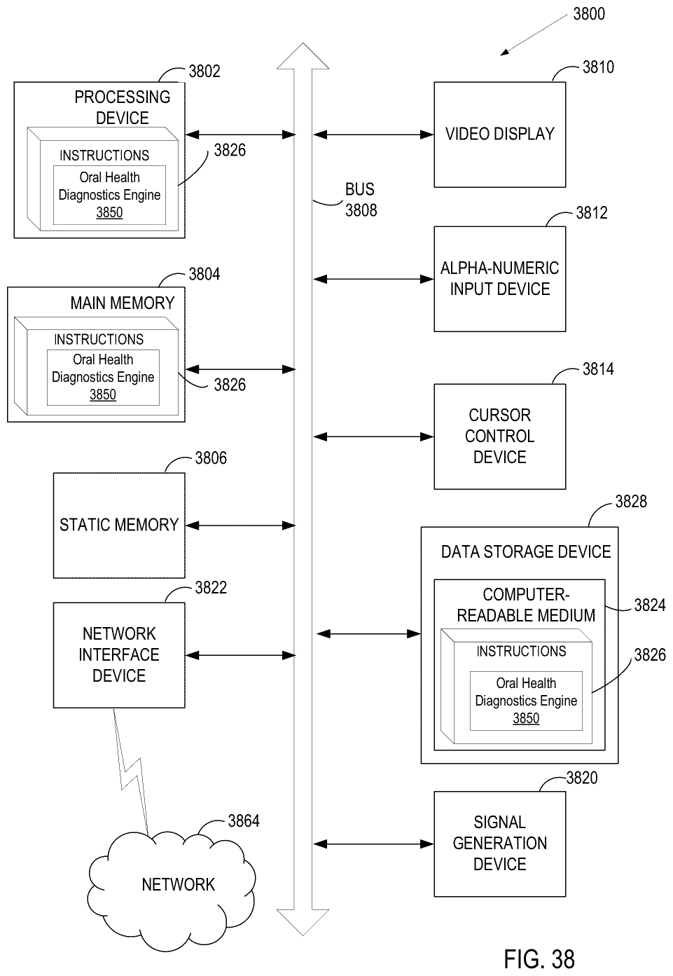

FIG. 18 illustrates a model training workflow 1805 and a model application workflow 1817 for an oral health diagnostics system, in accordance with an embodiment of the present disclosure. In embodiments, the model training workflow 1805 may be performed at a server which may be the same as, or different from, a server or computing device on which the oral health diagnostics system ultimately runs (e.g., on computing device 305 of FIG. 3), which may perform the model application workflow 1817. The model training workflow 1805 and the model application workflow 1817 may be performed by processing logic executed by one or more processors of one or more computing devices. One or more of these workflows 1805, 1817 may be implemented, for example, by one or more machine learning modules implemented in oral health diagnostics system 3826 or other software and/or firmware executing on a processing device of computing device 3800 shown in FIG. 38.

The model training workflow 1805 trains one or more machine learning models (e.g., deep learning models) to perform one or more classifying, segmenting (e.g., semantic segmentation and/or instance segmentation), detection, recognition, etc. tasks for image data from multiple different image modalities (e.g., intraoral scans, height maps, 2D color images, NIR images, radiographs, CBCT scans, ultrasound scans, etc.) and/or 3D surfaces generated based on intraoral scan data. The model application workflow 1817 is to apply the one or more trained machine learning models and additional logic to perform the classifying, segmenting, detection, recognition, etc. tasks for image data (e.g., intraoral scans, height maps, 2D color images, NIR images, radiographs, CBCT scans, ultrasound scans, etc.) and/or 3D surfaces generated based on intraoral scan data. One or more of the machine learning models may receive and process 3D data (e.g., 3D point clouds, 3D surfaces, portions of 3D models, etc.). One or more of the machine learning models may receive and process 2D data (e.g., 2D images, height maps, projections of 3D surfaces onto planes, radiographs, etc.). Different ML models may be trained to process different types of image data and/or to perform one or more different tasks.

Many different machine learning outputs are described herein. Particular numbers and arrangements of machine learning models are described and shown. However, it should be understood that the number and type of machine learning models that are used and the arrangement of such machine learning models can be modified to achieve the same or similar end results. Accordingly, the arrangements of machine learning models that are described and shown are merely examples and should not be construed as limiting.

In embodiments, one or more machine learning models are trained to perform one or more of the below tasks. Each task may be performed by a separate machine learning model. Alternatively, a single machine learning model may perform each of the tasks or a subset of the tasks and/or other tasks described herein. Additionally, or alternatively, different machine learning models may be trained to perform different combinations of the tasks. In an example, one or a few machine learning models may be trained, where the trained ML model is a single shared neural network that has multiple shared layers and multiple higher level distinct output layers, where each of the output layers outputs a different prediction, classification, identification, segmentation, etc. Additionally, each of the ML models may be trained to operate on a particular type of data, which may include one or more types of image data (e.g., a particular type of radiograph, a 2D color image, a CBCT, etc.) and/or other data (e.g., historical patient data, doctor observations, patient input, patient data from a DPMS, etc.) The tasks that the one or more trained machine learning models may be trained to perform are as follows:
I) Mandibular nerve segmentation—this can include segmenting image data into a mandibular nerve canal region and other regions that are not the mandibular nerve canal (e.g., based on semantic segmentation).
II) Tooth segmentation—this can include performing semantic segmentation of image data to assign tooth labels and/or one or more additional labels (e.g., gingiva labels, tooth root labels, tooth crown labels, etc.) to pixels in the image data. This may additionally include performing instance segmentation of image data to identify each individual tooth in the image data and generate pixel-wise segmentation masks for each individual tooth. Segmentation masks may also be generated for other oral structures, such as gingiva. In some instances, input to an ML model trained to perform tooth segmentation includes outputs of one or more other ML models (e.g., the output of an ML model that performs tooth detection and/or the output of an ML model that performs jaw side determination).
III) Tooth detection—this can include performing object detection to identify individual teeth in image data and assign bounding shapes (e.g., bounding boxes) around each identified tooth. This can also include assigning tooth numbers to each individual tooth.
IV) Jaw side determination—this may include performing classification of image data to classify the image as representing a left side of a patient's jaw or a right side of the patient's jaw.
V) Dentin segmentation—this can include performing semantic segmentation of image data to assign dentin labels and/or one or more additional labels (e.g., tooth root labels, tooth enamel labels, etc.) to pixels in the image data. This may additionally include performing instance segmentation of image data to identify the dentin of each individual tooth in the image data and generate pixel-wise segmentation masks for each instance of dentin. Segmentation masks may also be generated for other tooth parts, such as enamel regions, tooth roots, etc.
VI) Caries segmentation—this can include performing semantic segmentation of image data to assign caries labels to pixels in the image data. This may additionally include performing instance segmentation of image data to identify each caries in the image data and generate pixel-wise segmentation masks for each instance of caries. In some instances, the input to an ML model that performs caries segmentation includes a cropped image (e.g., a cropped radiograph) that has been cropped based on a determined ROI. In some instances, an input to an ML model that performs caries segmentation includes an image (e.g., a radiograph) plus a mask indicating a ROI of the image to consider for caries. Pixels of the image not in the ROI may not be processed in embodiments.
VII) Caries location determination—this can include processing caries segmentation information (e.g., as output by another ML model) to identify, for each instance of a caries, a location of the caries on a tooth.
VIII) Caries detection—this can include performing object detection to identify instances of caries in image data and assigning bounding shapes (e.g., bounding boxes) around each identified caries.
IX) Region of interest segmentation—this can include processing image data to identify one or more regions of interest in the image data. A mask may be generated for each ROI, and may be used to crop the image data and/or as a further input to one or more other ML models along with the image data. Examples of ROIs that may be determined include a tooth and jaw region, one or more tooth regions, a periodontal bone loss region (e.g., including the area between tooth crowns and tooth root apexes for a lower jaw and/or an upper jaw), and so on.
X) Region of interest detection—this can include performing object detection to identify instances of regions of interest in image data and assigning bounding shapes (e.g., bounding boxes) around each identified ROI.
XI) Apical lesion (periapical radiolucency) segmentation—this can include performing semantic segmentation of image data to assign apical lesion/periapical radiolucency labels to pixels in the image data. This may additionally include performing instance segmentation of image data to identify each apical lesion in the image data and generate pixel-wise segmentation masks for each instance of apical lesions. In some instances, the input to an ML model that performs apical lesion segmentation includes a cropped image (e.g., a cropped radiograph) that has been cropped based on a determined ROI. In some instances, an input to an ML model that performs apical lesion segmentation includes an image (e.g., a radiograph) plus a mask indicating a ROI of the image to consider for apical lesions. Pixels of the image not in the ROI may not be processed in embodiments.
XII) Apical lesion detection—this can include performing object detection to identify instances of apical lesions in image data and assign bounding shapes (e.g., bounding boxes) around each identified apical lesion.
XIII) Restoration segmentation—this can include performing semantic segmentation of image data to assign restoration labels to pixels in the image data. This may additionally include performing instance segmentation of image data to identify each restoration in the image data and generate pixel-wise segmentation masks for each instance of a restoration. In some instances, the input to an ML model that performs restoration segmentation includes a cropped image (e.g., a cropped radiograph) that has been cropped based on a determined ROI. In some instances, an input to an ML model that performs restoration segmentation includes an image (e.g., a radiograph) plus a mask indicating a ROI of the image to consider for restorations. Pixels of the image not in the ROI may not be processed in embodiments.
XIV) Restoration detection—this can include performing object detection to identify instances of restorations in image data and assigning bounding shapes (e.g., bounding boxes) around each identified restoration.
XV) Periodontal bone loss segmentation—this can include performing semantic segmentation of image data to assign periodontal bone line labels, CEJ labels and/or tooth root labels to pixels in the image data. This may additionally include performing instance segmentation of image data to identify, for example, each tooth root in the image data and generate pixel-wise segmentation masks for each instance of a tooth root. In some instances, the input to an ML model that performs periodontal bone loss segmentation includes a cropped image (e.g., a cropped radiograph) that has been cropped based on a determined ROI. In some instances, an input to an ML model that performs periodontal bone loss segmentation includes an image (e.g., a radiograph) plus a mask indicating a ROI of the image to consider for one or more oral structures relevant for determining periodontal bone loss (e.g., CEJ, PBL, etc.). Pixels of the image not in the ROI may not be processed in embodiments.
XVI) Impacted tooth segmentation—this can include performing semantic segmentation of image data to assign impacted tooth labels to pixels in the image data. This may additionally include performing instance segmentation of image data to identify each impacted tooth in the image data and generate pixel-wise segmentation masks for each instance of an impacted tooth. In some instances, the input to an ML model that performs impacted tooth segmentation includes a cropped image (e.g., a cropped radiograph) that has been cropped based on a determined ROI. In some instances, an input to an ML model that performs impacted tooth segmentation includes an image (e.g., a radiograph) plus a mask indicating a ROI of the image to consider for impacted teeth. Pixels of the image not in the ROI may not be processed in embodiments.
XVII) Impacted tooth detection—this can include performing object detection to identify instances of impacted teeth in image data and assign bounding shapes (e.g., bounding boxes) around each identified partially erupted tooth.
XVIII) Partially erupted tooth segmentation—this can include performing semantic segmentation of image data to assign partially erupted tooth labels to pixels in the image data. This may additionally include performing instance segmentation of image data to identify each partially erupted tooth in the image data and generate pixel-wise segmentation masks for each instance of a partially erupted tooth. In some instances, the input to an ML model that performs partially erupted tooth segmentation includes a cropped image (e.g., a cropped radiograph) that has been cropped based on a determined ROI. In some instances, an input to an ML model that performs partially erupted tooth segmentation includes an image (e.g., a radiograph) plus a mask indicating a ROI of the image to consider for partially erupted teeth. Pixels of the image not in the ROI may not be processed in embodiments.
XIX) Partially erupted tooth detection—this can include performing object detection to identify instances of partially erupted teeth in image data and assign bounding shapes (e.g., bounding boxes) around each identified partially erupted tooth.
XX) Calculus segmentation—this can include performing semantic segmentation of image data to assign calculus labels to pixels in the image data. This may additionally include performing instance segmentation of image data to identify each instance of calculus in the image data and generate pixel-wise segmentation masks for each instance of calculus.
XXI) Calculus detection—this can include performing object detection to identify individual instances of calculus in image data and assign bounding shapes (e.g., bounding boxes) around each identified instance of calculus.
XXII) Dental object segmentation—this can include performing point-level classification (e.g., pixel-level classification or voxel-level classification) of different types of dental objects from intraoral scans, sets of intraoral scans, 3D surfaces generated from multiple intraoral scans, 3D models generated from multiple intraoral scans, etc. The different types of dental objects may include, for example, teeth, gingiva, an upper palate, a preparation tooth, a restorative object other than a preparation tooth, an implant, a bracket, an attachment to a tooth, soft tissue, a retraction cord (dental wire), blood, saliva, and so on. In some embodiments, different types of restorative objects may be identified, different types of implants may be identified, different types of brackets may be identified, different types of attachments may be identified, different types of soft tissues (e.g., tongue, lips, cheek, etc.) may be identified, and so on.
XXIII) Actionable symptom generation—this can include processing image data and/or information on one or more types of detected oral conditions and/or oral structures (e.g., calculus, restorations, caries, apical lesions, mandibular nerve canal, tooth wear, tooth cracks, bleeding, gingival recession, periodontal bone loss, impacted teeth, partially erupted teeth, etc.) using one or more ML models to output one or more actionable symptom recommendations.
XXIV) Oral health problem diagnosis—this can include processing image data and/or information on one or more types of detected oral conditions and/or oral structures (e.g., calculus, restorations, caries, apical lesions, mandibular nerve canal, tooth wear, tooth cracks, bleeding, gingival recession, periodontal bone loss, impacted teeth, partially erupted teeth, etc.) using one or more ML models to output one or more diagnoses of oral health problems of a patient.
XXV) Treatment recommendation—this can include processing image data, information on one or more types of detected oral conditions and/or oral structures (e.g., calculus, restorations, caries, apical lesions, mandibular nerve canal, tooth wear, tooth cracks, bleeding, gingival recession, periodontal bone loss, impacted teeth, partially erupted teeth, etc.), one or more actionable symptom recommendations, and/or one or more oral health problem diagnoses using one or more ML models to output one or more treatment recommendations for a patient.
XXVI) Trends analysis—this can include processing image data, information on one or more types of detected oral conditions and/or oral structures (e.g., calculus, restorations, caries, apical lesions, mandibular nerve canal, tooth wear, tooth cracks, bleeding, gingival recession, periodontal bone loss, impacted teeth, partially erupted teeth, etc.), one or more actionable symptom recommendations, and/or one or more oral health problem diagnoses from multiple points in time using one or more ML models to identify trends associated with changes in the oral structures, oral conditions, oral health problems, and so on.
XXVII) Predictive analysis—this can include processing image data, information on one or more types of detected oral conditions and/or oral structures (e.g., calculus, restorations, caries, apical lesions, mandibular nerve canal, tooth wear, tooth cracks, bleeding, gingival recession, periodontal bone loss, impacted teeth, partially erupted teeth, etc.), one or more actionable symptom recommendations, and/or one or more oral health problem diagnoses from one or more points in time using one or more ML models to predict future conditions of the oral structures, oral conditions, oral health problems, and so on. In embodiments, one or more generative models are used to perform the predictive analysis. The one or more generative models may generate synthetic image data (e.g., synthetic radiographs, synthetic 3D models of dental arches, etc.) of the patient's oral cavity showing a predicted state of the patient's oral cavity at one or more points in the future.
XXVIII) Treatment simulation—this can include processing image data, information on one or more types of detected oral conditions and/or oral structures (e.g., calculus, restorations, caries, apical lesions, mandibular nerve canal, tooth wear, tooth cracks, bleeding, gingival recession, periodontal bone loss, impacted teeth, partially erupted teeth, etc.), one or more actionable symptom recommendations, one or more oral health problem diagnoses, and/or one or more treatments using one or more ML models to predict future conditions of the oral structures, oral conditions, oral health problems, and so on, after treatment. In embodiments, one or more generative models are used to perform the simulation. The one or more generative models may generate synthetic image data (e.g., synthetic radiographs, synthetic 3D models of dental arches, etc.) of the patient's oral cavity showing a predicted state of the patient's oral cavity at one or more points in the future after and/or during treatment.
XXIX) Prescription generation—this can include predicting parameters for a prescription based on image data, one or more determined oral condition(s) of a patient, determined diagnoses of one or more oral health problems of a patient, selected treatment(s) for the patient, and so on. Examples of prescription parameters that may be predicted include whether a prescription is for orthodontic treatment or restorative treatment, one or more teeth to be treated, a type of prosthodontic to be used, a color to be used for a prosthodontic, a material to be used for a prosthodontic, a lab to be used, and so on. Each of the different types of predictions/classifications associated with prescription generation may be determined by a separate ML model or by a ML model trained to generate multiple different outputs. For example, separate ML models may be trained to determine a dental lab, a type of dental prosthetic, a material for a dental prosthetic, a color for a dental prosthetic, and so on.
XXX) Case type classification—this can include determining whether orthodontic treatment and/or restorative treatment will be performed for a patient based on intraoral scans, sets of intraoral scans, 3D surfaces generated from multiple intraoral scans, 3D models generated from multiple intraoral scans, radiographs, a CBCT scan, and so on.
XXXI) Tooth number classification—this can include performing pixel level identification/classification and/or group/patch-level identification/classification of each tooth from 3D surface data and/or 2D image data. Teeth can be classified using one or more standard tooth numbering schemes, such as the American Dental Association (ADA) teeth numbering.
XXXII) Tooth to gum border identification/marking—this can include performing pixel-level identification/classification of a tooth to gum border around one or more tooth based on intraoral scans, sets of intraoral scans, 3D surfaces generated from multiple intraoral scans, 3D models generated from multiple intraoral scans, radiographs, CBCT scans, and so on.
XXXIII) Tooth to tooth (interproximal region) border identification/marking—this can include performing pixel-level identification/classification of a tooth to tooth border for one or more interproximal regions between teeth based on intraoral scans, sets of intraoral scans, 3D surfaces generated from multiple intraoral scans, 3D models generated from multiple intraoral scans, radiographs, CBCT scans, and so on.

Note that for any of the above identified tasks associated with radiographs, though they are described as being performed based on an input of radiographs, it should be understood that these tasks may also be performed based on 3D models, color images, NIRI images, CBCT scans, and so on. Any of these tasks may be performed using ML models with multiple input layers or channels, where a first layer may be for data of a first image modality and a second layer may be for data of a second image modality.

One type of machine learning model that may be used to perform some or all of the above asks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a machine learning model such as a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

For the model training workflow 1805, a training dataset containing hundreds, thousands, tens of thousands, hundreds of thousands or more intraoral scans, images (e.g., radiographs), and/or 3D models should be used. In embodiments, up to millions of cases of patient dentition that may have underwent a prosthodontic procedure and/or an orthodontic procedure may be available for forming a training dataset, where each case may include various labels of one or more types of useful information. Each case may include, for example, data showing a 3D model, intraoral scans, height maps, color images, NIRI images, radiographs, etc. of one or more dental sites, data showing pixel-level segmentation of the data (e.g., 3D model, intraoral scans, height maps, color images, NIRI images, radiographs, etc.) into various dental object classes and/or oral condition classes (e.g., tooth, restorative object, gingiva, moving tissue, upper palate, caries, dentin, periodontal bone line, CEJ, apical lesion, etc.), data showing one or more assigned classifications for the data (e.g., lingual view, buccal view, occlusal view, anterior view, left side view, right side view, etc.), and so on. This data may be processed to generate one or multiple training datasets 1836 for training of one or more machine learning models.

In one embodiment, generating one or more training datasets 1836 includes gathering image data with labels 1810 and/or additional data (e.g., doctor notes, patient input, data from a DPMS, etc.) with labels 1812. The labels that are used may depend on what a particular machine learning model will be trained to do.

Processing logic may gather a training dataset 1836 comprising 2D or 3D images, intraoral scans, 3D surfaces, 3D models, height maps, bite-wing radiographs, panoramic radiographs, periapical radiographs, CBCT scans, ultrasound scans, etc. of dental sites (e.g., of an oral cavity) having one or more associated labels (e.g., pixel-level labeled dental classes in the form of maps (e.g., probability maps), image level labels, etc.). One or more images, scans, surfaces, radiographs, and/or models and optionally associated probability maps in the training dataset 1836 may be resized in embodiments. For example, a machine learning model may be usable for images having certain pixel size ranges, and one or more image may be resized if they fall outside of those pixel size ranges. The images may be resized, for example, using methods such as nearest-neighbor interpolation or box sampling. The training dataset may additionally or alternatively be augmented. Training of large-scale neural networks generally uses tens of thousands of images, which are not easy to acquire in many real-world applications. Data augmentation can be used to artificially increase the effective sample size. Common techniques include random rotation, shifts, shear, flips and so on to existing images to increase the sample size.

To effectuate training, processing logic inputs the training dataset(s) 1836 into one or more untrained machine learning models. Prior to inputting a first input into a machine learning model, the machine learning model may be initialized. Processing logic trains the untrained machine learning model(s) based on the training dataset(s) to generate one or more trained machine learning models that perform various operations as set forth above.

Training may be performed by inputting one or more of the images, scans, radiographs, or 3D surfaces (or data from the images, scans or 3D surfaces) into the machine learning model one at a time. Each input may include data from an image, intraoral scan, radiograph, or 3D surface in a training data item from the training dataset. The training data item may include, for example, a radiograph and an associated segmentation map, which may be input into the machine learning model. As discussed above, training data items may also include color images, images generated under specific lighting conditions (e.g., UV or IR radiation), intraoral scans, CBCT scans, ultrasound scans, 3D models, and so on. The data that is input into the machine learning model may include a single layer (e.g., just intensity values from a single radiograph) or multiple layers. If multiple layers are used, then one layer may include the intensity values from the radiograph, and a second layer may include intensity values, color values, height values, etc. from other image data (e.g., a color image, intraoral scan, 3D surface, height map, etc.).

The machine learning model processes the input to generate an output. An artificial neural network includes an input layer that consists of values in a data point (e.g., intensity values in a radiograph). The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the machine learning model can produce. For example, for an artificial neural network being trained to perform caries segmentation, there may be a first class (caries) and a second class (not caries). The class, prediction, etc. may be determined for each pixel in the image data, may be determined for an entire image/scan/surface, or may be determined for each region or group of pixels of the image/scan/surface. For pixel level segmentation, for each pixel in the image/scan/surface, the final layer applies a probability that the pixel of the image/scan/surface belongs to the first class, a probability that the pixel belongs to the second class, and so on.

Accordingly, the output may include one or more prediction and/or one or more a probability map. For example, an output probability map may comprise, for each pixel in an input image/scan/surface, a first probability that the pixel belongs to a first dental class, a second probability that the pixel belongs to a second dental class, and so on. For example, the probability map may include probabilities of pixels belonging to dental classes representing a tooth, caries, gingiva, or a restorative object. In further embodiments, different dental classes may represent different types of restorative objects.

Processing logic may then compare the generated probability map and/or other output to the known probability map and/or label that was included in the training data item. Processing logic determines an error (i.e., a classification error) based on the differences between the output probability map and/or label(s) and the provided probability map and/or label(s). Processing logic adjusts weights of one or more nodes in the machine learning model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons”, where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the deep learning model. After one or more rounds of training, processing logic may determine whether a stopping criterion has been met. A stopping criterion may be a target level of accuracy, a target number of processed images from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria is met if accuracy of the machine learning model has stopped improving. If the stopping criterion has not been met, further training is performed. If the stopping criterion has been met, training may be complete. Once the machine learning model is trained, a reserved portion of the training dataset may be used to test the model.

Once one or more trained ML models 1838 are generated, they may be stored in model storage 1845, and may be added to an oral health diagnostics system (e.g., oral health diagnostics system 215). In some embodiments, trained ML models are added to one or more segmentation pipelines (e.g., first segmentation pipeline 1867A, second segmentation pipeline 1867B, through nth segmentation pipeline 1867N. Each segmentation pipeline 1867A-N may be for processing of a different type of image data in embodiments. For example, first segmentation pipeline 1867A may be for processing bite-wing radiographs, second segmentation pipeline 1867B may be for processing panoramic radiographs, and nth segmentation pipeline 1867N may be for processing periapical radiographs. Other segmentation pipelines may be for processing 3D models of dental arches, for processing intraoral scans, for processing 2D color images, and so on.

In one embodiment, model application workflow 1817 includes one or more segmentation pipelines (e.g., first segmentation pipeline 1867A, second segmentation pipeline 1867B, through nth segmentation pipeline 1867N. Each segmentation pipeline may include multiple trained ML models, arranged in parallel and/or in series, each trained to perform one or more segmentation, classification, object detection, etc. tasks. Segmentation pipelines may additionally include traditional logic and/or modules (e.g., other than ML or AI models) that may process data before it is input into ML models and/or to process the data output by one or more ML models. For example, segmentation pipelines may include one or more postprocessing modules for combining the outputs of multiple ML models, performing measurements, reconciling disagreements between the outputs of different ML models, performing image processing, and so on.

For model application workflow 1817, according to one embodiment, capture devices for one or more image modalities capture image data of a patient's oral cavity. Such capture devices may generate x-rays 1848, intraoral scans, 3D surfaces/models, and so on. Additionally, patient data 1852 may be received based on patient input, from a DPMS, from a doctor input, and so on. One or more of these oral state capture modalities may constitute input data 1862 that may be input into one or more of the segmentation pipelines 1867A-N.

The one or more segmentation pipelines 1867A-N may operate on data from one or more oral state capture modalities on which ML models of the segmentation pipeline(s) 1867A-N were trained to generate one or more outputs 1869A, 1869B, through 1869N. Each of the outputs may include information on one or more oral structures and/or one or more oral conditions.

The outputs 1869A-N may be input into one or more oral health diagnostics engines 1870A, 1870B, through 1870N. Each of the oral health diagnostics engines 1870A-N may include one more AI models or ML models trained to operation on a particular type of image data, on data from multiple oral state capture modalities, and/or on one or more particular types of output from one or more of the segmentation pipelines 1867A-N. For example, first oral health diagnostics engine 1870A may include a first ML model trained to operate on periodontal bone loss information and/or patient data to diagnose periodontitis. In embodiments, each oral health diagnostics engine 1870A-N may include logic (e.g., one or more trained ML models, rules-based logic, decision trees, etc.) for generating an output 1872A, 1872B, through 1872N comprising one or more actionable symptom recommendations and/or one or more diagnoses of oral health problems. The outputs 1869A-N and/or outputs 1872A-N may be input into an output aggregator 1876.

Output aggregator 1867 may combine outputs from multiple segmentation pipelines 1867A-N and/or multiple oral health diagnostics engines 1870A-N to determine improved and more accurate oral state condition estimations, actionable symptom recommendations, diagnoses, and so on. For example, output aggregator 1876 may receive first outputs 1869A on oral conditions generated by first segmentation pipeline 1867A based on processing of data from a first oral state capture modality, and may receive second outputs 1869B on oral conditions generated by second segmentation pipeline 1867A based on processing of data from a second oral state capture modality. Output aggregator 1876 may combine the multiple outputs to generate aggregated output 1878. In one embodiment, the first oral state capture modality is a first type of radiograph and the second oral state capture modality is a second type of radiograph. In one embodiment, the first oral state capture modality is a radiograph, and the second oral state capture modality is an intraoral scan, a three-dimensional model, a color image, a near infrared image, a CT scan, or a CBCT scan. In order to combine the outputs of the multiple segmentation pipelines, output aggregator 1876 may perform registration between image data of two or more different image modalities in embodiments (e.g., two different types of radiographs, a radiograph and a 3D model of a dental arch, etc.). Such registration may be performed based on shared features of a dental site in the different image data.

Output aggregator 1876 may provide an aggregated output 1878 including the improved and more accurate oral state condition estimations, actionable symptom recommendations, diagnoses, and so on. The aggregated output 1878 and/or any of the outputs 1869A-N and/or outputs 1872A-N may be displayed in a GUI of an oral health diagnostics system in embodiments.

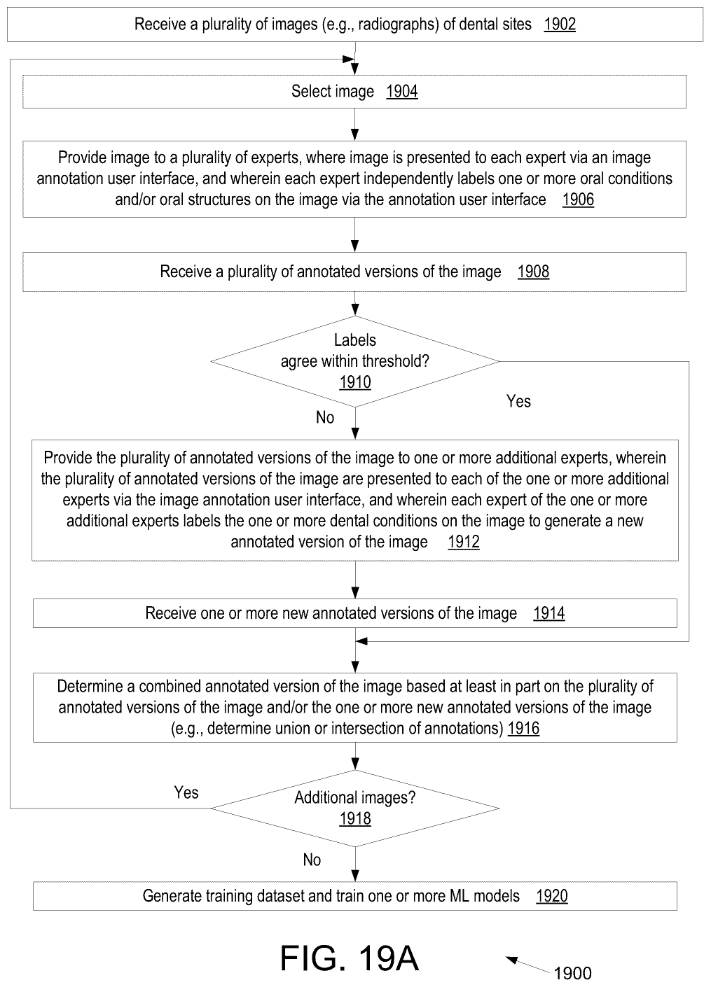

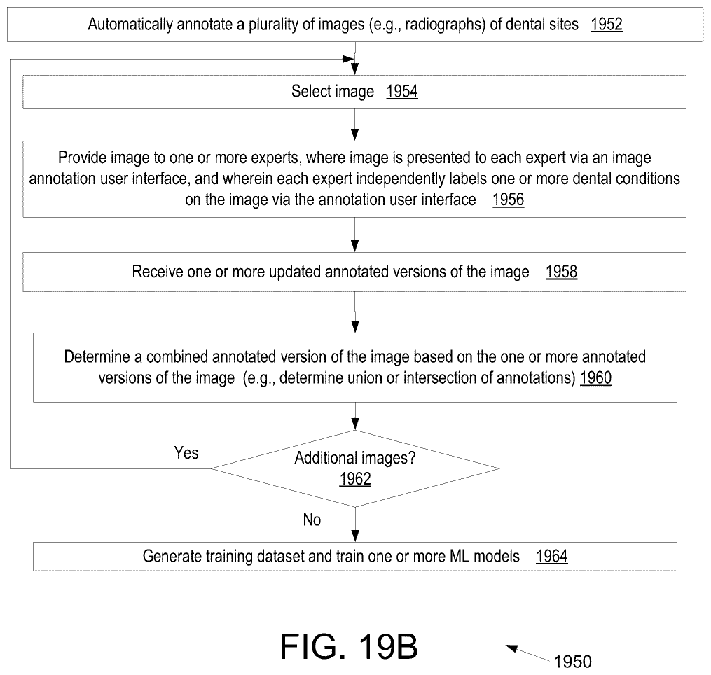

FIGS. 19A-21 illustrate flow diagrams of methods performed by an oral health diagnostics system and/or to train an ML models of an oral health diagnostics system, in accordance with embodiments of the present disclosure. These methods may be performed by processing logic that comprises hardware (e.g., circuitry, dedicated logic, programmable logic, microcode, etc.), software (such as instructions run on a processing device), or a combination thereof. In one embodiment, processing logic corresponds to computing device 305 of FIG. 3 (e.g., to a computing device 305 executing an oral health diagnostics system 215).

Since the oral health diagnostics system described in embodiments herein is medical software, it is important that the oral conditions, oral structures, actionable symptom recommendations, diagnoses of oral health problems, etc. be accurate. Accordingly, data used to train one or more ML models or AI models of the oral health diagnostics system may be manually labeled and/or checked by medical experts (e.g., doctors) to ensure that the labels for such data are accurate.

FIG. 19A illustrates a flow diagram for a method 1900 of generating a training dataset for training one or more machine learning models of a segmentation pipeline, in accordance with embodiments of the present disclosure. At block 1902 of method 1900, processing logic receives a plurality of images (e.g., radiographs) of dental sites (e.g., of one or more teeth and/or jaw of an oral cavity). At block 1904, processing logic may select an image from the multiple received images. In some embodiments, images may be selected based on properties of those images and/or of oral structures and/or oral conditions in those images. For example, certain oral conditions may be relatively rare. In order to train an ML model to accurately perform segmentation of an oral condition, the a training dataset should include a large number of examples of the oral condition. Accordingly, processing logic may determine particular oral conditions, patient case details, etc. that are underrepresented in a training dataset, and may select images that have the underrepresented oral conditions, patient case details, etc.

At block 1906, processing logic provides the image to a plurality of experts. The image may be presented to each expert via an image annotation user interface. Via the user interface, processing logic may receive user input that generates labels of one or more oral structures or oral conditions (e.g., dental conditions), and may then save annotated versions of the image that comprises the labels. The image annotation user interface may include controls for adding one or more types of labels to the image (e.g., labels for caries, restorations, apical lesions, CEJ, periodontal bone line, dentin, enamel, and so on. The image annotation user interface may additionally include controls for changing one or more parameters of the image (e.g., such as brightness, contrast, zoom setting, etc.), of labels on the image (e.g., marker opacity), of bounding boxes on the image (e.g., bounding box opacity), and so on. Processing logic may receive selection of an option to change a setting (e.g., brightness, contrast, marker opacity, box opacity, etc.), and may change the selected setting in accordance with the user input. Each expert may independently label one or more oral conditions and/or oral structures on the image via the annotation user interface. The user may also adjust parameters of the image, erase and/or modify existing labels added by the expert, and/or perform other options via interaction with the annotation user interface.

At block 1908, processing logic receives a plurality of annotated versions of the image. Processing logic may additionally save the annotated versions of the image to a data store.

At block 1910, processing logic may compare the labels of the plurality of annotated versions of the image. Processing logic may determine whether or not the labels of the different annotated versions of the image (each independently annotated by a different expert) agree to within a threshold. Labels may agree to within a threshold, for example, if the labels have a threshold amount of overlap (e.g., 70% overlap, 80% overlap, 90% overlap, 95% overlap, etc.). If the labels agree to within a threshold, the method continues to block 1916. If there is disagreement between two or more annotated versions of the image by more than a threshold amount (e.g., overlap is below an overlap threshold), then the method may proceed to block 1912.

At block 1912, processing logic provides the plurality of annotated versions of the image to one or more additional experts. Each of the plurality of annotated versions of the image may be presented to each of the one or more additional experts via the image annotation user interface. Each expert of the one or more additional experts may label the one or more dental conditions on the image to generate a new annotated version of the image. In one embodiment, an additional expert receives a version of the image that includes multiple overlay layers, where each overlay layer includes one or more labels of a particular earlier expert. The additional expert may turn on or off one or more of the layers to determine how to update a labeling of the image.

At block 1914, processing logic receives one or more new annotated versions of the image that were annotated by the one or more additional experts.

At block 1916, processing logic determines a combined annotated version of the image based at least in part on the plurality of annotated versions o the image and/or the one or more new annotated versions of the image. In one embodiment, only new annotated versions of the image are used if the original annotated versions of the image failed to agree withing a threshold. Alternatively, all annotated versions of the image may be used, but a weighting may be applied to weight the new annotated versions of the image more heavily than the original annotated versions of the image. In one embodiment, processing logic determines an intersection of the multiple annotated versions of the image and/or the one or more new annotated versions of the image. For each label of an instance of an oral structure or oral condition, the intersection of that label from multiple annotated versions of the image may be used to generate a definitive label for the oral structure or oral condition. In one embodiment, processing logic determines a union of the multiple annotated versions of the image and/or the one or more new annotated versions of the image. For each label of an instance of an oral structure or oral condition, the union of that label from multiple annotated versions of the image may be used to generate a definitive label for the oral structure or oral condition.

At block 1918, processing logic determines whether there are additional images to be labeled. If so, the method returns to block 1904 and a new image is selected. If all images have been labeled, the method proceeds to block 1920, and a training dataset may be generated for training one or more ML or AI models. The training dataset may be used to train or retrain one or more ML/AI models.

FIG. 19B illustrates a flow diagram for a method 1950 of generating a training dataset for training one or more machine learning models of a segmentation pipeline, in accordance with embodiments of the present disclosure. At block 1952 of method 1950, processing logic automatically annotates a plurality of images (e.g., radiographs) of dental sites. In embodiments, one or more trained ML models process each of the images to automatically annotate the images. Automatic annotations may include segmentation of teeth, caries, restorations, apical lesions, bone loss, tooth cracks, and so on.

At block 1954, processing logic selects an image. In some embodiments, images may be selected based on properties of those images and/or of oral structures and/or oral conditions in those images. For example, certain oral conditions may be relatively rare. In order to train an ML model to accurately perform segmentation of an oral condition, the a training dataset should include a large number of examples of the oral condition. Accordingly, processing logic may determine particular oral conditions, patient case details, etc. that are underrepresented in a training dataset, and may select images that have the underrepresented oral conditions, patient case details, etc.

At block 1956, processing logic provides the annotated image to one or more experts. The image may be presented to each expert via an image annotation user interface. Via the user interface, processing logic may receive user input that generates or updates labels of one or more oral structures or oral conditions (e.g., dental conditions) in the image, and may then save annotated versions of the image that comprises the labels.

At block 1958, processing logic receives one or more updated annotated versions of the image. Processing logic may additionally save the updated annotated versions of the image to a data store.

At block 1960, processing logic may determine a combined annotated version of the image is there were multiple updated annotated versions of the image. In one embodiment, processing logic determines an intersection of the multiple annotated versions of the image. For each label of an instance of an oral structure or oral condition, the intersection of that label from multiple annotated versions of the image may be used to generate a definitive label for the oral structure or oral condition. In one embodiment, processing logic determines a union of the multiple annotated versions of the image and/or the one or more new annotated versions of the image. For each label of an instance of an oral structure or oral condition, the union of that label from multiple annotated versions of the image may be used to generate a definitive label for the oral structure or oral condition.

At block 1962, processing logic determines whether there are additional images to be labeled. If so, the method returns to block 1954 and a new image is selected. If all images have been labeled, the method proceeds to block 1964, and a training dataset may be generated for training one or more ML or AI models. The training dataset may be used to train or retrain one or more ML/AI models.

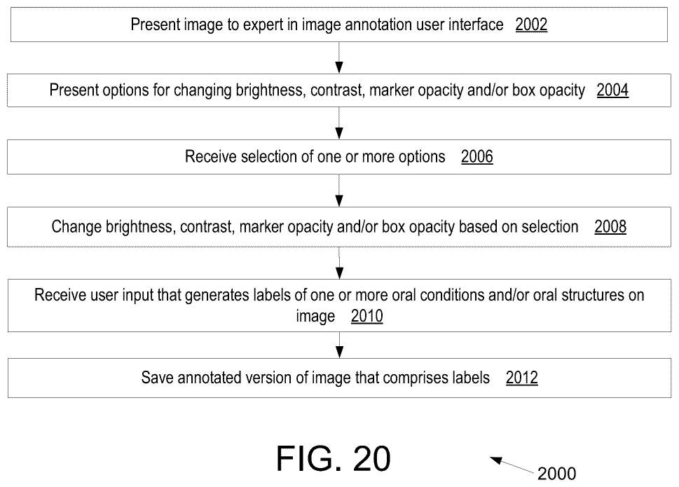

FIG. 20 illustrates a flow diagram for a method 2000 of altering images and/or radiographs to be included in a training dataset for training one or more machine learning models of a segmentation pipeline, in accordance with embodiments of the present disclosure. Method 2000 may be performed on computing devices executing an annotation user interface. At block 2002, processing logic presents an image to an expert in an image annotation user interface. At block 2004, processing logic presents options for changing brightness, contrast, marker opacity and/or bounding box opacity. At block 2006, processing logic receives selection of one or more options. At block 2008, processing logic changes a brightness, contrast, marker opacity and/or box opacity based on the received selection. At block 2010, processing logic receives user input that generates labels of one or more oral structures and/or oral conditions on the image. The user interface may include options to select multiple different types of oral conditions and/or oral structures. A user may select a type of oral structure or condition to mark, and may then use a pen or other drawing option to draw a shape on the image identifying the pixels that correspond to the oral structure or oral condition. At block 2012, processing logic saves the annotated version of the image that comprises the added labels.

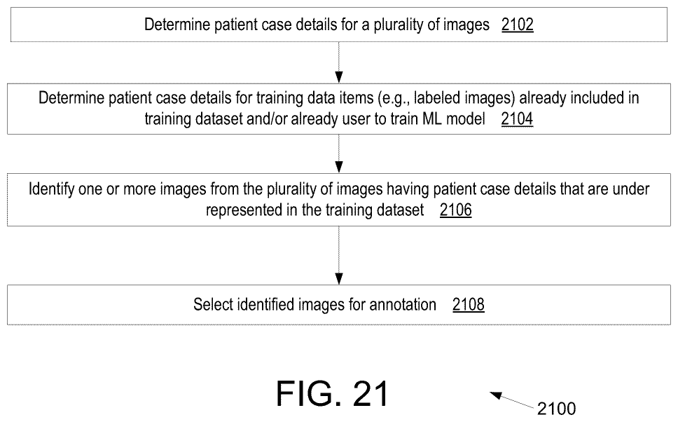

FIG. 21 illustrates a flow diagram for a method 2100 of selecting images and/or radiographs to be labeled and included in a training dataset for training one or more machine learning models of a segmentation pipeline, in accordance with embodiments of the present disclosure. At block 2102 of method 2100, processing logic determines patient case details for a plurality of images (e.g., a plurality of radiographs). At block 2104, processing logic determines patient case details for training data items (e.g., labeled images) already included in a training dataset and/or already used to train an ML model. At block 2106, processing logic identifies one or more images from the plurality of images that have patient case details that are underrepresented or not represented in the training dataset. At block 2108, processing logic selects the identified one or more images for annotation. The selected images may then be processed, for example, according to any of methods 1900, 1950 and/or 2000 to annotate the images and add them to a training dataset for training of one or more ML or AI models.

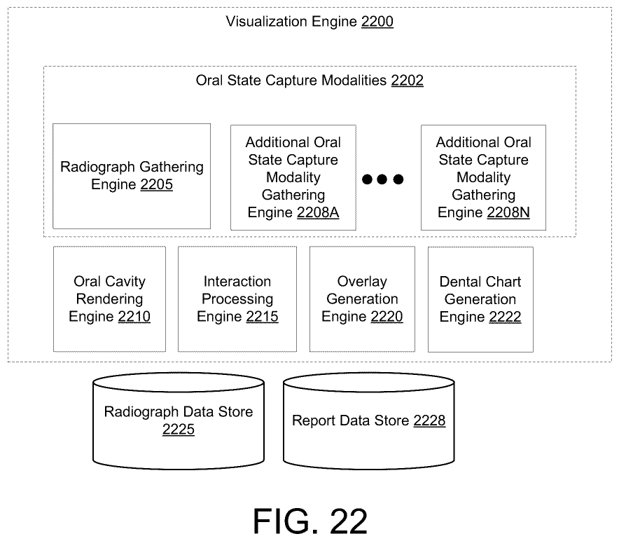

FIG. 22 illustrates a visualization engine 2200 of an oral health diagnostics system, in accordance with embodiments of the present disclosure. FIG. 22 may correspond to visualization engine 330 of FIG. 3 in an embodiment. In the example visualization engine 2200, a radiograph gathering engine 2205 can gather segmented radiographs from a relevant source. For instance, segmented radiographs can be gathered from a radiograph datastore 2225. That is, the radiograph gathering engine 2205 can retrieve stored annotated radiographs that have been annotated according to one or more segmentation pipelines in embodiments. In some implementations, the radiograph gathering engine 2205 receives radiographs from a networked resource, such as a website, intranet, or shared folder. In practice, the radiograph gathering engine 2205 may implement instructions that obtain radiographs by time, date, patient name, and/or another identifier. One or more additional oral state capture modality gathering engines 2208A-N can similarly receive annotated data of various other oral state capture modalities. The data can be received from and/or stored in data store 2225 and/or another data store. In some embodiments, a shared data store is used for multiple oral state capture modalities. Alternatively, different data stores may be used for different oral state capture modalities.

An oral cavity rendering engine 2210 can implement processes to render one or more images of a patient's oral cavity to a display of a user interface. An overlay generation engine 2220 may generate one or more overlays for the rendered image of the patient's oral cavity based on labels (e.g., segmentation masks) for one or more oral structures and/or oral conditions identified in the image by one or more segmentation pipelines. Overlay generation engine may render each overlay over the rendered image. In embodiments, different oral structures and/or oral conditions are rendered using different visualizations (e.g., different colors). This makes it easier for a doctor viewing the rendering of the image and the overlays to easily identify different oral conditions in the image. Additionally, different visualizations may be used for different severity levels of one or more oral conditions. For example, periodontal bone loss regions may be color coded based on severity of periodontal bone loss at those regions. Interaction processing engine 2215 may provide one or more interactive features (e.g., icons, images, overlays, etc.) in the user interface. In embodiments, each of the overlays for an oral structure and/or oral condition are interactive. Accordingly, a user may select on an overlay for an oral condition to bring up additional information about that oral condition. Additionally, a user may turn on or off layers for one or more oral conditions and/or oral structures via interaction with the user interface.

In embodiments, a dental chart generation engine 2222 may generate a dental chart for a patient and populate the dental chart with information from the annotated image(s). For example, dental chart generation engine 2222 may process the image to determine information on each of the oral conditions included in the information (e.g., labels, masks, segmentation information, etc.) for the image. Dental chart generation engine 2222 may, for example, read the labels for the image and determine which teeth are included in the image, what oral conditions have been identified for each tooth, severity of the oral conditions, and so on. Dental chart generation engine 2222 may then add the determined information to the generated dental chart.

In some embodiments, visualization engine 2200 generates a report for the patient that includes information on the patient's oral structures and their oral conditions, diagnoses of any oral health problems, treatment recommendations, etc. The report may be generated according to one or more report preferences of a dental practice, for example. The report may include an annotated version of the image with overlays for the different oral conditions, the dental chart, a list of oral conditions for each tooth, any diagnoses, treatment recommendations, doctor notes, patient feedback, and so on. The report may then be saved to a report data store 2228 in embodiments.

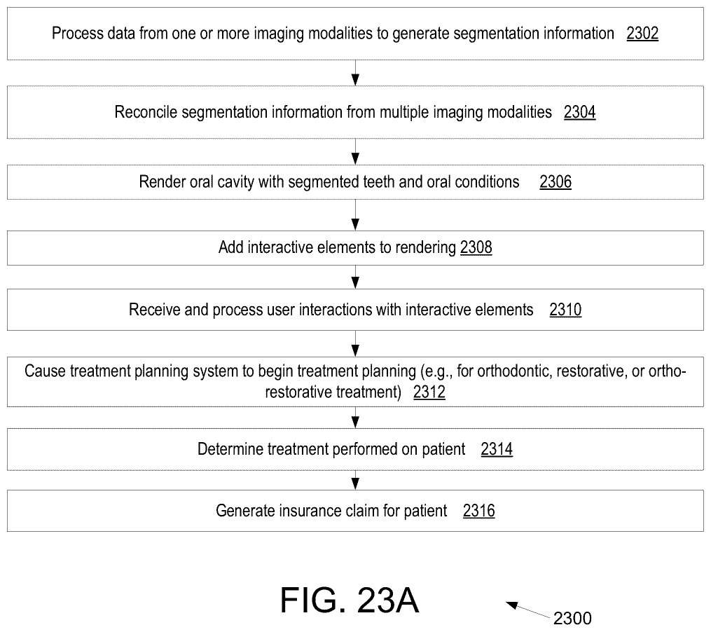

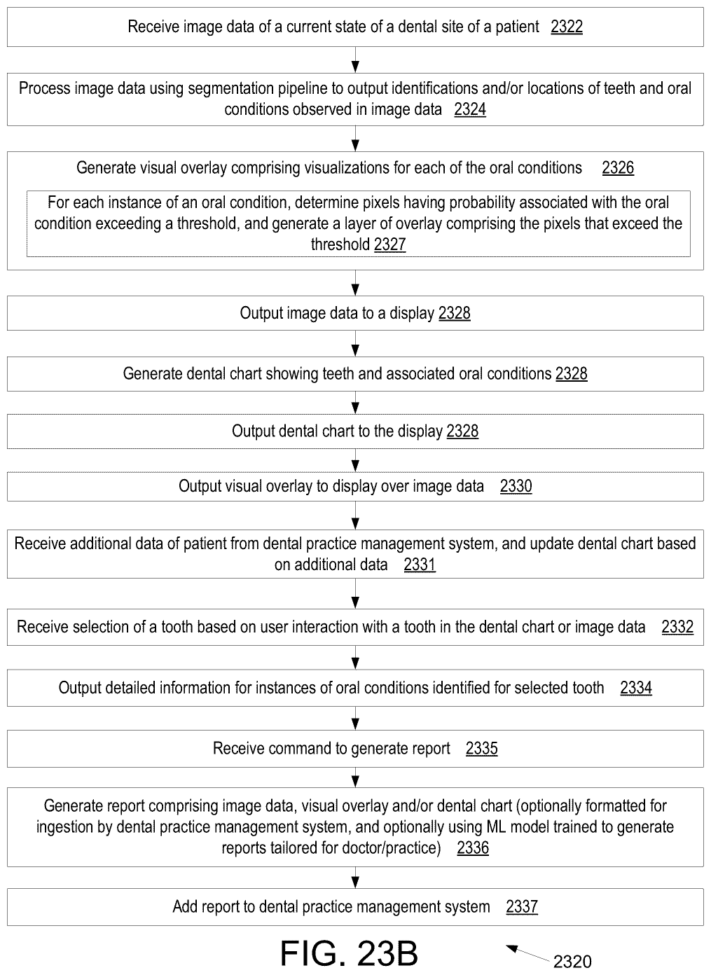

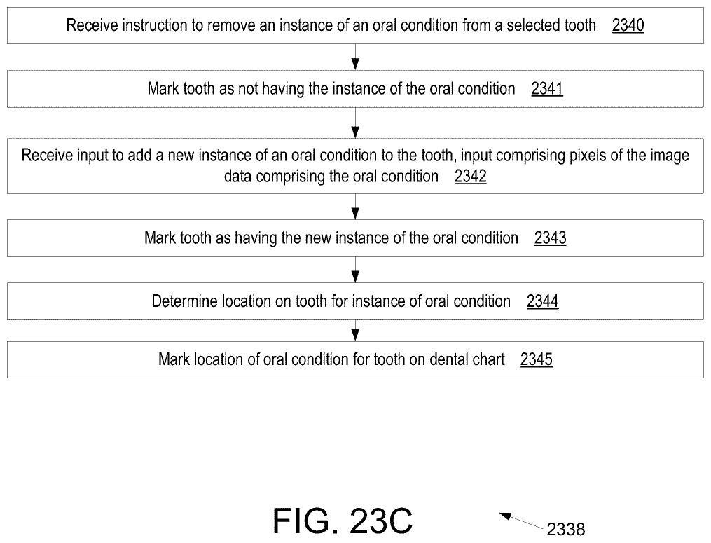

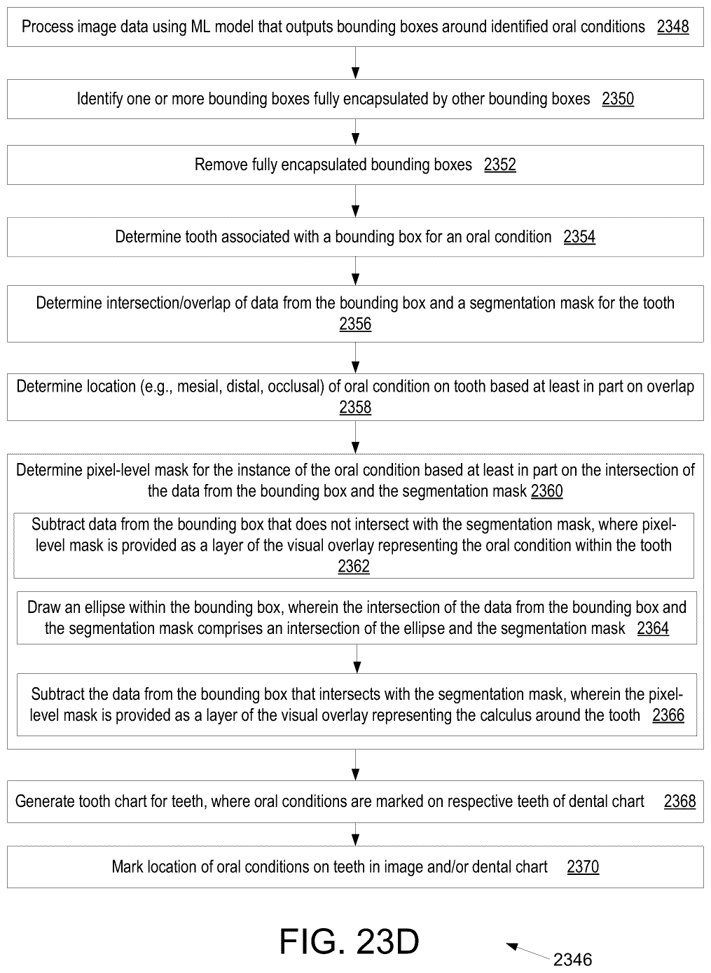

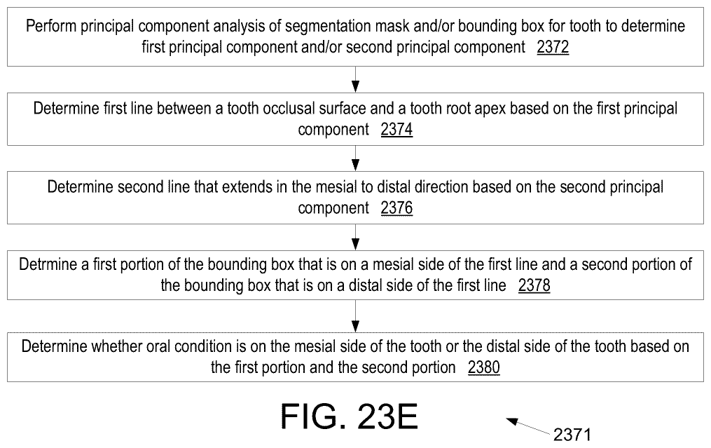

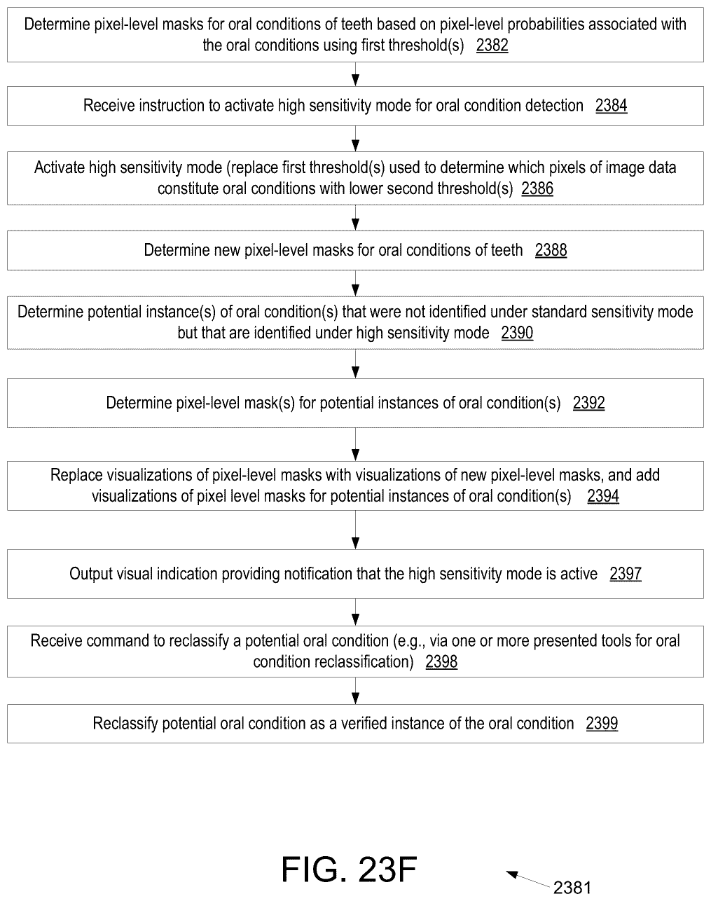

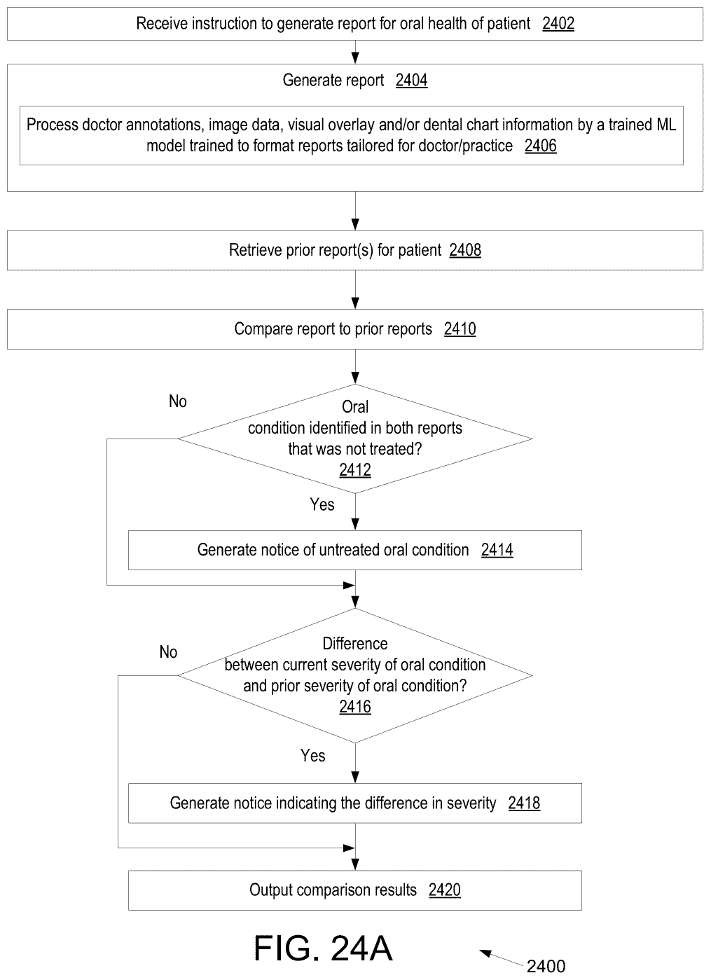

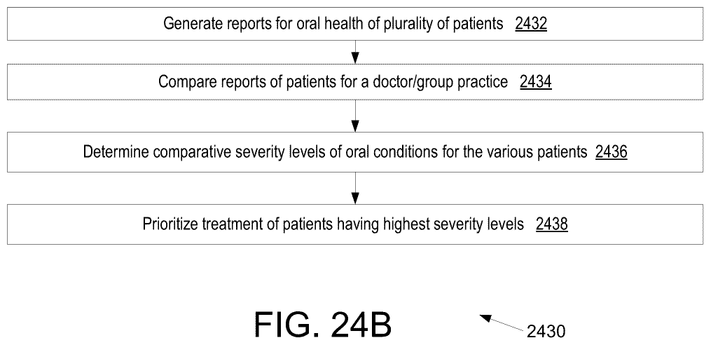

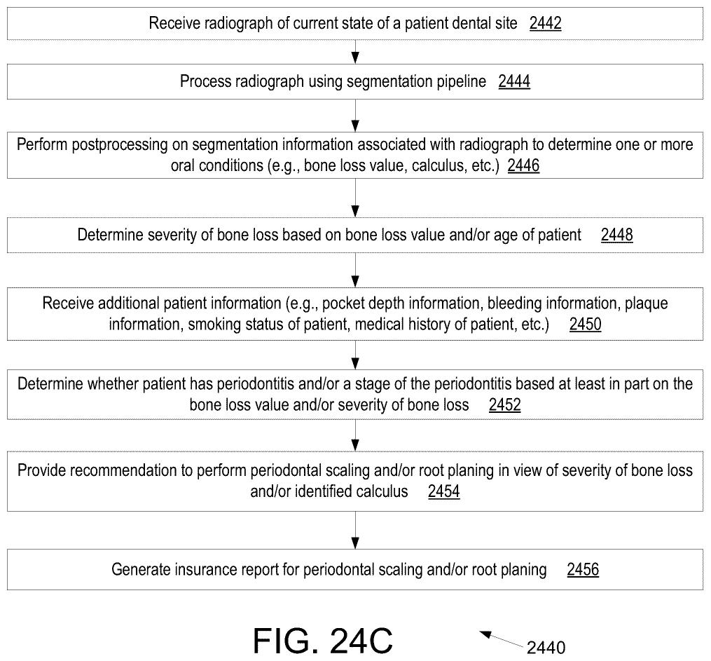

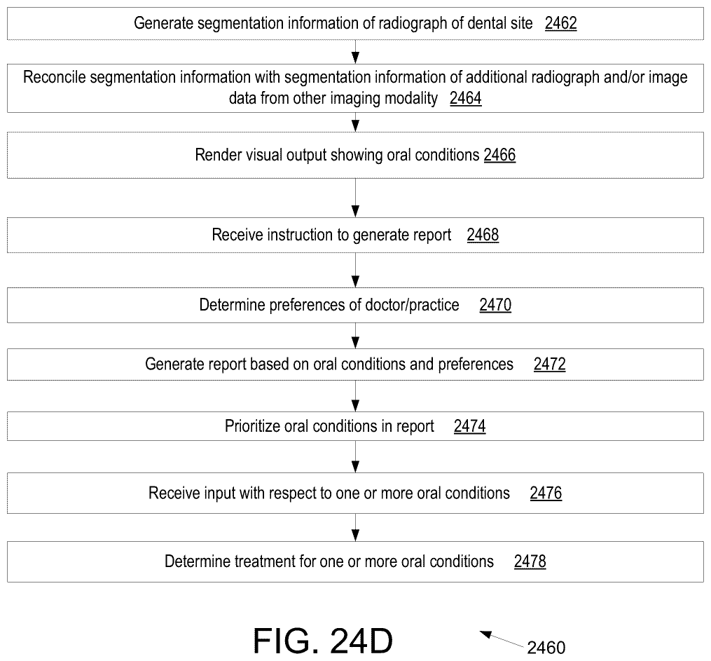

FIGS. 23A-24D illustrate flow diagrams of methods performed by an oral health diagnostics system, in accordance with embodiments of the present disclosure. These methods may be performed by processing logic that comprises hardware (e.g., circuitry, dedicated logic, programmable logic, microcode, etc.), software (such as instructions run on a processing device), or a combination thereof. In one embodiment, processing logic corresponds to computing device 305 of FIG. 3 (e.g., to a computing device 305 executing an oral health diagnostics system 215).

FIG. 23A illustrates a flow diagram for a method 2300 of providing visualizations of oral conditions of a patient, in accordance with embodiments of the present disclosure. At block 2302 of method 2300, processing logic processes data from one or more imaging modalities to generate segmentation information 2302. At block 2304, processing logic may reconcile segmentation information from multiple imaging modalities. This may include determining an intersection or union of an oral condition identified in multiple different imaging modalities, and using the intersection or union as a final state of the oral condition, for example.

At block 2306, processing logic renders an oral cavity with segmented teeth and oral conditions. This may include rendering an image (e.g., a radiograph) of the oral cavity as well as one or more overlays providing information on identified oral conditions.

At block 2308, processing logic may add interactive elements to the rendering. In one embodiment, one or more of the overlays are enabled as interactive elements. Accordingly, a user may click on an overlay of an oral condition over the image of the oral cavity to display additional information about the oral condition, to enable a doctor to add notes about the oral condition, to enable a doctor to change a classification of the oral condition, to enable a doctor to remove or hide the oral condition, and so on. The user interface may also add additional user interactive elements, such as buttons to initiate orthodontic treatment planning or restorative treatment planning, buttons to switch views of the oral cavity, and so on.

At block 2310, processing logic receives and processes user interactions with one or more interactive elements (e.g., one or more interactive overlays). In one embodiment, user interaction with the one or more interactive elements causes a treatment planning system to begin treatment planning (e.g., for orthodontic, restorative, or ortho-restorative treatment). Once treatment planning is complete, treatment may then commence for the patient.

At block 2314, processing logic may determine one or more treatments performed on the patient. At block 2316, processing logic may generate an insurance claim for the one or more performed treatments. The generated insurance claim may include a radiograph or other image of the oral cavity prior to and/or after treatment, labels of one or more oral conditions on the radiograph or other image, one or more insurance codes, a tooth chart, and/or other information. The insurance claim may be formatted based on an identification of an insurance provider to which the insurance claim will be submitted. In embodiment, the insurance claim is automatically submitted to the insurance provider.

FIG. 23B illustrates a flow diagram for a method 2320 of providing visualizations of oral conditions of a patient and of generating a report for the patient, in accordance with embodiments of the present disclosure. At block 2322 of method 2320, processing logic receives images data (e.g., a radiograph) of a current state of a dental site of a patient. At block 2324, processing logic processes the image data using a segmentation pipeline to output identifications and/or locations of teeth and oral conditions observed in the image data. At block 2326, processing logic generates one or more visual overlay comprising visualizations for each of the oral conditions. This may include at block 2327, for each instance of an oral condition, determining pixels having a probability associated with the oral condition that exceed a probability threshold, and generating a layer of the overlay comprising the pixels that exceed the threshold.

At block 2328, processing logic outputs the image data to a display. At block 2328, processing logic generates a dental chart showing teeth and associated oral conditions of the patient. At block 2328, processing logic outputs the dental chart to the display. At block 2330, processing logic outputs the visual overlay to the display over the image data. The output to the display may indicate severity of oral conditions using one or more different visualizations. For example, visualizations of oral conditions may differ based on severity. Different visualizations may also be used for different oral conditions. In some embodiments, processing logic may determine at least one of diagnoses or treatment options for one or more oral conditions. These diagnoses and/or treatment options may be shown in a dropdown menu in embodiments. For example, a user may select on an instance of an oral condition, and in response a dropdown menu showing diagnoses associated with the selected oral condition and/or treatment options for the selected oral condition may be shown. In some embodiments, processing logic may determine dental codes associated with one or more oral conditions, and may assign the dental codes to the one or more oral conditions.

At block 2331, processing logic receives additional data of the patient from a DPMS, and updates the dental chart based on the additional data. At block 2332, processing logic receives selection of a tooth based on an interaction with the tooth in the dental chart or the image data. At block 2334, processing logic outputs detailed information for instances of oral conditions identified for the selected tooth. Additional information for the selected tooth may additionally be displayed, such as pocket depth, patient feedback (e.g., pain at the tooth, bleeding around the tooth, etc.).

At block 2335, processing logic may receive a command to generate a report. The report can be for a selected tooth, for a region of the oral cavity of the patient, or for the entire oral cavity of the patient.

At block 2336, processing logic generates a report comprising the image data, the visual overlay(s) and/or the dental chart. The report may indicate severity of oral conditions using one or more different visualizations. For example, visualizations of oral conditions may differ based on severity. Different visualizations may also be used for different oral conditions. The report may additionally include one or more diagnoses of oral health problems, which may have been determined automatically, or may have been determined by a doctor based on the oral conditions and/or actionable information provided to the doctor. In some embodiments, processing logic may determine dental codes associated with one or more oral conditions, and may assign the dental codes to the one or more oral conditions. The report may include the dental codes.

In some embodiments, processing logic receives additional information on the patient from a DPMS. Such additional information may include, for example, pocket depths, patient age, or patient underlying health conditions. The additional data may be incorporated into the report.

The report may optionally be formatted for ingestion by a DPMS. For example, the report may be structured in a manner that can be understood by a DPMS. In embodiments, the report may be generated based on one or more preferences of a doctor or practice for which the report is generated. In one embodiment, patient information (e.g., image data, dental chart, information on oral conditions, information on diagnosed oral health problems, etc.) may be input into a trained ML model, which may be a generative model that outputs the report. The trained ML model may have been trained to generate reports formatted for a particular practice, or may receive as an additional input an ID of a practice and may format the report at least in part based on the ID and prior training.

In one embodiment, at block 2337 the report and/or details from the report are added to a DPMS. In some embodiments, the report is formatted in a structured data format that can be processed by the DPMS.

In some embodiments, the report may be input into a treatment planning system. The treatment planning system may then develop a treatment plan for treating one or more oral conditions of the patient based at least in part on the report.

FIG. 23C illustrates a flow diagram for a method 2338 of updating identified oral conditions of a patient based on doctor interaction with a user interface of an oral health diagnostics system, in accordance with embodiments of the present disclosure. In some cases, a doctor may disagree with an instance of an oral condition determined by an oral health diagnostics system. Accordingly, the oral health diagnostics system may include a mechanism for a doctor to override identified oral conditions and/or to update identified oral conditions. Additionally, the oral health diagnostics system may include a mechanism for the doctor to update, remove and/or replace automatic diagnosis of one or more oral health problems. Method 2338 may be performed after an oral health diagnostics system has identified and output information on one or more oral conditions for a patient.

At block 2340 of method 2338, processing logic receives an instruction to remove an instance of an oral condition from selected tooth. The doctor may select the instance of the oral condition for the tooth by clicking on an overlay for the instance of the oral condition rendered over an image and/or by clicking on the oral condition in a tooth chart or a list of oral conditions. Once the oral condition instance is selected, multiple options may be presented for the instance of the oral condition. The options may include an option to remove or update the oral condition. The doctor may select the option to remove the oral condition from the presented options.

At block 2341, processing logic marks the tooth as not having the instance of the oral condition.

At block 2342, processing logic may receive an input to add a new instance of an oral condition. This may include receiving a selection of a tooth to add the oral condition to and receiving a selection of a type of oral condition to add to the tooth. Subsequently, the doctor may manually mark the image (e.g., via a pen or draw feature of the user interface). The doctor may draw on the image to provide input comprising pixels of the image comprising the selected type of oral condition. At block 2343, processing logic marks the tooth as having the new instance of the oral condition.

At block 2344, processing logic may determine a location on the tooth for the new instance of the oral condition. At block 2345, processing logic may then mark the oral condition for the tooth on the dental chart, optionally with the location information. The location information may include, for example, left of tooth, right of tooth, tooth occlusal surface, tooth mesial surface, tooth lingual surface, and so on.

Various ML models in a segmentation pipeline may be trained to output bounding boxes around one or more identified types of oral conditions. In embodiments, postprocessing may be performed on such bounding boxes to improve quality, and/or to generate visualizations of the instances of the oral conditions.

FIG. 23D illustrates a flow diagram for a method 2346 of determining visualizations for one or more oral conditions to be presented in a user interface of an oral health diagnostics system, in accordance with embodiments of the present disclosure. At block 2348, processing logic processes image data (e.g., a radiograph) using a machine learning model that outputs bounding boxes around identified oral conditions. The oral conditions may be, for example, caries, calculus, periapical lesions, and so on.

Various operations may be performed on the bounding boxes to improve detections of oral conditions. At block 2350, processing logic identifies one or more bounding boxes fully encapsulated by other bounding boxes. At block 2352, processing logic removes any fully encapsulated bounding boxes.

At block 2354, processing logic determines a tooth associated with a bounding box for an oral condition. At block 2356, processing logic determines an intersection or overlap of data from the bounding box and a segmentation mask for the tooth. At block 2358, processing logic determines a location (e.g., mesial, distal, occlusal, etc.) of the oral condition on the tooth at least in part based on the overlap and/or based on processing of the image data using an ML model.

At block 2360, processing logic determines a pixel-level mask for the instance of the oral condition based at least in part on the intersection of the data from the bounding box and the segmentation mask for the tooth.

In one embodiment, at block 2362 processing logic subtracts data from the bounding box that does not intersect with the segmentation mask. The pixel-level mask may be provided as a layer of the visual overlay representing the oral condition within the tooth.

In one embodiment, at block 2364 processing logic draws an ellipse with in the bounding box. The intersection of the data from the ellipse and the segmentation mask for the tooth may be used as the intersection of the bounding box and the segmentation mask. At block 2366, processing logic may subtract the data from the bounding box that intersects with the segmentation mask. In an embodiment, the bounding box may represent object detection of calculus associated with a tooth. The pixel level mask may be provided as a layer of the visual overlay representing the calculus around the tooth.

At block 2368, processing logic generates a tooth chart for teeth of a patient. The oral conditions may be marked on respective teeth of the tooth chart. At block 2370, processing logic may mark the determined location(s) of one or more oral conditions on teeth in the image and/or dental chart (e.g., left of tooth, right of tooth, at mesial surface, at occlusal surface, at buccal surface, etc.).

FIG. 23E illustrates a flow diagram for a method 2371 of determining a region of a tooth that an oral condition is associated with, in accordance with embodiments of the present disclosure. In one embodiment, method 2371 is performed at block 2358 of method 2346.

At block 2372 of method 2371, processing logic performs principal component analysis of a segmentation mask and/or a bounding box for a tooth to determine a first principal component and/or a second principal component of the tooth. At block 1374, processing logic determines a first line between a tooth occlusal surface and a tooth root apex based on the first principal component. At block 2376, processing logic may determine a second line that extends in the mesial to distal direction based on the second principal component. At block 2378, processing logic determines a first portion of the bounding box that is on a mesial side of the first line and a second portion of the bounding box that is on a distal side of the first line. At block 2380, processing logic determines whether the oral condition is on the mesial side of the tooth or the distal side of the tooth based on the first portion and the second portion. For example, if the first portion is larger than the second portion, then the oral condition may be determined to be on the mesial side of the tooth. If the second portion is larger than the first portion, then the oral condition may be determined to be on the distal side of the tooth.

In embodiments, an oral health diagnostics system may include multiple thresholds for determining whether oral conditions are existent for a patient and/or for determining size and/or severity of such oral conditions. In some instances, processing logic may determine that there are not quite enough pixels classified as belonging to an instance of an oral condition to report that oral condition as present. If the decision is close (e.g., there are almost, but not quite, enough pixels with sufficiently high probabilities of representing an instance of an oral condition to report the oral condition), then an a minor instance of the oral condition may not be reported. In another example, a first probability threshold may be used generally to determine which pixels to include in a mask for an instance of an oral condition. A user may wish to adjust the sensitivity of the oral health diagnostics system to make it more sensitive to oral conditions. This may include lowering the thresholds that are used to determine whether pixels belong to instances of oral conditions and/or lowering the thresholds of sizes of instances of oral conditions that warrant reporting. However, it may be unwise to permit a user to manually select thresholds for oral condition detection. Accordingly, in some embodiments an oral health diagnostics system includes a high sensitivity mode that has been clinically tested and that provides results that are nearly as accurate as a standard sensitivity mode, but that is more likely to show borderline oral conditions and/or to show larger instances of oral conditions than the standard sensitivity mode. A user may toggle between the high sensitivity mode and the standard sensitivity mode to see how that changes estimations of one or more oral conditions for a patient.

FIG. 23F illustrates a flow diagram for a method 2381 of a high sensitivity mode for detection of oral conditions, in accordance with embodiments of the present disclosure. At block 2382 of method 2381, processing logic may determine pixel-level masks for oral conditions of teeth based on pixel-level probabilities associated with the oral conditions using one or more first thresholds (e.g., associated with a standard sensitivity mode). Processing logic may output visualizations for the pixel-level masks over an image of an oral cavity in an overlay.

At block 2384, processing logic may receive an instruction to activate a high sensitivity mode for oral condition detection. At block 2384, processing logic may activate the high sensitivity mode. This may cause the first thresholds to be replaced with second thresholds with lower second thresholds. At block 2388, processing logic determines new pixel-level masks for the oral conditions of the teeth based on the new thresholds. Since the thresholds are lower, the sizes of the pixel-level masks (e.g., the number of pixels included in the masks) will generally increase. Additionally, there may be thresholds on the number of pixels that should be included in a mask for the mask to warrant being identified as an instance of an oral condition. Accordingly, the new pixel-level masks may cause instances or oral conditions that were originally too small to be identified as oral conditions to now satisfy the size that causes them to be identified as oral condition instances.

At block 2390, processing logic determines one or more instances of oral conditions that were not identified under standard sensitivity mode but that are identified under high sensitivity mode. At block 2392, processing logic determines the pixel-level masks for the potential instances of oral conditions.

At block 2394, processing logic replaces visualizations of the original pixel level masks (from the standard visualization mode) with the visualizations of the new pixel-level masks. Additionally, processing logic may add visualizations for the potential instances of oral conditions that were not identified under the standard visualization mode. In one embodiment, the potential instances of oral conditions (those only identified in high sensitivity mode) are shown using a different visualization than the determined instances of oral conditions (those also identified in the standard sensitivity mode).

At block 2397, processing logic outputs a visual indication providing notification that the high sensitivity mode is active. This may include, for example, displaying a color band or border around the perimeter of the display.

At block 2398, processing logic may receive a command to reclassify a potential oral condition as an actual oral condition. In one embodiment, a user may click on the visual overlay for the instance of the potential oral condition, and an option to reclassify the potential oral condition may be presented and selected by the user. At block 2399, the potential oral condition is reclassified as a verified instance of the oral condition. Thereafter, the verified instance of the oral condition will be visible even if the user disables the high sensitivity mode.

FIG. 24A illustrates a flow diagram for a method 2400 of comparing reports for a patient generated by an oral health diagnostics system, in accordance with embodiments of the present disclosure. At block 2402 of method 2400, processing logic receives an instruction to generate a report on the oral health of a patient. At block 2404, processing logic generates the report. The report may include one or more images (e.g., radiographs) of the patient's oral cavity along with overlays on the image for each instance of an identified oral condition. The report may additionally include a dental chart showing oral conditions on teeth of the oral chart and/or a list of oral conditions arranged, for example, based on tooth number. The report may additionally include diagnosed oral health problems, suggested or planned treatments, doctor notes, and/or other information. In one embodiment, at block 2406 processing logic processes doctor annotations, image data, visual overlay(s) and/or dental chart information using a trained ML model trained to format report tailored for a doctor or practice.

At block 2408, processing logic retrieves one or more prior reports for the patient. At block 2410, processing logic may compare the current report to the one or more prior reports.

At block 2412, processing logic determines, based on the comparison, whether there were any oral conditions identified in both reports that have not been treated. If so, then the method continues to block 2414 and a notice of the one or more untreated oral conditions is generated. If there are no untreated oral conditions identified in the multiple reports, the method continued to block 2416.

At block 2416, processing logic determines whether there is a different between a current severity for one or more oral conditions and a past severity for the one or more oral conditions based on the comparison. If a difference in severity for one or more oral conditions are identified, then the method continues to block 2418 and a notice of the change in severity of the oral condition(s) is generated. Otherwise the method continues to block 2420.

At block 2420, processing logic outputs comparison results. The output may include the notices generated at block 2414 and/or 2418. The output may also include notices on new oral conditions that were not previously present and/or oral conditions that were present previously but that have resolved themselves and/or been treated.

FIG. 24B illustrates a flow diagram for a method 2430 of prioritizing patient treatments based on generated reports of different patients of a dental practice, in accordance with embodiments of the present disclosure. At block 2432 of method 2430, processing logic generates reports on the oral health of a plurality of patients. The patients may all be patients of the same doctor, the same group practice, group practices in the same geographic area, or may be reports for all patients. At block 2434, processing logic compares reports of patients for a doctor or group practice. At block 2436, processing logic determines comparative severity levels of oral conditions for the various patients. At block 2438, processing logic prioritizes treatment for patients of the doctor or practice that have the highest severity levels.

FIG. 24C illustrates a flow diagram for a method 2440 of identifying periodontitis, in accordance with embodiments of the present disclosure. At block 1442 of method 2440, processing logic receives a radiograph of a current state of a patient dental site (e.g., of a patient's oral cavity). At block 2444, processing logic processes the radiograph using a segmentation pipeline. At block 2446, processing logic performs postprocessing on segmentation information associated with the radiograph to determine one or more oral conditions (e.g., bone loss value, calculus, caries, periapical lesions, etc.).

At block 2448, processing logic determines a severity of periodontal bone loss for the patient based on the bone loss value and/or an age of the patient. At block 2450, processing logic receives additional patient information, such as pocket depth information, bleeding information, plaque/calculus information, smoking status of patient, medical history of patient (e.g., indication of diabetes), and so on. In some embodiments, the additional data is received from a DPMS. At block 2452, processing logic determines whether the patient has periodontitis and/or a stage of periodontitis based at least in part on the bone loss value and/or the severity of the periodontal bone loss. Processing logic may also take into account the additional information received at block 2450.

At block 2454, processing logic provides a recommendation to perform periodontal scaling and/or root planning to treat the periodontitis in view of the severity of the bone loss, identified calculus, and/or other information. At block 2456, processing logic may generate an insurance report/claim for periodontal scaling and/or root planning. The insurance report/claim may include the radiograph with an overlay of the area of periodontal bone loss and/or calculus. The report/claim may also include a tooth chart, pocket depth information, additional patient information, and an indication of the operations performed. In embodiments, the insurance report/claim is automatically submitted to an insurance carrier.

FIG. 24D illustrates a flow diagram for a method 2460 of generating a report of oral conditions of a patient, in accordance with embodiments of the present disclosure. At block 2462 of method 2460, processing logic generates segmentation information of a radiograph of a dental site. At block 2464, processing logic reconciles the segmentation information with segmentation information of an additional radiograph and/or image data from another imaging modality. This may include segmenting the other image data, registering the other image data to the segmented radiograph, comparing oral conditions between the different image modalities, and determining updated instances of oral conditions based on the comparison.

At block 2466, processing logic renders a visual output showing the determined oral conditions. At block 2468, processing logic receives an instruction to generate a report. At block 2470, processing logic determines report preferences of a doctor or practice. At block 2472, processing logic generates a report based on the oral conditions and preferences. In one embodiment, at block 2474 processing logic prioritizes oral conditions in the report based on doctor preferences, based on relative severity of the different oral conditions, and/or based on a combination thereof.

At block 2476, processing logic may receive an input with respect to one or more of the oral conditions. The input may include a selection of oral conditions to be treated, for example. At block 2478, processing logic may determine treatment for the one or more selected oral conditions.

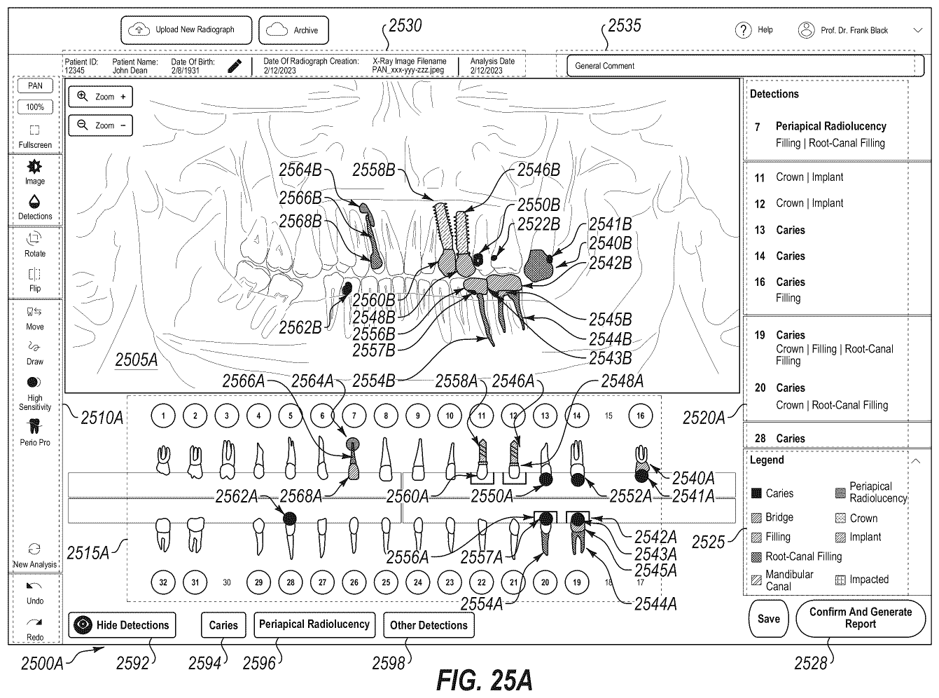

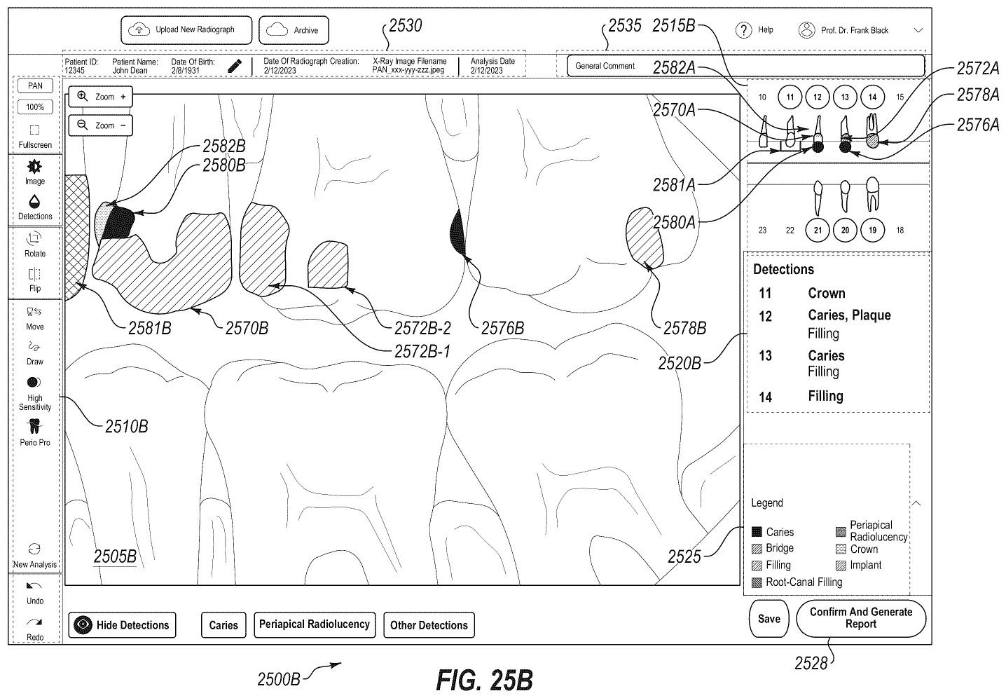

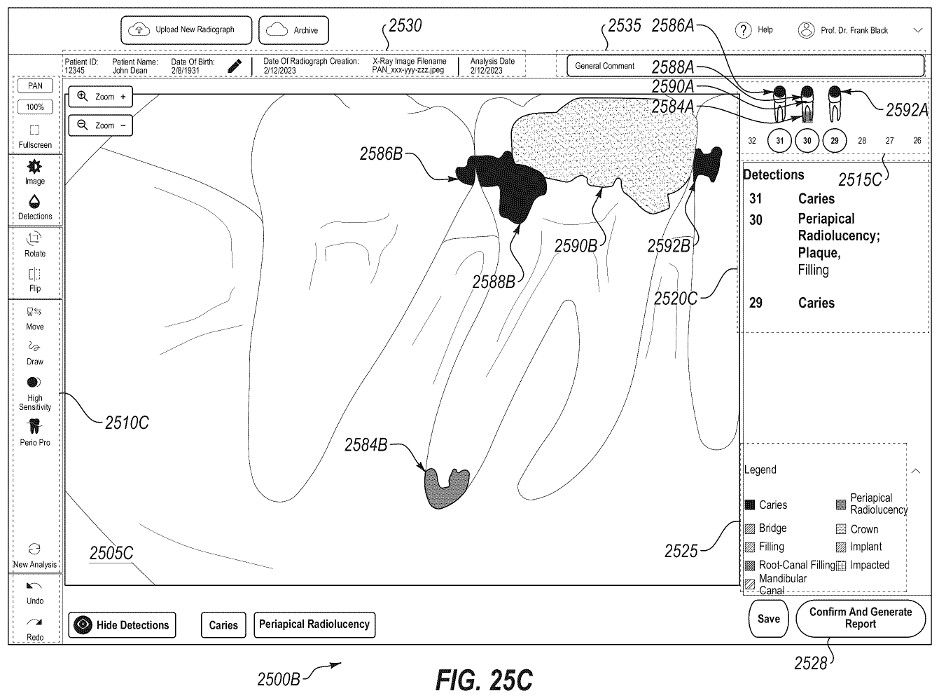

FIG. 25A illustrates a user interface 2500A of an oral health diagnostics system showing a panoramic x-ray 2505A, in accordance with embodiments of the present disclosure. FIG. 25B illustrates a user interface 2500B of an oral health diagnostics system showing a bitewing dental x-ray 2505B, in accordance with embodiments of the present disclosure. FIG. 25C illustrates a user interface 2500C of an oral health diagnostics system showing a periapical dental x-ray 2505C, in accordance with embodiments of the present disclosure. A user may access a radiograph 2505A-C to come to an informed decision about the dental status of a patient.

A radiograph 2505A-C of a patient's teeth may be generated by an x-ray machine and input into the oral health diagnostics system. The oral health diagnostics system may process the radiograph and/or other dental data (e.g., 3D models of dental arches, intraoral scans, CBCT images, 2D images of teeth, other radiographs, etc.) to determine detected oral conditions, oral health problems, etc., one or more of which may be shown in the user interface 2505A-C. The user interface 2505A-C may include the radiograph 2505A-C, a tool bar 2510A-C, an interactive tooth chart 2515A-C, a list of detected oral conditions 2520A-C, a report generation button 2528, and/or a status bar 2530. Tool bar 2510A-C includes multiple tools for controlling and/or interfacing with the user interface 2500A-C. Examples of tools include tools to alternate between full screen and window screen, for changing radiograph properties, such as contrast, brightness, etc., for turning on/off one or more detections (e.g., of oral conditions), for rotating the radiograph, for flipping the radiograph, for moving one or more detections, for drawing on the radiograph (e.g., to add one or more additional oral conditions, edit masks/overlays for existing detections (which may cause the underlying detections for the oral conditions to also change), for switching between a high sensitivity and standard sensitivity mode, for turning on or off a periodontal bone loss feature, for performing a new analysis of the radiograph or a different radiograph, fur undoing one or more operations, and so on. The status bar 2530 may include patient information (e.g., patient ID, patient name, patient date of birth, patient health information, and so on. The status bar 2530 may additionally include radiograph information for the displayed radiograph, such as date of radiograph creation, a radiograph file name, a data of analysis of the radiograph, and so on. An input window 2535 may enable a user to input comments, annotations, and so on about the patient and/or radiograph. Selecting the input window 2535 (e.g., comment button) may bring up a text field which the user may type comments in in embodiments.

The radiograph 2505A-C may be processed using a segmentation engine (e.g., one or more segmentation pipelines, oral structure determination engines, oral condition detection engines, oral condition mediation engines, etc.), oral health diagnostics engines, treatment recommendation engines, visualization engines, and so on to generate information on oral conditions, actionable symptom recommendations, diagnoses of oral health problems, treatment recommendations, and so on. In embodiments, an output of the processing is segmentation information on multiple different oral conditions. Overlays and/or masks (e.g., pixel-level masks) may be generated for each instance one or more of the oral conditions, oral health problems, etc. For example, a separate mask may be generated for each instance of an oral condition. The masks may be displayed over the radiograph 2505A-C to call attention to the oral conditions. The masks/overlays may be coded based on oral condition, severity, and/or other information. In embodiments, different visualizations (e.g., different colors, hatch patterns, line types, etc.) are used for different categories of oral conditions. The individual masks/overlay layers for each instance of an oral condition may be turned on or off (e.g., shown or hidden) responsive to user input. Additionally, a transparency of one or more masks/overlay layers may be adjusted based on user input. A user may select to toggle on or off and/or change the transparency of a single mask/overlay layer, masks/overlay layers associated with a particular type of oral condition, or all masks (e.g., the entire overlay) in embodiments.

In embodiments, a legend 2525 may indicate the visualizations (e.g., colors, hatch patterns, etc.) associated with the different types of oral conditions. For example, legend may indicate that caries is shown with a first visualization, bridges are shown with a second visualization, fillings are shown with a third visualization, root-canal fillings are shown with a fourth visualization, the mandibular canal is shown with a fifth visualization, periapical radiolucency (e.g., periapical lesions) are shown with a sixth visualization, crowns are shown with a seventh visualization, implants are shown with an eighth visualization, impacted teeth are shown with a ninth visualization, and calculus is shown with a tenth visualization. Other types of oral conditions and/or oral health problems may be shown with other visualizations.

A tooth chart 2515A-C may indicate the teeth shown in the radiograph 2505A-C. Alternatively, the tooth chart 2515A-C may indicate all teeth of a patient. Tooth chart 2515A shows all teeth of the patient, each of which is also shown in panoramic radiograph 2505A. Tooth chart 2515B shows just four upper teeth and three lower of the patient, corresponding to those shown in bite-wing radiograph 2505B. Tooth chart 2515C shows three lower teeth of the patient, corresponding to those shown in periapical radiograph 2505C.

In embodiments, each of the teeth in the radiograph 2505A-C are automatically identified and classified, such that tooth numbers are determined for each of the teeth. Oral conditions and/or oral health problems may be automatically identified for one or more teeth. The tooth chart 2515A-C may be automatically populated with information on the oral conditions, actionable symptom recommendations, diagnoses of oral health problems, and so on. Overlays may be generated and presented over the appropriate teeth in the dental chart based on the determined oral conditions, actionable symptom recommendations, oral health problems, etc. While the overlays on the radiograph 2505A-C may show actual shapes of oral conditions, the overlays on the tooth chart 2515A-C may not show the shape of oral conditions. However, overlays on the tooth chart may provide additional information not included in the overlays on the radiograph 2505A-C. For example, overlays on the tooth chart may show location on a tooth at which an oral condition has been detected (e.g., occlusal surface, left surface, right surface, etc.).

A list 2520A-C of oral conditions and/or oral health problems may be generated and output to the user interface 2500A-C. The list may indicate the identified oral conditions and/or oral health problems in text format in embodiments. The list 2520A-C may present in order (e.g., tooth order) a list of teeth for which oral conditions and/or oral health problems were detected, with an indication of the respective detected oral conditions and/or oral health problems for each of the teeth.

Via the user interface 2500A-C of the oral health diagnostics system, a practitioner may view the radiograph 2505A-C, the tooth chart 2515A-C, and an overlay of oral conditions (e.g., the masks generated for each of the identified oral conditions) overlaid on the radiograph 2505A-C.

In embodiments, user interface 2500A-C may include buttons to switch on and off detections. In some embodiments, these buttons may be used to turn on or off overlays on the radiograph, but not on the tooth chart. For example, a hide detections button 2592 may be selected (e.g., clicked on) to hide or unhide all overlays on the radiograph 2505A-C. A caries button 2594 may be selected to hide or unhide all caries overlays on the radiograph 2505A-C. A periapical radiolucency button 2596 may be selected to hide or unhide all periapical radiolucency overlays on the radiograph 2505A-C. Other types of detection overlays may also be shown or hidden via one or more other detections buttons 2598.

In the example user interface 2500A of FIG. 25A, a periapical radiolucency, filling and root canal filling were identified for tooth seven, a crown and implant were detected for tooth eleven, a crown and implant were detected for tooth twelve, a caries was detected for tooth thirteen, a caries was detected for tooth fourteen, a caries and filling were detected for tooth sixteen, a caries, crown, filling, and root canal filling were detected for tooth nineteen, a caries, crown and root canal filling were detected for tooth twenty, and a caries was detected for tooth twenty eight. List 2520A shows these detections textually, indicating that a periapical radiolucency, filling and root canal filling were identified for tooth seven, a crown and implant were detected for tooth eleven, a crown and implant were detected for tooth twelve, a caries was detected for tooth thirteen, a caries was detected for tooth fourteen, a caries and filling were detected for tooth sixteen, a caries, crown, filling, and root canal filling were detected for tooth nineteen, a caries, crown and root canal filling were detected for tooth twenty, and a caries was detected for tooth twenty eight.

Multiple overlay layers are shown on radiograph 2505A. An overlay 2564B shows a periapical radiolucency for tooth seven, an overlay 2568B shows a filling for tooth seven, an overlay 2566B shows a root canal filling for tooth seven. An overlay 2560B shows a crown for tooth eleven. An overlay 2558B shows an implant for tooth eleven. An overlay 2548B shows a crown for tooth twelve, and an overlay 2546B shows an implant for tooth twelve. An overlay 2550B shows a caries for tooth thirteen. An overlay 2552B shows a caries for tooth fourteen. An overlay 2541B shows a caries for tooth sixteen. An overlay 2540B shows a filling for tooth sixteen. An overlay 2543B shows a caries for tooth nineteen. An overlay 2542B shows a crown for tooth nineteen. An overlay 2545B shows a filling for tooth nineteen. An overlay 2544B shows a root canal filling for tooth nineteen. An overlay 2557B shows a caries for tooth twenty. An overlay 2556B shows a crown for tooth twenty. An overlay 2554B shows a root canal filling for tooth twenty. An overlay 2562B shows a caries for tooth twenty eight.

Additionally, multiple overlay layers are shown on tooth chart 2515A. An overlay 2564A shows a periapical radiolucency for tooth seven, an overlay 2568A shows a filling for tooth seven, an overlay 2566A shows a root canal filling for tooth seven. An overlay 2560A shows a crown for tooth eleven. An overlay 2558A shows an implant for tooth eleven. An overlay 2548A shows a crown for tooth twelve, and an overlay 2546A shows an implant for tooth twelve. An overlay 2550A shows a caries for tooth thirteen. An overlay 2552A shows a caries for tooth fourteen. An overlay 2541A shows a caries for tooth sixteen. An overlay 2540A shows a filling for tooth sixteen. An overlay 2543A shows a caries for tooth nineteen. An overlay 2542A shows a crown for tooth nineteen. An overlay 2545A shows a filling for tooth nineteen. An overlay 2544A shows a root canal filling for tooth nineteen. An overlay 2557A shows a caries for tooth twenty. An overlay 2556A shows a crown for tooth twenty. An overlay 2554A shows a root canal filling for tooth twenty. An overlay 2562A shows a caries for tooth twenty eight.

In the example user interface 2500B of FIG. 25B, a crown was detected for tooth eleven, caries, a filling, and calculus (plaque) were detected for tooth twelve, a caries and two fillings were detected for tooth thirteen, and a filling was detected for tooth fourteen. List 2520B shows these detections textually.

Multiple overlay layers are shown on radiograph 2505B. An overlay 2581B shows a crown for tooth eleven. An overlay 2570B shows a filling for tooth twelve, and an overlay 2580B shows a caries for tooth twelve, and overlay 2582B shows calculus for tooth twelve. An overlay 2576B shows a caries for tooth thirteen. An overlay 2572B-1 shows a first filling for tooth thirteen. An overlay 2572B-2 shows a second filling for tooth thirteen. An overlay 2578B shows a filling for tooth fourteen.

Additionally, an overlay 2581A shows a crown for tooth eleven. An overlay 2570A shows a filling for tooth twelve, and an overlay 2580A shows a caries for tooth twelve, and overlay 2582A shows calculus for tooth twelve. An overlay 2576A shows a caries for tooth thirteen. An overlay 2572A shows one or more fillings for tooth thirteen. An overlay 2578A shows a filling for tooth fourteen.

In the example user interface 2500C of FIG. 25C, a caries was detected for tooth twenty nine, caries, a filling, and periapical radiolucency were detected for tooth thirty, and a caries was detected for tooth thirty one. List 2520C shows these detections textually.

Multiple overlay layers are shown on radiograph 2505C. An overlay 2592B shows a caries for tooth twenty nine. An overlay 2590B shows a filling for tooth thirty, an overlay 2588B shows a caries for tooth thirty, and an overlay 2584B shows a periapical radiolucency for tooth thirty. An overlay 2586B shows a caries for tooth thirty one.

Additionally, an overlay 2592A shows a caries for tooth twenty nine. An overlay 2590A shows a filling for tooth thirty, an overlay 2588A shows a caries for tooth thirty, and an overlay 2584A shows a periapical radiolucency for tooth thirty. An overlay 2586A shows a caries for tooth thirty one.

Referring to FIGS. 25A-C, a user may select a tooth in any of the radiograph 2505A-C, tooth chart 2515A-C and/or list of detections 2520A-C to bring up further information about the selected tooth. Additionally, a user may select a particular oral condition by clicking on the text for the oral condition in the list of detections 2520A-C, an overlay for the oral condition on radiograph 2505A-C, or an overlay for the oral condition in the tooth chart 2515A-C. Responsive to selection of a dental condition, additional information may be shown for the condition, such as condition severity, location, associated oral health problems, suggested treatment options, and so on.

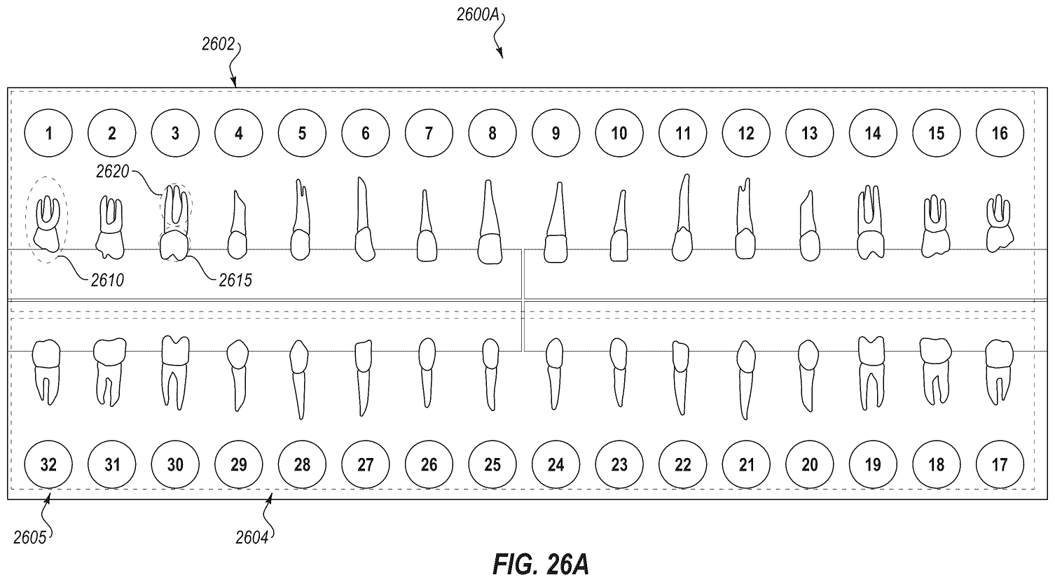

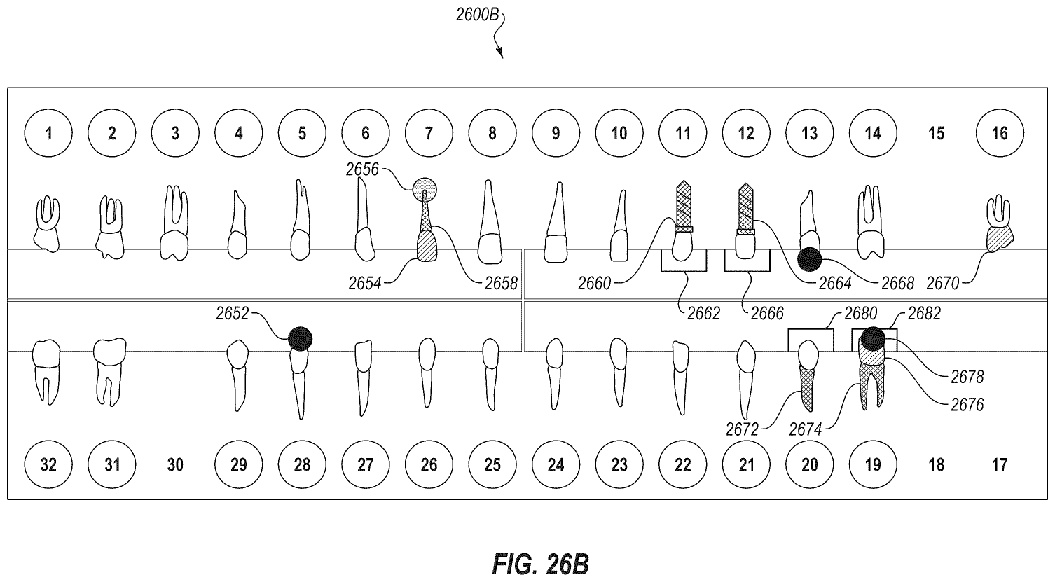

FIG. 26A illustrates a tooth chart 2600A of a set of teeth without oral health condition detections, in accordance with embodiments of the present disclosure. FIG. 26B illustrates a tooth chart 2600B of a set of teeth with oral health condition detections, in accordance with embodiments of the present disclosure. Oral health condition detections may be toggled on or off via the user interface. Tooth charts 2600A-B may correspond to tooth charts presented in any of the user interfaces 2500A-C in embodiments.

Tooth chart 2600A-B may be an interactive tooth chart that shows tooth status according to a tooth numbering scheme (e.g., the Universal Numbering Scheme). In one examples, the numbers 1-32 may be used for permanent teeth. The tooth designated “1” may be the maxillary right third molar (e.g., wisdom tooth) and the count may continue along the upper teeth to the left side. Then the count may begin at the mandibular left third molar, designated tooth number 17, and may continue along the bottom teeth to the right side.

The tooth chart 2600A-B may show individual tooth numbering 2605, and show graphics for each tooth 2610 of a patient. In embodiments, crowns 2615 and roots 2620 of each tooth of the patient are shown. Additionally, teeth in the upper dental arch 2602 and teeth in the lower dental arch 2604 may be shown.

With reference to FIG. 26B, when detections are enabled, different visualizations may be shown for each of caries, periapical radiolucency, calculus, different types of restorations (e.g., implants, fillings, root canal fillings, crowns, bridges, etc.), and so on. In the illustrated example, caries 2652, 2668, 2678 are shown for tooth thirteen, tooth nineteen, and tooth twenty eight. A periapical radiolucency 2656 is shown for tooth seven. Implants 2660, 2664 with crowns 2662, 2666 are shown for tooth eleven and tooth twelve. A root canal filling 2672, 2676 with a crown 2680, 2682 are shown for tooth twenty and tooth nineteen. Fillings 2654, 2670, 2676 are shown for tooth seven, tooth sixteen and tooth nineteen. Root canal fillings 2658, 2672, 2674 are shown for tooth seven, tooth nineteen, and tooth twenty.

In the example shown in FIGS. 26A-B, teeth fifteen, seventeen, eighteen and thirty are missing. This may be because the patient has lost these teeth, or because these teeth were not detected. In embodiments, a user may interact with the user interface to manually add teeth to the tooth chart 2600A-B. To add a tooth, a user may click on the tooth chart where the new tooth should be added. A dialogue may appear in which the tooth can be created by selecting a “create tooth” option. A user may be presented with one or more checkbox options for detections that can be added to the tooth once it is created. For example, the user may be able to select any of the types of oral conditions discussed herein to add to a tooth. A user may also select on any other detected tooth to add new detections, remove existing detections and/or edit existing detections.

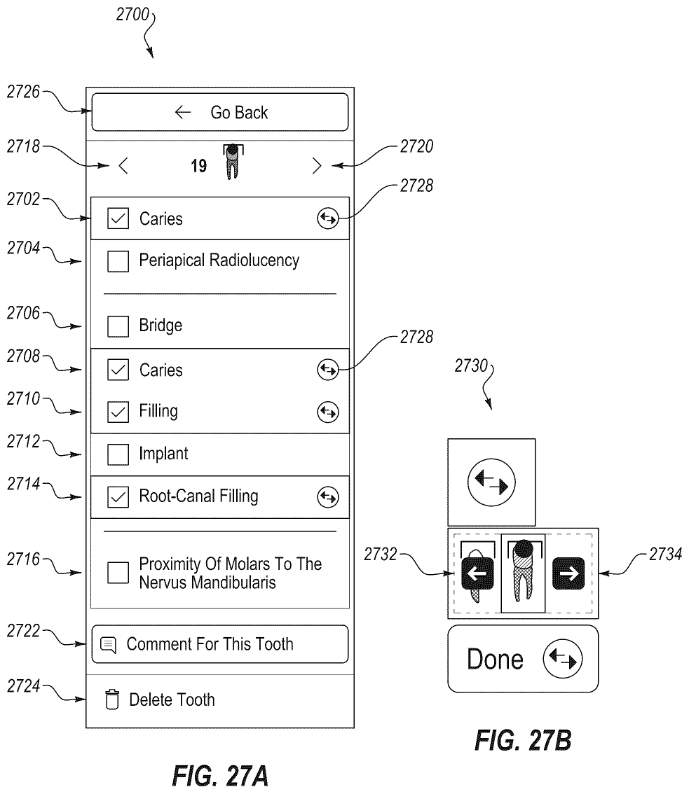

FIG. 27A illustrates a detailed view 2700 of oral conditions for a selected tooth, in accordance with embodiments of the present disclosure. When a user selects a tooth from the tooth chart or in the radiograph (e.g., clicking on the tooth in the radiograph) or in the list of detections, the detailed view 2700 for the tooth may be shown. In the detailed view 2700, a user may add, change, or delete any tooth specific findings. In embodiments, a menu of detection options for that tooth may be displayed in the detailed view 2700, such as shown in the example of FIG. 27A. The detailed view 2700 may show checkboxes indicating whether or not oral conditions of caries 2702, periapical radiolucency 2704, bridge 2706, crown 2708, filling 2710, implant 2712, root-canal filling 2714 and/or proximity of molars to the nervus mandibularis 2716 are present for a selected tooth of a patient. For example, as shown a user clicked on tooth nineteen, and caries, a crown, a filling and a root canal filling were automatically detected for tooth nineteen. Accordingly, checkmarks are shown for each of these findings. A user may click on buttons for caries, crown, filling, and/or root canal filling to remove any of these detected oral conditions. Additionally, or alternatively, the user may click on buttons for periapical radiolucency, bridge, implant, and/or proximity of molars to the nervus mandibularis to add detections for one of these types of oral conditions for the selected tooth. In some embodiments, unchecking a checkbox will remove the appropriate findings from the tooth chart as well as from the radiograph or other image data. In some embodiments, adding a checkbox will add a finding for the associated oral condition to the tooth chart but not to the radiograph or other image. In embodiments, to add a finding to a radiograph or other image, a drawing mode may be used, which is explained in greater detail below.

From the detailed view 2700, a user may click a left arrow 2718 to show the detections for a preceding tooth (e.g., tooth eighteen) or click on a right arrow 2720 to show detections for a next tooth (e.g., tooth twenty). A user may additionally click on a comments button 2722 to add doctor comments or annotations for the tooth. A user may also click on a delete tooth button 2724 to remove the tooth entirely (e.g., if the tooth is missing in the patient's oral cavity). A user may also click on a “go back” button 2726 to return to a previous view (e.g., a view of the tooth chart, radiograph, etc.).

One or more of the detected dental conditions may include a tooth reassignment button 2728. Clicking on the tooth reassignment button 2728 may bring up a tooth reassignment view for reassigning a detected oral condition to an adjacent tooth.

FIG. 27B illustrates a user interface for reassigning oral conditions to neighboring teeth (e.g., a tooth reassignment view 2730), in accordance with embodiments of the present disclosure. In the tooth reassignment view 2730, a user may select a preceding tooth button 2732 to move the detected oral condition to a preceding tooth or may select a next tooth button 2734 to move the detected oral condition to a next tooth. A user may then select a “done” button once the tooth reassignment for the oral condition is complete.

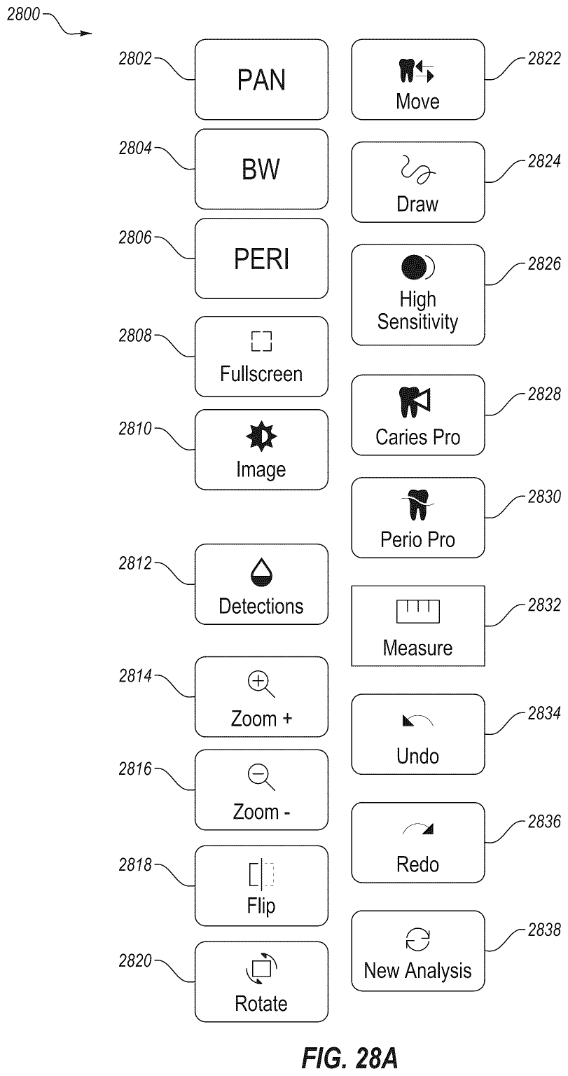

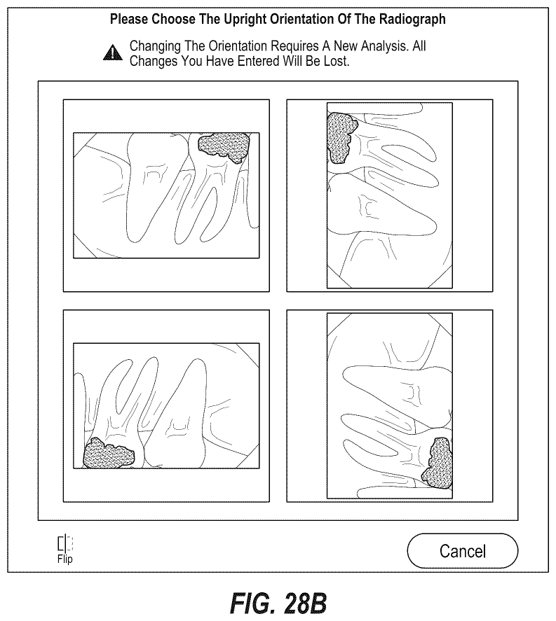

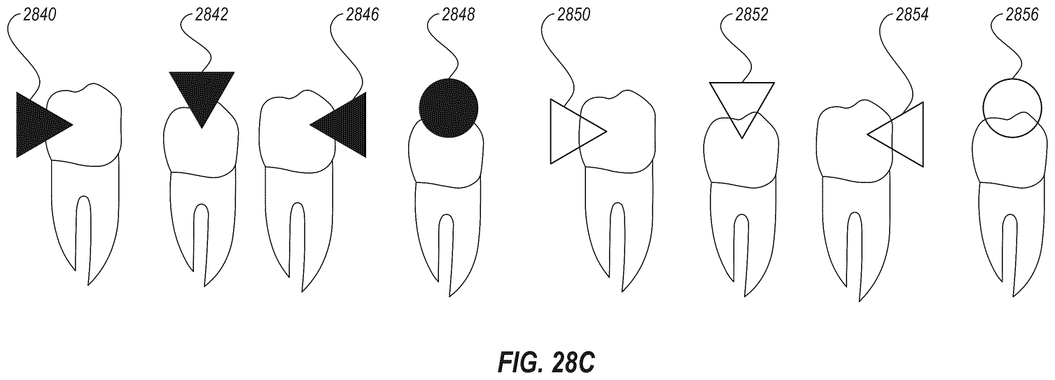

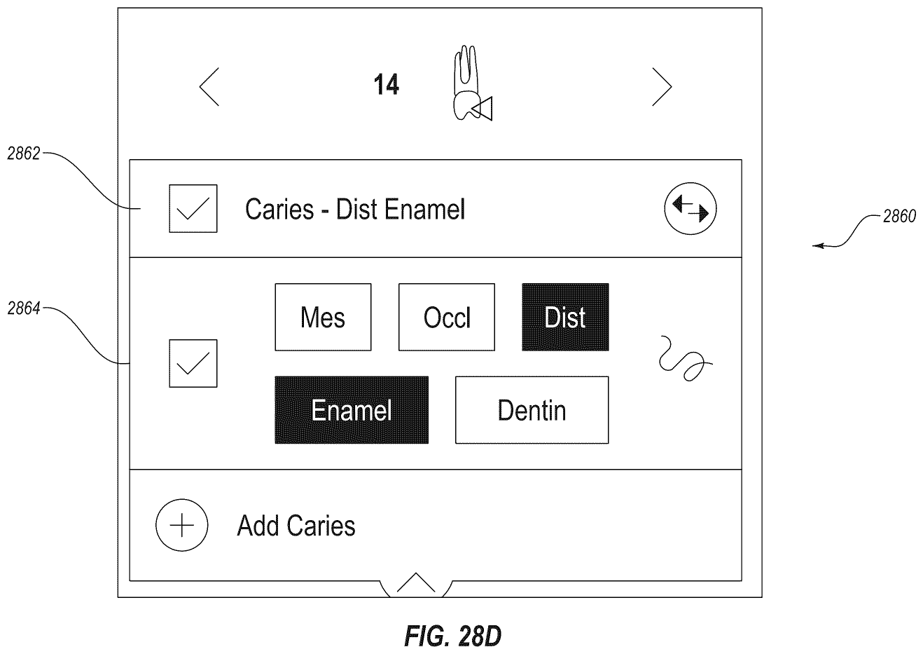

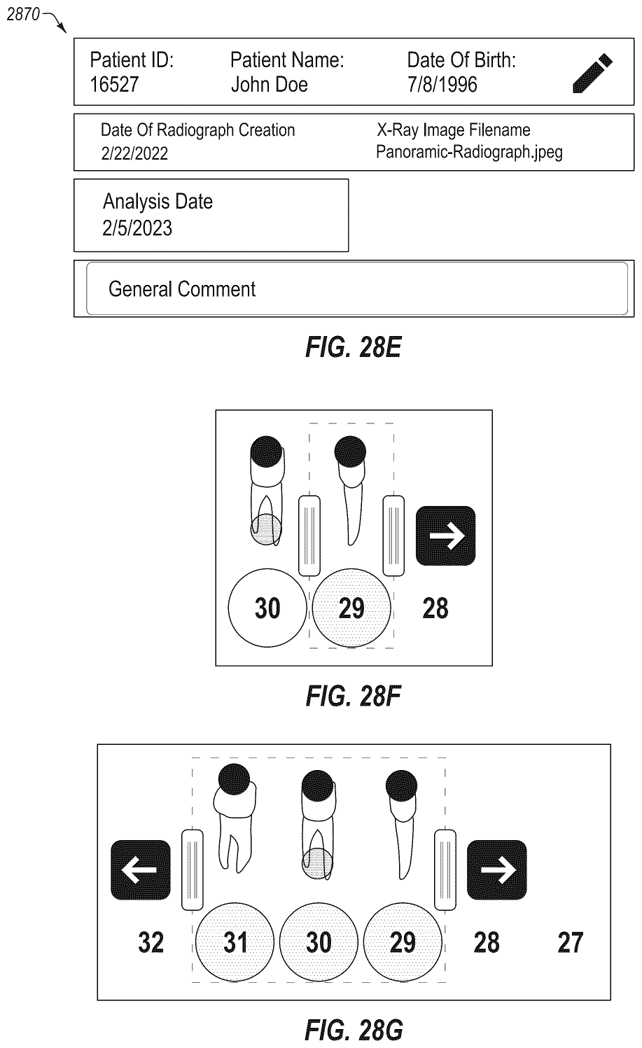

FIG. 28A illustrates a tool bar 2800 for an oral health diagnostics system showing a plurality of virtual buttons, in accordance with embodiments of the present disclosure. The tool bar 2800 may correspond to tool bar 2510A-C of FIGS. 25A-C in embodiments.

Tool bar 2800 may include buttons for alternating between different types of radiographs and/or other image modalities in embodiments. For example, tool bar 2800 may include a panoramic radiograph button 2802 that can be selected to display a captured panoramic radiograph, a bitewing radiograph button 2804 that can be selected to display a captured bitewing radiograph, and a periapical radiograph button 2806 that can be selected to display a captured bitewing radiograph. Other buttons may also be included for displaying a 3D model of an upper and/or dental arch of a patient generated based on intraoral scanning of the patient's oral cavity, for displaying intraoral scans of the patient's oral cavity, for showing a CBCT scan, for showing 2D images of the patient's oral cavity (e.g., as captured by a camera), and so on.

A full screen button 2808 may be pressed to activate a full screen mode.

One or more buttons may be provided for changing image properties such as brightness, contrast, opacity of detections, image size, and so on. For example, an image button 2810 may be selected to change image properties such as contrast and brightness. After selection of the image button 2810, contrast and brightness can be changed, for example, by holding down a right or left mouse button and moving a mouse or other input device from right to left (e.g., for contrast changes) and/or from top to bottom (e.g., for brightness changes).

A flip view button 2818 may be selected to flip a displayed radiograph or other image horizontally.

A detections button 2812 may be selected to increase or decrease the opacity of detections (e.g., of masks/overlays) on a radiograph or other image or 3D model. A zoom in button 2814 and/or zoom out button 2816 may be selected to change a zoom setting for a radiograph or other image being displayed. A rotate button 2820 may be selected to rotate a view of the radiograph or other image by 90 degrees or 180 degrees. In one embodiment, panoramic and bitewing radiographs can be rotated by 180 degree increments. In one embodiment, periapical radiographs can be rotated by 90 degree increments. In some embodiments, a new analysis may be run on a radiograph or other image after rotation (e.g., to generate new detections of oral conditions, oral health problems, and so on).

FIG. 28B illustrates different rotation options for an x-ray, in accordance with embodiments of the present disclosure. These different rotation options may be achieved by using the rotate button 2820 in embodiments.

Referring back to FIG. 28A, a draw button 2824 may be selected to draw one or more types of oral conditions on the radiograph or other image. For example, a user may draw a caries, periapical radiolucency, bridge, crown, or filling on a radiograph. The draw button 2824 may be selected to enable a drawing mode in embodiments.

In embodiments, the drawing mode allows to user to add freehand annotations. Examples of freehand annotations include frames, arrows, exclamation and question marks, or any kind of text. In addition, the drawing mode allows the user to add findings on the radiograph, which will then become part of the dental status. For example, a user may add user-drawn overlays of caries, periapical radiolucencies, bridges, crowns, fillings and so on to the radiograph 2505A-C via the drawing mode. Once the drawing mode is activated, a new set of buttons may appear, while other buttons, that are not allowed to be used in the drawing mode, may be hidden. To draw freehand annotations no tooth shall be selected, whereas for additions to the dental status, a tooth may be selected first. A tooth can be selected by clicking on the tooth in the tooth chart or in the list of detections, for example.

A workflow for the drawing mode may include a user clicking on the drawing mode button 2824. A tooth may be selected before or after the drawing mode button is selected if overlays for one or more oral conditions are to be added to a tooth. Alternatively, no tooth may be selected. For example, processing logic may automatically determine a tooth associated with a user-added overlay based on use of image processing and/or one or more AI models (e.g., ML models). A user may then select a drawing tool, such as annotate, or an oral condition (e.g., caries, periapical radiolucency, bridge, crown, filling), or delete. A user may then draw on the radiograph using a drawing tool pointer. The user may then review and approve or reject the drawn overlay. Once the overlay is approved, the user may select a “done” button to cause the user-added overlay to the findings. Once the drawing is approved by the user, the added finding is also added to the tooth chart and to the list of detections.

A move tooth button 2822 may be selected to move a tooth. FIGS. 28F-G show the move tooth function in greater detail.

A high sensitivity mode button 2826 may be selected to active a high sensitivity mode or to deactivate the high sensitivity mode. In the high sensitivity mode, the sensitivity of the system may be increased, and further possible areas of dental conditions may be displayed (e.g., for carious regions and/or regions or periapical lesions).

A caries pro button 2828 may be selected to provide more detailed information about caries of a patient. Selection of the caries pro button 2828 may cause localization and depth information of caries to be shown, for example, such as information on whether a caries is on the occlusal surface, left surface, right surface, mesial surface, distal surface, etc. of a tooth, a depth of the caries in the tooth, whether the caries is a dentin caries or enamel caries. The caries pro mode is discussed in greater detail below.

FIG. 28C shows a legend of more detailed caries information that is shown on a tooth chart when a caries pro mode is active, in accordance with embodiments of the present disclosure. As shown, a left filled triangle 2840 may indicate a mesial dentin caries, a top filled in triangle 2842 may indicate an occlusal dentin caries, a right filled in triangle 2846 may indicate a distal dentin caries, and a filled in circle 2848 may indicate a dentin caries at an unknown location. A left outline triangle 2850 may indicate a mesial enamel caries, a top outline in triangle 2852 may indicate an occlusal enamel caries, a right filled in triangle 2854 may indicate a distal enamel caries, and an outline circle 2856 may indicate an enamel caries at an unknown location.

While the caries pro mode is active, the detailed view 2700 for a selected tooth may provide additional caries information about that tooth. For example, in addition to the information shown on FIG. 27A, the detailed view while the caries pro mode is active may indicate caries location on the tooth and whether the caries is an enamel or dentin caries. For example, FIG. 28D shows a portion of a detailed view 2860 for a tooth while a caries pro mode is active, in accordance with embodiments of the present disclosure. In detailed view 2860, a selected tooth (e.g., tooth fourteen) is shown to have a distal enamel caries 2862. Detailed view 2860 further includes buttons 2864 for adding or changing information about a caries for a tooth, such as whether the caries is a mesial, occlusal and/or distal caries, and for whether the caries is an enamel caries or a dentin caries.

A perio pro button 2830 may be selected to provide more detailed information about periodontal bone loss of a patient. Selection of the perio pro button 2830 may cause additional overlays to be shown on a radiograph that identify amounts of periodontal bone loss, for example. The perio pro mode is discussed in greater detail below.

A measure button 2832 may be selected to enable a dentist to measure between two or more points on a radiograph or image. A user may measure the distance in pixels between two dots that the user places within the radiograph or image. If the radiograph or image has been registered with a 3D model of the patient's dental arch(es), then the distance in pixels may be converted to distance in physical units based on the registration and known size of features in the 3D model.

In embodiments, the tool bar 2800 additionally includes an undo button 2834 to undo a last action, a redo button 2836 to repeat a last action, and/or a new analysis button 2838 to restart an analysis of a current radiograph or other image, or to start an analysis of a new radiograph or other image.

FIG. 28E illustrates a status bar 2870 for a patient record of an oral health diagnostics system, in accordance with embodiments of the present disclosure. The status bar provides information and metadata of the patient, such as the patient ID, the patient's name, and the date of birth. Note that this information may be provided by the user or may be extracted from a DICOM file or be provided by another dental software system (e.g., DPMS). The patient related information may be edited. The status bar 2870 shows the date, when the radiograph was taken, if it was provided via DICOM or another dental software system and the name of the X-ray file. The status bar also informs the user of the date the analysis with oral health diagnostics system was conducted. By clicking on the “Analysis date” field the user can review historical analysis, either of detections of previous versions of oral health diagnostics system or the user interactions with the results provided by the oral health diagnostics system. The status bar 2870 may additionally include a free text field, where notes of comments can be added. These will be saved and are available for later revisits of the software by the user. These comments may also be included in generated reports.

FIG. 28F illustrates a user interface for moving a detected tooth, in accordance with embodiments of the present disclosure. Teeth can only be moved into an existing tooth gap. An entire row of teeth can also be moved. If desired, tooth gaps can also be moved. Moving or renaming a tooth may be useful if the software has classified the tooth name incorrectly. The mode is activated by activating the Move button with a click. To move a tooth, a user may select it by clicking on the tooth symbol or the tooth name in the tooth chart. The tooth can then be moved one position at a time. Note that the selected tooth can only be moved to a free position in some embodiments.

FIG. 28G illustrates a user interface for moving a set of teeth, in accordance with embodiments of the present disclosure. It may be necessary to move a row of teeth if the software has incorrectly classified several teeth in the group. The software might have missed a tooth gap, and several teeth may need to be moved by one position. The mode is also activated by the “Move” button. To move a row of teeth, a user may select several teeth by clicking on the gray bar next to the selected tooth (move mode activated and tooth selected). The row of teeth can then be moved one position at a time. Note that the selected row of teeth can only be moved in the direction of a free tooth position in some embodiments. In principle, tooth gaps can also be moved with the row of teeth.

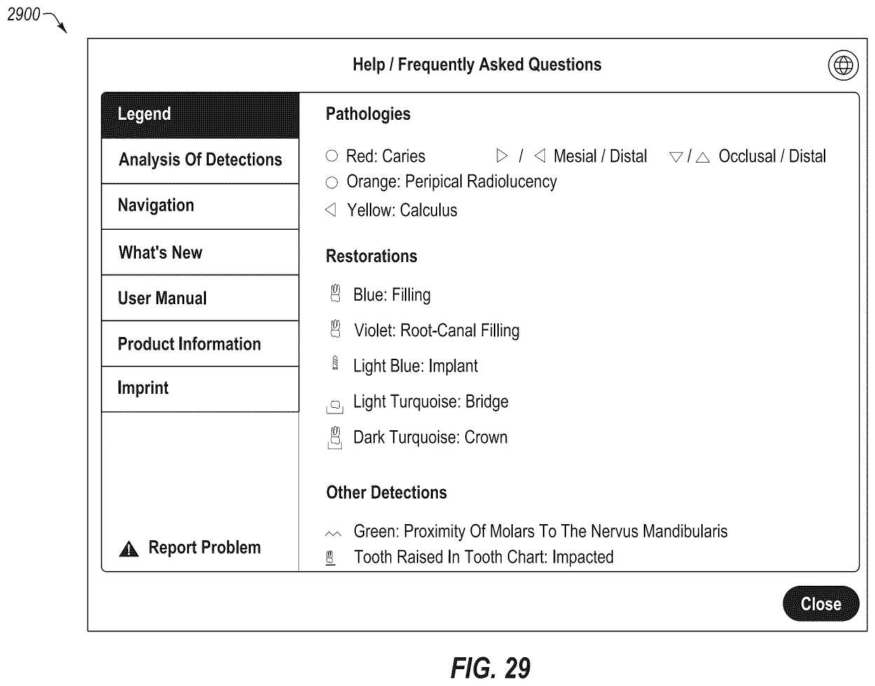

FIG. 29 illustrates a legend 2900 for an oral health diagnostics system, in accordance with embodiments of the present disclosure. From any view, a user may select a help button, which may bring up the legend 2900 as well as additional information such as a frequently asked questions (FAQs) about analysis and detections, navigation, new features, an electronic version of a user manual, product information, and so on.

The legend may show visualizations used to indicate different types of oral conditions. For example, the legend may show that red overlays indicate caries, that orange overlays indicate periapical radiolucency, and that yellow overlays indicate calculus.

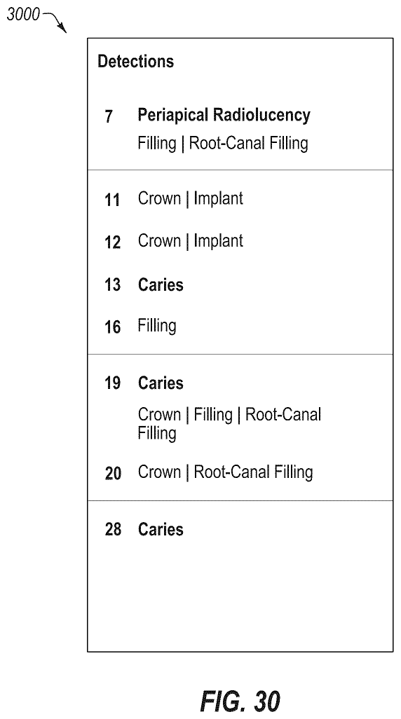

FIG. 30 illustrates an example list of oral health condition detections 3000 for a patient made by an oral health diagnostics system, in accordance with embodiments of the present disclosure. The list of oral health condition detections 3000 may correspond to the list of detections 2520A-C of FIGS. 25A-C in embodiments. The list of oral health condition detections 3000 may show all detected oral conditions (e.g., radiological abnormalities) and other findings. From the list of oral health condition detections 3000, a tooth number may be clicked on to being up a detailed view about a tooth (e.g., as shown in FIG. 27A).

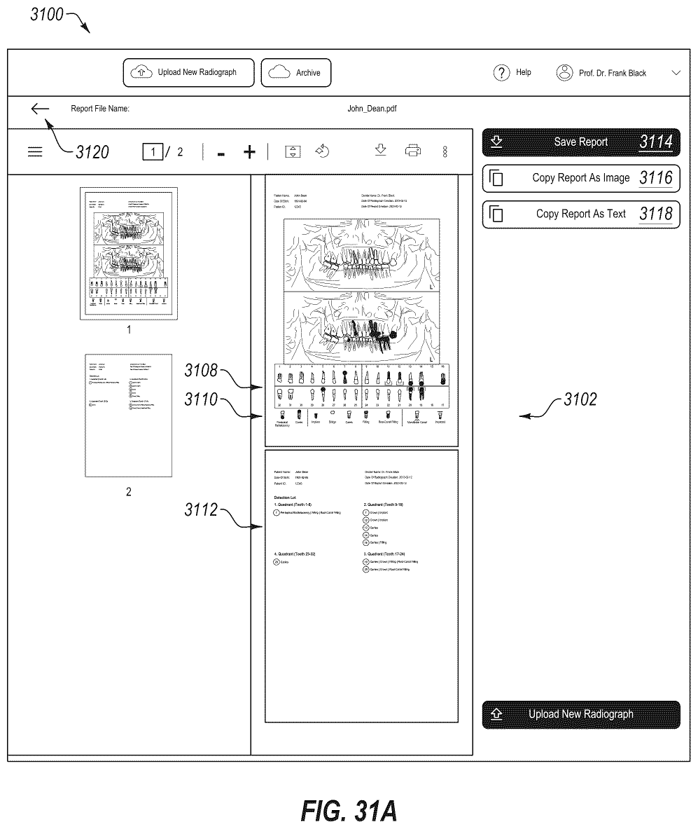

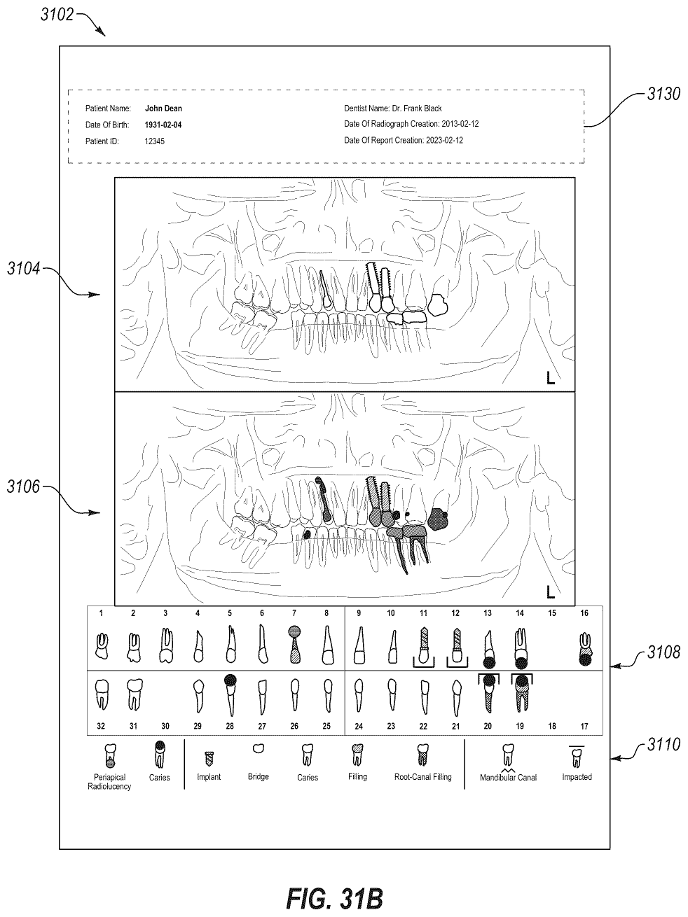

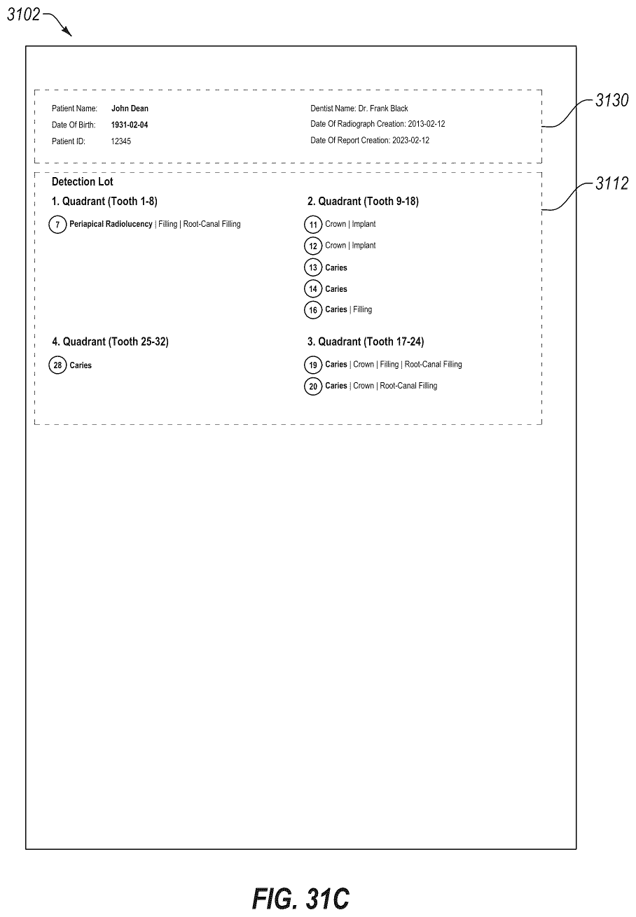

FIG. 31A illustrates a report view 3100 for reviewing an oral health conditions report for a patient, in accordance with embodiments of the present disclosure. A report 3102 can be created by clicking on the “Confirm and generate report” button 2528 in the user interface 2500A-C of FIGS. 25A-C in embodiments. The report 3102 summarizes all findings (e.g., oral conditions, oral health problems, recommended treatments, and so on) and includes any comments related to a tooth or to the patient in general. The report view 3100 shows the input radiograph 3104, the radiograph with overlays 3106, the tooth chart 3108 with findings, a legend 3110, and a list 3112 of all findings and comments by the user. The report view 3100 provides a save report button 3114 that can be selected to save the report (e.g., to a file such as a pdf file). A copy report as image button 3116 may be selected to save an image of a copy of the report to a clipboard. A copy report as text button 3118 may be selected to save a copy of the report to a clipboard as text. The radiograph itself (with or without overlays) may also be saved to the clipboard. A return to analysis button 3120 may be selected to return to an analysis screen (e.g., as shown in FIGS. 25A-C).

FIGS. 31B-C illustrate an oral conditions report 3102 for a patient, in accordance with embodiments of the present disclosure. As shown in the illustrated example, the oral conditions report 3102 includes patient information 3130, input radiograph 3104, the radiograph with overlays 3106, the tooth chart 3108 with findings, the legend 3110, and the list 3112 of detections. While not shown in this example, the report may also include other images (e.g., projections of a 3D model of the patient's dental arch from one or more view angles, intraoral scans, 2D images, etc. Additionally, the report may include actionable symptom recommendations, diagnoses of oral health problems, and/or treatment recommendations in embodiments.

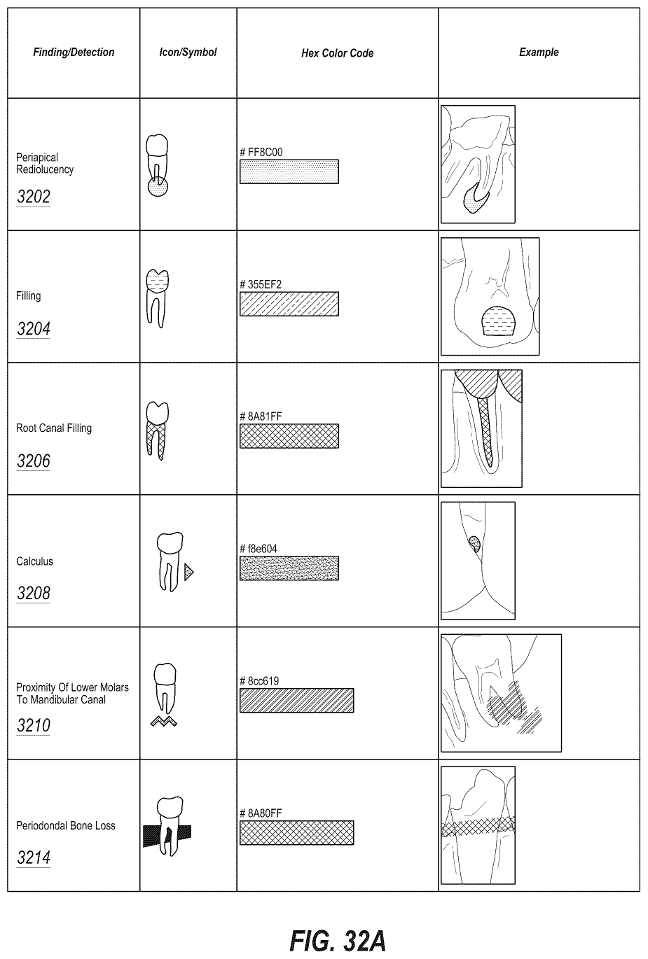

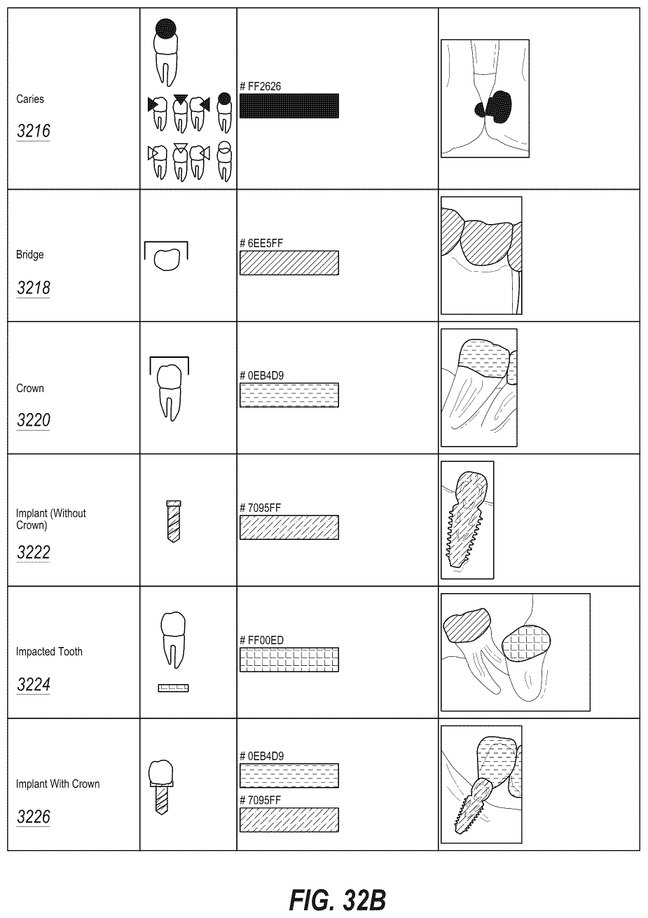

FIGS. 32A-B provide a legend of different types of oral condition overlays usable by an oral health diagnostics system, in accordance with embodiments of the present disclosure. For each type of oral condition overlay, a finding, associated icon, visualization (e.g., color, hatch pattern, etc.) and an example are shown. Examples are provided for periapical radiolucency 3202, filling 3204, root canal filling 3206, calculus 3208, proximity of lower molars to the mandibular canal 3210, periodontal bone loss 3214, caries 3216, bridge 3218, crown 3220, implant 3222, impacted tooth 3224, and implant with crown 3226.

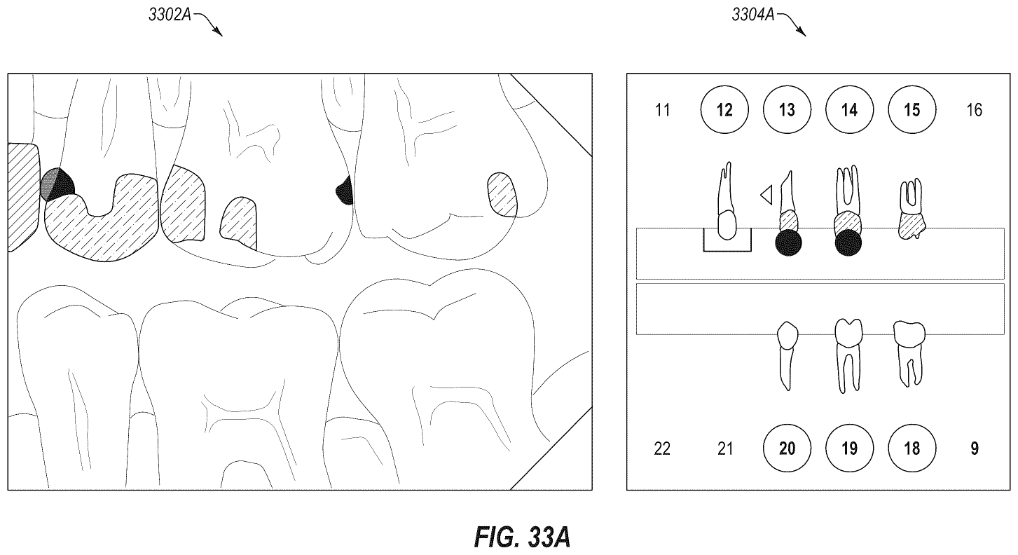

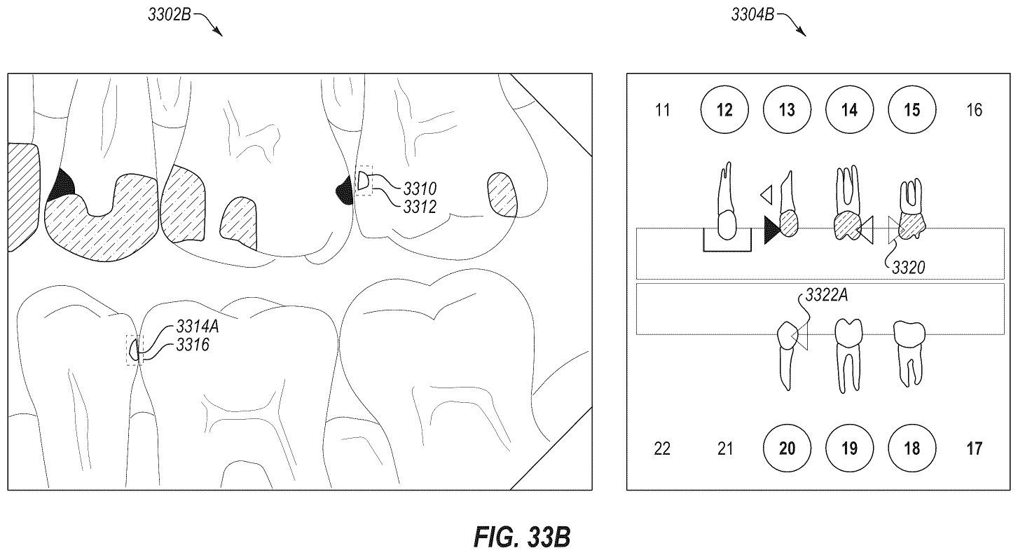

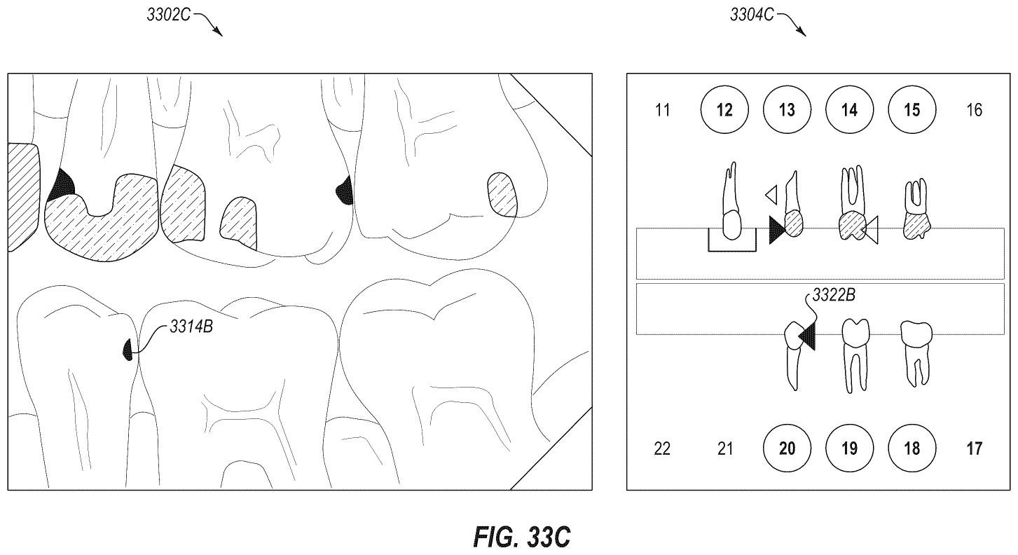

FIGS. 33A-C illustrate use of the high sensitivity mode, in accordance with embodiments of the present disclosure. The high sensitivity mode increases the sensitivity of the detection algorithms for one or more oral conditions (e.g., caries and apical lesions), by modifying the threshold at which pixels in the image are identified as positive for a pathological finding, such that more pixels are identified as “detections”. This means that as compared to the default setting, regions are newly identified as potentially abnormal, and the probability of false positives increases. Accordingly, to mitigate the risk of false positive findings, the user may be invited to confirm each newly found detection before they are recorded for further processing. Users of the software can evaluate newly added (detected) lesions and may accept or reject them in the list of detections. As with the regular detection, rejected or pending (neither accepted nor rejected) lesions may not be shown in the radiograph nor in the report.

FIG. 33A illustrates a bitewing radiograph 3302A with overlays of detected oral conditions and an associated tooth chart 3304A in a standard sensitivity mode, in accordance with embodiments of the present disclosure.

FIG. 33B illustrates the bitewing radiograph 3302B with overlays of detected oral conditions and an associated tooth chart 3304B of FIG. 33A, in a high sensitivity mode, in accordance with embodiments of the present disclosure. As shown, detections for two new caries 3310, 3314A are shown in the high sensitivity mode, which are not shown in the standard sensitivity mode. In embodiments, a different visualization is used to show potential oral conditions that have been identified in the high sensitivity mode but not in the standard sensitivity mode. For example, a solid or dashed outline of an oral condition may be shown for potential dental conditions, as opposed to a solid overlay for a dental condition identified in the standard sensitivity mode. In some instances, a bounding box (e.g., a dashed bounding box) is shown around potential dental conditions. In the illustrated example, bounding boxes 3312, 3316 are shown around the detected potential caries 3310, 3314A. An outline (e.g., dashed outline) of a caries 3320, 3322A is shown on the appropriate tooth where it was detected in the tooth chart 3304B as well. Once a user accepts (e.g., confirms or verifies) a potential dental condition, then the dental condition may be shown in the same way as standard detections of oral conditions of that type. If a potential dental condition is rejected, then it may be hidden.

FIG. 33C illustrates the bitewing radiograph 3302C with overlays of detected oral conditions and an associated tooth chart 3304C of FIG. 33A, in a standard sensitivity mode after a caries detected in the high sensitivity mode has been verified, in accordance with embodiments of the present disclosure. As shown, caries 3314A was verified, and so is shown as a detected caries 3314B in radiograph 3302C. Similarly, it is shown as a detected caries 3322B in the tooth chart 3304C.

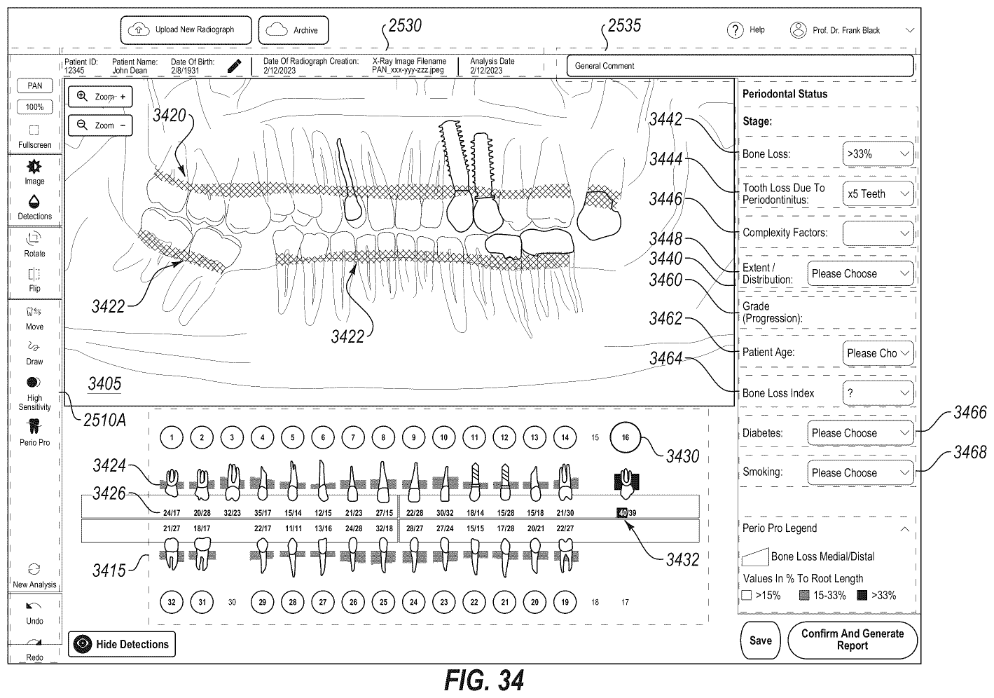

FIG. 34 illustrates a periodontal mode (also referred to as a perio pro mode) for an oral health diagnostics system, in accordance with embodiments of the present disclosure. The “Perio Pro Mode” detects periodontal bone loss on tooth surfaces, which may be expressed as a percentage of the root length. Bone loss may be measured in the radiograph mesially and/or distally for each tooth. This helps to determine periodontal staging and grading according to classification of periodontitis.

The Perio Pro Mode can be activated by clicking on the “Perio Pro” button 2830 of FIG. 28A. In the Perio Pro Mode, the overlays shown in the user interface of the oral health diagnostics system may change as compared to a standard mode. In the illustrated example, FIG. 34 shows the same radiograph 2505A of FIG. 25A (radiograph 3405 in FIG. 34) and tooth chart 2515A (tooth chart 3415 in FIG. 34), but with different overlays on the radiograph 3405 and on the tooth chart 3415. For example, overlays for detected caries, restorations, and periapical radiolucencies are hidden for radiograph 3405. Similarly, overlays for caries, restorations, and periapical radiolucencies are hidden in tooth chart 3415. Instead, overlays 3420 for periodontal bone loss on the upper dental arch and overlays 3422 for periodontal bone loss on the lower dental arch are shown on radiograph 3405. Additionally overlays 3424 or other graphics may be provided to represent the amount of periodontal bone loss at each individual tooth. The overlay or graphic for each tooth may be uniquely determined for that tooth based on calculated periodontal bone loss information for that tooth, and may show angle of bone loss, horizontal bone loss, amount of bone loss, and so on for the associated tooth. Additionally, numerical values 3426 may be provided indicating numerically the amount of periodontal bone loss at each tooth. The numerical values may be provided as a percent of bone loss per tooth surface (mesial and distal) in the tooth chart 3415. For example, a first bone loss value may be provided for the distal side of the tooth, and a second bone loss value may be provided for the mesial side of the tooth. Different values may also be provided for left vs. right sides of teeth. In some embodiments, differences between amounts of bone loss on different sides of a tooth are indicated in the tooth chart by trapezoids.

While the periodontal mode is active, a legend 3428 associated with periodontal bone loss visualizations may be displayed. As show in the legend 3428, different colors (or other visualization differences) may indicate different severity classes of bone loss. In one embodiment, bone loss severity may be divided into a low bone loss level (e.g., <15%), a medium bone loss level (e.g., 15-33%), and a high bone loss level (e.g., >33%).

In embodiments, teeth that have an amount of bone loss that is over a particular threshold (e.g., that is identified as severe) may be highlighted in the tooth chart and/or radiograph (e.g., with a circle 3430 around the tooth number for that tooth and/or with a highlight of the bone loss value 3432 for that tooth).

In some embodiments, on the right side of the screen two sections are present where the user can add additional clinical findings to compute a stage of periodontal bone loss and/or to grade a severity of the periodontal bone loss. Periodontitis may be divided into three stages according to the severity and the complexity of the periodontal bone loss. The severity may be based on the percentage of bone loss and/or on the number of teeth lost due to periodontitis. The complexity may be based on factors such as horizontal bone loss, deep vertical bone defects, and need for complex rehabilitation due to masticatory dysfunction. A grade or progression of periodontal bone loss may contains information on the rate of disease progression and the presence of patient-specific risk factors, such as nicotine use and diabetes/HbA1c.

A periodontal bone loss stage section 3240 may include a threshold field 3422 for adjusting a threshold to use for amount of bone loss that is associated with one or more severity levels (e.g., low, medium or high severity). For example, the threshold field 3422 currently shows that high severity threshold is 33%. However, a user may click on a drop down menu of the threshold filed 3422 to select a different threshold.

Other fields in the stage section 3420 include a tooth loss due to periodontitis field 3444 which may be used to set a number of lost tooth associated with different severity levels of periodontal bone loss, a complexity factors field 3446 in which a user may input one or more complexity factors, and an extent/distribution field 3448, which may indicate whether bone loss is horizontal bone loss, vertical bone loss, includes deep vertical bone defects, and so on. Dropdown fields may be prepopulated in case where the values can be inferred by the oral health diagnostics system. In one embodiment, prepopulated fields are marked with a first visualization (e.g., border having a first color), fields that need user input are marked with a second visualization (e.g., border having a second color), and fields for which information is missing but is not required may be marked by a third visualization (e.g., border having a third color).

The grade section 2460 may include fields in which patient information may be entered, such as a patient age field 3462 for inputting a patient age, a bone loss index field 3464 for inputting a bone loss index (e.g., between 0 and 1), a diabetes field 3466 for inputting whether the patient has diabetes, and a smoking field 3468 for inputting whether the patient smokes and/or an amount that a patient smokes (e.g., less than 10 cigarettes per day). Some of the information for these fields may be automatically populated (e.g., based information received from a DPMS).

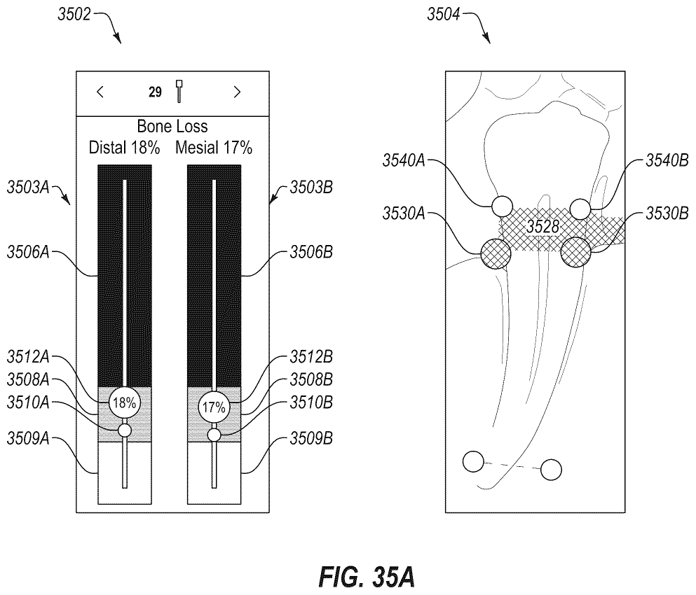

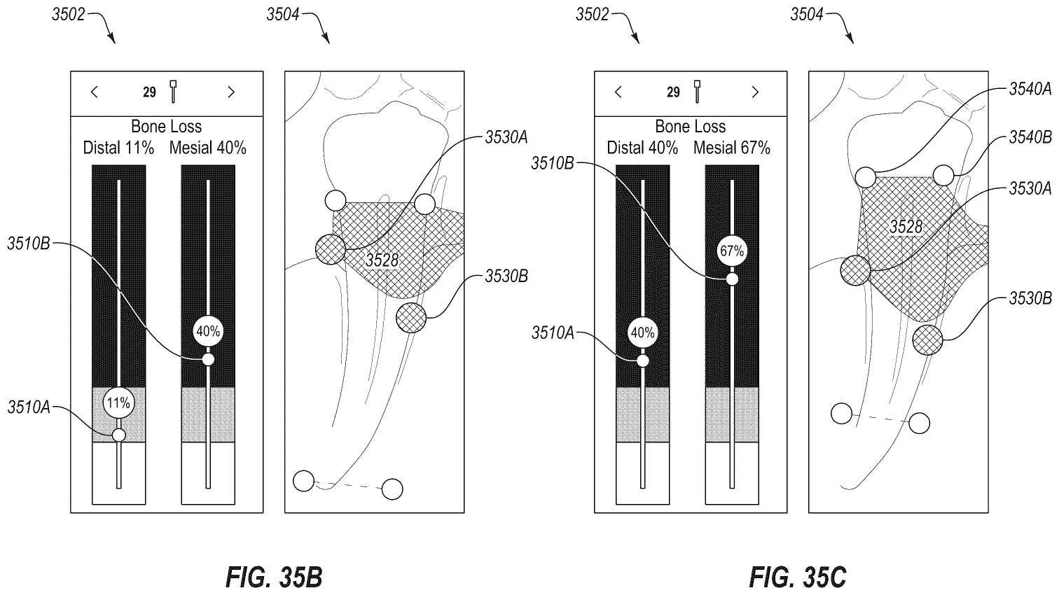

FIGS. 35A-C illustrate interactive elements of a periodontal mode loss mode of an oral health diagnostics system, in accordance with embodiments of the present disclosure. A user may enter the detailed view for a tooth as described earlier. While in the detailed view and the periodontal bone loss mode, user interaction elements 3502 (e.g., sliders, points that can be dragged, etc.), a zoomed in view of a radiograph 3504 of the selected tooth with a periodontal bone loss overlay for that tooth, etc. may be displayed. A user may edit the periodontal bone loss information interactively via the detailed view via the user interaction elements 3502 and/or on the radiograph 3504 itself. To edit the bone loss for a tooth, a user may select a tooth.

With reference to FIG. 35A, in the detailed view, a selected tooth may be indicated. In the illustrated example, tooth twenty nine is selected. A first scrollbar 3503A and a second scrollbar 3503B may be shown that reflect the periodontal bone loss per surface (e.g., in %) of the tooth, such as for the mesial surface and distal surface. The sliders 3503A-B may each be divided into three sections, including a high severity section 3506A-B, a medium severity section 3508A-B, and a low severity section 3509A-B. A position 3510A-B of the sliders 3503A-B in the low severity section 3509A-B may indicate that bone loss severity is low, a position 3510A-B of the sliders 3503A-B in the medium severity section 3508A-B may indicate that bone loss severity is medium, and a position 3510A-B of the sliders 3503A-B in the high severity section 3506A-B may indicate that bone loss severity is high. This may be separately determined and shown for the mesial and distal surfaces of the selected tooth.

As shown, the selected tooth has a distal bone loss of 18% and a mesial bone loss of 17%. Each slider 3503A-B may correspond to the intersection of the bone level and the tooth surface, which may be indicated by a corresponding landmark 3530A, 3530B on the radiograph 3504. The user can move a slider 3502A-B up and downwards to adjust the periodontal bone loss (e.g., in %) per surface, and the corresponding landmark 3530A-B will move accordingly and thereby also adapt the overlaying periodontal bone loss overlay/band 3528 on the radiograph. Alternatively, a landmark 3530A-B may be moved, which may cause the position 3510A, 1310B of the corresponding slider 3503A-B to be similarly moved. Other landmarks 3540A-B on the radiograph 3504 may indicate the mesial and distal side of the CEJ for the selected tooth. These landmarks 3540A-B may also be moved by a user in embodiments to adjust the detected positions of the CEJ for the tooth.

With reference to FIG. 35B, the distal bone loss slider position 3510A has been moved to reduce the distal bone loss to 11% and the mesial bone loss slider position 3510B has been moved to increase the mesial bone loss to 40%. This caused the distal bone loss landmark 3530A and mesial bone loss landmark 3530B to move by a corresponding amount to that shown in the sliders, which caused a shape of the periodontal bone loss overlay 3528 to change.

With reference to FIG. 35C, the distal bone loss landmark 3530A and mesial bone loss landmark 3530B are in the same positions as shown in FIG. 35B. However, a user has moved the distal CEJ landmark 3540A and the mesial CEJ landmark 3540B. The amount of periodontal bone loss at the distal and mesial surfaces is then recomputed based on the updated CEJ positions, which caused the amount of distal bone loss and mesial bone loss to change, and thus caused the distal bone loss slider position 3510A and mesial bone loss slider position 3510B to adjust accordingly. Additionally, the shape of the periodontal bone loss overlay 3528 changed based on the updated CEJ landmark positions.

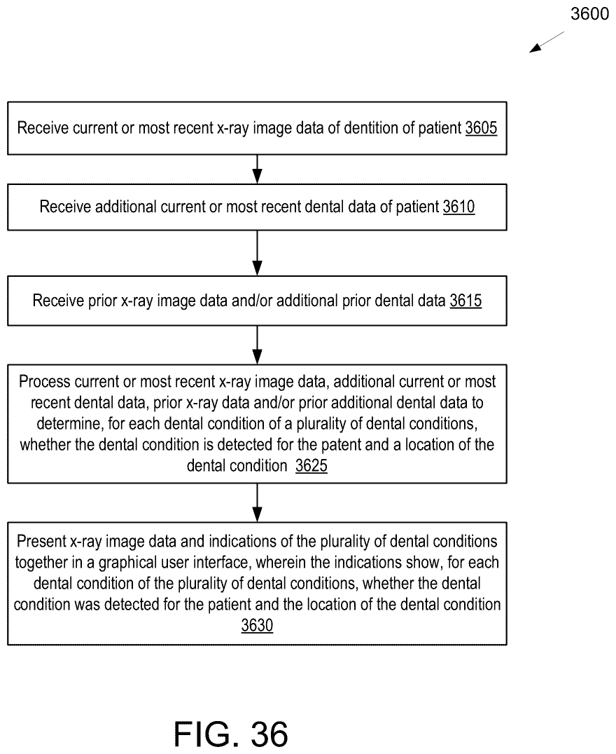

FIG. 36 illustrates a flow diagram for a method 3600 of analyzing a patient's dentition, in accordance with embodiments of the present disclosure. At block 3605 of method 3600, processing logic receives current or most recent x-ray image data (e.g., one or more recent radiographs) of a patient's dentition (teeth). At block 3610, processing logic may receive additional current or most recent dental data of the patient, such as color images, 3D models, intraoral scans, NIRI images, other x-ray images, and so on.

At block 3615, processing logic receives prior x-ray image data and/or additional prior dental data of a patient's dentition.

At block 3625, processing logic processes the current or most recent x-ray image data, additional current or most recent dental data, current or most recent 3D model(s), prior x-ray image data, prior dental data, and/or prior 3D model(s) to determine, for each oral health condition of a plurality of oral conditions, whether the oral health condition is detected for the patient, a location of the oral health condition, and/or a severity of the oral health condition for the patient. The operations of block 3625 may be performed by inputting data associated with the current or most x-ray image data, additional current or most recent dental data, current or most recent 3D model(s), prior x-ray image data, prior dental data, and/or prior 3D model(s) into one or more trained machine learning models. The data input into the trained machine learning model(s) may include images (which may be cropped), 3D surface data and/or projections of 3D models onto 2D planes in embodiments. The one or more trained machine learning models may be trained to receive the input data and to output segmentation information, which may include classifications of AOIs and associated oral conditions for those AOIs. In one embodiment, the output of the one or more trained machine learning models includes one or more probability maps or classification maps that indicate, for each point or pixel from the input data, a probability of that point or pixel belonging to one or more oral health condition classifications and forming part of an AOI associated with a oral health condition. The trained machine learning models may additionally be trained to output severity levels of the oral conditions associated with the AOIs. Alternatively, processing logic may perform additional processing using the output of the one or more trained machine learning models (and optionally the data that was input into the trained machine learning models) to determine severity levels of the oral conditions at the AOIs. In embodiments, processing logic may output identified changes in one or more oral conditions.

At block 3630, processing logic presents indications of the plurality of oral conditions and/or changes in oral conditions together in a graphical user interface or other display (e.g., in a dental diagnostics summary). The indications may show, for each oral health condition, whether the oral health condition was detected for the patient, the tooth at which the condition was detected, and the size and/or severity of the oral health condition, and so on. For example, for each tooth that may be an indication of each oral health condition that was detected.

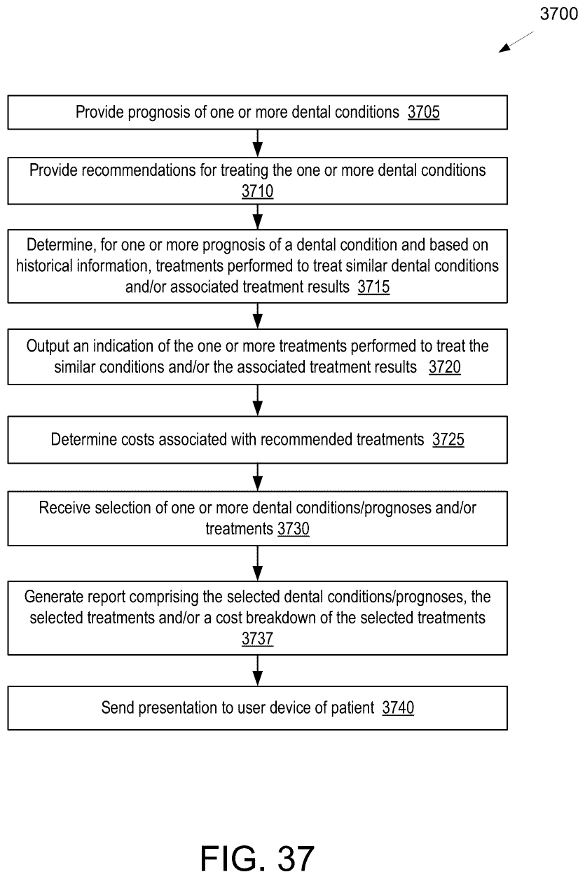

FIG. 37 illustrates a flow diagram for a method of generating a report of identified oral conditions (e.g., dental and/or gum conditions) of a patient, in accordance with embodiments of the present disclosure. At block 3705 of method 3700, processing logic provides a prognosis of one or more detected oral conditions at one or more AOIs on the patient's dental arch(es). As part of providing the prognosis, processing logic may determine the prognosis, such as by using machine learning and/or by projecting a detected progression and/or rate of change of each of the oral conditions into the future. At block 3710, processing logic provides one or more recommendations for treating the one or more oral conditions. The recommendations may be based on data gathered from many doctors over many patients, and may indicate for oral conditions similar to those detected for the current patient, specific treatment(s) that was performed. For example, at block 3715 processing logic may determine, for the one or more prognosis and the oral health condition (and optionally based on historical information about the patient at hand), treatments that were performed to treat similar oral conditions as well as associated treatment results. Processing logic may determine, based on historical information, at least one of one or more treatments performed to treat similar oral conditions or associated treatment results. At block 3720, processing logic may output an indication of the one or more treatments performed to treat the similar conditions and/or the associated treatment results.

At block 3725, processing logic may determine the costs associated with the recommended treatments. At block 3730, processing logic receives a selection of one or more oral conditions and/or treatments. At block 3735, processing logic automatically generates a presentation comprising the selected oral conditions, the associated prognoses for the selected oral conditions, the selected treatments and/or a cost breakdown of the selected treatments. At block 3740, the generated presentation may be shown to the patient and/or may be sent to a user device of the patient (e.g., to a mobile computing device or a traditionally stationary computing device of the patient). This may include sending a link to access the presentation to the user device of the patient.

FIG. 38 illustrates a diagrammatic representation of a machine in the example form of a computing device 3800 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In one embodiment, the computing device 3800 corresponds to computing device 305 of FIG. 3.

The example computing device 3800 includes a processing device 3802, a main memory 3804 (e.g., read-only memory (ROM), flash memory, dynamic random access memory (DRAM) such as synchronous DRAM (SDRAM), etc.), a static memory 3806 (e.g., flash memory, static random access memory (SRAM), etc.), and a secondary memory (e.g., a data storage device 3828), which communicate with each other via a bus 3808.

Processing device 3802 represents one or more general-purpose processors such as a microprocessor, central processing unit, or the like. More particularly, the processing device 3802 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 3802 may also be one or more special-purpose processing devices such as an application specific integrated circuit (ASIC), a field programmable gate array (FPGA), a digital signal processor (DSP), network processor, or the like. Processing device 3802 is configured to execute the processing logic (instructions 3826) for performing operations and steps discussed herein.

The computing device 3800 may further include a network interface device 3822 for communicating with a network 3864. The computing device 3800 also may include a video display unit 3810 (e.g., a liquid crystal display (LCD) or a cathode ray tube (CRT)), an alphanumeric input device 3812 (e.g., a keyboard), a cursor control device 3814 (e.g., a mouse), and a signal generation device 3820 (e.g., a speaker).

The data storage device 3828 may include a machine-readable storage medium (or more specifically a non-transitory computer-readable storage medium) 3824 on which is stored one or more sets of instructions 3826 embodying any one or more of the methodologies or functions described herein. A non-transitory storage medium refers to a storage medium other than a carrier wave. The instructions 3826 may also reside, completely or at least partially, within the main memory 3804 and/or within the processing device 3802 during execution thereof by the computer device 3800, the main memory 3804 and the processing device 3802 also constituting computer-readable storage media.

The computer-readable storage medium 3824 may also be used to store an oral health diagnostics system 3850, which may correspond to the similarly named component of FIG. 3. The computer readable storage medium 3824 may also store a software library containing methods for a oral health diagnostics system 3850. While the computer-readable storage medium 3824 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium (e.g., a medium other than a carrier wave) that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A method comprising:
receiving image data of a current state of a dental site of a patient;
processing the image data using a segmentation pipeline to generate an output comprising segmentation information for one or more teeth in the image data and at least one of identifications or locations of one or more oral conditions observed in the image data, wherein each of the one or more oral conditions is associated with a tooth of the one or more teeth;
generating a visual overlay comprising visualizations for each of the one or more oral conditions;
outputting the image data to a display; and
outputting the visual overlay to the display over the image data.

2. The method of claim 1, wherein the image data comprises a radiograph.

3. The method of claim 2, wherein processing the image data using the segmentation pipeline comprises:
processing the image data using one or more first trained machine learning models to generate a first output comprising the segmentation information for the one or more teeth in the image data; and
processing the image data using one or more additional trained machine learning models to generate a second output comprising at least one of the identifications or the locations of the one or more oral conditions.

4. The method of claim 3, wherein for an oral condition of the one or more oral conditions an additional trained machine learning model of the one or more additional trained machine learning models outputs a bounding box for an instance of the oral condition, the method further comprising:
determining a tooth associated with the bounding box;
determining an intersection of data from the bounding box and a segmentation mask for the tooth from the segmentation information; and
determining a pixel-level mask for the instance of the oral condition based at least in part on the intersection of the data from the bounding box and the segmentation mask.

5. The method of claim 4, wherein the oral condition comprises a caries or a restoration, the method further comprising:
subtracting the data from the bounding box that does not intersect with the segmentation mask;
wherein the pixel-level mask is provided as a layer of the visual overlay representing the oral condition within the tooth.

6. The method of claim 4, wherein the oral condition comprises calculus, the method further comprising:
drawing an ellipse within the bounding box, wherein the intersection of the data from the bounding box and the segmentation mask comprises an intersection of the ellipse and the segmentation mask; and
subtracting the data from the bounding box that intersects with the segmentation mask;
wherein the pixel-level mask is provided as a layer of the visual overlay representing the calculus around the tooth.

7. The method of claim 3, wherein for an oral condition of the one or more oral conditions an additional trained machine learning model of the one or more additional trained machine learning models outputs a plurality of bounding boxes for an instance of the oral condition, the method further comprising:
determining that a first bounding box of the plurality of bounding boxes encapsulates one or more additional bounding boxes of the plurality of bounding boxes; and
removing the one or more additional bounding boxes.

8. The method of claim 3, wherein for an oral condition of the one or more oral conditions an additional trained machine learning model of the one or more additional trained machine learning models outputs a bounding box for an instance of the oral condition, the method further comprising:
determining a tooth associated with the bounding box;
determining an overlap between the bounding box and a segmentation mask for the tooth from the segmentation information; and
determining a location of the oral condition on the tooth based at least in part on the overlap.

9. The method of claim 8, wherein determining the location comprises:
performing principal component analysis of the segmentation mask or a bounding box for the tooth from the segmentation information to determine at least a first principal component;
determining a first line between a tooth occlusal surface and a tooth root apex based on the first principal component; and
determining a first portion of the bounding box that is on a mesial side of the first line and a second portion of the bounding box that is on a distal side of the first line; and
determining whether the oral condition is on the mesial side of the tooth or the distal side of the tooth based on the first portion and the second portion.

10. The method of claim 2, herein each instance of one or more oral conditions is provided as a distinct layer of the visual overlay, the method further comprising:
generating a dental chart for the patient;
populating the dental chart based on data for the one or more oral conditions; and
outputting the dental chart to the display.

11. The method of claim 10, wherein each instance of one or more oral conditions is provided as a distinct layer of the visual overlay, the method further comprising:
receiving a selection of a tooth based on user interaction with at least one of the tooth in the dental chart or the tooth in the image data;
outputting detailed information for instances of each of the one or more oral conditions identified for the selected tooth;
receiving an instruction to remove an instance of an oral condition of the tooth; and
marking the tooth as not having the instance of the oral condition.

12. The method of claim 1, wherein the one or more oral conditions comprise one or more instances of caries, the method further comprising:
for each instance of caries, determining whether the instance of the caries is an enamel caries or a dentin caries;
marking instances of caries identified as enamel caries using a first visualization; and
marking instances of caries identified as dentin caries using a second visualization.

13. The method of claim 1, further comprising:
determining dental codes associated with the one or more oral conditions;
assigning the dental codes to the one or more oral conditions;
determining a treatment that was performed on the patient; and
automatically generating an insurance claim for the treatment, the insurance claim comprising the image data, at least a portion of the visual overlay comprising the one or more oral conditions that were treated, and the dental codes associated with the one or more oral conditions.

14. The method of claim 1, wherein the at least one of the identifications or the locations of the one or more oral conditions comprises a probability map indicating, for each pixel of the image data, a probability of the pixel corresponding to at least one oral condition of the one or more oral conditions, the method further comprising:
determining, for the at least one oral condition and for a first tooth, a pixel-level mask indicating pixels having a probability that exceeds a first threshold, wherein the visual overlay comprises the pixel-level mask for the first tooth.

15. The method of claim 14, further comprising:
receiving an instruction to activate a high sensitivity mode for oral condition detection;
activating the high sensitivity mode, wherein activating the high sensitivity mode comprises replacing the first threshold with a second threshold that is lower than the first threshold; and
determining, for the at least one oral condition and for the first tooth, a new pixel-level mask indicating pixels having a probability that exceeds the second threshold, wherein the visual overlay comprises the new pixel-level mask in the high sensitivity mode.

16. The method of claim 15, wherein prior to activating the high sensitivity mode the at least one oral condition was not identified for a second tooth, the method further comprising:
determining, for the at least one oral condition and for the second tooth, a second new pixel-level mask indicating additional pixels having the probability that exceeds the second threshold, wherein the visual overlay comprises the second new pixel-level mask for the second tooth in the high sensitivity mode;
wherein the at least one oral condition for the second tooth is identified as a potential instance of the at least one oral condition in view of the at least one oral condition being identified only in the high sensitivity mode.

17. The method of claim 1, further comprising:
receiving a command to generate a report;
generating the report comprising the image data, the visual overlay, and a dental chart showing, for each tooth of the patient, any oral conditions identified for that tooth; formatting the report in a structured data format ingestible by a dental practice management system; and
adding the report to the dental practice management system.

18. The method of claim 1, further comprising:
generating a report comprising the image data, the visual overlay, and a dental chart showing, for each tooth of the patient, any oral conditions identified for that tooth;
comparing the report to reports of the one or more oral conditions for a plurality of additional patients;
determining comparative severity levels of the one or more oral conditions between the patient and the plurality of additional patients based on the comparing; and
prioritizing treatment of the patient based on the comparative severity levels.

19. The method of claim 1, wherein the one or more oral conditions comprise a bone loss value, wherein determining the bone loss value for a tooth comprises:
determining a centoenamel junction (CEJ) for the tooth;
determining a periodontal bone line (PBL) for the tooth;
determining a root apex of the tooth;
determining a first distance between the CEJ and the PBL;
determining a second distance between the CEJ and the root apex; and
determining a ratio between the first distance and the second distance.

20. The method of claim 1, wherein the one or more oral conditions comprise a bone loss value, the method further comprising:
determining bone loss values for each of the one or more teeth of the patient; and
determining, based on the bone loss values, whether the patient has at least one of horizontal bone loss, vertical bone loss, generalized bone loss, or localized bone loss.

21. The method of claim 1, wherein the image data comprises a bite-wing x-ray that fails to show a root of a tooth of the one or more teeth, the method further comprising:
determining a tooth number of the tooth in the bite-wing x-ray;
determining a tooth length from a previous periapical x-ray, a previous CBCT or a previous panoramic x-ray comprising a representation of the tooth;
determining a centoenamel junction (CEJ) for the tooth from the bite-wing x-ray;
determining a periodontal bone line (PBL) for the tooth from the bite-wing x-ray;
determining a bone loss length between the CEJ and the PBL from the bite-wing x-ray; and
determining a ratio between the bone loss length and the tooth length.

22. The method of claim 1, wherein the image data comprises a bite-wing x-ray that fails to show a root of a tooth of the one or more teeth, the method further comprising:
determining a tooth number of the tooth in the bite-wing x-ray;
determining a tooth size from a three-dimensional model of the dental site generated from intraoral scanning of the dental site;
registering the bite-wing x-ray to the three-dimensional model for the tooth;
determining a conversion between pixels of the bite-wing x-ray and physical units of measurement based on the registration;
determining a centoenamel junction (CEJ) for the tooth from the bite-wing x-ray;
determining a periodontal bone line (PBL) for the tooth from the bite-wing x-ray;
determining a distance between the CEJ and the PBL in pixels from the bite-wing x-ray; and
converting the distance in pixels into a distance in the physical units of measurement based on the conversion.

23. A non-transitory computer readable medium comprising instructions that, when executed by one or more processing devices, cause the one or more processing devices to perform operations comprising:
receiving image data of a current state of a dental site of a patient;
processing the image data using a segmentation pipeline to generate an output comprising segmentation information for one or more teeth in the image data and at least one of identifications or locations of one or more oral conditions observed in the image data, wherein each of the one or more oral conditions is associated with a tooth of the one or more teeth;
generating a visual overlay comprising visualizations for each of the one or more oral conditions;
outputting the image data to a display; and
outputting the visual overlay to the display over the image data.

24. A system comprising:
a computing device comprising a memory and one or more processing devices, wherein the computing device is configured to:
receive image data of a current state of a dental site of a patient;
process the image data using a segmentation pipeline to generate an output comprising segmentation information for one or more teeth in the image data and at least one of identifications or locations of one or more oral conditions observed in the image data, wherein each of the one or more oral conditions is associated with a tooth of the one or more teeth;
generate a visual overlay comprising visualizations for each of the one or more oral conditions;
output the image data to a display; and
output the visual overlay to the display over the image data.
